SEQUENCE LISTING (2095 sequences)

SEQ ID NO: 1 - RNA, 20 nt, synthetic construct
  misc_feature 1..20: note = Synthetic
  source 1..20: mol_type = other RNA
tcctacctgtcagagcccca

SEQ ID NO: 2 - RNA, 20 nt, synthetic construct
  misc_feature 1..20: note = Synthetic
  source 1..20: mol_type = other RNA
gagccccaaggtaaaaaggc

SEQ ID NO: 3 - RNA, 20 nt, synthetic construct
  misc_feature 1..20: note = Synthetic
  source 1..20: mol_type = other RNA
agccccaaggtaaaaaggcc

SEQ ID NO: 4 - RNA, 20 nt, synthetic construct
  misc_feature 1..20: note = Synthetic
  source 1..20: mol_type = other RNA
tttcccggcctttttacctt

SEQ ID NO: 5 - RNA, 20 nt, synthetic construct
  misc_feature 1..20: note = Synthetic
  source 1..20: mol_type = other RNA
ctttcccggcctttttacct

SEQ ID NO: 6 - RNA, 20 nt, synthetic construct
  misc_feature 1..20: note = Synthetic
  source 1..20: mol_type = other RNA
acactgtgagctgcctggga

SEQ ID NO: 7 - RNA, 20 nt, synthetic construct
  misc_feature 1..20: note = Synthetic
  source 1..20: mol_type = other RNA
cacactgtgagctgcctggg

SEQ ID NO: 8 - RNA, 20 nt, synthetic construct
  misc_feature 1..20: note = Synthetic
  source 1..20: mol_type = other RNA
gtggcacactgtgagctgcc

SEQ ID NO: 9 - RNA, 20 nt, synthetic construct
  misc_feature 1..20: note = Synthetic
  source 1..20: mol_type = other RNA
gagattgagctctactcagg

SEQ ID NO: 10 - RNA, 20 nt, synthetic construct
  misc_feature 1..20: note = Synthetic
  source 1..20: mol_type = other RNA
agattgagctctactcaggt

SEQ ID NO: 11 - RNA, 20 nt, synthetic construct
  misc_feature 1..20: note = Synthetic
  source 1..20: mol_type = other RNA
caggtgggccctcctccctc

SEQ ID NO: 12 - RNA, 20 nt, synthetic construct
  misc_feature 1..20: note = Synthetic
  source 1..20: mol_type = other RNA
cggaagagaccagagggagg

SEQ ID NO: 13 - RNA, 20 nt, synthetic construct
  misc_feature 1..20: note = Synthetic
  source 1..20: mol_type = other RNA
taccggaagagaccagaggg

SEQ ID NO: 14 - RNA, 20 nt, synthetic construct
  misc_feature 1..20: note = Synthetic
  source 1..20: mol_type = other RNA
agggaggcttatgccaatat

SEQ ID NO: 15 - RNA, 20 nt, synthetic construct
  misc_feature 1..20: note = Synthetic
  source 1..20: mol_type = other RNA
ggcttatgccaatatcggtg

SEQ ID NO: 16 - RNA, 20 nt, synthetic construct
  misc_feature 1..20: note = Synthetic
  source 1..20: mol_type = other RNA
tgactgcgcttttccttgtc

SEQ ID NO: 17 - RNA, 20 nt, synthetic construct
  misc_feature 1..20: note = Synthetic
  source 1..20: mol_type = other RNA
gactgcgcttttccttgtct

SEQ ID NO: 18 - RNA, 20 nt, synthetic construct
  misc_feature 1..20: note = Synthetic
  source 1..20: mol_type = other RNA
gcttttccttgtctgggcag

SEQ ID NO: 19 - RNA, 20 nt, synthetic construct
  misc_feature 1..20: note = Synthetic
  source 1..20: mol_type = other RNA
ccagttccgctgcccagaca

SEQ ID NO: 20 - RNA, 20 nt, synthetic construct
  misc_feature 1..20: note = Synthetic
  source 1..20: mol_type = other RNA
gacctgaagcactggaagcc

SEQ ID NO: 21 - RNA, 20 nt, synthetic construct
  misc_feature 1..20: note = Synthetic
  source 1..20: mol_type = other RNA
gcactggaagccaggtgtgc

SEQ ID NO: 22 - RNA, 20 nt, synthetic construct
  misc_feature 1..20: note = Synthetic
  source 1..20: mol_type = other RNA
cactggaagccaggtgtgca

SEQ ID NO: 23 - RNA, 20 nt, synthetic construct
  misc_feature 1..20: note = Synthetic
  source 1..20: mol_type = other RNA
ggaagccaggtgtgcagggc

SEQ ID NO: 24 - RNA, 20 nt, synthetic construct
  misc_feature 1..20: note = Synthetic
  source 1..20: mol_type = other RNA
agccaggtgtgcagggcagg

SEQ ID NO: 25 - RNA, 20 nt, synthetic construct
  misc_feature 1..20: note = Synthetic
  source 1..20: mol_type = other RNA
gcccacctgccctgcacacc

SEQ ID NO: 26 - RNA, 20 nt, synthetic construct
  misc_feature 1..20: note = Synthetic
  source 1..20: mol_type = other RNA
gggctcagctgtgaggaagt

SEQ ID NO: 27 - RNA, 20 nt, synthetic construct
  misc_feature 1..20: note = Synthetic
  source 1..20: mol_type = other RNA
ggggctcagctgtgaggaag

SEQ ID NO: 28 - RNA, 20 nt, synthetic construct
  misc_feature 1..20: note = Synthetic
  source 1..20: mol_type = other RNA
gaccagattcccagtatgtt

SEQ ID NO: 29 - RNA, 20 nt, synthetic construct
  misc_feature 1..20: note = Synthetic
  source 1..20: mol_type = other RNA
ccagattcccagtatgttag

SEQ ID NO: 30 - RNA, 20 nt, synthetic construct
  misc_feature 1..20: note = Synthetic
  source 1..20: mol_type = other RNA
cagattcccagtatgttagg

SEQ ID NO: 31 - RNA, 20 nt, synthetic construct
  misc_feature 1..20: note = Synthetic
  source 1..20: mol_type = other RNA
tcccagtatgttagggggct

SEQ ID NO: 32 - RNA, 20 nt, synthetic construct
  misc_feature 1..20: note = Synthetic
  source 1..20: mol_type = other RNA
tccaagccccctaacatact

SEQ ID NO: 33 - RNA, 20 nt, synthetic construct
  misc_feature 1..20: note = Synthetic
  source 1..20: mol_type = other RNA
ctccaagccccctaacatac

SEQ ID NO: 34 - RNA, 20 nt, synthetic construct
  misc_feature 1..20: note = Synthetic
  source 1..20: mol_type = other RNA
aaaggcactgcaagagacaa

SEQ ID NO: 35 - RNA, 20 nt, synthetic construct
  misc_feature 1..20: note = Synthetic
  source 1..20: mol_type = other RNA
tccagtatattcatctacca

SEQ ID NO: 36 - RNA, 20 nt, synthetic construct
  misc_feature 1..20: note = Synthetic
  source 1..20: mol_type = other RNA
ttcatctaccatggtgagtg

SEQ ID NO: 37 - RNA, 20 nt, synthetic construct
  misc_feature 1..20: note = Synthetic
  source 1..20: mol_type = other RNA
tcatctaccatggtgagtgc

SEQ ID NO: 38 - RNA, 20 nt, synthetic construct
  misc_feature 1..20: note = Synthetic
  source 1..20: mol_type = other RNA
catctaccatggtgagtgcg

SEQ ID NO: 39 - RNA, 20 nt, synthetic construct
  misc_feature 1..20: note = Synthetic
  source 1..20: mol_type = other RNA
accatggtgagtgcggggcc

SEQ ID NO: 40 - RNA, 20 nt, synthetic construct
  misc_feature 1..20: note = Synthetic
  source 1..20: mol_type = other RNA
gccaggccccgcactcacca

SEQ ID NO: 41 - RNA, 20 nt, synthetic construct
  misc_feature 1..20: note = Synthetic
  source 1..20: mol_type = other RNA
ccactctccacccccaatgt

SEQ ID NO: 42 - RNA, 20 nt, synthetic construct
  misc_feature 1..20: note = Synthetic
  source 1..20: mol_type = other RNA
ctccacccccaatgtaggtg

SEQ ID NO: 43 - RNA, 20 nt, synthetic construct
  misc_feature 1..20: note = Synthetic
  source 1..20: mol_type = other RNA
cacctcacctacattggggg

SEQ ID NO: 44 - RNA, 20 nt, synthetic construct
  misc_feature 1..20: note = Synthetic
  source 1..20: mol_type = other RNA
gggcacctcacctacattgg

SEQ ID NO: 45 - RNA, 20 nt, synthetic construct
  misc_feature 1..20: note = Synthetic
  source 1..20: mol_type = other RNA
tggggcacctcacctacatt

SEQ ID NO: 46 - RNA, 20 nt, synthetic construct
  misc_feature 1..20: note = Synthetic
  source 1..20: mol_type = other RNA
ctggggcacctcacctacat

SEQ ID NO: 47 - RNA, 20 nt, synthetic construct
  misc_feature 1..20: note = Synthetic
  source 1..20: mol_type = other RNA
acctcccgagcaaacatgac

SEQ ID NO: 48 - RNA, 20 nt, synthetic construct
  misc_feature 1..20: note = Synthetic
  source 1..20: mol_type = other RNA
ccgagcaaacatgacaggta

SEQ ID NO: 49 - RNA, 20 nt, synthetic construct
  misc_feature 1..20: note = Synthetic
  source 1..20: mol_type = other RNA
gtacctctcacaggccctaa

SEQ ID NO: 50 - RNA, 20 nt, synthetic construct
  misc_feature 1..20: note = Synthetic
  source 1..20: mol_type = other RNA
agtacctctcacaggcccta

SEQ ID NO: 51 - RNA, 20 nt, synthetic construct
  misc_feature 1..20: note = Synthetic
  source 1..20: mol_type = other RNA
gacgtcttgtgctctggaga

SEQ ID NO: 52 - RNA, 20 nt, synthetic construct
  misc_feature 1..20: note = Synthetic
  source 1..20: mol_type = other RNA
aacaagcttccaaaatggcc

SEQ ID NO: 53 - RNA, 20 nt, synthetic construct
  misc_feature 1..20: note = Synthetic
  source 1..20: mol_type = other RNA
gagatcccgcatcactcacc

SEQ ID NO: 54 - RNA, 20 nt, synthetic construct
  misc_feature 1..20: note = Synthetic
  source 1..20: mol_type = other RNA
gcccctggcctttgcagagc

SEQ ID NO: 55 - RNA, 20 nt, synthetic construct
  misc_feature 1..20: note = Synthetic
  source 1..20: mol_type = other RNA
accggctctgcaaaggccag

SEQ ID NO: 56 - RNA, 20 nt, synthetic construct
  misc_feature 1..20: note = Synthetic
  source 1..20: mol_type = other RNA
caccggctctgcaaaggcca

SEQ ID NO: 57 - RNA, 20 nt, synthetic construct
  misc_feature 1..20: note = Synthetic
  source 1..20: mol_type = other RNA
ccaccggctctgcaaaggcc

SEQ ID NO: 58 - RNA, 20 nt, synthetic construct
  misc_feature 1..20: note = Synthetic
  source 1..20: mol_type = other RNA
ctgctccaccggctctgcaa

SEQ ID NO: 59 - RNA, 20 nt, synthetic construct
  misc_feature 1..20: note = Synthetic
  source 1..20: mol_type = other RNA
tcagctgtgtcacccgtttc

SEQ ID NO: 60 - RNA, 20 nt, synthetic construct
  misc_feature 1..20: note = Synthetic
  source 1..20: mol_type = other RNA
gctgtgtcacccgtttcagg

SEQ ID NO: 61 - RNA, 20 nt, synthetic construct
  misc_feature 1..20: note = Synthetic
  source 1..20: mol_type = other RNA
ctgtgtcacccgtttcaggt

SEQ ID NO: 62 - RNA, 20 nt, synthetic construct
  misc_feature 1..20: note = Synthetic
  source 1..20: mol_type = other RNA
tgtgtcacccgtttcaggtg

SEQ ID NO: 63 - RNA, 20 nt, synthetic construct
  misc_feature 1..20: note = Synthetic
  source 1..20: mol_type = other RNA
cacccgtttcaggtggggtg

SEQ ID NO: 64 - RNA, 20 nt, synthetic construct
  misc_feature 1..20: note = Synthetic
  source 1..20: mol_type = other RNA
acccgtttcaggtggggtga

SEQ ID NO: 65 - RNA, 20 nt, synthetic construct
  misc_feature 1..20: note = Synthetic
  source 1..20: mol_type = other RNA
cccgtttcaggtggggtgag

SEQ ID NO: 66 - RNA, 20 nt, synthetic construct
  misc_feature 1..20: note = Synthetic
  source 1..20: mol_type = other RNA
gtctgaggccctccctccac

SEQ ID NO: 67 - RNA, 20 nt, synthetic construct
  misc_feature 1..20: note = Synthetic
  source 1..20: mol_type = other RNA
tctgaggccctccctccaca

SEQ ID NO: 68 - RNA, 20 nt, synthetic construct
  misc_feature 1..20: note = Synthetic
  source 1..20: mol_type = other RNA
caaggcagccctgtggaggg

SEQ ID NO: 69 - RNA, 20 nt, synthetic construct
  misc_feature 1..20: note = Synthetic
  source 1..20: mol_type = other RNA
gctcaaggcagccctgtgga

SEQ ID NO: 70 - RNA, 20 nt, synthetic construct
  misc_feature 1..20: note = Synthetic
  source 1..20: mol_type = other RNA
cgctcaaggcagccctgtgg

SEQ ID NO: 71 - RNA, 20 nt, synthetic construct
  misc_feature 1..20: note = Synthetic
  source 1..20: mol_type = other RNA
tgtgcagactcagaggtgag

SEQ ID NO: 72 - RNA, 20 nt, synthetic construct
  misc_feature 1..20: note = Synthetic
  source 1..20: mol_type = other RNA
taacattgcctgttctctcc

SEQ ID NO: 73 - RNA, 20 nt, synthetic construct
  misc_feature 1..20: note = Synthetic
  source 1..20: mol_type = other RNA
cttctcgtcctggagagaac

SEQ ID NO: 74 - RNA, 20 nt, synthetic construct
  misc_feature 1..20: note = Synthetic
  source 1..20: mol_type = other RNA
ttccgaggaacttctcgtcc

SEQ ID NO: 75 - RNA, 20 nt, synthetic construct
  misc_feature 1..20: note = Synthetic
  source 1..20: mol_type = other RNA
acccttgctctttgcctcct

SEQ ID NO: 76 - RNA, 20 nt, synthetic construct
  misc_feature 1..20: note = Synthetic
  source 1..20: mol_type = other RNA
ttgctctttgcctcctaggc

SEQ ID NO: 77 - RNA, 20 nt, synthetic construct
  misc_feature 1..20: note = Synthetic
  source 1..20: mol_type = other RNA
tgctctttgcctcctaggct

SEQ ID NO: 78 - RNA, 20 nt, synthetic construct
  misc_feature 1..20: note = Synthetic
  source 1..20: mol_type = other RNA
cctgagacagggcccagcct

SEQ ID NO: 79 - RNA, 20 nt, synthetic construct
  misc_feature 1..20: note = Synthetic
  source 1..20: mol_type = other RNA
atctgattccacctgcagcc

SEQ ID NO: 80 - RNA, 20 nt, synthetic construct
  misc_feature 1..20: note = Synthetic
  source 1..20: mol_type = other RNA
cagcgcatccaggctgcagg

SEQ ID NO: 81 - RNA, 20 nt, synthetic construct
  misc_feature 1..20: note = Synthetic
  source 1..20: mol_type = other RNA
actcagcgcatccaggctgc

SEQ ID NO: 82 - RNA, 20 nt, synthetic construct
  misc_feature 1..20: note = Synthetic
  source 1..20: mol_type = other RNA
aaaccctcaagtgagtgagc

SEQ ID NO: 83 - RNA, 20 nt, synthetic construct
  misc_feature 1..20: note = Synthetic
  source 1..20: mol_type = other RNA
aaccctcaagtgagtgagct

SEQ ID NO: 84 - RNA, 20 nt, synthetic construct
  misc_feature 1..20: note = Synthetic
  source 1..20: mol_type = other RNA
ggcccagctcactcacttga

SEQ ID NO: 85 - RNA, 20 nt, synthetic construct
  misc_feature 1..20: note = Synthetic
  source 1..20: mol_type = other RNA
aggcccagctcactcacttg

SEQ ID NO: 86 - RNA, 20 nt, synthetic construct
  misc_feature 1..20: note = Synthetic
  source 1..20: mol_type = other RNA
cagactgcggggacacagtg

SEQ ID NO: 87 - RNA, 20 nt, synthetic construct
  misc_feature 1..20: note = Synthetic
  source 1..20: mol_type = other RNA
ctgcatccctgctcaggcta

SEQ ID NO: 88 - RNA, 20 nt, synthetic construct
  misc_feature 1..20: note = Synthetic
  source 1..20: mol_type = other RNA
cctgctcaggctaaggtgag

SEQ ID NO: 89 - RNA, 20 nt, synthetic construct
  misc_feature 1..20: note = Synthetic
  source 1..20: mol_type = other RNA
ctgctcaggctaaggtgagt

SEQ ID NO: 90 - RNA, 20 nt, synthetic construct
  misc_feature 1..20: note = Synthetic
  source 1..20: mol_type = other RNA
cagcacctgaccggtatccg

SEQ ID NO: 91 - RNA, 20 nt, synthetic construct
  misc_feature 1..20: note = Synthetic
  source 1..20: mol_type = other RNA
gtacaagctgtcggaaacag

SEQ ID NO: 92 - RNA, 20 nt, synthetic construct
  misc_feature 1..20: note = Synthetic
  source 1..20: mol_type = other RNA
ggagacgctggcgtaagtcc

SEQ ID NO: 93 - RNA, 20 nt, synthetic construct
  misc_feature 1..20: note = Synthetic
  source 1..20: mol_type = other RNA
ggcgtaagtccaggcaaccc

SEQ ID NO: 94 - RNA, 20 nt, synthetic construct
  misc_feature 1..20: note = Synthetic
  source 1..20: mol_type = other RNA
ctccacccaccagggttgcc

SEQ ID NO: 95 - RNA, 20 nt, synthetic construct
  misc_feature 1..20: note = Synthetic
  source 1..20: mol_type = other RNA
tgagtcccatccccccttgc

SEQ ID NO: 96 - RNA, 20 nt, synthetic construct
  misc_feature 1..20: note = Synthetic
  source 1..20: mol_type = other RNA
ccacatcctgcaagggggga

SEQ ID NO: 97 - RNA, 20 nt, synthetic construct
  misc_feature 1..20: note = Synthetic
  source 1..20: mol_type = other RNA
gcgtccacatcctgcaaggg

SEQ ID NO: 98 - RNA, 20 nt, synthetic construct
  misc_feature 1..20: note = Synthetic
  source 1..20: mol_type = other RNA
ggcgtccacatcctgcaagg

SEQ ID NO: 99 - RNA, 20 nt, synthetic construct
  misc_feature 1..20: note = Synthetic
  source 1..20: mol_type = other RNA
gggcgtccacatcctgcaag

SEQ ID NO: 100 - RNA, 20 nt, synthetic construct
  misc_feature 1..20: note = Synthetic
  source 1..20: mol_type = other RNA
tgggcgtccacatcctgcaa

SEQ ID NO: 101 - RNA, 20 nt, synthetic construct
  misc_feature 1..20: note = Synthetic
  source 1..20: mol_type = other RNA
gtgggcgtccacatcctgca

SEQ ID NO: 102-106 - skipped (5 entries)

SEQ ID NO: 107 - RNA, 20 nt, synthetic construct
  misc_feature 1..20: note = Synthetic
  source 1..20: mol_type = other RNA
acacaaataccagtccagcg

SEQ ID NO: 108-169 - skipped (62 entries)

SEQ ID NO: 170 - RNA, 22 nt, synthetic construct
  misc_feature 1..22: note = Synthetic
  source 1..22: mol_type = other RNA
gttttagagctatgctgttttg

SEQ ID NO: 171 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 172 - RNA, 76 nt, synthetic construct
  misc_feature 1..76: note = Synthetic
  source 1..76: mol_type = other RNA
gttttagagctagaaatagcaagttaaaataaggctagtccgttatcaacttgaaaaagtggcaccgagtcggtgc

SEQ ID NO: 173 - RNA, 68 nt, synthetic construct
  misc_feature 1..68: note = Synthetic
  source 1..68: mol_type = other RNA
gttttagagctagaaatagcaagttaaaataaggctagtccgttatcaacttggcaccgagtcggtgc

SEQ ID NO: 174-199 - skipped (26 entries)

SEQ ID NO: 200 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tcctacctgtcagagccccagttttagagctagaaatagcaagttaaaataaggctagtccgttatcaacttgaaaaagtggcaccgagtcggtgctttt

SEQ ID NO: 201 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gagccccaaggtaaaaaggcgttttagagctagaaatagcaagttaaaataaggctagtccgttatcaacttgaaaaagtggcaccgagtcggtgctttt

SEQ ID NO: 202 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
agccccaaggtaaaaaggccgttttagagctagaaatagcaagttaaaataaggctagtccgttatcaacttgaaaaagtggcaccgagtcggtgctttt

SEQ ID NO: 203 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tttcccggcctttttaccttgttttagagctagaaatagcaagttaaaataaggctagtccgttatcaacttgaaaaagtggcaccgagtcggtgctttt

SEQ ID NO: 204 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctttcccggcctttttacctgttttagagctagaaatagcaagttaaaataaggctagtccgttatcaacttgaaaaagtggcaccgagtcggtgctttt

SEQ ID NO: 205 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
acactgtgagctgcctgggagttttagagctagaaatagcaagttaaaataaggctagtccgttatcaacttgaaaaagtggcaccgagtcggtgctttt

SEQ ID NO: 206 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cacactgtgagctgcctggggttttagagctagaaatagcaagttaaaataaggctagtccgttatcaacttgaaaaagtggcaccgagtcggtgctttt

SEQ ID NO: 207 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gtggcacactgtgagctgccgttttagagctagaaatagcaagttaaaataaggctagtccgttatcaacttgaaaaagtggcaccgagtcggtgctttt

SEQ ID NO: 208 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gagattgagctctactcagggttttagagctagaaatagcaagttaaaataaggctagtccgttatcaacttgaaaaagtggcaccgagtcggtgctttt

SEQ ID NO: 209 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
agattgagctctactcaggtgttttagagctagaaatagcaagttaaaataaggctagtccgttatcaacttgaaaaagtggcaccgagtcggtgctttt

SEQ ID NO: 210 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
caggtgggccctcctccctcgttttagagctagaaatagcaagttaaaataaggctagtccgttatcaacttgaaaaagtggcaccgagtcggtgctttt

SEQ ID NO: 211 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cggaagagaccagagggagggttttagagctagaaatagcaagttaaaataaggctagtccgttatcaacttgaaaaagtggcaccgagtcggtgctttt

SEQ ID NO: 212 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
taccggaagagaccagaggggttttagagctagaaatagcaagttaaaataaggctagtccgttatcaacttgaaaaagtggcaccgagtcggtgctttt

SEQ ID NO: 213 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
agggaggcttatgccaatatgttttagagctagaaatagcaagttaaaataaggctagtccgttatcaacttgaaaaagtggcaccgagtcggtgctttt

SEQ ID NO: 214 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggcttatgccaatatcggtggttttagagctagaaatagcaagttaaaataaggctagtccgttatcaacttgaaaaagtggcaccgagtcggtgctttt

SEQ ID NO: 215 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tgactgcgcttttccttgtcgttttagagctagaaatagcaagttaaaataaggctagtccgttatcaacttgaaaaagtggcaccgagtcggtgctttt

SEQ ID NO: 216 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gactgcgcttttccttgtctgttttagagctagaaatagcaagttaaaataaggctagtccgttatcaacttgaaaaagtggcaccgagtcggtgctttt

SEQ ID NO: 217 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gcttttccttgtctgggcaggttttagagctagaaatagcaagttaaaataaggctagtccgttatcaacttgaaaaagtggcaccgagtcggtgctttt

SEQ ID NO: 218 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ccagttccgctgcccagacagttttagagctagaaatagcaagttaaaataaggctagtccgttatcaacttgaaaaagtggcaccgagtcggtgctttt

SEQ ID NO: 219 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gacctgaagcactggaagccgttttagagctagaaatagcaagttaaaataaggctagtccgttatcaacttgaaaaagtggcaccgagtcggtgctttt

SEQ ID NO: 220 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gcactggaagccaggtgtgcgttttagagctagaaatagcaagttaaaataaggctagtccgttatcaacttgaaaaagtggcaccgagtcggtgctttt

SEQ ID NO: 221 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cactggaagccaggtgtgcagttttagagctagaaatagcaagttaaaataaggctagtccgttatcaacttgaaaaagtggcaccgagtcggtgctttt

SEQ ID NO: 222 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggaagccaggtgtgcagggcgttttagagctagaaatagcaagttaaaataaggctagtccgttatcaacttgaaaaagtggcaccgagtcggtgctttt

SEQ ID NO: 223 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
agccaggtgtgcagggcagggttttagagctagaaatagcaagttaaaataaggctagtccgttatcaacttgaaaaagtggcaccgagtcggtgctttt

SEQ ID NO: 224 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gcccacctgccctgcacaccgttttagagctagaaatagcaagttaaaataaggctagtccgttatcaacttgaaaaagtggcaccgagtcggtgctttt

SEQ ID NO: 225 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gggctcagctgtgaggaagtgttttagagctagaaatagcaagttaaaataaggctagtccgttatcaacttgaaaaagtggcaccgagtcggtgctttt

SEQ ID NO: 226 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggggctcagctgtgaggaaggttttagagctagaaatagcaagttaaaataaggctagtccgttatcaacttgaaaaagtggcaccgagtcggtgctttt

SEQ ID NO: 227 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gaccagattcccagtatgttgttttagagctagaaatagcaagttaaaataaggctagtccgttatcaacttgaaaaagtggcaccgagtcggtgctttt

SEQ ID NO: 228 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ccagattcccagtatgttaggttttagagctagaaatagcaagttaaaataaggctagtccgttatcaacttgaaaaagtggcaccgagtcggtgctttt

SEQ ID NO: 229 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cagattcccagtatgttagggttttagagctagaaatagcaagttaaaataaggctagtccgttatcaacttgaaaaagtggcaccgagtcggtgctttt

SEQ ID NO: 230 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tcccagtatgttagggggctgttttagagctagaaatagcaagttaaaataaggctagtccgttatcaacttgaaaaagtggcaccgagtcggtgctttt

SEQ ID NO: 231 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tccaagccccctaacatactgttttagagctagaaatagcaagttaaaataaggctagtccgttatcaacttgaaaaagtggcaccgagtcggtgctttt

SEQ ID NO: 232 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctccaagccccctaacatacgttttagagctagaaatagcaagttaaaataaggctagtccgttatcaacttgaaaaagtggcaccgagtcggtgctttt

SEQ ID NO: 233 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
aaaggcactgcaagagacaagttttagagctagaaatagcaagttaaaataaggctagtccgttatcaacttgaaaaagtggcaccgagtcggtgctttt

SEQ ID NO: 234 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tccagtatattcatctaccagttttagagctagaaatagcaagttaaaataaggctagtccgttatcaacttgaaaaagtggcaccgagtcggtgctttt

SEQ ID NO: 235 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ttcatctaccatggtgagtggttttagagctagaaatagcaagttaaaataaggctagtccgttatcaacttgaaaaagtggcaccgagtcggtgctttt

SEQ ID NO: 236 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tcatctaccatggtgagtgcgttttagagctagaaatagcaagttaaaataaggctagtccgttatcaacttgaaaaagtggcaccgagtcggtgctttt

SEQ ID NO: 237 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
catctaccatggtgagtgcggttttagagctagaaatagcaagttaaaataaggctagtccgttatcaacttgaaaaagtggcaccgagtcggtgctttt

SEQ ID NO: 238 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
accatggtgagtgcggggccgttttagagctagaaatagcaagttaaaataaggctagtccgttatcaacttgaaaaagtggcaccgagtcggtgctttt

SEQ ID NO: 239 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gccaggccccgcactcaccagttttagagctagaaatagcaagttaaaataaggctagtccgttatcaacttgaaaaagtggcaccgagtcggtgctttt

SEQ ID NO: 240 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ccactctccacccccaatgtgttttagagctagaaatagcaagttaaaataaggctagtccgttatcaacttgaaaaagtggcaccgagtcggtgctttt

SEQ ID NO: 241 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctccacccccaatgtaggtggttttagagctagaaatagcaagttaaaataaggctagtccgttatcaacttgaaaaagtggcaccgagtcggtgctttt

SEQ ID NO: 242 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cacctcacctacattggggggttttagagctagaaatagcaagttaaaataaggctagtccgttatcaacttgaaaaagtggcaccgagtcggtgctttt

SEQ ID NO: 243 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gggcacctcacctacattgggttttagagctagaaatagcaagttaaaataaggctagtccgttatcaacttgaaaaagtggcaccgagtcggtgctttt

SEQ ID NO: 244 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tggggcacctcacctacattgttttagagctagaaatagcaagttaaaataaggctagtccgttatcaacttgaaaaagtggcaccgagtcggtgctttt

SEQ ID NO: 245 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctggggcacctcacctacatgttttagagctagaaatagcaagttaaaataaggctagtccgttatcaacttgaaaaagtggcaccgagtcggtgctttt

SEQ ID NO: 246 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
acctcccgagcaaacatgacgttttagagctagaaatagcaagttaaaataaggctagtccgttatcaacttgaaaaagtggcaccgagtcggtgctttt

SEQ ID NO: 247 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ccgagcaaacatgacaggtagttttagagctagaaatagcaagttaaaataaggctagtccgttatcaacttgaaaaagtggcaccgagtcggtgctttt

SEQ ID NO: 248 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gtacctctcacaggccctaagttttagagctagaaatagcaagttaaaataaggctagtccgttatcaacttgaaaaagtggcaccgagtcggtgctttt

SEQ ID NO: 249 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
agtacctctcacaggccctagttttagagctagaaatagcaagttaaaataaggctagtccgttatcaacttgaaaaagtggcaccgagtcggtgctttt

SEQ ID NO: 250 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gacgtcttgtgctctggagagttttagagctagaaatagcaagttaaaataaggctagtccgttatcaacttgaaaaagtggcaccgagtcggtgctttt

SEQ ID NO: 251 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
aacaagcttccaaaatggccgttttagagctagaaatagcaagttaaaataaggctagtccgttatcaacttgaaaaagtggcaccgagtcggtgctttt

SEQ ID NO: 252 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gagatcccgcatcactcaccgttttagagctagaaatagcaagttaaaataaggctagtccgttatcaacttgaaaaagtggcaccgagtcggtgctttt

SEQ ID NO: 253 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gcccctggcctttgcagagcgttttagagctagaaatagcaagttaaaataaggctagtccgttatcaacttgaaaaagtggcaccgagtcggtgctttt

SEQ ID NO: 254 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
accggctctgcaaaggccaggttttagagctagaaatagcaagttaaaataaggctagtccgttatcaacttgaaaaagtggcaccgagtcggtgctttt

SEQ ID NO: 255 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
caccggctctgcaaaggccagttttagagctagaaatagcaagttaaaataaggctagtccgttatcaacttgaaaaagtggcaccgagtcggtgctttt

SEQ ID NO: 256 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ccaccggctctgcaaaggccgttttagagctagaaatagcaagttaaaataaggctagtccgttatcaacttgaaaaagtggcaccgagtcggtgctttt

SEQ ID NO: 257 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctgctccaccggctctgcaagttttagagctagaaatagcaagttaaaataaggctagtccgttatcaacttgaaaaagtggcaccgagtcggtgctttt

SEQ ID NO: 258 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tcagctgtgtcacccgtttcgttttagagctagaaatagcaagttaaaataaggctagtccgttatcaacttgaaaaagtggcaccgagtcggtgctttt

SEQ ID NO: 259 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gctgtgtcacccgtttcagggttttagagctagaaatagcaagttaaaataaggctagtccgttatcaacttgaaaaagtggcaccgagtcggtgctttt

SEQ ID NO: 260 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctgtgtcacccgtttcaggtgttttagagctagaaatagcaagttaaaataaggctagtccgttatcaacttgaaaaagtggcaccgagtcggtgctttt

SEQ ID NO: 261 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tgtgtcacccgtttcaggtggttttagagctagaaatagcaagttaaaataaggctagtccgttatcaacttgaaaaagtggcaccgagtcggtgctttt

SEQ ID NO: 262 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cacccgtttcaggtggggtggttttagagctagaaatagcaagttaaaataaggctagtccgttatcaacttgaaaaagtggcaccgagtcggtgctttt

SEQ ID NO: 263 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
acccgtttcaggtggggtgagttttagagctagaaatagcaagttaaaataaggctagtccgttatcaacttgaaaaagtggcaccgagtcggtgctttt

SEQ ID NO: 264 - RNA, 100 nt, synthetic construct
  misc_feature 1..100: note = Synthetic
  variation 1..100: note = 2'-O-methylation and phosphorothioate internucleoside linkage modifications are present or absent for detailed description of substitutions and preferred
  source 1..100: mol_type = other RNA
cccgtttcaggtggggtgaggttttagagctagaaatagcaagttaaaataaggctagtccgttatcaacttgaaaaagtggcaccgagtcggtgctttt

SEQ ID NO: 265 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gtctgaggccctccctccacgttttagagctagaaatagcaagttaaaataaggctagtccgttatcaacttgaaaaagtggcaccgagtcggtgctttt

SEQ ID NO: 266 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tctgaggccctccctccacagttttagagctagaaatagcaagttaaaataaggctagtccgttatcaacttgaaaaagtggcaccgagtcggtgctttt

SEQ ID NO: 267 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
caaggcagccctgtggaggggttttagagctagaaatagcaagttaaaataaggctagtccgttatcaacttgaaaaagtggcaccgagtcggtgctttt

SEQ ID NO: 268 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gctcaaggcagccctgtggagttttagagctagaaatagcaagttaaaataaggctagtccgttatcaacttgaaaaagtggcaccgagtcggtgctttt

SEQ ID NO: 269 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cgctcaaggcagccctgtgggttttagagctagaaatagcaagttaaaataaggctagtccgttatcaacttgaaaaagtggcaccgagtcggtgctttt

SEQ ID NO: 270 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tgtgcagactcagaggtgaggttttagagctagaaatagcaagttaaaataaggctagtccgttatcaacttgaaaaagtggcaccgagtcggtgctttt

SEQ ID NO: 271 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
taacattgcctgttctctccgttttagagctagaaatagcaagttaaaataaggctagtccgttatcaacttgaaaaagtggcaccgagtcggtgctttt

SEQ ID NO: 272 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cttctcgtcctggagagaacgttttagagctagaaatagcaagttaaaataaggctagtccgttatcaacttgaaaaagtggcaccgagtcggtgctttt

SEQ ID NO: 273 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ttccgaggaacttctcgtccgttttagagctagaaatagcaagttaaaataaggctagtccgttatcaacttgaaaaagtggcaccgagtcggtgctttt

SEQ ID NO: 274 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
acccttgctctttgcctcctgttttagagctagaaatagcaagttaaaataaggctagtccgttatcaacttgaaaaagtggcaccgagtcggtgctttt

SEQ ID NO: 275 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ttgctctttgcctcctaggcgttttagagctagaaatagcaagttaaaataaggctagtccgttatcaacttgaaaaagtggcaccgagtcggtgctttt

SEQ ID NO: 276 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tgctctttgcctcctaggctgttttagagctagaaatagcaagttaaaataaggctagtccgttatcaacttgaaaaagtggcaccgagtcggtgctttt

SEQ ID NO: 277 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cctgagacagggcccagcctgttttagagctagaaatagcaagttaaaataaggctagtccgttatcaacttgaaaaagtggcaccgagtcggtgctttt

SEQ ID NO: 278 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
atctgattccacctgcagccgttttagagctagaaatagcaagttaaaataaggctagtccgttatcaacttgaaaaagtggcaccgagtcggtgctttt

SEQ ID NO: 279 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cagcgcatccaggctgcagggttttagagctagaaatagcaagttaaaataaggctagtccgttatcaacttgaaaaagtggcaccgagtcggtgctttt

SEQ ID NO: 280 - RNA, 100 nt, synthetic construct
  misc_feature 1..100: note = Synthetic
  source 1..100: mol_type = other RNA
actcagcgcatccaggctgcgttttagagctagaaatagcaagttaaaataaggctagtccgttatcaacttgaaaaagtggcaccgagtcggtgctttt

SEQ ID NO: 281 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
aaaccctcaagtgagtgagcgttttagagctagaaatagcaagttaaaataaggctagtccgttatcaacttgaaaaagtggcaccgagtcggtgctttt

SEQ ID NO: 282 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
aaccctcaagtgagtgagctgttttagagctagaaatagcaagttaaaataaggctagtccgttatcaacttgaaaaagtggcaccgagtcggtgctttt

SEQ ID NO: 283 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggcccagctcactcacttgagttttagagctagaaatagcaagttaaaataaggctagtccgttatcaacttgaaaaagtggcaccgagtcggtgctttt

SEQ ID NO: 284 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
aggcccagctcactcacttggttttagagctagaaatagcaagttaaaataaggctagtccgttatcaacttgaaaaagtggcaccgagtcggtgctttt

SEQ ID NO: 285 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cagactgcggggacacagtggttttagagctagaaatagcaagttaaaataaggctagtccgttatcaacttgaaaaagtggcaccgagtcggtgctttt

SEQ ID NO: 286 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctgcatccctgctcaggctagttttagagctagaaatagcaagttaaaataaggctagtccgttatcaacttgaaaaagtggcaccgagtcggtgctttt

SEQ ID NO: 287 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cctgctcaggctaaggtgaggttttagagctagaaatagcaagttaaaataaggctagtccgttatcaacttgaaaaagtggcaccgagtcggtgctttt

SEQ ID NO: 288 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctgctcaggctaaggtgagtgttttagagctagaaatagcaagttaaaataaggctagtccgttatcaacttgaaaaagtggcaccgagtcggtgctttt

SEQ ID NO: 289 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cagcacctgaccggtatccggttttagagctagaaatagcaagttaaaataaggctagtccgttatcaacttgaaaaagtggcaccgagtcggtgctttt

SEQ ID NO: 290 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gtacaagctgtcggaaacaggttttagagctagaaatagcaagttaaaataaggctagtccgttatcaacttgaaaaagtggcaccgagtcggtgctttt

SEQ ID NO: 291 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggagacgctggcgtaagtccgttttagagctagaaatagcaagttaaaataaggctagtccgttatcaacttgaaaaagtggcaccgagtcggtgctttt

SEQ ID NO: 292 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggcgtaagtccaggcaacccgttttagagctagaaatagcaagttaaaataaggctagtccgttatcaacttgaaaaagtggcaccgagtcggtgctttt

SEQ ID NO: 293 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctccacccaccagggttgccgttttagagctagaaatagcaagttaaaataaggctagtccgttatcaacttgaaaaagtggcaccgagtcggtgctttt

SEQ ID NO: 294 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tgagtcccatccccccttgcgttttagagctagaaatagcaagttaaaataaggctagtccgttatcaacttgaaaaagtggcaccgagtcggtgctttt

SEQ ID NO: 295 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ccacatcctgcaaggggggagttttagagctagaaatagcaagttaaaataaggctagtccgttatcaacttgaaaaagtggcaccgagtcggtgctttt

SEQ ID NO: 296 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gcgtccacatcctgcaaggggttttagagctagaaatagcaagttaaaataaggctagtccgttatcaacttgaaaaagtggcaccgagtcggtgctttt

SEQ ID NO: 297 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggcgtccacatcctgcaagggttttagagctagaaatagcaagttaaaataaggctagtccgttatcaacttgaaaaagtggcaccgagtcggtgctttt

SEQ ID NO: 298 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gggcgtccacatcctgcaaggttttagagctagaaatagcaagttaaaataaggctagtccgttatcaacttgaaaaagtggcaccgagtcggtgctttt

SEQ ID NO: 299 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tgggcgtccacatcctgcaagttttagagctagaaatagcaagttaaaataaggctagtccgttatcaacttgaaaaagtggcaccgagtcggtgctttt

SEQ ID NO: 300 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gtgggcgtccacatcctgcagttttagagctagaaatagcaagttaaaataaggctagtccgttatcaacttgaaaaagtggcaccgagtcggtgctttt

SEQ ID NO: 301 - RNA, 100 nt, synthetic construct; identical to SEQ ID NO: 200
  misc_feature 1..100: note = Synthetic
  source 1..100: mol_type = other RNA

SEQ ID NO: 302 - RNA, 100 nt, synthetic construct; identical to SEQ ID NO: 201
  misc_feature 1..100: note = Synthetic
  source 1..100: mol_type = other RNA

SEQ ID NO: 303 - RNA, 100 nt, synthetic construct; identical to SEQ ID NO: 202
  misc_feature 1..100: note = Synthetic
  source 1..100: mol_type = other RNA

SEQ ID NO: 304 - RNA, 100 nt, synthetic construct; identical to SEQ ID NO: 203
  misc_feature 1..100: note = Synthetic
  source 1..100: mol_type = other RNA

SEQ ID NO: 305 - RNA, 100 nt, synthetic construct; identical to SEQ ID NO: 204
  misc_feature 1..100: note = Synthetic
  source 1..100: mol_type = other RNA

SEQ ID NO: 306 - RNA, 100 nt, synthetic construct; identical to SEQ ID NO: 205
  misc_feature 1..100: note = Synthetic
  source 1..100: mol_type = other RNA

SEQ ID NO: 307 - RNA, 100 nt, synthetic construct; identical to SEQ ID NO: 206
  misc_feature 1..100: note = Synthetic
  source 1..100: mol_type = other RNA

SEQ ID NO: 308 - RNA, 100 nt, synthetic construct; identical to SEQ ID NO: 207
  misc_feature 1..100: note = Synthetic
  source 1..100: mol_type = other RNA

SEQ ID NO: 309 - RNA, 100 nt, synthetic construct; identical to SEQ ID NO: 208
  misc_feature 1..100: note = Synthetic
  source 1..100: mol_type = other RNA

SEQ ID NO: 310 - RNA, 100 nt, synthetic construct; identical to SEQ ID NO: 209
  misc_feature 1..100: note = Synthetic
  source 1..100: mol_type = other RNA

SEQ ID NO: 311 - RNA, 100 nt, synthetic construct; identical to SEQ ID NO: 210
  misc_feature 1..100: note = Synthetic
  source 1..100: mol_type = other RNA

SEQ ID NO: 312 - RNA, 100 nt, synthetic construct; identical to SEQ ID NO: 211
  misc_feature 1..100: note = Synthetic
  source 1..100: mol_type = other RNA

SEQ ID NO: 313 - RNA, 100 nt, synthetic construct; identical to SEQ ID NO: 212
  misc_feature 1..100: note = Synthetic
  source 1..100: mol_type = other RNA

SEQ ID NO: 314 - RNA, 100 nt, synthetic construct; identical to SEQ ID NO: 213
  misc_feature 1..100: note = Synthetic
  source 1..100: mol_type = other RNA

SEQ ID NO: 315 - RNA, 100 nt, synthetic construct; identical to SEQ ID NO: 214
  misc_feature 1..100: note = Synthetic
  source 1..100: mol_type = other RNA

SEQ ID NO: 316 - RNA, 100 nt, synthetic construct; identical to SEQ ID NO: 215
  misc_feature 1..100: note = Synthetic
  source 1..100: mol_type = other RNA

SEQ ID NO: 317 - RNA, 100 nt, synthetic construct; identical to SEQ ID NO: 216
  misc_feature 1..100: note = Synthetic
  source 1..100: mol_type = other RNA

SEQ ID NO: 318 - RNA, 100 nt, synthetic construct; identical to SEQ ID NO: 217
  misc_feature 1..100: note = Synthetic
  source 1..100: mol_type = other RNA

SEQ ID NO: 319 - RNA, 100 nt, synthetic construct; identical to SEQ ID NO: 218
  misc_feature 1..100: note = Synthetic
  source 1..100: mol_type = other RNA

SEQ ID NO: 320 - RNA, 100 nt, synthetic construct; identical to SEQ ID NO: 219
  misc_feature 1..100: note = Synthetic
  source 1..100: mol_type = other RNA

SEQ ID NO: 321 - RNA, 100 nt, synthetic construct; identical to SEQ ID NO: 220
  misc_feature 1..100: note = Synthetic
  source 1..100: mol_type = other RNA

SEQ ID NO: 322 - RNA, 100 nt, synthetic construct; identical to SEQ ID NO: 221
  misc_feature 1..100: note = Synthetic
  source 1..100: mol_type = other RNA

SEQ ID NO: 323 - RNA, 100 nt, synthetic construct; identical to SEQ ID NO: 222
  misc_feature 1..100: note = Synthetic
  source 1..100: mol_type = other RNA

SEQ ID NO: 324 - RNA, 100 nt, synthetic construct; identical to SEQ ID NO: 223
  misc_feature 1..100: note = Synthetic
  source 1..100: mol_type = other RNA

SEQ ID NO: 325 - RNA, 100 nt, synthetic construct; identical to SEQ ID NO: 224
  misc_feature 1..100: note = Synthetic
  source 1..100: mol_type = other RNA

SEQ ID NO: 326 - RNA, 100 nt, synthetic construct; identical to SEQ ID NO: 225
  misc_feature 1..100: note = Synthetic
  source 1..100: mol_type = other RNA

SEQ ID NO: 327 - RNA, 100 nt, synthetic construct; identical to SEQ ID NO: 226
  misc_feature 1..100: note = Synthetic
  source 1..100: mol_type = other RNA

SEQ ID NO: 328 - RNA, 100 nt, synthetic construct; identical to SEQ ID NO: 227
  misc_feature 1..100: note = Synthetic
  source 1..100: mol_type = other RNA

SEQ ID NO: 329 - RNA, 100 nt, synthetic construct; identical to SEQ ID NO: 228
  misc_feature 1..100: note = Synthetic
  source 1..100: mol_type = other RNA

SEQ ID NO: 330 - RNA, 100 nt, synthetic construct; identical to SEQ ID NO: 229
  misc_feature 1..100: note = Synthetic
  source 1..100: mol_type = other RNA

SEQ ID NO: 331 - RNA, 100 nt, synthetic construct; identical to SEQ ID NO: 230
  misc_feature 1..100: note = Synthetic
  source 1..100: mol_type = other RNA

SEQ ID NO: 332 - RNA, 100 nt, synthetic construct; identical to SEQ ID NO: 231
  misc_feature 1..100: note = Synthetic
  source 1..100: mol_type = other RNA

SEQ ID NO: 333 - RNA, 100 nt, synthetic construct; identical to SEQ ID NO: 232
  misc_feature 1..100: note = Synthetic
  source 1..100: mol_type = other RNA

SEQ ID NO: 334 - RNA, 100 nt, synthetic construct; identical to SEQ ID NO: 233
  misc_feature 1..100: note = Synthetic
  source 1..100: mol_type = other RNA

SEQ ID NO: 335 - RNA, 100 nt, synthetic construct; identical to SEQ ID NO: 234
  misc_feature 1..100: note = Synthetic
  source 1..100: mol_type = other RNA

SEQ ID NO: 336 - RNA, 100 nt, synthetic construct; identical to SEQ ID NO: 235
  misc_feature 1..100: note = Synthetic
  source 1..100: mol_type = other RNA

SEQ ID NO: 337 - RNA, 100 nt, synthetic construct; identical to SEQ ID NO: 236
  misc_feature 1..100: note = Synthetic
  source 1..100: mol_type = other RNA

SEQ ID NO: 338 - RNA, 100 nt, synthetic construct; identical to SEQ ID NO: 237
  misc_feature 1..100: note = Synthetic
  source 1..100: mol_type = other RNA

SEQ ID NO: 339 - RNA, 100 nt, synthetic construct; identical to SEQ ID NO: 238
  misc_feature 1..100: note = Synthetic
  source 1..100: mol_type = other RNA

SEQ ID NO: 340 - RNA, 100 nt, synthetic construct; identical to SEQ ID NO: 239
  misc_feature 1..100: note = Synthetic
  source 1..100: mol_type = other RNA

SEQ ID NO: 341 - RNA, 100 nt, synthetic construct; identical to SEQ ID NO: 240
  misc_feature 1..100: note = Synthetic
  source 1..100: mol_type = other RNA

SEQ ID NO: 342 - RNA, 100 nt, synthetic construct; identical to SEQ ID NO: 241
  misc_feature 1..100: note = Synthetic
  source 1..100: mol_type = other RNA

SEQ ID NO: 343 - RNA, 100 nt, synthetic construct; identical to SEQ ID NO: 242
  misc_feature 1..100: note = Synthetic
  source 1..100: mol_type = other RNA

SEQ ID NO: 344 - RNA, 100 nt, synthetic construct; identical to SEQ ID NO: 243
  misc_feature 1..100: note = Synthetic
  source 1..100: mol_type = other RNA

SEQ ID NO: 345 - RNA, 100 nt, synthetic construct; identical to SEQ ID NO: 244
  misc_feature 1..100: note = Synthetic
  source 1..100: mol_type = other RNA

SEQ ID NO: 346 - RNA, 100 nt, synthetic construct; identical to SEQ ID NO: 245
  misc_feature 1..100: note = Synthetic
  source 1..100: mol_type = other RNA

SEQ ID NO: 347 - RNA, 100 nt, synthetic construct; identical to SEQ ID NO: 246
  misc_feature 1..100: note = Synthetic
  source 1..100: mol_type = other RNA

SEQ ID NO: 348 - RNA, 100 nt, synthetic construct; identical to SEQ ID NO: 247
  misc_feature 1..100: note = Synthetic
  source 1..100: mol_type = other RNA

SEQ ID NO: 349 - RNA, 100 nt, synthetic construct; identical to SEQ ID NO: 248
  misc_feature 1..100: note = Synthetic
  source 1..100: mol_type = other RNA

SEQ ID NO: 350 - RNA, 100 nt, synthetic construct; identical to SEQ ID NO: 249
  misc_feature 1..100: note = Synthetic
  source 1..100: mol_type = other RNA

SEQ ID NO: 351 - RNA, 100 nt, synthetic construct; identical to SEQ ID NO: 250
  misc_feature 1..100: note = Synthetic
  source 1..100: mol_type = other RNA

SEQ ID NO: 352 - RNA, 100 nt, synthetic construct; identical to SEQ ID NO: 251
  misc_feature 1..100: note = Synthetic
  source 1..100: mol_type = other RNA

SEQ ID NO: 353 - RNA, 100 nt, synthetic construct; identical to SEQ ID NO: 252
  misc_feature 1..100: note = Synthetic
  source 1..100: mol_type = other RNA

SEQ ID NO: 354 - RNA, 100 nt, synthetic construct; identical to SEQ ID NO: 253
  misc_feature 1..100: note = Synthetic
  source 1..100: mol_type = other RNA

SEQ ID NO: 355 - RNA, 100 nt, synthetic construct; identical to SEQ ID NO: 254
  misc_feature 1..100: note = Synthetic
  source 1..100: mol_type = other RNA

SEQ ID NO: 356 - RNA, 100 nt, synthetic construct; identical to SEQ ID NO: 255
  misc_feature 1..100: note = Synthetic
  source 1..100: mol_type = other RNA

SEQ ID NO: 357 - RNA, 100 nt, synthetic construct; identical to SEQ ID NO: 256
  misc_feature 1..100: note = Synthetic
  source 1..100: mol_type = other RNA

SEQ ID NO: 358 - RNA, 100 nt, synthetic construct; identical to SEQ ID NO: 257
  misc_feature 1..100: note = Synthetic
  source 1..100: mol_type = other RNA

SEQ ID NO: 359 - RNA, 100 nt, synthetic construct; identical to SEQ ID NO: 258
  misc_feature 1..100: note = Synthetic
  source 1..100: mol_type = other RNA

SEQ ID NO: 360 - RNA, 100 nt, synthetic construct; identical to SEQ ID NO: 259
  misc_feature 1..100: note = Synthetic
  source 1..100: mol_type = other RNA

SEQ ID NO: 361 - RNA, 100 nt, synthetic construct; identical to SEQ ID NO: 260
  misc_feature 1..100: note = Synthetic
  source 1..100: mol_type = other RNA

SEQ ID NO: 362 - RNA, 100 nt, synthetic construct; identical to SEQ ID NO: 261
  misc_feature 1..100: note = Synthetic
  source 1..100: mol_type = other RNA

SEQ ID NO: 363 - RNA, 100 nt, synthetic construct; identical to SEQ ID NO: 262
  misc_feature 1..100: note = Synthetic
  source 1..100: mol_type = other RNA

SEQ ID NO: 364 - RNA, 100 nt, synthetic construct; identical to SEQ ID NO: 263
  misc_feature 1..100: note = Synthetic
  source 1..100: mol_type = other RNA

SEQ ID NO: 365 - RNA, 100 nt, synthetic construct; identical to SEQ ID NO: 264
  misc_feature 1..100: note = Synthetic
  source 1..100: mol_type = other RNA

SEQ ID NO: 366 - RNA, 100 nt, synthetic construct; identical to SEQ ID NO: 265
  misc_feature 1..100: note = Synthetic
  source 1..100: mol_type = other RNA

SEQ ID NO: 367 - RNA, 100 nt, synthetic construct; identical to SEQ ID NO: 266
  misc_feature 1..100: note = Synthetic
  source 1..100: mol_type = other RNA

SEQ ID NO: 368 - RNA, 100 nt, synthetic construct; identical to SEQ ID NO: 267
  misc_feature 1..100: note = Synthetic
  source 1..100: mol_type = other RNA

SEQ ID NO: 369 - RNA, 100 nt, synthetic construct; identical to SEQ ID NO: 268
  misc_feature 1..100: note = Synthetic
  source 1..100: mol_type = other RNA

SEQ ID NO: 370 - RNA, 100 nt, synthetic construct; identical to SEQ ID NO: 269
  misc_feature 1..100: note = Synthetic
  source 1..100: mol_type = other RNA

SEQ ID NO: 371 - RNA, 100 nt, synthetic construct; identical to SEQ ID NO: 270
  misc_feature 1..100: note = Synthetic
  source 1..100: mol_type = other RNA

SEQ ID NO: 372 - RNA, 100 nt, synthetic construct; identical to SEQ ID NO: 271
  misc_feature 1..100: note = Synthetic
  source 1..100: mol_type = other RNA

SEQ ID NO: 373 - RNA, 100 nt, synthetic construct; identical to SEQ ID NO: 272
  misc_feature 1..100: note = Synthetic
  source 1..100: mol_type = other RNA

SEQ ID NO: 374 - RNA, 100 nt, synthetic construct; identical to SEQ ID NO: 273
  misc_feature 1..100: note = Synthetic
  source 1..100: mol_type = other RNA

SEQ ID NO: 375 - RNA, 100 nt, synthetic construct; identical to SEQ ID NO: 274
  misc_feature 1..100: note = Synthetic
  source 1..100: mol_type = other RNA

SEQ ID NO: 376 - RNA, 100 nt, synthetic construct; identical to SEQ ID NO: 275
  misc_feature 1..100: note = Synthetic
  source 1..100: mol_type = other RNA

SEQ ID NO: 377 - RNA, 100 nt, synthetic construct; identical to SEQ ID NO: 276
  misc_feature 1..100: note = Synthetic
  source 1..100: mol_type = other RNA

SEQ ID NO: 378 - RNA, 100 nt, synthetic construct; identical to SEQ ID NO: 277
  misc_feature 1..100: note = Synthetic
  source 1..100: mol_type = other RNA

SEQ ID NO: 379 - RNA, 100 nt, synthetic construct; identical to SEQ ID NO: 278
  misc_feature 1..100: note = Synthetic
  source 1..100: mol_type = other RNA

SEQ ID NO: 380 - RNA, 100 nt, synthetic construct; identical to SEQ ID NO: 279
  misc_feature 1..100: note = Synthetic
  source 1..100: mol_type = other RNA

SEQ ID NO: 381 - RNA, 100 nt, synthetic construct; identical to SEQ ID NO: 280
  misc_feature 1..100: note = Synthetic
  source 1..100: mol_type = other RNA

SEQ ID NO: 382 - RNA, 100 nt, synthetic construct; identical to SEQ ID NO: 281
  misc_feature 1..100: note = Synthetic
  source 1..100: mol_type = other RNA

SEQ ID NO: 383 - RNA, 100 nt, synthetic construct; identical to SEQ ID NO: 282
  misc_feature 1..100: note = Synthetic
  source 1..100: mol_type = other RNA

SEQ ID NO: 384 - RNA, 100 nt, synthetic construct; identical to SEQ ID NO: 283
  misc_feature 1..100: note = Synthetic
  source 1..100: mol_type = other RNA

SEQ ID NO: 385 - RNA, 100 nt, synthetic construct; identical to SEQ ID NO: 284
  misc_feature 1..100: note = Synthetic
  source 1..100: mol_type = other RNA

SEQ ID NO: 386 - RNA, 100 nt, synthetic construct; identical to SEQ ID NO: 285
  misc_feature 1..100: note = Synthetic
  source 1..100: mol_type = other RNA

SEQ ID NO: 387 - RNA, 100 nt, synthetic construct; identical to SEQ ID NO: 286
  misc_feature 1..100: note = Synthetic
  source 1..100: mol_type = other RNA

SEQ ID NO: 388 - RNA, 100 nt, synthetic construct; identical to SEQ ID NO: 287
  misc_feature 1..100: note = Synthetic
  source 1..100: mol_type = other RNA

SEQ ID NO: 389 - RNA, 100 nt, synthetic construct; identical to SEQ ID NO: 288
  misc_feature 1..100: note = Synthetic
  source 1..100: mol_type = other RNA

SEQ ID NO: 390 - RNA, 100 nt, synthetic construct; identical to SEQ ID NO: 289
  misc_feature 1..100: note = Synthetic
  source 1..100: mol_type = other RNA

SEQ ID NO: 391 - RNA, 100 nt, synthetic construct; identical to SEQ ID NO: 290
  misc_feature 1..100: note = Synthetic
  source 1..100: mol_type = other RNA

SEQ ID NO: 392 - RNA, 100 nt, synthetic construct; identical to SEQ ID NO: 291
  misc_feature 1..100: note = Synthetic
  source 1..100: mol_type = other RNA

SEQ ID NO: 393 - RNA, 100 nt, synthetic construct; identical to SEQ ID NO: 292
  misc_feature 1..100: note = Synthetic
  source 1..100: mol_type = other RNA

SEQ ID NO: 394 - RNA, 100 nt, synthetic construct; identical to SEQ ID NO: 293
  misc_feature 1..100: note = Synthetic
  source 1..100: mol_type = other RNA

SEQ ID NO: 395 - RNA, 100 nt, synthetic construct; identical to SEQ ID NO: 294
  misc_feature 1..100: note = Synthetic
  source 1..100: mol_type = other RNA

SEQ ID NO: 396 - RNA, 100 nt, synthetic construct; identical to SEQ ID NO: 295
  misc_feature 1..100: note = Synthetic
  source 1..100: mol_type = other RNA

SEQ ID NO: 397 - RNA, 100 nt, synthetic construct; identical to SEQ ID NO: 296
  misc_feature 1..100: note = Synthetic
  source 1..100: mol_type = other RNA

SEQ ID NO: 398 - RNA, 100 nt, synthetic construct; identical to SEQ ID NO: 297
  misc_feature 1..100: note = Synthetic
  source 1..100: mol_type = other RNA

SEQ ID NO: 399 - RNA, 100 nt, synthetic construct; identical to SEQ ID NO: 298
  misc_feature 1..100: note = Synthetic
  source 1..100: mol_type = other RNA

SEQ ID NO: 400 - RNA, 100 nt, synthetic construct; identical to SEQ ID NO: 299
  misc_feature 1..100: note = Synthetic
  source 1..100: mol_type = other RNA

SEQ ID NO: 401 - RNA, 100 nt, synthetic construct; identical to SEQ ID NO: 300
  misc_feature 1..100: note = Synthetic
  source 1..100: mol_type = other RNA

SEQ ID NO: 402-599 - skipped (198 entries)

SEQ ID NO: 600 - protein, 7 aa, synthetic construct
  REGION 1..7: note = Synthetic
PKKKRKV

SEQ ID NO: 601 - protein, 7 aa, synthetic construct
  REGION 1..7: note = Synthetic
PKKKRRV

SEQ ID NO: 602 - protein, 16 aa, synthetic construct
  REGION 1..16: note = Synthetic
KRPAATKKAGQAKKKK

SEQ ID NO: 603-699 - skipped (97 entries)

SEQ ID NO: 700 - RNA, 20 nt, synthetic construct
  misc_feature 1..20: note = Synthetic
  source 1..20: mol_type = other RNA
ggccacggagcgagacatct

SEQ ID NO: 701 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggccacggagcgagacatctgttttagagctagaaatagcaagttaaaataaggctagtccgttatcaacttgaaaaagtggcaccgagtcggtgctttt

SEQ ID NO: 702 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ttctaggggccccaactccagttttagagctagaaatagcaagttaaaataaggctagtccgttatcaacttgaaaaagtggcaccgagtcggtgctttt

SEQ ID NO: 703 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
agagtctctcagctggtacagttttagagctagaaatagcaagttaaaataaggctagtccgttatcaacttgaaaaagtggcaccgagtcggtgctttt

SEQ ID NO: 704 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ttaccccacttaactatcttgttttagagctagaaatagcaagttaaaataaggctagtccgttatcaacttgaaaaagtggcaccgagtcggtgctttt

SEQ ID NO: 705 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ccactctgccccatgggctcgttttagagctagaaatagcaagttaaaataaggctagtccgttatcaacttgaaaaagtggcaccgagtcggtgctttt

SEQ ID NO: 706 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cgctgtcaagtccagttctagttttagagctagaaatagcaagttaaaataaggctagtccgttatcaacttgaaaaagtggcaccgagtcggtgctttt

SEQ ID NO: 707 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gagtccgagcagaagaagaagttttagagctagaaatagcaagttaaaataaggctagtccgttatcaacttgaaaaagtggcaccgagtcggtgctttt

SEQ ID NO: 708 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gaccccctccaccccgcctcgttttagagctagaaatagcaagttaaaataaggctagtccgttatcaacttgaaaaagtggcaccgagtcggtgctttt

SEQ ID NO: 709 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gacttgttttcattgttctcgttttagagctagaaatagcaagttaaaataaggctagtccgttatcaacttgaaaaagtggcaccgagtcggtgctttt

SEQ ID NO: 710-799 - skipped (90 entries)

SEQ ID NO: 800 - protein, 1379 aa, synthetic construct
  REGION 1..1379: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 801 - DNA, 4140 bp, synthetic construct
  misc_feature 1..4140: note = Synthetic
  source 1..4140: mol_type = other DNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 802 - DNA, 4140 bp, synthetic construct
  misc_feature 1..4140: note = Synthetic
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 803 - RNA, 4197 nt, synthetic construct
  misc_feature 1..4197: note = Synthetic
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 804 - RNA, 4782 nt, synthetic construct
  misc_feature 1..4782: note = Synthetic
  source 1..4782: mol_type = other RNA
atggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 805 - RNA, 4839 nt, synthetic construct
  misc_feature 1..4839: note = Synthetic
  source 1..4839: mol_type = other RNA
atggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 806 - RNA, 5043 nt, synthetic construct
  misc_feature 1..5043: note = Synthetic
  source 1..5043: mol_type = other RNA
atggaagcaagcccggcaagcggaccgagacacctgatggacccgcacatcttcacaagcaacttcaacaacggaatcggaagacacaagacatacctgtgctacgaagtcgaaagactggacaacggaacaagcgtcaagatggaccagcacagaggattcctgcacaaccaggcaaagaacctgctgtgcggattctacggaagacacgcagaactgagattcctggacctggtcccgagcctgcagctggacccggcacagatctacagagtcacatggttcatcagctggagcccgtgcttcagctggggatgcgcaggagaagtcagagcatttctgcaggaaaacacacacgtcagactgagaatcttcgcagcaagaatctacgactacgacccgctgtacaaggaagcactgcagatgctgagagacgcaggagcacaggtcagcatcatgacatacgacgaattcaagcactgctgggacacattcgtcgaccaccagggatgcccgttccagccgtgggacggactggacgaacacagccaggcactgagcggaagactgagagcaatcctgcagaaccagggaaacagcggaagcgaaacaccgggaacaagcgaaagcgcaacaccggaaagcgacaagaagtacagcatcggactggccatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgatctgagccagctgggaggagacagcggaggaagcacaaacctgagcgacatcatcgaaaaggaaacaggaaagcagctggtcatccaggaaagcatcctgatgctgccggaagaagtcgaagaagtcatcggaaacaagccggaaagcgacatcctggtccacacagcatacgacgaaagcacagacgaaaacgtcatgctgctgacaagcgacgcaccggaatacaagccgtgggcactggtcatccaggacagcaacggagaaaacaagatcaagatgctgagcggaggaagcccgaagaagaagagaaaggtctaa

SEQ ID NO: 807 - RNA, 291 nt, synthetic construct
  misc_feature 1..291: note = Synthetic
  source 1..291: mol_type = other RNA
atgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtga

SEQ ID NO: 808 - RNA, 324 nt, synthetic construct
  misc_feature 1..324: note = Synthetic
  source 1..324: mol_type = other RNA
atgaccaacctgtccgacatcatcgagaaggagaccggcaagcagctggtgatccaggagtccatcctgatgctgcccgaggaggtggaggaggtgatcggcaacaagcccgagtccgacatcctggtgcacaccgcctacgacgagtccaccgacgagaacgtgatgctgctgacctccgacgcccccgagtacaagccctgggccctggtgatccaggactccaacggcgagaacaagatcaagatgctgtccggcggctccaagcggaccgccgacggctccgagttcgagtcccccaagaagaagcggaaggtggagtga

SEQ ID NO: 809 - protein, 1379 aa, synthetic construct; identical to SEQ ID NO: 800
  REGION 1..1379: note = Synthetic

SEQ ID NO: 810 - protein, 1398 aa, synthetic construct
  REGION 1..1398: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 811 - protein, 1593 aa, synthetic construct
  REGION 1..1593: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 812 - protein, 1612 aa, synthetic construct
  REGION 1..1612: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 813 - protein, 1680 aa, synthetic construct
  REGION 1..1680: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 814 - protein, 107 aa, synthetic construct
  REGION 1..107: note = Synthetic
MTNLSDIIEKETGKQLVIQESILMLPEEVEEVIGNKPESDILVHTAYDESTDENVMLLTSDAPEYKPWALVIQDSNGENKIKMLSGGSKRTADGSEFESPKKKRKVE

SEQ ID NO: 815-899 - skipped (85 entries)

SEQ ID NO: 900 - protein, 16 aa, synthetic construct
  REGION 1..16: note = Synthetic
SGSETPGTSESATPES

SEQ ID NO: 901 - protein, 12 aa, synthetic construct
  REGION 1..12: note = Synthetic
SGSETPGTSESA

SEQ ID NO: 902 - protein, 21 aa, synthetic construct
  REGION 1..21: note = Synthetic
SGSETPGTSESATPEGGSGGS

SEQ ID NO: 903 - protein, 15 aa, synthetic construct
  REGION 1..15: note = Synthetic
GGGGSEAAAKEAAAK

SEQ ID NO: 904 - protein, 15 aa, synthetic construct
  REGION 1..15: note = Synthetic
EAAAKGGGGSGGGGS

SEQ ID NO: 905 - protein, 15 aa, synthetic construct
  REGION 1..15: note = Synthetic
EAAAKEAAAKEAAAK

SEQ ID NO: 906 - protein, 20 aa, synthetic construct
  REGION 1..20: note = Synthetic
GGGGSGGGGSGGGGSGGGGS

SEQ ID NO: 907 - protein, 20 aa, synthetic construct
  REGION 1..20: note = Synthetic
GGGGSGGGGSEAAAKEAAAK

SEQ ID NO: 908 - protein, 20 aa, synthetic construct
  REGION 1..20: note = Synthetic
GGGGSEAAAKGGGGSGGGGS

SEQ ID NO: 909 - protein, 25 aa, synthetic construct
  REGION 1..25: note = Synthetic
EAAAKEAAAKEAAAKGGGGSGGGGS

SEQ ID NO: 910 - protein, 20 aa, synthetic construct
  REGION 1..20: note = Synthetic
EAAAKEAAAKEAAAKEAAAK

SEQ ID NO: 911 - protein, 25 aa, synthetic construct
  REGION 1..25: note = Synthetic
GGGGSEAAAKEAAAKGGGGSEAAAK

SEQ ID NO: 912 - protein, 25 aa, synthetic construct
  REGION 1..25: note = Synthetic
EAAAKEAAAKGGGGSGGGGSGGGGS

SEQ ID NO: 913 - protein, 25 aa, synthetic construct
  REGION 1..25: note = Synthetic
EAAAKEAAAKGGGGSGGGGSEAAAK

SEQ ID NO: 914 - skipped

SEQ ID NO: 915 - protein, 5 aa, synthetic construct
  REGION 1..5: note = Synthetic
GGGGS

SEQ ID NO: 916 - protein, 5 aa, synthetic construct
  REGION 1..5: note = Synthetic
EAAAK

SEQ ID NO: 917 - protein, 5 aa, synthetic construct
  REGION 1..5: note = Synthetic
SEGSA

SEQ ID NO: 918 - protein, 9 aa, synthetic construct
  REGION 1..9: note = Synthetic
SEGSAGTST

SEQ ID NO: 919 - protein, 10 aa, synthetic construct
  REGION 1..10: note = Synthetic
GGGGSGGGGS

SEQ ID NO: 920 - protein, 10 aa, synthetic construct
  REGION 1..10: note = Synthetic
GGGGSEAAAK

SEQ ID NO: 921 - protein, 10 aa, synthetic construct
  REGION 1..10: note = Synthetic
EAAAKGGGGS

SEQ ID NO: 922 - protein, 10 aa, synthetic construct
  REGION 1..10: note = Synthetic
EAAAKEAAAK

SEQ ID NO: 923 - protein, 15 aa, synthetic construct
  REGION 1..15: note = Synthetic
SEGSAGTSTESEGSA

SEQ ID NO: 924 - protein, 15 aa, synthetic construct
  REGION 1..15: note = Synthetic
GGGGSGGGGSGGGGS

SEQ ID NO: 925 - protein, 15 aa, synthetic construct
  REGION 1..15: note = Synthetic
GGGGSGGGGSEAAAK

SEQ ID NO: 926 - protein, 15 aa, synthetic construct
  REGION 1..15: note = Synthetic
GGGGSEAAAKGGGGS

SEQ ID NO: 927 - protein, 15 aa, synthetic construct
  REGION 1..15: note = Synthetic
EAAAKGGGGSEAAAK

SEQ ID NO: 928 - protein, 15 aa, synthetic construct
  REGION 1..15: note = Synthetic
EAAAKEAAAKGGGGS

SEQ ID NO: 929 - protein, 20 aa, synthetic construct
  REGION 1..20: note = Synthetic
SEGSAGTSTESEGSAGTSTE

SEQ ID NO: 930 - protein, 20 aa, synthetic construct
  REGION 1..20: note = Synthetic
GGGGSGGGGSGGGGSEAAAK

SEQ ID NO: 931 - protein, 20 aa, synthetic construct
  REGION 1..20: note = Synthetic
GGGGSGGGGSEAAAKGGGGS

SEQ ID NO: 932 - protein, 20 aa, synthetic construct
  REGION 1..20: note = Synthetic
GGGGSEAAAKGGGGSEAAAK

SEQ ID NO: 933 - protein, 20 aa, synthetic construct
  REGION 1..20: note = Synthetic
GGGGSEAAAKEAAAKGGGGS

SEQ ID NO: 934 - protein, 20 aa, synthetic construct
  REGION 1..20: note = Synthetic
GGGGSEAAAKEAAAKEAAAK

SEQ ID NO: 935 - protein, 20 aa, synthetic construct
  REGION 1..20: note = Synthetic
EAAAKGGGGSGGGGSGGGGS

SEQ ID NO: 936 - protein, 20 aa, synthetic construct
  REGION 1..20: note = Synthetic
EAAAKGGGGSGGGGSEAAAK

SEQ ID NO: 937 - protein, 20 aa, synthetic construct
  REGION 1..20: note = Synthetic
EAAAKGGGGSEAAAKGGGGS

SEQ ID NO: 938 - protein, 20 aa, synthetic construct
  REGION 1..20: note = Synthetic
EAAAKGGGGSEAAAKEAAAK

SEQ ID NO: 939 - protein, 20 aa, synthetic construct
  REGION 1..20: note = Synthetic
EAAAKEAAAKGGGGSGGGGS

SEQ ID NO: 940 - protein, 20 aa, synthetic construct
  REGION 1..20: note = Synthetic
EAAAKEAAAKGGGGSEAAAK

SEQ ID NO: 941 - protein, 20 aa, synthetic construct
  REGION 1..20: note = Synthetic
EAAAKEAAAKEAAAKGGGGS

SEQ ID NO: 942 - protein, 25 aa, synthetic construct
  REGION 1..25: note = Synthetic
SEGSAGTSTESEGSAGTSTESEGSA

SEQ ID NO: 943 - protein, 25 aa, synthetic construct
  REGION 1..25: note = Synthetic
GGGGSGGGGSGGGGSGGGGSGGGGS

SEQ ID NO: 944 - protein, 25 aa, synthetic construct
  REGION 1..25: note = Synthetic
GGGGSGGGGSGGGGSGGGGSEAAAK

SEQ ID NO: 945 - protein, 25 aa, synthetic construct
  REGION 1..25: note = Synthetic
GGGGSGGGGSGGGGSEAAAKGGGGS

SEQ ID NO: 946 - protein, 25 aa, synthetic construct
  REGION 1..25: note = Synthetic
GGGGSGGGGSGGGGSEAAAKEAAAK

SEQ ID NO: 947 - protein, 25 aa, synthetic construct
  REGION 1..25: note = Synthetic
GGGGSGGGGSEAAAKGGGGSGGGGS

SEQ ID NO: 948 - protein, 25 aa, synthetic construct
  REGION 1..25: note = Synthetic
GGGGSGGGGSEAAAKGGGGSEAAAK

SEQ ID NO: 949 - protein, 25 aa, synthetic construct
  REGION 1..25: note = Synthetic
GGGGSGGGGSEAAAKEAAAKGGGGS

SEQ ID NO: 950 - protein, 25 aa, synthetic construct
  REGION 1..25: note = Synthetic
GGGGSGGGGSEAAAKEAAAKEAAAK

SEQ ID NO: 951 - protein, 25 aa, synthetic construct
  REGION 1..25: note = Synthetic
GGGGSEAAAKGGGGSGGGGSGGGGS

SEQ ID NO: 952 - protein, 25 aa, synthetic construct
  REGION 1..25: note = Synthetic
GGGGSEAAAKGGGGSGGGGSEAAAK

SEQ ID NO: 953 - protein, 25 aa, synthetic construct
  REGION 1..25: note = Synthetic
GGGGSEAAAKGGGGSEAAAKGGGGS

SEQ ID NO: 954 - protein, 25 aa, synthetic construct
  REGION 1..25: note = Synthetic
GGGGSEAAAKGGGGSEAAAKEAAAK

SEQ ID NO: 955 - protein, 25 aa, synthetic construct
  REGION 1..25: note = Synthetic
GGGGSEAAAKEAAAKGGGGSGGGGS

SEQ ID NO: 956 - protein, 25 aa, synthetic construct
  REGION 1..25: note = Synthetic
GGGGSEAAAKEAAAKEAAAKGGGGS

SEQ ID NO: 957 - protein, 25 aa, synthetic construct
  REGION 1..25: note = Synthetic
GGGGSEAAAKEAAAKEAAAKEAAAK

SEQ ID NO: 958 - protein, 25 aa, synthetic construct
  REGION 1..25: note = Synthetic
EAAAKGGGGSGGGGSGGGGSGGGGS

SEQ ID NO: 959 - protein, 25 aa, synthetic construct
  REGION 1..25: note = Synthetic
EAAAKGGGGSGGGGSGGGGSEAAAK

SEQ ID NO: 960 - protein, 25 aa, synthetic construct
  REGION 1..25: note = Synthetic
EAAAKGGGGSGGGGSEAAAKGGGGS

SEQ ID NO: 961 - protein, 25 aa, synthetic construct
  REGION 1..25: note = Synthetic
EAAAKGGGGSGGGGSEAAAKEAAAK

SEQ ID NO: 962 - protein, 25 aa, synthetic construct
  REGION 1..25: note = Synthetic
EAAAKGGGGSEAAAKGGGGSGGGGS

SEQ ID NO: 963 - protein, 25 aa, synthetic construct
  REGION 1..25: note = Synthetic
EAAAKGGGGSEAAAKGGGGSEAAAK

SEQ ID NO: 964 - protein, 25 aa, synthetic construct
  REGION 1..25: note = Synthetic
EAAAKGGGGSEAAAKEAAAKGGGGS

SEQ ID NO: 965 - protein, 25 aa, synthetic construct
  REGION 1..25: note = Synthetic
EAAAKGGGGSEAAAKEAAAKEAAAK

SEQ ID NO: 966 - protein, 25 aa, synthetic construct
  REGION 1..25: note = Synthetic
EAAAKEAAAKGGGGSEAAAKGGGGS

SEQ ID NO: 967 - protein, 25 aa, synthetic construct
  REGION 1..25: note = Synthetic
EAAAKEAAAKGGGGSEAAAKEAAAK

SEQ ID NO: 968 - protein, 25 aa, synthetic construct
  REGION 1..25: note = Synthetic
EAAAKEAAAKEAAAKGGGGSEAAAK

SEQ ID NO: 969 - protein, 25 aa, synthetic construct
  REGION 1..25: note = Synthetic
EAAAKEAAAKEAAAKEAAAKGGGGS

SEQ ID NO: 970 - protein, 25 aa, synthetic construct
  REGION 1..25: note = Synthetic
EAAAKEAAAKEAAAKEAAAKEAAAK

SEQ ID NO: 971 - protein, 4 aa, synthetic construct
  REGION 1..4: note = Synthetic
SGGS

SEQ ID NO: 972 - RNA, 5342 nt, synthetic construct
  misc_feature 1..5342: note = Synthetic
  source 1..5342: mol_type = other RNA
gggaagctcagaataaacgctcaactttggccggatctgccaccatggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 973 - RNA, 5399 nt, synthetic construct
  misc_feature 1..5399: note = Synthetic
  source 1..5399: mol_type = other RNA
gggaagctcagaataaacgctcaactttggccggatctgccaccatggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 974 - RNA, 5458 nt, synthetic construct
  misc_feature 1..5458: note = Synthetic
  source 1..5458: mol_type = other RNA
gggagacccaagctggctagcgtttaaacttaagctttcccgcagtcggcgtccagcggctctgcttgttcgtgtgtgtgtcgttgcaggccttattcggatccgccaccatggaagcaagcccggcaagcggaccgagacacctgatggacccgcacatcttcacaagcaacttcaacaacggaatcggaagacacaagacatacctgtgctacgaagtcgaaagactggacaacggaacaagcgtcaagatggaccagcacagaggattcctgcacaaccaggcaaagaacctgctgtgcggattctacggaagacacgcagaactgagattcctggacctggtcccgagcctgcagctggacccggcacagatctacagagtcacatggttcatcagctggagcccgtgcttcagctggggatgcgcaggagaagtcagagcatttctgcaggaaaacacacacgtcagactgagaatcttcgcagcaagaatctacgactacgacccgctgtacaaggaagcactgcagatgctgagagacgcaggagcacaggtcagcatcatgacatacgacgaattcaagcactgctgggacacattcgtcgaccaccagggatgcccgttccagccgtgggacggactggacgaacacagccaggcactgagcggaagactgagagcaatcctgcagaaccagggaaacagcggaagcgaaacaccgggaacaagcgaaagcgcaacaccggaaagcgacaagaagtacagcatcggactggccatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgatctgagccagctgggaggagacagcggaggaagcacaaacctgagcgacatcatcgaaaaggaaacaggaaagcagctggtcatccaggaaagcatcctgatgctgccggaagaagtcgaagaagtcatcggaaacaagccggaaagcgacatcctggtccacacagcatacgacgaaagcacagacgaaaacgtcatgctgctgacaagcgacgcaccggaatacaagccgtgggcactggtcatccaggacagcaacggagaaaacaagatcaagatgctgagcggaggaagcccgaagaagaagagaaaggtctaatagtctagacatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaagcgaaaaaaaaaaaaaaaaaaaaaaaaaaaaaaccgaaaaaaaaaaaaaaaaaaaaaaaaaaaaaaaaaaaaaaat

SEQ ID NO: 975 - RNA, 589 nt, synthetic construct
  misc_feature 1..589: note = Synthetic
  source 1..589: mol_type = other RNA
gggagacccaagctggctagctcccgcagtcggcgtccagcggctctgcttgttcgtgtgtgtgtcgttgcaggccttattcggatccgccaccatgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtgatagtctagacatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagtctag

SEQ ID NO: 976 - RNA, 91 nt, synthetic construct
  misc_feature 1..91: note = Synthetic
  variation 1..20: note = n is a, c, g, or u
  source 1..91: mol_type = other RNA
nnnnnnnnnnnnnnnnnnnngttttagagctagaaatagcaagttaaaataaggctagtccgttatcacgaaagggcaccgagtcggtgct

SEQ ID NO: 977 - RNA, 91 nt, synthetic construct; identical to SEQ ID NO: 976
  misc_feature 1..91: note = Synthetic
  variation 1..91: note = 2'-O-methylation and phosphorothioate internucleoside linkage modifications are present or absent
  variation 1..20: note = n is a, c, g, or u
  source 1..91: mol_type = other RNA

SEQ ID NO: 978 - RNA, 68 nt, synthetic construct
  misc_feature 1..68: note = Synthetic
  variation 1..68: note = 2'-O-methylation and phosphorothioate internucleoside linkage modifications are present or absent
  source 1..68: mol_type = other RNA
gttttagagctagaaatagcaagttaaaataaggctagtccgttatcaaaatggcaccgagtcggtgc

SEQ ID NO: 979 - RNA, 80 nt, synthetic construct; identical to SEQ ID NO: 171
  misc_feature 1..80: note = Synthetic
  variation 1..80: note = 2'-O-methylation and phosphorothioate internucleoside linkage modifications are present or absent
  source 1..80: mol_type = other RNA

SEQ ID NO: 980 - RNA, 100 nt, synthetic construct
  misc_feature 1..100: note = Synthetic
  variation 1..100: note = 2'-O-methylation and phosphorothioate internucleoside linkage modifications are present or absent
  variation 1..20: note = n is a, c, g, or u
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 981-1810 - skipped (830 entries)

SEQ ID NO: 1811 - RNA, 100 nt, synthetic construct
  misc_feature 1..100: note = Synthetic
  source 1..100: mol_type = other RNA
tggagggcctgatgtgtgttgttttagagctagaaatagcaagttaaaataaggctagtccgttatcaacttgaaaaagtggcaccgagtcggtgctttt

SEQ ID NO: 1812 - RNA, 100 nt, synthetic construct
  misc_feature 1..100: note = Synthetic
  source 1..100: mol_type = other RNA
gcctgatgtgtgttgggtgtgttttagagctagaaatagcaagttaaaataaggctagtccgttatcaacttgaaaaagtggcaccgagtcggtgctttt

SEQ ID NO: 1813 - RNA, 100 nt, synthetic construct
  misc_feature 1..100: note = Synthetic
  source 1..100: mol_type = other RNA
cctgatgtgtgttgggtgttgttttagagctagaaatagcaagttaaaataaggctagtccgttatcaacttgaaaaagtggcaccgagtcggtgctttt

SEQ ID NO: 1814 - RNA, 100 nt, synthetic construct
  misc_feature 1..100: note = Synthetic
  source 1..100: mol_type = other RNA
cccaacacccaacacacatcgttttagagctagaaatagcaagttaaaataaggctagtccgttatcaacttgaaaaagtggcaccgagtcggtgctttt

SEQ ID NO: 1815 - RNA, 100 nt, synthetic construct
  misc_feature 1..100: note = Synthetic
  source 1..100: mol_type = other RNA
tcaggaaacatgaagaaagcgttttagagctagaaatagcaagttaaaataaggctagtccgttatcaacttgaaaaagtggcaccgagtcggtgctttt

SEQ ID NO: 1816 - RNA, 100 nt, synthetic construct
  misc_feature 1..100: note = Synthetic
  source 1..100: mol_type = other RNA
aggcgcctgggcctctcccggttttagagctagaaatagcaagttaaaataaggctagtccgttatcaacttgaaaaagtggcaccgagtcggtgctttt

SEQ ID NO: 1817 - RNA, 100 nt, synthetic construct
  misc_feature 1..100: note = Synthetic
  source 1..100: mol_type = other RNA
cgggctggcctcccacaagggttttagagctagaaatagcaagttaaaataaggctagtccgttatcaacttgaaaaagtggcaccgagtcggtgctttt

SEQ ID NO: 1818 - RNA, 100 nt, synthetic construct
  misc_feature 1..100: note = Synthetic
  source 1..100: mol_type = other RNA
acggccatcctcggcgtctggttttagagctagaaatagcaagttaaaataaggctagtccgttatcaacttgaaaaagtggcaccgagtcggtgctttt

SEQ ID NO: 1819 - RNA, 100 nt, synthetic construct
  misc_feature 1..100: note = Synthetic
  source 1..100: mol_type = other RNA
gacggccatcctcggcgtctgttttagagctagaaatagcaagttaaaataaggctagtccgttatcaacttgaaaaagtggcaccgagtcggtgctttt

SEQ ID NO: 1820 - RNA, 100 nt, synthetic construct
  misc_feature 1..100: note = Synthetic
  source 1..100: mol_type = other RNA
gacgccgaggatggccgtcagttttagagctagaaatagcaagttaaaataaggctagtccgttatcaacttgaaaaagtggcaccgagtcggtgctttt

SEQ ID NO: 1821 - RNA, 100 nt, synthetic construct
  misc_feature 1..100: note = Synthetic
  source 1..100: mol_type = other RNA
tgacggccatcctcggcgtcgttttagagctagaaatagcaagttaaaataaggctagtccgttatcaacttgaaaaagtggcaccgagtcggtgctttt

SEQ ID NO: 1822 - RNA, 100 nt, synthetic construct
  misc_feature 1..100: note = Synthetic
  source 1..100: mol_type = other RNA
ggcgccatgacggccatcctgttttagagctagaaatagcaagttaaaataaggctagtccgttatcaacttgaaaaagtggcaccgagtcggtgctttt

SEQ ID NO: 1823 - RNA, 100 nt, synthetic construct
  misc_feature 1..100: note = Synthetic
  source 1..100: mol_type = other RNA
acagcgacgccgcgagccaggttttagagctagaaatagcaagttaaaataaggctagtccgttatcaacttgaaaaagtggcaccgagtcggtgctttt

SEQ ID NO: 1824 - RNA, 100 nt, synthetic construct
  misc_feature 1..100: note = Synthetic
  source 1..100: mol_type = other RNA
cgacgccgcgagccagaggagttttagagctagaaatagcaagttaaaataaggctagtccgttatcaacttgaaaaagtggcaccgagtcggtgctttt

SEQ ID NO: 1825 - RNA, 100 nt, synthetic construct
  misc_feature 1..100: note = Synthetic
  source 1..100: mol_type = other RNA
cgagccagaggatggagccggttttagagctagaaatagcaagttaaaataaggctagtccgttatcaacttgaaaaagtggcaccgagtcggtgctttt

SEQ ID NO: 1826 - RNA, 100 nt, synthetic construct
  misc_feature 1..100: note = Synthetic
  source 1..100: mol_type = other RNA
cggctccatcctctggctcggttttagagctagaaatagcaagttaaaataaggctagtccgttatcaacttgaaaaagtggcaccgagtcggtgctttt

SEQ ID NO: 1827 - RNA, 100 nt, synthetic construct
  misc_feature 1..100: note = Synthetic
  source 1..100: mol_type = other RNA
gagccagaggatggagccgcgttttagagctagaaatagcaagttaaaataaggctagtccgttatcaacttgaaaaagtggcaccgagtcggtgctttt

SEQ ID NO: 1828 - RNA, 100 nt, synthetic construct
  misc_feature 1..100: note = Synthetic
  source 1..100: mol_type = other RNA
gcgcccgcggctccatcctcgttttagagctagaaatagcaagttaaaataaggctagtccgttatcaacttgaaaaagtggcaccgagtcggtgctttt

SEQ ID NO: 1829 - RNA, 100 nt, synthetic construct
  misc_feature 1..100: note = Synthetic
  source 1..100: mol_type = other RNA
gcccgtccgtgggggatgaggttttagagctagaaatagcaagttaaaataaggctagtccgttatcaacttgaaaaagtggcaccgagtcggtgctttt

SEQ ID NO: 1830 - RNA, 100 nt, synthetic construct
  misc_feature 1..100: note = Synthetic
  source 1..100: mol_type = other RNA
tcatcccccacggacgggccgttttagagctagaaatagcaagttaaaataaggctagtccgttatcaacttgaaaaagtggcaccgagtcggtgctttt

SEQ ID NO: 1831 - RNA, 100 nt, synthetic construct
  misc_feature 1..100: note = Synthetic
  source 1..100: mol_type = other RNA
atctcggacccggagactgtgttttagagctagaaatagcaagttaaaataaggctagtccgttatcaacttgaaaaagtggcaccgagtcggtgctttt

SEQ ID NO: 1832 - RNA, 100 nt, synthetic construct
  misc_feature 1..100: note = Synthetic
  source 1..100: mol_type = other RNA
ggggtcccgcggcttcgggggttttagagctagaaatagcaagttaaaataaggctagtccgttatcaacttgaaaaagtggcaccgagtcggtgctttt

SEQ ID NO: 1833 - RNA, 100 nt, synthetic construct
  misc_feature 1..100: note = Synthetic
  source 1..100: mol_type = other RNA
ctcggggtcccgcggcttcggttttagagctagaaatagcaagttaaaataaggctagtccgttatcaacttgaaaaagtggcaccgagtcggtgctttt

SEQ ID NO: 1834 - RNA, 100 nt, synthetic construct
  misc_feature 1..100: note = Synthetic
  source 1..100: mol_type = other RNA
tctcggggtcccgcggcttcgttttagagctagaaatagcaagttaaaataaggctagtccgttatcaacttgaaaaagtggcaccgagtcggtgctttt

SEQ ID NO: 1835 - RNA, 100 nt, synthetic construct
  misc_feature 1..100: note = Synthetic
  source 1..100: mol_type = other RNA
gtctcggggtcccgcggcttgttttagagctagaaatagcaagttaaaataaggctagtccgttatcaacttgaaaaagtggcaccgagtcggtgctttt

SEQ ID NO: 1836 - RNA, 100 nt, synthetic construct
  misc_feature 1..100: note = Synthetic
  source 1..100: mol_type = other RNA
gcaagggtctcggggtcccggttttagagctagaaatagcaagttaaaataaggctagtccgttatcaacttgaaaaagtggcaccgagtcggtgctttt

SEQ ID NO: 1837 - RNA, 100 nt, synthetic construct
  misc_feature 1..100: note = Synthetic
  source 1..100: mol_type = other RNA
ggaccccgagacccttgcccgttttagagctagaaatagcaagttaaaataaggctagtccgttatcaacttgaaaaagtggcaccgagtcggtgctttt

SEQ ID NO: 1838 - RNA, 100 nt, synthetic construct
  misc_feature 1..100: note = Synthetic
  source 1..100: mol_type = other RNA
gaccccgagacccttgccccgttttagagctagaaatagcaagttaaaataaggctagtccgttatcaacttgaaaaagtggcaccgagtcggtgctttt

SEQ ID NO: 1839 - RNA, 100 nt, synthetic construct
  misc_feature 1..100: note = Synthetic
  source 1..100: mol_type = other RNA
cgagacccttgccccgggaggttttagagctagaaatagcaagttaaaataaggctagtccgttatcaacttgaaaaagtggcaccgagtcggtgctttt

SEQ ID NO: 1840 - RNA, 100 nt, synthetic construct
  misc_feature 1..100: note = Synthetic
  source 1..100: mol_type = other RNA
ctcccggggcaagggtctcggttttagagctagaaatagcaagttaaaataaggctagtccgttatcaacttgaaaaagtggcaccgagtcggtgctttt

SEQ ID NO: 1841 - RNA, 100 nt, synthetic construct
  misc_feature 1..100: note = Synthetic
  source 1..100: mol_type = other RNA
tctcccggggcaagggtctcgttttagagctagaaatagcaagttaaaataaggctagtccgttatcaacttgaaaaagtggcaccgagtcggtgctttt

SEQ ID NO: 1842 - RNA, 100 nt, synthetic construct
  misc_feature 1..100: note = Synthetic
  source 1..100: mol_type = other RNA
ctctcccggggcaagggtctgttttagagctagaaatagcaagttaaaataaggctagtccgttatcaacttgaaaaagtggcaccgagtcggtgctttt

SEQ ID NO: 1843 - RNA, 100 nt, synthetic construct
  misc_feature 1..100: note = Synthetic
  source 1..100: mol_type = other RNA
ccttgccccgggagaggcccgttttagagctagaaatagcaagttaaaataaggctagtccgttatcaacttgaaaaagtggcaccgagtcggtgctttt

SEQ ID NO: 1844 - RNA, 100 nt, synthetic construct
  misc_feature 1..100: note = Synthetic
  source 1..100: mol_type = other RNA
ctgggcctctcccggggcaagttttagagctagaaatagcaagttaaaataaggctagtccgttatcaacttgaaaaagtggcaccgagtcggtgctttt

SEQ ID NO: 1845 - RNA, 100 nt, synthetic construct
  misc_feature 1..100: note = Synthetic
  source 1..100: mol_type = other RNA
cctgggcctctcccggggcagttttagagctagaaatagcaagttaaaataaggctagtccgttatcaacttgaaaaagtggcaccgagtcggtgctttt

SEQ ID NO: 1846 - RNA, 100 nt, synthetic construct
  misc_feature 1..100: note = Synthetic
  source 1..100: mol_type = other RNA
tttaggccaaaaatccccccgttttagagctagaaatagcaagttaaaataaggctagtccgttatcaacttgaaaaagtggcaccgagtcggtgctttt

SEQ ID NO: 1847 - RNA, 100 nt, synthetic construct
  misc_feature 1..100: note = Synthetic
  source 1..100: mol_type = other RNA
cgctgcagcgcacgggtaccgttttagagctagaaatagcaagttaaaataaggctagtccgttatcaacttgaaaaagtggcaccgagtcggtgctttt

SEQ ID NO: 1848 - RNA, 100 nt, synthetic construct
  misc_feature 1..100: note = Synthetic
  source 1..100: mol_type = other RNA
gctgcagcgcacgggtaccagttttagagctagaaatagcaagttaaaataaggctagtccgttatcaacttgaaaaagtggcaccgagtcggtgctttt

SEQ ID NO: 1849 - RNA, 100 nt, synthetic construct
  misc_feature 1..100: note = Synthetic
  source 1..100: mol_type = other RNA
ctgcagcgcacgggtaccaggttttagagctagaaatagcaagttaaaataaggctagtccgttatcaacttgaaaaagtggcaccgagtcggtgctttt

SEQ ID NO: 1850 - RNA, 100 nt, synthetic construct
  misc_feature 1..100: note = Synthetic
  source 1..100: mol_type = other RNA
cgcacgggtaccaggggccagttttagagctagaaatagcaagttaaaataaggctagtccgttatcaacttgaaaaagtggcaccgagtcggtgctttt

SEQ ID NO: 1851 - RNA, 100 nt, synthetic construct
  misc_feature 1..100: note = Synthetic
  source 1..100: mol_type = other RNA
gcacgggtaccaggggccacgttttagagctagaaatagcaagttaaaataaggctagtccgttatcaacttgaaaaagtggcaccgagtcggtgctttt

SEQ ID NO: 1852 - RNA, 100 nt, synthetic construct
  misc_feature 1..100: note = Synthetic
  source 1..100: mol_type = other RNA
cacgggtaccaggggccacggttttagagctagaaatagcaagttaaaataaggctagtccgttatcaacttgaaaaagtggcaccgagtcggtgctttt

SEQ ID NO: 1853 - RNA, 100 nt, synthetic construct
  misc_feature 1..100: note = Synthetic
  source 1..100: mol_type = other RNA
gggaggcgccccgtggccccgttttagagctagaaatagcaagttaaaataaggctagtccgttatcaacttgaaaaagtggcaccgagtcggtgctttt

SEQ ID NO: 1854 - RNA, 100 nt, synthetic construct
  misc_feature 1..100: note = Synthetic
  source 1..100: mol_type = other RNA
gcgatcagggaggcgccccggttttagagctagaaatagcaagttaaaataaggctagtccgttatcaacttgaaaaagtggcaccgagtcggtgctttt

SEQ ID NO: 1855 - RNA, 100 nt, synthetic construct
  misc_feature 1..100: note = Synthetic
  source 1..100: mol_type = other RNA
tccttgtgggaggccagcccgttttagagctagaaatagcaagttaaaataaggctagtccgttatcaacttgaaaaagtggcaccgagtcggtgctttt

SEQ ID NO: 1856 - RNA, 100 nt, synthetic construct
  misc_feature 1..100: note = Synthetic
  source 1..100: mol_type = other RNA
ctccttgtgggaggccagccgttttagagctagaaatagcaagttaaaataaggctagtccgttatcaacttgaaaaagtggcaccgagtcggtgctttt

SEQ ID NO: 1857 - RNA, 100 nt, synthetic construct
  misc_feature 1..100: note = Synthetic
  source 1..100: mol_type = other RNA
ggctggcctcccacaaggaggttttagagctagaaatagcaagttaaaataaggctagtccgttatcaacttgaaaaagtggcaccgagtcggtgctttt

SEQ ID NO: 1858 - RNA, 100 nt, synthetic construct
  misc_feature 1..100: note = Synthetic
  source 1..100: mol_type = other RNA
ttgtctcccctccttgtggggttttagagctagaaatagcaagttaaaataaggctagtccgttatcaacttgaaaaagtggcaccgagtcggtgctttt

SEQ ID NO: 1859 - RNA, 100 nt, synthetic construct
  misc_feature 1..100: note = Synthetic
  source 1..100: mol_type = other RNA
ccacaaggaggggagacaatgttttagagctagaaatagcaagttaaaataaggctagtccgttatcaacttgaaaaagtggcaccgagtcggtgctttt

SEQ ID NO: 1860 - RNA, 100 nt, synthetic construct
  misc_feature 1..100: note = Synthetic
  source 1..100: mol_type = other RNA
cacaaggaggggagacaattgttttagagctagaaatagcaagttaaaataaggctagtccgttatcaacttgaaaaagtggcaccgagtcggtgctttt

SEQ ID NO: 1861 - RNA, 100 nt, synthetic construct
  misc_feature 1..100: note = Synthetic
  source 1..100: mol_type = other RNA
caattgtctcccctccttgtgttttagagctagaaatagcaagttaaaataaggctagtccgttatcaacttgaaaaagtggcaccgagtcggtgctttt

SEQ ID NO: 1862 - RNA, 100 nt, synthetic construct
  misc_feature 1..100: note = Synthetic
  source 1..100: mol_type = other RNA
ccaattgtctcccctccttggttttagagctagaaatagcaagttaaaataaggctagtccgttatcaacttgaaaaagtggcaccgagtcggtgctttt

SEQ ID NO: 1863 - RNA, 100 nt, synthetic construct
  misc_feature 1..100: note = Synthetic
  source 1..100: mol_type = other RNA
atccctcgaatactgatgaggttttagagctagaaatagcaagttaaaataaggctagtccgttatcaacttgaaaaagtggcaccgagtcggtgctttt

SEQ ID NO: 1864 - RNA, 100 nt, synthetic construct
  misc_feature 1..100: note = Synthetic
  source 1..100: mol_type = other RNA
aaccactcatcagtattcgagttttagagctagaaatagcaagttaaaataaggctagtccgttatcaacttgaaaaagtggcaccgagtcggtgctttt

SEQ ID NO: 1865 - RNA, 100 nt, synthetic construct
  misc_feature 1..100: note = Synthetic
  source 1..100: mol_type = other RNA
gaaccactcatcagtattcggttttagagctagaaatagcaagttaaaataaggctagtccgttatcaacttgaaaaagtggcaccgagtcggtgctttt

SEQ ID NO: 1866 - RNA, 100 nt, synthetic construct
  misc_feature 1..100: note = Synthetic
  source 1..100: mol_type = other RNA
gaggaaaagtcacgggcccagttttagagctagaaatagcaagttaaaataaggctagtccgttatcaacttgaaaaagtggcaccgagtcggtgctttt

SEQ ID NO: 1867 - RNA, 100 nt, synthetic construct
  misc_feature 1..100: note = Synthetic
  source 1..100: mol_type = other RNA
ggcccgtgacttttcctctcgttttagagctagaaatagcaagttaaaataaggctagtccgttatcaacttgaaaaagtggcaccgagtcggtgctttt

SEQ ID NO: 1868 - RNA, 100 nt, synthetic construct
  misc_feature 1..100: note = Synthetic
  source 1..100: mol_type = other RNA
tgcttcacactcaatgtgtggttttagagctagaaatagcaagttaaaataaggctagtccgttatcaacttgaaaaagtggcaccgagtcggtgctttt

SEQ ID NO: 1869 - RNA, 100 nt, synthetic construct
  misc_feature 1..100: note = Synthetic
  source 1..100: mol_type = other RNA
gcttcacactcaatgtgtgtgttttagagctagaaatagcaagttaaaataaggctagtccgttatcaacttgaaaaagtggcaccgagtcggtgctttt

SEQ ID NO: 1870 - RNA, 100 nt, synthetic construct
  misc_feature 1..100: note = Synthetic
  source 1..100: mol_type = other RNA
cttcacactcaatgtgtgtggttttagagctagaaatagcaagttaaaataaggctagtccgttatcaacttgaaaaagtggcaccgagtcggtgctttt

SEQ ID NO: 1871 - RNA, 100 nt, synthetic construct
  misc_feature 1..100: note = Synthetic
  source 1..100: mol_type = other RNA
ttcacactcaatgtgtgtgggttttagagctagaaatagcaagttaaaataaggctagtccgttatcaacttgaaaaagtggcaccgagtcggtgctttt

SEQ ID NO: 1872 - RNA, 100 nt, synthetic construct
  misc_feature 1..100: note = Synthetic
  source 1..100: mol_type = other RNA
ttgagaatggacaggacaccgttttagagctagaaatagcaagttaaaataaggctagtccgttatcaacttgaaaaagtggcaccgagtcggtgctttt

SEQ ID NO: 1873 - RNA, 100 nt, synthetic construct
  misc_feature 1..100: note = Synthetic
  source 1..100: mol_type = other RNA
aggcattttgcatctgtcatgttttagagctagaaatagcaagttaaaataaggctagtccgttatcaacttgaaaaagtggcaccgagtcggtgctttt

SEQ ID NO: 1874 - RNA, 100 nt, synthetic construct
  misc_feature 1..100: note = Synthetic
  source 1..100: mol_type = other RNA
caggcattttgcatctgtcagttttagagctagaaatagcaagttaaaataaggctagtccgttatcaacttgaaaaagtggcaccgagtcggtgctttt

SEQ ID NO: 1875 - RNA, 100 nt, synthetic construct
  misc_feature 1..100: note = Synthetic
  source 1..100: mol_type = other RNA
aggggccctgaccctgctaagttttagagctagaaatagcaagttaaaataaggctagtccgttatcaacttgaaaaagtggcaccgagtcggtgctttt

SEQ ID NO: 1876 - RNA, 100 nt, synthetic construct
  misc_feature 1..100: note = Synthetic
  source 1..100: mol_type = other RNA
tgggaaaagaggggaaggtggttttagagctagaaatagcaagttaaaataaggctagtccgttatcaacttgaaaaagtggcaccgagtcggtgctttt

SEQ ID NO: 1877 - RNA, 100 nt, synthetic construct
  misc_feature 1..100: note = Synthetic
  source 1..100: mol_type = other RNA
tggaggaggaagagctcagggttttagagctagaaatagcaagttaaaataaggctagtccgttatcaacttgaaaaagtggcaccgagtcggtgctttt

SEQ ID NO: 1878 - RNA, 100 nt, synthetic construct
  misc_feature 1..100: note = Synthetic
  source 1..100: mol_type = other RNA
tgagatttcttgtctcactggttttagagctagaaatagcaagttaaaataaggctagtccgttatcaacttgaaaaagtggcaccgagtcggtgctttt

SEQ ID NO: 1879 - RNA, 100 nt, synthetic construct
  misc_feature 1..100: note = Synthetic
  source 1..100: mol_type = other RNA
gagatttcttgtctcactgagttttagagctagaaatagcaagttaaaataaggctagtccgttatcaacttgaaaaagtggcaccgagtcggtgctttt

SEQ ID NO: 1880 - RNA, 100 nt, synthetic construct
  misc_feature 1..100: note = Synthetic
  source 1..100: mol_type = other RNA
taaagcacctgttaaaatgagttttagagctagaaatagcaagttaaaataaggctagtccgttatcaacttgaaaaagtggcaccgagtcggtgctttt

SEQ ID NO: 1881 - RNA, 100 nt, synthetic construct
  misc_feature 1..100: note = Synthetic
  source 1..100: mol_type = other RNA
aatctgtccttcattttaacgttttagagctagaaatagcaagttaaaataaggctagtccgttatcaacttgaaaaagtggcaccgagtcggtgctttt

SEQ ID NO: 1882 - RNA, 100 nt, synthetic construct
  misc_feature 1..100: note = Synthetic
  source 1..100: mol_type = other RNA
gtcacaggggaaggtccctggttttagagctagaaatagcaagttaaaataaggctagtccgttatcaacttgaaaaagtggcaccgagtcggtgctttt

SEQ ID NO: 1883 - RNA, 100 nt, synthetic construct
  misc_feature 1..100: note = Synthetic
  source 1..100: mol_type = other RNA
aaacatgaagaaagcaggtggttttagagctagaaatagcaagttaaaataaggctagtccgttatcaacttgaaaaagtggcaccgagtcggtgctttt

SEQ ID NO: 1884 - RNA, 100 nt, synthetic construct
  misc_feature 1..100: note = Synthetic
  source 1..100: mol_type = other RNA
tgtcctgtgagataccagaagttttagagctagaaatagcaagttaaaataaggctagtccgttatcaacttgaaaaagtggcaccgagtcggtgctttt

SEQ ID NO: 1885 - RNA, 100 nt, synthetic construct
  misc_feature 1..100: note = Synthetic
  source 1..100: mol_type = other RNA
atgaaggaggctgatgcctggttttagagctagaaatagcaagttaaaataaggctagtccgttatcaacttgaaaaagtggcaccgagtcggtgctttt

SEQ ID NO: 1886 - RNA, 100 nt, synthetic construct
  misc_feature 1..100: note = Synthetic
  source 1..100: mol_type = other RNA
aggctgatgcctgaggtcctgttttagagctagaaatagcaagttaaaataaggctagtccgttatcaacttgaaaaagtggcaccgagtcggtgctttt

SEQ ID NO: 1887 - RNA, 100 nt, synthetic construct
  misc_feature 1..100: note = Synthetic
  source 1..100: mol_type = other RNA
ggctgatgcctgaggtccttgttttagagctagaaatagcaagttaaaataaggctagtccgttatcaacttgaaaaagtggcaccgagtcggtgctttt

SEQ ID NO: 1888 - RNA, 100 nt, synthetic construct
  misc_feature 1..100: note = Synthetic
  source 1..100: mol_type = other RNA
cacaatatcccaaggacctcgttttagagctagaaatagcaagttaaaataaggctagtccgttatcaacttgaaaaagtggcaccgagtcggtgctttt

SEQ ID NO: 1889 - RNA, 100 nt, synthetic construct
  misc_feature 1..100: note = Synthetic
  source 1..100: mol_type = other RNA
ggtccttgggatattgtgttgttttagagctagaaatagcaagttaaaataaggctagtccgttatcaacttgaaaaagtggcaccgagtcggtgctttt

SEQ ID NO: 1890 - RNA, 100 nt, synthetic construct
  misc_feature 1..100: note = Synthetic
  source 1..100: mol_type = other RNA
gtccttgggatattgtgtttgttttagagctagaaatagcaagttaaaataaggctagtccgttatcaacttgaaaaagtggcaccgagtcggtgctttt

SEQ ID NO: 1891 - RNA, 100 nt, synthetic construct
  misc_feature 1..100: note = Synthetic
  source 1..100: mol_type = other RNA
ctcccaaacacaatatcccagttttagagctagaaatagcaagttaaaataaggctagtccgttatcaacttgaaaaagtggcaccgagtcggtgctttt

SEQ ID NO: 1892 - RNA, 100 nt, synthetic construct
  misc_feature 1..100: note = Synthetic
  source 1..100: mol_type = other RNA
tcctctagccacatcttctggttttagagctagaaatagcaagttaaaataaggctagtccgttatcaacttgaaaaagtggcaccgagtcggtgctttt

SEQ ID NO: 1893 - RNA, 100 nt, synthetic construct
  misc_feature 1..100: note = Synthetic
  source 1..100: mol_type = other RNA
acagaagatgtggctagagggttttagagctagaaatagcaagttaaaataaggctagtccgttatcaacttgaaaaagtggcaccgagtcggtgctttt

SEQ ID NO: 1894 - RNA, 100 nt, synthetic construct
  misc_feature 1..100: note = Synthetic
  source 1..100: mol_type = other RNA
cctctagccacatcttctgtgttttagagctagaaatagcaagttaaaataaggctagtccgttatcaacttgaaaaagtggcaccgagtcggtgctttt

SEQ ID NO: 1895 - RNA, 100 nt, synthetic construct
  misc_feature 1..100: note = Synthetic
  source 1..100: mol_type = other RNA
cccacagaagatgtggctaggttttagagctagaaatagcaagttaaaataaggctagtccgttatcaacttgaaaaagtggcaccgagtcggtgctttt

SEQ ID NO: 1896 - RNA, 100 nt, synthetic construct
  misc_feature 1..100: note = Synthetic
  source 1..100: mol_type = other RNA
gtcagatcccacagaagatggttttagagctagaaatagcaagttaaaataaggctagtccgttatcaacttgaaaaagtggcaccgagtcggtgctttt

SEQ ID NO: 1897 - RNA, 100 nt, synthetic construct
  misc_feature 1..100: note = Synthetic
  source 1..100: mol_type = other RNA
atcttctgtgggatctgaccgttttagagctagaaatagcaagttaaaataaggctagtccgttatcaacttgaaaaagtggcaccgagtcggtgctttt

SEQ ID NO: 1898 - RNA, 100 nt, synthetic construct
  misc_feature 1..100: note = Synthetic
  source 1..100: mol_type = other RNA
cccaggcagtgacagtgcccgttttagagctagaaatagcaagttaaaataaggctagtccgttatcaacttgaaaaagtggcaccgagtcggtgctttt

SEQ ID NO: 1899 - RNA, 100 nt, synthetic construct
  misc_feature 1..100: note = Synthetic
  source 1..100: mol_type = other RNA
ctgggcactgtcactgcctggttttagagctagaaatagcaagttaaaataaggctagtccgttatcaacttgaaaaagtggcaccgagtcggtgctttt

SEQ ID NO: 1900 - RNA, 100 nt, synthetic construct
  misc_feature 1..100: note = Synthetic
  source 1..100: mol_type = other RNA
cctgggcactgtcactgcctgttttagagctagaaatagcaagttaaaataaggctagtccgttatcaacttgaaaaagtggcaccgagtcggtgctttt

SEQ ID NO: 1901 - RNA, 100 nt, synthetic construct
  misc_feature 1..100: note = Synthetic
  source 1..100: mol_type = other RNA
ccctgggcactgtcactgccgttttagagctagaaatagcaagttaaaataaggctagtccgttatcaacttgaaaaagtggcaccgagtcggtgctttt

SEQ ID NO: 1902 - RNA, 100 nt, synthetic construct
  misc_feature 1..100: note = Synthetic
  source 1..100: mol_type = other RNA
ttgggtgttgggcggaacaggttttagagctagaaatagcaagttaaaataaggctagtccgttatcaacttgaaaaagtggcaccgagtcggtgctttt

SEQ ID NO: 1903 - RNA, 100 nt, synthetic construct
  misc_feature 1..100: note = Synthetic
  source 1..100: mol_type = other RNA
tggatgtattgagcatgcgagttttagagctagaaatagcaagttaaaataaggctagtccgttatcaacttgaaaaagtggcaccgagtcggtgctttt

SEQ ID NO: 1904 - RNA, 100 nt, synthetic construct
  misc_feature 1..100: note = Synthetic
  source 1..100: mol_type = other RNA
ggatgtattgagcatgcgatgttttagagctagaaatagcaagttaaaataaggctagtccgttatcaacttgaaaaagtggcaccgagtcggtgctttt

SEQ ID NO: 1905 - RNA, 100 nt, synthetic construct
  misc_feature 1..100: note = Synthetic
  source 1..100: mol_type = other RNA
aacatgaagaaagcaggtgtgttttagagctagaaatagcaagttaaaataaggctagtccgttatcaacttgaaaaagtggcaccgagtcggtgctttt

SEQ ID NO: 1906 - RNA, 100 nt, synthetic construct; identical to SEQ ID NO: 1811
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1907 - RNA, 100 nt, synthetic construct; identical to SEQ ID NO: 1812
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1908 - RNA, 100 nt, synthetic construct; identical to SEQ ID NO: 1813
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1909 - RNA, 100 nt, synthetic construct; identical to SEQ ID NO: 1814
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1910 - RNA, 100 nt, synthetic construct; identical to SEQ ID NO: 1815
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1911 - RNA, 100 nt, synthetic construct; identical to SEQ ID NO: 1816
  misc_feature 1..100: note = Synthetic
  variation 1..100: note = 2'-O-methylation and phosphorothioate internucleoside linkage modifications are present or absent for detailed description of substitutions and preferred
  source 1..100: mol_type = other RNA

SEQ ID NO: 1912 - RNA, 100 nt, synthetic construct; identical to SEQ ID NO: 1817
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1913 - RNA, 100 nt, synthetic construct; identical to SEQ ID NO: 1818
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1914 - RNA, 100 nt, synthetic construct; identical to SEQ ID NO: 1819
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1915 - RNA, 100 nt, synthetic construct; identical to SEQ ID NO: 1820
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1916 - RNA, 100 nt, synthetic construct; identical to SEQ ID NO: 1821
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1917 - RNA, 100 nt, synthetic construct; identical to SEQ ID NO: 1822
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1918 - RNA, 100 nt, synthetic construct; identical to SEQ ID NO: 1823
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1919 - RNA, 100 nt, synthetic construct; identical to SEQ ID NO: 1824
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1920 - RNA, 100 nt, synthetic construct; identical to SEQ ID NO: 1825
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1921 - RNA, 100 nt, synthetic construct; identical to SEQ ID NO: 1826
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1922 - RNA, 100 nt, synthetic construct; identical to SEQ ID NO: 1827
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1923 - RNA, 100 nt, synthetic construct; identical to SEQ ID NO: 1828
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1924 - RNA, 100 nt, synthetic construct; identical to SEQ ID NO: 1829
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1925 - RNA, 100 nt, synthetic construct; identical to SEQ ID NO: 1830
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1926 - RNA, 100 nt, synthetic construct; identical to SEQ ID NO: 1831
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1927 - RNA, 100 nt, synthetic construct; identical to SEQ ID NO: 1832
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1928 - RNA, 100 nt, synthetic construct; identical to SEQ ID NO: 1833
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1929 - RNA, 100 nt, synthetic construct; identical to SEQ ID NO: 1834
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1930 - RNA, 100 nt, synthetic construct; identical to SEQ ID NO: 1835
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1931 - RNA, 100 nt, synthetic construct; identical to SEQ ID NO: 1836
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1932 - RNA, 100 nt, synthetic construct; identical to SEQ ID NO: 1837
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1933 - RNA, 100 nt, synthetic construct; identical to SEQ ID NO: 1838
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1934 - RNA, 100 nt, synthetic construct; identical to SEQ ID NO: 1839
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1935 - RNA, 100 nt, synthetic construct; identical to SEQ ID NO: 1840
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1936 - RNA, 100 nt, synthetic construct; identical to SEQ ID NO: 1841
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1937 - RNA, 100 nt, synthetic construct; identical to SEQ ID NO: 1842
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1938 - RNA, 100 nt, synthetic construct; identical to SEQ ID NO: 1843
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1939 - RNA, 100 nt, synthetic construct; identical to SEQ ID NO: 1844
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1940 - RNA, 100 nt, synthetic construct; identical to SEQ ID NO: 1845
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1941 - RNA, 100 nt, synthetic construct; identical to SEQ ID NO: 1846
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1942 - RNA, 100 nt, synthetic construct; identical to SEQ ID NO: 1847
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1943 - RNA, 100 nt, synthetic construct; identical to SEQ ID NO: 1848
  misc_feature 1..100: note = Synthetic
  variation 1..100: note = 2'-O-methylation and phosphorothioate internucleoside linkage modifications are present or absent for detailed description of substitutions and preferred
  source 1..100: mol_type = other RNA

SEQ ID NO: 1944 - RNA, 100 nt, synthetic construct; identical to SEQ ID NO: 1849
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1945 - RNA, 100 nt, synthetic construct; identical to SEQ ID NO: 1850
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1946 - RNA, 100 nt, synthetic construct; identical to SEQ ID NO: 1851
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1947 - RNA, 100 nt, synthetic construct; identical to SEQ ID NO: 1852
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1948 - RNA, 100 nt, synthetic construct; identical to SEQ ID NO: 1853
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1949 - RNA, 100 nt, synthetic construct; identical to SEQ ID NO: 1854
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1950 - RNA, 100 nt, synthetic construct; identical to SEQ ID NO: 1855
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1951 - RNA, 100 nt, synthetic construct; identical to SEQ ID NO: 1856
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1952 - RNA, 100 nt, synthetic construct; identical to SEQ ID NO: 1857
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1953 - RNA, 100 nt, synthetic construct; identical to SEQ ID NO: 1858
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1954 - RNA, 100 nt, synthetic construct; identical to SEQ ID NO: 1859
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1955 - RNA, 100 nt, synthetic construct; identical to SEQ ID NO: 1860
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1956 - RNA, 100 nt, synthetic construct; identical to SEQ ID NO: 1861
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1957 - RNA, 100 nt, synthetic construct; identical to SEQ ID NO: 1862
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1958 - RNA, 100 nt, synthetic construct; identical to SEQ ID NO: 1863
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1959 - RNA, 100 nt, synthetic construct; identical to SEQ ID NO: 1864
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1960 - RNA, 100 nt, synthetic construct; identical to SEQ ID NO: 1865
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1961 - RNA, 100 nt, synthetic construct; identical to SEQ ID NO: 1866
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1962 - RNA, 100 nt, synthetic construct; identical to SEQ ID NO: 1867
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1963 - RNA, 100 nt, synthetic construct; identical to SEQ ID NO: 1868
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1964 - RNA, 100 nt, synthetic construct; identical to SEQ ID NO: 1869
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1965 - RNA, 100 nt, synthetic construct; identical to SEQ ID NO: 1870
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1966 - RNA, 100 nt, synthetic construct; identical to SEQ ID NO: 1871
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1967 - RNA, 100 nt, synthetic construct; identical to SEQ ID NO: 1872
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1968 - RNA, 100 nt, synthetic construct; identical to SEQ ID NO: 1873
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1969 - RNA, 100 nt, synthetic construct; identical to SEQ ID NO: 1874
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1970 - RNA, 100 nt, synthetic construct; identical to SEQ ID NO: 1875
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1971 - RNA, 100 nt, synthetic construct; identical to SEQ ID NO: 1876
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1972 - RNA, 100 nt, synthetic construct; identical to SEQ ID NO: 1877
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1973 - RNA, 100 nt, synthetic construct; identical to SEQ ID NO: 1878
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1974 - RNA, 100 nt, synthetic construct; identical to SEQ ID NO: 1879
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1975 - RNA, 100 nt, synthetic construct; identical to SEQ ID NO: 1880
  misc_feature 1..100: note = Synthetic
  variation 1..100: note = 2'-O-methylation and phosphorothioate internucleoside linkage modifications are present or absent for detailed description of substitutions and preferred
  source 1..100: mol_type = other RNA

SEQ ID NO: 1976 - RNA, 100 nt, synthetic construct; identical to SEQ ID NO: 1881
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1977 - RNA, 100 nt, synthetic construct; identical to SEQ ID NO: 1882
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1978 - RNA, 100 nt, synthetic construct; identical to SEQ ID NO: 1883
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1979 - RNA, 100 nt, synthetic construct; identical to SEQ ID NO: 1884
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1980 - RNA, 100 nt, synthetic construct; identical to SEQ ID NO: 1885
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1981 - RNA, 100 nt, synthetic construct; identical to SEQ ID NO: 1886
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1982 - RNA, 100 nt, synthetic construct; identical to SEQ ID NO: 1887
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1983 - RNA, 100 nt, synthetic construct; identical to SEQ ID NO: 1888
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1984 - RNA, 100 nt, synthetic construct; identical to SEQ ID NO: 1889
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1985 - RNA, 100 nt, synthetic construct; identical to SEQ ID NO: 1890
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1986 - RNA, 100 nt, synthetic construct; identical to SEQ ID NO: 1891
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1987 - RNA, 100 nt, synthetic construct; identical to SEQ ID NO: 1892
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1988 - RNA, 100 nt, synthetic construct; identical to SEQ ID NO: 1893
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1989 - RNA, 100 nt, synthetic construct; identical to SEQ ID NO: 1894
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1990 - RNA, 100 nt, synthetic construct; identical to SEQ ID NO: 1895
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1991 - RNA, 100 nt, synthetic construct; identical to SEQ ID NO: 1896
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1992 - RNA, 100 nt, synthetic construct; identical to SEQ ID NO: 1897
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1993 - RNA, 100 nt, synthetic construct; identical to SEQ ID NO: 1898
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1994 - RNA, 100 nt, synthetic construct; identical to SEQ ID NO: 1899
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1995 - RNA, 100 nt, synthetic construct; identical to SEQ ID NO: 1900
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1996 - RNA, 100 nt, synthetic construct; identical to SEQ ID NO: 1901
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1997 - RNA, 100 nt, synthetic construct; identical to SEQ ID NO: 1902
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1998 - RNA, 100 nt, synthetic construct; identical to SEQ ID NO: 1903
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 1999 - RNA, 100 nt, synthetic construct; identical to SEQ ID NO: 1904
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 2000 - RNA, 100 nt, synthetic construct; identical to SEQ ID NO: 1905
  misc_feature 1..100: note = Synthetic
  source 1..100: mol_type = other RNA

SEQ ID NO: 2001 - RNA, 20 nt, synthetic construct
  misc_feature 1..20: note = Synthetic
  source 1..20: mol_type = other RNA
tggagggcctgatgtgtgtt

SEQ ID NO: 2002 - RNA, 20 nt, synthetic construct
  misc_feature 1..20: note = Synthetic
  source 1..20: mol_type = other RNA
gcctgatgtgtgttgggtgt

SEQ ID NO: 2003 - RNA, 20 nt, synthetic construct
  misc_feature 1..20: note = Synthetic
  source 1..20: mol_type = other RNA
cctgatgtgtgttgggtgtt

SEQ ID NO: 2004 - RNA, 20 nt, synthetic construct
  misc_feature 1..20: note = Synthetic
  source 1..20: mol_type = other RNA
cccaacacccaacacacatc

SEQ ID NO: 2005 - RNA, 20 nt, synthetic construct
  misc_feature 1..20: note = Synthetic
  source 1..20: mol_type = other RNA
tcaggaaacatgaagaaagc

SEQ ID NO: 2006 - RNA, 20 nt, synthetic construct
  misc_feature 1..20: note = Synthetic
  source 1..20: mol_type = other RNA
aggcgcctgggcctctcccg

SEQ ID NO: 2007 - RNA, 20 nt, synthetic construct
  misc_feature 1..20: note = Synthetic
  source 1..20: mol_type = other RNA
cgggctggcctcccacaagg

SEQ ID NO: 2008 - RNA, 20 nt, synthetic construct
  misc_feature 1..20: note = Synthetic
  source 1..20: mol_type = other RNA
acggccatcctcggcgtctg

SEQ ID NO: 2009 - RNA, 20 nt, synthetic construct
  misc_feature 1..20: note = Synthetic
  source 1..20: mol_type = other RNA
gacggccatcctcggcgtct

SEQ ID NO: 2010 - RNA, 20 nt, synthetic construct
  misc_feature 1..20: note = Synthetic
  source 1..20: mol_type = other RNA
gacgccgaggatggccgtca

SEQ ID NO: 2011 - RNA, 20 nt, synthetic construct
  misc_feature 1..20: note = Synthetic
  source 1..20: mol_type = other RNA
tgacggccatcctcggcgtc

SEQ ID NO: 2012 - RNA, 20 nt, synthetic construct
  misc_feature 1..20: note = Synthetic
  source 1..20: mol_type = other RNA
ggcgccatgacggccatcct

SEQ ID NO: 2013 - RNA, 20 nt, synthetic construct
  misc_feature 1..20: note = Synthetic
  source 1..20: mol_type = other RNA
acagcgacgccgcgagccag

SEQ ID NO: 2014 - RNA, 20 nt, synthetic construct
  misc_feature 1..20: note = Synthetic
  source 1..20: mol_type = other RNA
cgacgccgcgagccagagga

SEQ ID NO: 2015 - RNA, 20 nt, synthetic construct
  misc_feature 1..20: note = Synthetic
  source 1..20: mol_type = other RNA
cgagccagaggatggagccg

SEQ ID NO: 2016 - RNA, 20 nt, synthetic construct
  misc_feature 1..20: note = Synthetic
  source 1..20: mol_type = other RNA
cggctccatcctctggctcg

SEQ ID NO: 2017 - RNA, 20 nt, synthetic construct
  misc_feature 1..20: note = Synthetic
  source 1..20: mol_type = other RNA
gagccagaggatggagccgc

SEQ ID NO: 2018 - RNA, 20 nt, synthetic construct
  misc_feature 1..20: note = Synthetic
  source 1..20: mol_type = other RNA
gcgcccgcggctccatcctc

SEQ ID NO: 2019 - RNA, 20 nt, synthetic construct
  misc_feature 1..20: note = Synthetic
  source 1..20: mol_type = other RNA
gcccgtccgtgggggatgag

SEQ ID NO: 2020 - RNA, 20 nt, synthetic construct
  misc_feature 1..20: note = Synthetic
  source 1..20: mol_type = other RNA
tcatcccccacggacgggcc

SEQ ID NO: 2021 - RNA, 20 nt, synthetic construct
  misc_feature 1..20: note = Synthetic
  source 1..20: mol_type = other RNA
atctcggacccggagactgt

SEQ ID NO: 2022 - RNA, 20 nt, synthetic construct
  misc_feature 1..20: note = Synthetic
  source 1..20: mol_type = other RNA
ggggtcccgcggcttcgggg

SEQ ID NO: 2023 - RNA, 20 nt, synthetic construct
  misc_feature 1..20: note = Synthetic
  source 1..20: mol_type = other RNA
ctcggggtcccgcggcttcg

SEQ ID NO: 2024 - RNA, 20 nt, synthetic construct
  misc_feature 1..20: note = Synthetic
  source 1..20: mol_type = other RNA
tctcggggtcccgcggcttc

SEQ ID NO: 2025 - RNA, 20 nt, synthetic construct
  misc_feature 1..20: note = Synthetic
  source 1..20: mol_type = other RNA
gtctcggggtcccgcggctt

SEQ ID NO: 2026 - RNA, 20 nt, synthetic construct
  misc_feature 1..20: note = Synthetic
  source 1..20: mol_type = other RNA
gcaagggtctcggggtcccg

SEQ ID NO: 2027 - RNA, 20 nt, synthetic construct
  misc_feature 1..20: note = Synthetic
  source 1..20: mol_type = other RNA
ggaccccgagacccttgccc

SEQ ID NO: 2028 - RNA, 20 nt, synthetic construct
  misc_feature 1..20: note = Synthetic
  source 1..20: mol_type = other RNA
gaccccgagacccttgcccc

SEQ ID NO: 2029 - RNA, 20 nt, synthetic construct
  misc_feature 1..20: note = Synthetic
  source 1..20: mol_type = other RNA
cgagacccttgccccgggag

SEQ ID NO: 2030 - RNA, 20 nt, synthetic construct
  misc_feature 1..20: note = Synthetic
  source 1..20: mol_type = other RNA
ctcccggggcaagggtctcg

SEQ ID NO: 2031 - RNA, 20 nt, synthetic construct
  misc_feature 1..20: note = Synthetic
  source 1..20: mol_type = other RNA
tctcccggggcaagggtctc

SEQ ID NO: 2032 - RNA, 20 nt, synthetic construct
  misc_feature 1..20: note = Synthetic
  source 1..20: mol_type = other RNA
ctctcccggggcaagggtct

SEQ ID NO: 2033 - RNA, 20 nt, synthetic construct
  misc_feature 1..20: note = Synthetic
  source 1..20: mol_type = other RNA
ccttgccccgggagaggccc

SEQ ID NO: 2034 - RNA, 20 nt, synthetic construct
  misc_feature 1..20: note = Synthetic
  source 1..20: mol_type = other RNA
ctgggcctctcccggggcaa

SEQ ID NO: 2035 - RNA, 20 nt, synthetic construct
  misc_feature 1..20: note = Synthetic
  source 1..20: mol_type = other RNA
cctgggcctctcccggggca

SEQ ID NO: 2036 - RNA, 20 nt, synthetic construct
  misc_feature 1..20: note = Synthetic
  source 1..20: mol_type = other RNA
tttaggccaaaaatcccccc

SEQ ID NO: 2037 - RNA, 20 nt, synthetic construct
  misc_feature 1..20: note = Synthetic
  source 1..20: mol_type = other RNA
cgctgcagcgcacgggtacc

SEQ ID NO: 2038 - RNA, 20 nt, synthetic construct
  misc_feature 1..20: note = Synthetic
  source 1..20: mol_type = other RNA
gctgcagcgcacgggtacca

SEQ ID NO: 2039 - RNA, 20 nt, synthetic construct
  misc_feature 1..20: note = Synthetic
  source 1..20: mol_type = other RNA
ctgcagcgcacgggtaccag

SEQ ID NO: 2040 - RNA, 20 nt, synthetic construct
  misc_feature 1..20: note = Synthetic
  source 1..20: mol_type = other RNA
cgcacgggtaccaggggcca

SEQ ID NO: 2041 - RNA, 20 nt, synthetic construct
  misc_feature 1..20: note = Synthetic
  source 1..20: mol_type = other RNA
gcacgggtaccaggggccac

SEQ ID NO: 2042 - RNA, 20 nt, synthetic construct
  misc_feature 1..20: note = Synthetic
  source 1..20: mol_type = other RNA
cacgggtaccaggggccacg

SEQ ID NO: 2043 - RNA, 20 nt, synthetic construct
  misc_feature 1..20: note = Synthetic
  source 1..20: mol_type = other RNA
gggaggcgccccgtggcccc

SEQ ID NO: 2044 - RNA, 20 nt, synthetic construct
  misc_feature 1..20: note = Synthetic
  source 1..20: mol_type = other RNA
gcgatcagggaggcgccccg

SEQ ID NO: 2045 - RNA, 20 nt, synthetic construct
  misc_feature 1..20: note = Synthetic
  source 1..20: mol_type = other RNA
tccttgtgggaggccagccc

SEQ ID NO: 2046 - RNA, 20 nt, synthetic construct
  misc_feature 1..20: note = Synthetic
  source 1..20: mol_type = other RNA
ctccttgtgggaggccagcc

SEQ ID NO: 2047 - RNA, 20 nt, synthetic construct
  misc_feature 1..20: note = Synthetic
  source 1..20: mol_type = other RNA
ggctggcctcccacaaggag

SEQ ID NO: 2048 - RNA, 20 nt, synthetic construct
  misc_feature 1..20: note = Synthetic
  source 1..20: mol_type = other RNA
ttgtctcccctccttgtggg

SEQ ID NO: 2049 - RNA, 20 nt, synthetic construct
  misc_feature 1..20: note = Synthetic
  source 1..20: mol_type = other RNA
ccacaaggaggggagacaat

SEQ ID NO: 2050 - RNA, 20 nt, synthetic construct
  misc_feature 1..20: note = Synthetic
  source 1..20: mol_type = other RNA
cacaaggaggggagacaatt

SEQ ID NO: 2051 - RNA, 20 nt, synthetic construct
  misc_feature 1..20: note = Synthetic
  source 1..20: mol_type = other RNA
caattgtctcccctccttgt

SEQ ID NO: 2052 - RNA, 20 nt, synthetic construct
  misc_feature 1..20: note = Synthetic
  source 1..20: mol_type = other RNA
ccaattgtctcccctccttg

SEQ ID NO: 2053 - RNA, 20 nt, synthetic construct
  misc_feature 1..20: note = Synthetic
  source 1..20: mol_type = other RNA
atccctcgaatactgatgag

SEQ ID NO: 2054 - RNA, 20 nt, synthetic construct
  misc_feature 1..20: note = Synthetic
  source 1..20: mol_type = other RNA
aaccactcatcagtattcga

SEQ ID NO: 2055 - RNA, 20 nt, synthetic construct
  misc_feature 1..20: note = Synthetic
  source 1..20: mol_type = other RNA
gaaccactcatcagtattcg

SEQ ID NO: 2056 - RNA, 20 nt, synthetic construct
  misc_feature 1..20: note = Synthetic
  source 1..20: mol_type = other RNA
gaggaaaagtcacgggccca

SEQ ID NO: 2057 - RNA, 20 nt, synthetic construct
  misc_feature 1..20: note = Synthetic
  source 1..20: mol_type = other RNA
ggcccgtgacttttcctctc

SEQ ID NO: 2058 - RNA, 20 nt, synthetic construct
  misc_feature 1..20: note = Synthetic
  source 1..20: mol_type = other RNA
tgcttcacactcaatgtgtg

SEQ ID NO: 2059 - RNA, 20 nt, synthetic construct
  misc_feature 1..20: note = Synthetic
  source 1..20: mol_type = other RNA
gcttcacactcaatgtgtgt

SEQ ID NO: 2060 - RNA, 20 nt, synthetic construct
  misc_feature 1..20: note = Synthetic
  source 1..20: mol_type = other RNA
cttcacactcaatgtgtgtg

SEQ ID NO: 2061 - RNA, 20 nt, synthetic construct
  misc_feature 1..20: note = Synthetic
  source 1..20: mol_type = other RNA
ttcacactcaatgtgtgtgg

SEQ ID NO: 2062 - RNA, 20 nt, synthetic construct
  misc_feature 1..20: note = Synthetic
  source 1..20: mol_type = other RNA
ttgagaatggacaggacacc

SEQ ID NO: 2063 - RNA, 20 nt, synthetic construct
  misc_feature 1..20: note = Synthetic
  source 1..20: mol_type = other RNA
aggcattttgcatctgtcat

SEQ ID NO: 2064 - RNA, 20 nt, synthetic construct
  misc_feature 1..20: note = Synthetic
  source 1..20: mol_type = other RNA
caggcattttgcatctgtca

SEQ ID NO: 2065 - RNA, 20 nt, synthetic construct
  misc_feature 1..20: note = Synthetic
  source 1..20: mol_type = other RNA
aggggccctgaccctgctaa

SEQ ID NO: 2066 - RNA, 20 nt, synthetic construct
  misc_feature 1..20: note = Synthetic
  source 1..20: mol_type = other RNA
tgggaaaagaggggaaggtg

SEQ ID NO: 2067 - RNA, 20 nt, synthetic construct
  misc_feature 1..20: note = Synthetic
  source 1..20: mol_type = other RNA
tggaggaggaagagctcagg

SEQ ID NO: 2068 - RNA, 20 nt, synthetic construct
  misc_feature 1..20: note = Synthetic
  source 1..20: mol_type = other RNA
tgagatttcttgtctcactg

SEQ ID NO: 2069 - RNA, 20 nt, synthetic construct
  misc_feature 1..20: note = Synthetic
  source 1..20: mol_type = other RNA
gagatttcttgtctcactga

SEQ ID NO: 2070 - RNA, 20 nt, synthetic construct
  misc_feature 1..20: note = Synthetic
  source 1..20: mol_type = other RNA
taaagcacctgttaaaatga

SEQ ID NO: 2071 - RNA, 20 nt, synthetic construct
  misc_feature 1..20: note = Synthetic
  source 1..20: mol_type = other RNA
aatctgtccttcattttaac

SEQ ID NO: 2072 - RNA, 20 nt, synthetic construct
  misc_feature 1..20: note = Synthetic
  source 1..20: mol_type = other RNA
gtcacaggggaaggtccctg

SEQ ID NO: 2073 - RNA, 20 nt, synthetic construct
  misc_feature 1..20: note = Synthetic
  source 1..20: mol_type = other RNA
aaacatgaagaaagcaggtg

SEQ ID NO: 2074 - RNA, 20 nt, synthetic construct
  misc_feature 1..20: note = Synthetic
  source 1..20: mol_type = other RNA
tgtcctgtgagataccagaa

SEQ ID NO: 2075 - RNA, 20 nt, synthetic construct
  misc_feature 1..20: note = Synthetic
  source 1..20: mol_type = other RNA
atgaaggaggctgatgcctg

SEQ ID NO: 2076 - RNA, 20 nt, synthetic construct
  misc_feature 1..20: note = Synthetic
  source 1..20: mol_type = other RNA
aggctgatgcctgaggtcct

SEQ ID NO: 2077 - RNA, 20 nt, synthetic construct
  misc_feature 1..20: note = Synthetic
  source 1..20: mol_type = other RNA
ggctgatgcctgaggtcctt

SEQ ID NO: 2078 - RNA, 20 nt, synthetic construct
  misc_feature 1..20: note = Synthetic
  source 1..20: mol_type = other RNA
cacaatatcccaaggacctc

SEQ ID NO: 2079 - RNA, 20 nt, synthetic construct
  misc_feature 1..20: note = Synthetic
  source 1..20: mol_type = other RNA
ggtccttgggatattgtgtt

SEQ ID NO: 2080 - RNA, 20 nt, synthetic construct
  misc_feature 1..20: note = Synthetic
  source 1..20: mol_type = other RNA
gtccttgggatattgtgttt

SEQ ID NO: 2081 - RNA, 20 nt, synthetic construct
  misc_feature 1..20: note = Synthetic
  source 1..20: mol_type = other RNA
ctcccaaacacaatatccca

SEQ ID NO: 2082 - RNA, 20 nt, synthetic construct
  misc_feature 1..20: note = Synthetic
  source 1..20: mol_type = other RNA
tcctctagccacatcttctg

SEQ ID NO: 2083 - RNA, 20 nt, synthetic construct
  misc_feature 1..20: note = Synthetic
  source 1..20: mol_type = other RNA
acagaagatgtggctagagg

SEQ ID NO: 2084 - RNA, 20 nt, synthetic construct
  misc_feature 1..20: note = Synthetic
  source 1..20: mol_type = other RNA
cctctagccacatcttctgt

SEQ ID NO: 2085 - RNA, 20 nt, synthetic construct
  misc_feature 1..20: note = Synthetic
  source 1..20: mol_type = other RNA
cccacagaagatgtggctag

SEQ ID NO: 2086 - RNA, 20 nt, synthetic construct
  misc_feature 1..20: note = Synthetic
  source 1..20: mol_type = other RNA
gtcagatcccacagaagatg

SEQ ID NO: 2087 - RNA, 20 nt, synthetic construct
  misc_feature 1..20: note = Synthetic
  source 1..20: mol_type = other RNA
atcttctgtgggatctgacc

SEQ ID NO: 2088 - RNA, 20 nt, synthetic construct
  misc_feature 1..20: note = Synthetic
  source 1..20: mol_type = other RNA
cccaggcagtgacagtgccc

SEQ ID NO: 2089 - RNA, 20 nt, synthetic construct
  misc_feature 1..20: note = Synthetic
  source 1..20: mol_type = other RNA
ctgggcactgtcactgcctg

SEQ ID NO: 2090 - RNA, 20 nt, synthetic construct
  misc_feature 1..20: note = Synthetic
  source 1..20: mol_type = other RNA
cctgggcactgtcactgcct

SEQ ID NO: 2091 - RNA, 20 nt, synthetic construct
  misc_feature 1..20: note = Synthetic
  source 1..20: mol_type = other RNA
ccctgggcactgtcactgcc

SEQ ID NO: 2092 - RNA, 20 nt, synthetic construct
  misc_feature 1..20: note = Synthetic
  source 1..20: mol_type = other RNA
ttgggtgttgggcggaacag

SEQ ID NO: 2093 - RNA, 20 nt, synthetic construct
  misc_feature 1..20: note = Synthetic
  source 1..20: mol_type = other RNA
tggatgtattgagcatgcga

SEQ ID NO: 2094 - RNA, 20 nt, synthetic construct
  misc_feature 1..20: note = Synthetic
  source 1..20: mol_type = other RNA
ggatgtattgagcatgcgat

SEQ ID NO: 2095 - RNA, 20 nt, synthetic construct
  misc_feature 1..20: note = Synthetic
  source 1..20: mol_type = other RNA
aacatgaagaaagcaggtgt

(19) United States
(12) Patent Application Publication
(10) Pub. No.: US 2024/0016934 A1
(43) Pub. Date: Jan. 18, 2024

(54) Compositions and Methods for Reducing MHC Class II in a Cell

(71) Applicant: Intellia Therapeutics, Inc., Cambridge, MA (US)

(72) Inventors: Srijani Sridhar, Cambridge, MA (US); Yong Zhang, Bedford, MA (US); William Frederick Harrington, Cambridge, MA (US); Surbhi Goel, Winchester, MA (US)

(73) Assignee: Intellia Therapeutics, Inc., Cambridge, MA (US)

(21) Appl. No.: 18/332,390

(22) Filed: Jun. 9, 2023

Related U.S. Application Data
(63) Continuation of application No. PCT/US2021/062946, filed on Dec. 10, 2021.
(60) Provisional application No. 63/130,106, filed on Dec. 23, 2020.
(60) Provisional application No. 63/124,064, filed on Dec. 11, 2020.

(51) Int. Cl. A61K 39/00; A61P 37/02; C12N 5/0783; A61K 35/17; C12N 15/86

(52) U.S. Cl. CPC A61K 39/4632; A61P 37/02; C12N 5/0636; A61K 35/17; C12N 15/86; C12N 2510/00; C12N 2750/14143

(57) ABSTRACT

Compositions and methods for reducing MHC class II protein expression in a cell comprising genetically modifying CIITA for use e.g., in adoptive cell transfer therapies.

This application is a continuation application of International Application No. PCT/US2021/062946, filed Dec. 10, 2021, which claims the benefit under 35 U.S.C. 119(e) of U.S. Provisional Application No. 63/124,064, filed Dec. 11, 2020 and U.S. Provisional Application No. 63/130,106, filed Dec. 23, 2020; all of which disclosures is herein incorporated by reference in its entirety.

This application is filed with a Sequence Listing in electronic format. The Sequence Listing is provided as a file entitled “01155-0034-00US.xml” which was created on Aug. 9, 2023, which is 1,344,666 bytes in size. The information in the electronic format of the sequence listing is incorporated herein by reference in its entirety.

### INTRODUCTION AND SUMMARY

The ability to downregulate MHC class II is critical for many in vivo and ex vivo utilities, e.g., when using allogeneic cells (originating from a donor) for transplantation and/or e.g., for creating a cell population in vitro that does not activate T cells. In particular, the transfer of allogeneic cells into a subject is of great interest to the field of cell therapy. The use of allogeneic cells has been limited due to the problem of rejection by the recipient subject's immune cells, which recognize the transplanted cells as foreign and mount an attack. To avoid the problem of immune rejection, cell-based therapies have focused on autologous approaches that use a subject's own cells as the cell source for therapy, an approach that is time-consuming and costly.

Typically, immune rejection of allogeneic cells results from a mismatching of major histocompatibility complex (MHC) molecules between the donor and recipient. Within the human population, MHC molecules exist in various forms, including e.g., numerous genetic variants of any given MHC gene, i.e., alleles, encoding different forms of MHC protein. The primary classes of MHC molecules are referred to as MHC class I and MHC class II. MHC class I molecules (e.g., HLA-A, HLA-B, and HLA-C in humans) are expressed on all nucleated cells and present antigens to activate cytotoxic T cells (CD8+ T cells or CTLs). MHC class II molecules (e.g., HLA-DP, HLA-DQ, and HLA-DR in humans) are expressed on only certain cell types (e.g., B cells, dendritic cells, and macrophages) and present antigens to activate helper T cells (CD4+ T cells or Th cells), which in turn provide signals to B cells to produce antibodies.

Slight differences, e.g., in MHC alleles between individuals can cause the T cells in a recipient to become activated. During T cell development, an individual's T cell repertoire is tolerized to one's own MHC molecules, but T cells that recognize another individual's MHC molecules may persist in circulation and are referred to as alloreactive T cells. Alloreactive T cells can become activated e.g., by the presence of another individual's cells expressing MHC molecules in the body, causing e.g., graft versus host disease and transplant rejection.

Methods and compositions for reducing the susceptibility of an allogeneic cell to rejection are of interest, including e.g., reducing the cell's expression of MHC protein to avoid recipient T cell responses. In practice, the ability to genetically modify an allogeneic cell for transplantation into a subject has been hampered by the requirement for multiple gene edits to reduce all MHC protein expression, while at the same time, avoiding other harmful recipient immune responses. For example, while strategies to deplete MHC class I protein may reduce activation of CTLs, cells that lack MHC class I on their surface are susceptible to lysis by natural killer (NK) cells of the immune system because NK cell activation is regulated by MHC class I-specific inhibitory receptors. Gene editing strategies to deplete MHC class II molecules have also proven difficult particularly in certain cell types for reasons including low editing efficiencies and low cell survival rates, preventing practical application as a cell therapy.

Thus, there exists a need for improved methods and compositions for modifying allogeneic cells to overcome the problem of recipient immune rejection and the technical difficulties associated with the multiple genetic modifications required to produce a safer cell for transplant.

The present disclosure provides engineered cells with reduced or eliminated surface expression of MHC class II. The engineered cell comprises a genetic modification in the CIITA gene (class II major histocompatibility complex transactivator), which may be useful in cell therapy. The disclosure further provides compositions and methods to reduce or eliminate surface expression of MHC class II protein in a cell by genetically modifying the CIITA gene. The CIITA protein functions as a transcriptional activator (activating the MHC class II promoter) and is essential for MHC class II protein expression. In some embodiments, the disclosure further provides compositions and methods to reduce or eliminate surface expression of MHC class I protein in the cell by genetically modifying B2M (β-2-microgloblin). The B2M protein forms a heterodimer with MHC class I molecules and is required for MHC class I protein expression on the cell surface. The disclosure further provides expression of an NK cell inhibitor molecule by the cell to reduce or eliminate the lytic activity of NK cells. In some embodiments, the methods and compositions further provide for insertion of an exogenous nucleic acid, e.g., encoding a targeting receptor, other polypeptide expressed on the cell surface, or a polypeptide that is secreted from the cell. Thus, in some embodiments, the engineered cell is useful as a “cell factory” for secreting an exogenous protein in a recipient. In some embodiments, the engineered cell is useful as an adoptive cell therapy.

In some embodiments, an engineered cell, which has reduced or eliminated surface expression of MHC class II relative to an unmodified cell, is provided, the engineered cell comprising a genetic modification in the CIITA gene, wherein the genetic modification comprises at least one nucleotide of a splice site within the genomic coordinates chr16:10902171-10923242. In some embodiments, the genetic modification comprises a modification of at least one nucleotide of a splice acceptor site. In some embodiments, the one nucleotide is A. In some embodiments, the one nucleotide is G. In some embodiments, the genetic modification comprises a modification of at least one nucleotide of a splice donor site. In some embodiments, the one nucleotide is G. In some embodiments, the one nucleotide is T. In some embodiments, the genetic modification comprises a modification of a splice site boundary nucleotide.

In some embodiments, an engineered cell, which has reduced or eliminated surface expression of MHC class II relative to an unmodified cell, is provided, the engineered cell comprising a genetic modification in the CIITA gene, wherein the genetic modification comprises an indel, a C to T substitution, or an A to G substitution within the genomic coordinates chosen from: chr16:10895410-10895430, chr16:10898649-10898669, chr16:10898658-10898678, chr16:10902171-10902191, chr16:10902173-10902193, chr16:10902174-10902194, chr16:10902179-10902199, chr16:10902183-10902203, chr16:10902184-10902204, chr16:10902644-10902664, chr16:10902779-10902799, chr16:10902788-10902808, chr16:10902789-10902809, chr16:10902790-10902810, chr16:10902795-10902815, chr16:10902799-10902819, chr16:10903708-10903728, chr16:10903713-10903733, chr16:10903718-10903738, chr16:10903721-10903741, chr16:10903723-10903743, chr16:10903724-10903744, chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242. In some embodiments, the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150. In some embodiments, the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, chr16:10916450-10916470, chr16:10923222-10923242, chr16:10916449-10916469, and chr16:10923214-10923234. In some embodiments, the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, and chr16:10916450-10916470.

In some embodiments, a composition is provided, the composition comprising: a) a CIITA guide RNA comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide; wherein the CIITA guide RNA targets a CIITA genomic target sequence comprising at least 10 contiguous nucleotides within the genomic coordinates chr16:10902171-10923242.

In some embodiments, a composition is provided, the composition comprising: a) a CIITA guide RNA (gRNA) comprising i) a guide sequence selected from SEQ ID NOs: 1-101; or ii) at least 17, 18, 19, or 20 contiguous nucleotides of a sequence selected from SEQ ID NOs: 1-101; or iii) a guide sequence at least 95%, 90%, or 85% identical to a sequence selected from SEQ ID NOs: 1-101; or iv) a sequence that comprises 10 contiguous nucleotides ±10 nucleotides of a genomic coordinate listed in Table 1; or v) at least 17, 18, 19, or 20 contiguous nucleotides of a sequence from (iv); or vi) a guide sequence that is at least 95%, 90%, or 85% identical to a sequence selected from (v).

In some embodiments, a composition is provided, the composition comprising: a) a CIITA guide RNA that is a single-guide RNA (sgRNA) comprising i) a guide sequence selected from SEQ ID NOs: 1-101; or ii) at least 17, 18, 19, or 20 contiguous nucleotides of a sequence selected from SEQ ID NOs: 1-101; or iii) a guide sequence at least 95%, 90%, or 85% identical to a sequence selected from SEQ ID NOs: 1-101; or iv) a sequence that comprises 10 contiguous nucleotides ±10 nucleotides of a genomic coordinate listed in Table 1; or v) at least 17, 18, 19, or 20 contiguous nucleotides of a sequence from (iv); or vi) a guide sequence that is at least 95%, 90%, or 85% identical to a sequence selected from (v).

In some embodiments, a method of making an engineered cell, which has reduced or eliminated surface expression of MHC class II protein relative to an unmodified cell, is provided, the method comprising contacting a cell with a composition of any of the embodiments provided herein. In some embodiments, the composition comprises a CIITA guide RNA, comprising a nucleotide chosen from: SEQ ID NO: 47, SEQ ID NO: 55, SEQ ID NO: 71, SEQ ID NO: 80, SEQ ID NO: 82, SEQ ID NO: 83, SEQ ID NO: 87, SEQ ID NO: 91, SEQ ID NO: 96, SEQ ID NO: 97, SEQ ID NO: 98, SEQ ID NO: 100, and SEQ ID NO: 101. In some embodiments, the composition comprises a CIITA guide RNA, comprising a nucleotide chosen from: SEQ ID NO: 47, SEQ ID NO: 55, SEQ ID NO: 71, SEQ ID NO: 80, SEQ ID NO: 83, SEQ ID NO: 87, SEQ ID NO: 91, SEQ ID NO: 97, SEQ ID NO: 98, and SEQ ID NO: 100.

In some embodiments, a method of reducing surface expression of MHC class II protein in an engineered cell relative to an unmodified cell, is provided, the method comprising contacting a cell with a composition of any of embodiments provided herein.

### BRIEF DESCRIPTION OF THE DRAWINGS

FIGS. 1A-D show results of a screen in T cells comparing editing of CIITA guide RNAs (FIG. 1A (for Cas9 cleavase (Cas9)) and FIG. 1B (a deaminase (BC22)) with the mean percentage of T cells negative for cell surface expression of MHC class II (% MHC II negative”) by flow cytometry for Cas9 (FIG. 1C) and BC22 (FIG. 1D).

FIG. 2 shows the mean percentage of T cells negative for cell surface expression of MHC class II (% MHC II negative”) for several guides using Cas9 and BC22 in relation to the distance from the cut site to the splice site boundary nucleotide shown as base pairs (“bp”). Positive numerical values indicate a splice site boundary nucleotide 3′ of the cut site, whereas the negative numerical values indicate a splice site boundary nucleotide 5′ of the cut site.

FIGS. 3A-3D show the editing profile of T cells as percent of total reads while varying levels of BC22n (“BC22n,” as used herein, refers to BC22 without UGI) mRNA and Cas9 mRNAs. Cells were edited with individual guide RNAs G015995 (FIG. 3A), G016017 (FIG. 3B), G016206 (FIG. 3C), and G018117 (FIG. 3D).

FIGS. 4A-4D show the editing profile for T cells as percent of total reads while varying levels of BC22n mRNA and Cas9 mRNAs, when four guide RNAs were used simultaneously for editing. The percentage of total reads with multi-guide delivery is shown for each of the four loci targeted by G015995 (FIG. 4A), G016017 (FIG. 4B), G016206 (FIG. 4C), and G018117 (FIG. 4D).

FIGS. 5A-5H show phenotyping results as percent of cells negative for antibody binding with increasing total RNA for both BC22n and Cas9 samples (as shown in Table 14). FIG. 5A shows the percentage of B2M negative cells when B2M guide G015995 was used for editing. FIG. 5B shows the percentage of B2M negative cells when multi guides were used for editing. FIG. 5C shows the percentage of CD3 negative cells when TRAC guide G016017 was used for editing. FIG. 5D shows the percentage of CD3 negative cells when TRBC guide G016206 was used for editing. FIG. 5E shows the percentage of CD3 negative cells when multiple guides were used for editing. FIG. 5F shows the percentage of MHC class II negative cells when CIITA guide G018117 was used for editing. FIG. 5G shows the percentage of MHC class II negative cells when multiple guides were used for editing. FIG. 5H shows the percentage of triple (B2M, CD3, MHC II) negative cells when multiple guides were used for editing.

FIGS. 6A-6B show the editing profile in T cells following treatment with different mRNA constructs and CIITA-targeting sgRNAs (FIG. 6A) and MHC class II negative cells assessed by flow cytometry analysis of T cells treated with different mRNA constructs and CIITA guide RNAs (FIG. 6B).

FIGS. 7A-7D show scatter plots showing statistically significant (*=p. adj.<0.05) differential gene expression events (black dots) in T cells treated with a first guide, UGI mRNA and either Cas9 mRNA (FIG. 7A) or BC22n mRNA (FIG. 7B), or with a second guide, UGI mRNA and either Cas9 mRNA (FIG. 7C) or BC22n mRNA (FIG. 7D).

FIGS. 8A-8D show protein-protein interaction networks enriched among the list of differentially expressed genes in T cells treated with a first guide, UGI mRNA and either Cas9 mRNA (FIG. 8A) or BC22n mRNA (FIG. 8B), or a with a second guide, UGI mRNA and either Cas9 mRNA (FIG. 8C) or BC22n mRNA (FIG. 8D).

FIGS. 9A-9C show survival of B2M knockout T cells and B2M knockout/HLA-E T cells at 90 days post injection (FIG. 9A), over a 90-day time course (FIG. 9B), and over a 30-day time course (FIG. 9C), in a murine model of NK cell killing by an in vivo imaging system (IVIS); the IVIS signal was quantitated as average radiance. Data points for individual mice (1-8) are shown.

FIGS. 10A-10B show the percentage of editing of CIITA, B2M, and TRAC in T cells by NGS sequencing before magnetic cell separation (MACS®) processing (FIG. 10A) and after MACS® processing (FIG. 10B).

FIGS. 11A-111B show the mean percentage of T cells negative for cell surface expression of MHC class II, B2M, and TRAC by flow cytometry before MACS© processing (FIG. 11A) and after MACS© processing (FIG. 11B).

FIG. 12 shows the chromosomal structural variations in genetically modified cells treated with electroporation, a simultaneous LNP process, or a sequential LNP process, by KromaTiD dGH assay.

### DETAILED DESCRIPTION

The present disclosure provides engineered cells, as well as methods and compositions for genetically modifying a cell to make an engineered cell and populations of engineered cells, that are useful, for example, for adoptive cell transfer (ACT) therapies. The disclosure provided herein overcomes certain hurdles of prior methods by providing methods and compositions for genetically modifying CIITA to reduce expression of MHC class II protein on the surface of a cell. In some embodiments, the disclosure provides engineered cells with reduced or eliminated surface expression of MHC class II as a result of a genetic modification in the CIITA gene. In some embodiments, the disclosure provides compositions and methods for reducing or eliminating expression of MHC class II protein and compositions and methods to further reduce the cell's susceptibility to immune rejection. For example, in some embodiments, the methods and compositions comprise reducing or eliminating surface expression of MHC class II protein by genetically modifying CIITA, and reducing or eliminating surface expression of MHC class I protein and/or inserting an exogenous nucleic acid encoding an NK cell inhibitor molecule, or a targeting receptor, or other polypeptide (expressed on the cell surface or secreted) into the cell by genetic modification. The engineered cell compositions produced by the methods disclosed herein have desirable properties, including e.g., reduced expression of MHC molecules, reduced immunogenicity in vitro and in vivo, increased survival, and increased genetic compatibility with greater subjects for transplant.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### I. Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, CBBA, CABA, and so forth. The skilled artisan will understand that typically there is no limit on the number of items or terms in any combination, unless otherwise apparent from the context.

As used herein, the term “kit” refers to a packaged set of related components, such as one or more polynucleotides or compositions and one or more related materials such as delivery devices (e.g., syringes), solvents, solutions, buffers, instructions, or desiccants.

An “allogeneic” cell, as used herein, refers to a cell originating from a donor subject of the same species as a recipient subject, wherein the donor subject and recipient subject have genetic dissimilarity, e.g., genes at one or more loci that are not identical. Thus, e.g., a cell is allogeneic with respect to the subject to be administered the cell. As used herein, a cell that is removed or isolated from a donor, that will not be re-introduced into the original donor, is considered an allogeneic cell.

An “autologous” cell, as used herein, refers to a cell derived from the same subject to whom the material will later be re-introduced. Thus, e.g., a cell is considered autologous if it is removed from a subject and it will then be re-introduced into the same subject.

“β2M” or “B2M,” as used herein, refers to nucleic acid sequence or protein sequence of “β-2 microglobulin”; the human gene has accession number NC_000015 (range 44711492..44718877), reference GRCh38.p13. The B2M protein is associated with MHC class I molecules as a heterodimer on the surface of nucleated cells and is required for MHC class I protein expression.

“CIITA” or “CIITA” or “C2TA,” as used herein, refers to the nucleic acid sequence or protein sequence of “class II major histocompatibility complex transactivator;” the human gene has accession number NC_000016.10 (range 10866208..10941562), reference GRCh38.p13. The CIITA protein in the nucleus acts as a positive regulator of MHC class II gene transcription and is required for MHC class II protein expression.

As used herein, “MHC” or “MHC molecule(s)” or “MHC protein” or “MHC complex(es),” refers to a major histocompatibility complex molecule (or plural), and includes e.g., MHC class I and MHC class II molecules. In humans, MHC molecules are referred to as “human leukocyte antigen” complexes or “HLA molecules” or “HLA protein.” The use of terms “MHC” and “HLA” are not meant to be limiting; as used herein, the term “MHC” may be used to refer to human MHC molecules, i.e., HLA molecules. Therefore, the terms “MHC” and “HLA” are used interchangeably herein.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility, A alpha chain;” the human gene has accession number NC_000006.12 (29942532..29945870). The HLA-A gene is known to have thousands of different versions (also referred to as “alleles”) across the population (and an individual may receive two different alleles of the HLA-A gene). A public database for HLA-A alleles, including sequence information, may be accessed at IPD-IMGT/HLA: https://www.ebi.ac.uk/ipd/imgt/hla/. All alleles of HLA-A are encompassed by the terms “HLA-A” and “HLA-A gene.”

“HLA-B” as used herein in the context of nucleic acids refers to the gene encoding the heavy chain of the HLA-B protein molecule. The HLA-B is also referred to as “HLA class I histocompatibility, B alpha chain;” the human gene has accession number NC_000006.12 (31353875..31357179).

“HLA-C” as used herein in the context of nucleic acids refers to the gene encoding the heavy chain of the HLA-C protein molecule. The HLA-C is also referred to as “HLA class I histocompatibility, C alpha chain;” the human gene has accession number NC_000006.12 (31268749..31272092).

As used herein, the term “within the genomic coordinates” includes the boundaries of the genomic coordinate range given. For example, if chr6:29942854-chr6:29942913 is given, the coordinates chr6:29942854-chr6:29942913 are encompassed. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

As used herein, the term “homozygous” refers to having two identical alleles of a particular gene.

A “splice site,” as used herein, refers to the three nucleotides that make up an acceptor splice site or a donor splice site (defined below), or any other nucleotides known in the art that are part of a splice site. See e.g., Burset et al., Nucleic Acids Research 28(21):4364-4375 (2000) (describing canonical and non-canonical splice sites in mammalian genomes). The three nucleotides that make up an “acceptor splice site” are two conserved residues (e.g., AG in humans) at the 3′ of an intron and a boundary nucleotide (i.e., the first nucleotide of the exon 3′ of the AG). The “splice site boundary nucleotide” of an acceptor splice site is designated as “Y” in the diagram below and may also be referred to herein as the “acceptor splice site boundary nucleotide,” or “splice acceptor site boundary nucleotide.” The terms “acceptor splice site,” “splice acceptor site,” “acceptor splice sequence,” or “splice acceptor sequence” may be used interchangeably herein.

The three nucleotides that make up a “donor splice site” are two conserved residues (e.g., GT (gene) or GU (in RNA such as pre-mRNA) in human) at the 5′ end of an intron and a boundary nucleotide (i.e., the first nucleotide of the exon 5′ of the GT). The “splice site boundary nucleotide” of a donor splice site is designated as “X” in the diagram below and may also be referred to herein as the “donor splice site boundary nucleotide,” or “splice donor site boundary nucleotide.” The terms “donor splice site,” “splice donor site,” “donor splice sequence,” or “splice donor sequence” may be used interchangeably herein.

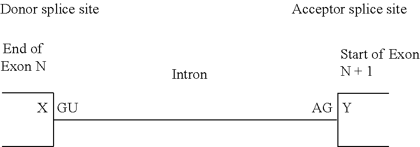

As used herein, “splice site region,” includes the nucleotides of the splice site, as well as nucleotides that are in proximity to the splice site.

As used herein, the term “subject” is intended to include living organisms in which an immune response can be elicited, including e.g., mammals, primates, humans.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to, for example, the guide that directs an RNA-guided DNA binding agent to a target DNA and can be a single guide RNA, or the combination of a crRNA and a trRNA (also known as tracrRNA). Exemplary gRNAs include Class II Cas nuclease guide RNAs, in modified or unmodified forms. The crRNA and trRNA may be associated as a single RNA molecule (single guide RNA, sgRNA) or in two separate RNA strands (dual guide RNA, dgRNA). “Guide RNA” or “gRNA” refers to each type. The trRNA may be a naturally occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9 (SpCas9)) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

As used herein, the term “editor” refers to an agent comprising a polypeptide that is capable of making a modification within a DNA sequence. In some embodiments, the editor is a cleavase, such as a Cas9 cleavase. In some embodiments, the editor is capable of deaminating a base within a DNA molecule. In some embodiments, the editor is capable of deaminating a cytosine (C) in DNA. In some embodiments, the editor is a fusion protein comprising an RNA-guided nickase fused to a cytidine deaminase. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A). In some embodiments, the editor is a fusion protein comprising an RNA-guided nickase fused to a cytidine deaminase and a UGI. In some embodiments, the editor lacks a UGI.

As used herein, a “cytidine deaminase” means a polypeptide or complex of polypeptides that is capable of cytidine deaminase activity, that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); Carrington et al., Cells 9:1690 (2020)).

As used herein, the term “APOBEC3” refers to a APOBEC3 protein, such as an APOBEC3 protein expressed by any of the seven genes (A3A-A3H) of the human APOBEC3 locus. The APOBEC3 may have catalytic DNA or RNA editing activity. An amino acid sequence of APOBEC3A has been described (UniPROT accession ID: p31941) and is included herein as SEQ ID NO: 40. In some embodiments, the APOBEC3 protein is a human APOBEC3 protein and/or a wild-type protein. Variants include proteins having a sequence that differs from wild-type APOBEC3 protein by one or several mutations (i.e. substitutions, deletions, insertions), such as one or several single point substitutions. For instance, a shortened APOBEC3 sequence could be used, e.g. by deleting several N-term or C-term amino acids, preferably one to four amino acids at the C-terminus of the sequence. As used herein, the term “variant” refers to allelic variants, splicing variants, and natural or artificial mutants, which are homologous to a APOBEC3 reference sequence. The variant is “functional” in that it shows a catalytic activity of DNA or RNA editing. In some embodiments, an APOBEC3 (such as a human APOBEC3A) has a wild-type amino acid position 57 (as numbered in the wild-type sequence). In some embodiments, an APOBEC3 (such as a human APOBEC3A) has an asparagine at amino acid position 57 (as numbered in the wild-type sequence).

As used herein, a “nickase” is an enzyme that creates a single-strand break (also known as a “nick”) in double strand DNA, i.e., cuts one strand but not the other of the DNA double helix. As used herein, an “RNA-guided DNA nickase” means a polypeptide or complex of polypeptides having DNA nickase activity, wherein the DNA nickase activity is sequence-specific and depends on the sequence of the RNA. Exemplary RNA-guided DNA nickases include Cas nickases. Cas nickases include nickase forms of a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. Class 2 Cas nickases include variants in which only one of the two catalytic domains is inactivated, which have RNA-guided DNA nickase activity. Class 2 Cas nickases include, for example, Cas9 (e.g., H840A, D10A, or N863A variants of SpyCas9), Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like protein domain. Cpf1 sequences of Zetsche are incorporated by reference in their entirety. See, e.g., Zetsche, Tables S1 and S3. “Cas9” encompasses S. pyogenes (Spy) Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 900), SGSETPGTSESA (SEQ ID NO: 901), or SGSETPGTSESATPEGGSGGS (SEQ ID NO: 902). In some embodiments, the linker is a peptide linker comprising one or more sequences selected from SEQ ID NOs: 903-971.

As used herein, the term “uracil glycosylase inhibitor” or “UGI” refers to a protein that is capable of inhibiting a uracil-DNA glycosylase (UDG) base-excision repair enzyme.

As used herein, “open reading frame” or “ORF” of a gene refers to a sequence consisting of a series of codons that specify the amino acid sequence of the protein that the gene codes for. The ORF begins with a start codon (e.g., ATG in DNA or AUG in RNA) and ends with a stop codon, e.g., TAA, TAG or TGA in DNA or UAA, UAG, or UGA in RNA.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, “indels” refer to insertion/deletion mutations consisting of a number of nucleotides that are either inserted or deleted, e.g. at the site of double-stranded breaks (DSBs), in a target nucleic acid.

As used herein, “reduced or eliminated” expression of a protein on a cell refers to a partial or complete loss of expression of the protein relative to an unmodified cell. In some embodiments, the surface expression of a protein on a cell is measured by flow cytometry and has “reduced or eliminated” surface expression relative to an unmodified cell as evidenced by a reduction in fluorescence signal upon staining with the same antibody against the protein. A cell that has “reduced or eliminated” surface expression of a protein by flow cytometry relative to an unmodified cell may be referred to as “negative” for expression of that protein as evidenced by a fluorescence signal similar to a cell stained with an isotype control antibody. The “reduction or elimination” of protein expression can be measured by other known techniques in the field with appropriate controls known to those skilled in the art.

As used herein, “knockdown” refers to a decrease in expression of a particular gene product (e.g., protein, mRNA, or both), e.g., as compared to expression of an unedited target sequence. Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include analyzing mRNA isolated from a sample of interest. In some embodiments, “knockdown” may refer to some loss of expression of a particular gene product, for example a decrease in the amount of mRNA transcribed or a decrease in the amount of protein expressed by a cell or population of cells (including in vivo populations such as those found in tissues).

As used herein, “knockout” refers to a loss of expression from a particular gene or of a particular protein in a cell. Knockout can result in a decrease in expression below the level of detection of the assay. Knockout can be measured either by detecting total cellular amount of a protein in a cell, a tissue or a population of cells.

As used herein, a “target sequence” or “genomic target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including recurrence of the symptom.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### II. Genetically Modified Cells

A. Engineered Cell Compositions

The present disclosure provides engineered cell compositions which have reduced or eliminated surface expression of MHC class II relative to an unmodified cell. In some embodiments, the engineered cell composition comprises a genetic modification in the CIITA gene. In some embodiments, the engineered cell is an allogeneic cell. In some embodiments, the engineered cell with reduced MHC class II expression is useful for adoptive cell transfer therapies. In some embodiments, the engineered cell comprises additional genetic modifications in the genome of the cell to yield a cell that is desirable for allogeneic transplant purposes.

In some embodiments, an engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10877360-10923242. In some embodiments, an engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10902171-10923242. In some embodiments, the genetic modification comprises a modification of at least one nucleotide of a splice acceptor site. In some embodiments, the genetic modification comprises a modification of at least one nucleotide of a splice acceptor site, wherein the one nucleotide is adenine (A). In some embodiments, the genetic modification comprises a modification of at least one nucleotide of a splice acceptor site, wherein the one nucleotide is guanine (G). In some embodiments, the genetic modification comprises a modification of at least one nucleotide of a splice donor site. In some embodiments, the genetic modification comprises a modification of at least one nucleotide of a splice donor site, wherein the one nucleotide is guanine (G). In some embodiments, the genetic modification comprises a modification of at least one nucleotide of a splice donor site, wherein the one nucleotide is thymine (T). In some embodiments, the genetic modification comprises a modification of a splice site boundary nucleotide.

In some embodiments, an engineered cell which has reduced or eliminated surface expression of MHC class II relative to an unmodified cell is provided, comprising a genetic modification in the CIITA gene, wherein the modification comprises at least 5 contiguous nucleotides within the genomic coordinates chr16: 10902171-10923242. In some embodiments, the engineered cell which has reduced or eliminated surface expression of MHC class II relative to an unmodified cell, comprises a genetic modification in the CIITA gene, wherein the modification comprises at least 6, 7, 8, 9, or 10 contiguous nucleotides within the genomic coordinates chr16: 10902171-10923242.

In some embodiments, an engineered cell which has reduced or eliminated surface expression of MHC class II relative to an unmodified cell is provided, comprising a genetic modification in the CIITA gene, wherein the modification comprises at least one C to T substitution or at least one A to G substitution within the genomic coordinates chr16: 10902171-10923242.

In some embodiments, an engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10903873-chr:10923242

In some embodiments, an engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr:16:10906485-chr:10923242.

In some embodiments, an engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10908130-chr:10923242.

In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150.

In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, chr16:10916450-10916470, chr16:10923222-10923242, chr16:10916449-10916469, and chr16:10923214-10923234. In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, and chr16:10916450-10916470.

In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, and chr16:10922478-10922498.

In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chosen from: chr16:10908132-10908152, chr16:10908131-10908151, chr16:10916456-10916476, and chr16:10918504-10918524.

In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chr16:10908132-10908152. In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chr16:10908131-10908151. In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chr16:10916456-10916476. In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chr16:10918504-10918524.

In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-10906507. In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, and chr16:10922153-10922173.

In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chosen from: chr16:10918504-10918524, chr16:10923218-10923238, and chr16:10923219-10923239.

In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chr16:10918504-10918524. In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chr16:10923218-10923238. In some embodiments, an engineered cell, which has reduced or eliminated surface expression of MHC class II relative to an unmodified cell, comprises a genetic modification in the CIITA gene, the genetic modification comprises at least one nucleotide of a splice site within the genomic coordinates chr16:10923219-10923239.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: chr16:10895410-10895430, chr16:10898649-10898669, chr16:10898658-10898678, chr16:10902171-10902191, chr16:10902173-10902193, chr16:10902174-10902194, chr16:10902179-10902199, chr16:10902183-10902203, chr16:10902184-10902204, chr16:10902644-10902664, chr16:10902779-10902799, chr16:10902788-10902808, chr16:10902789-10902809, chr16:10902790-10902810, chr16:10902795-10902815, chr16:10902799-10902819, chr16:10903708-10903728, chr16:10903713-10903733, chr16:10903718-10903738, chr16:10903721-10903741, chr16:10903723-10903743, chr16:10903724-10903744, chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, chr16:10916450-10916470, chr16:10923222-10923242, chr16:10916449-10916469, and chr16:10923214-10923234. In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, and chr16:10916450-10916470. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, and chr16:10922478-10922498. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: chr16:10908132-10908152, chr16:10908131-10908151, chr16:10916456-10916476, and chr16:10918504-10918524.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chr16:10908132-10908152. In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chr16:10908131-10908151. In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chr16:10916456-10916476. In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chr16:10918504-10918524. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-10906507. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, and chr16:10922153-10922173. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chosen from: chr16:10918504-10918524, chr16:10923218-10923238, and chr16:10923219-10923239.

In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chr16:10918504-10918524. In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chr16:10923218-10923238. In some embodiments, an engineered cell, which has reduced or eliminated surface expression of MHC class II relative to an unmodified cell, comprises a genetic modification in the CIITA gene, wherein the genetic modification comprises an indel, a C to T substitution, or an A to G substitution within the genomic coordinates chr16:10923219-10923239. In some embodiments, the genetic modification comprises at least 5 contiguous nucleotides within the genomic coordinates. In some embodiments, the genetic modification comprises at least 6, 7, 8, 9, or 10 contiguous nucleotides within the genomic coordinates. In some embodiments, the genetic modification comprises at least one C to T substitution or at least one A to G substitution within the genomic coordinates.

In some embodiments, an engineered cell is provided that has reduced or eliminated surface expression of MHC class II by a gene editing system that binds to a CIITA genomic target sequence comprising at least 10 contiguous nucleotides within the genomic coordinates chosen from: chr16:10895410-10895430, chr16:10898649-10898669, chr16:10898658-10898678, chr16:10902171-10902191, chr16:10902173-10902193, chr16:10902174-10902194, chr16:10902179-10902199, chr16:10902183-10902203, chr16:10902184-10902204, chr16:10902644-10902664, chr16:10902779-10902799, chr16:10902788-10902808, chr16:10902789-10902809, chr16:10902790-10902810, chr16:10902795-10902815, chr16:10902799-10902819, chr16:10903708-10903728, chr16:10903713-10903733, chr16:10903718-10903738, chr16:10903721-10903741, chr16:10903723-10903743, chr16:10903724-10903744, chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242. In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10895410-10895430, chr16:10898649-10898669, chr16:10898658-10898678, chr16:10902171-10902191, chr16:10902173-10902193, chr16:10902174-10902194, chr16:10902179-10902199, chr16:10902183-10902203, chr16:10902184-10902204, chr16:10902644-10902664, chr16:10902779-10902799, chr16:10902788-10902808, chr16:10902789-10902809, chr16:10902790-10902810, chr16:10902795-10902815, chr16:10902799-10902819, chr16:10903708-10903728, chr16:10903713-10903733, chr16:10903718-10903738, chr16:10903721-10903741, chr16:10903723-10903743, chr16:10903724-10903744, chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided that has reduced or eliminated surface expression of MHC class II by a gene editing system that binds to a CIITA genomic target sequence comprising at least 10 contiguous nucleotides within the genomic coordinates chosen from: chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242. In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided that has reduced or eliminated surface expression of MHC class II by a gene editing system that binds to a CIITA genomic target sequence comprising at least 10 contiguous nucleotides within the genomic coordinates chosen from: chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242. In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided that has reduced or eliminated surface expression of MHC class II by a gene editing system that binds to a CIITA genomic target sequence comprising at least 10 contiguous nucleotides within the genomic coordinates chosen from: chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242. In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, chr16:10916450-10916470, chr16:10923222-10923242, chr16:10916449-10916469, and chr16:10923214-10923234. In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, and chr16:10916450-10916470. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, and chr16:10922478-10922498. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10908132-10908152, chr16:10908131-10908151, chr16:10916456-10916476, and chr16:10918504-10918524. In some embodiments, an engineered cell is provided that has reduced or eliminated surface expression of MHC class II by a gene editing system that binds to a CIITA genomic target sequence comprising at least 5 contiguous nucleotides within the genomic coordinates chr16:10908132-10908152. In some embodiments, an engineered cell is provided that has reduced or eliminated surface expression of MHC class II by a gene editing system that binds to a CIITA genomic target sequence comprising at least 5 contiguous nucleotides within the genomic coordinates chr16:10908131-10908151. In some embodiments, an engineered cell is provided that has reduced or eliminated surface expression of MHC class II by a gene editing system that binds to a CIITA genomic target sequence comprising at least 5 contiguous nucleotides within the genomic coordinates chr16:10916456-10916476. In some embodiments, an engineered cell is provided that has reduced or eliminated surface expression of MHC class II by a gene editing system that binds to a CIITA genomic target sequence comprising at least 5 contiguous nucleotides within the genomic coordinates chr16:10918504-10918524. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA-binding agent comprises a Cas9 protein, such as an S. pyogenes Cas9.

In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-10906507. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA binding agent comprises a deaminase. In some embodiments, the RNA-guided DNA-binding agent comprises a deaminase and an RNA-guided nickase. In some embodiments, the deaminase is a APOBEC3 deaminase, such as APOBEC3A (A3A).

In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, and chr16:10922153-10922173. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA binding agent comprises a deaminase. In some embodiments, the RNA-guided DNA-binding agent comprises a deaminase and an RNA-guided nickase. In some embodiments, the deaminase is a APOBEC3 deaminase, such as APOBEC3A (A3A).

In some embodiments, an engineered cell is provided that has reduced or eliminated surface expression of MHC class II by a gene editing system that binds to a CIITA genomic target sequence comprising at least 5 contiguous nucleotides within the genomic coordinates chosen from: chr16:10918504-10918524, chr16:10923218-10923238, and chr16:10923219-10923239. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA binding agent comprises a deaminase. In some embodiments, the RNA-guided DNA-binding agent comprises a deaminase and an RNA-guided nickase. In some embodiments, the deaminase is a APOBEC3 deaminase, such as APOBEC3A (A3A).

In some embodiments, an engineered cell is provided that has reduced or eliminated surface expression of MHC class II by a gene editing system that binds to a CIITA genomic target sequence comprising at least 5 contiguous nucleotides within the genomic coordinates:10918504-10918524. In some embodiments, an engineered cell is provided that has reduced or eliminated surface expression of MHC class II by a gene editing system that binds to a CIITA genomic target sequence comprising at least 5 contiguous nucleotides within the genomic coordinates chr16:10923218-10923238. In some embodiments, an engineered cell is provided that has reduced or eliminated surface expression of MHC class II by a gene editing system that binds to a CIITA genomic target sequence comprising at least 5 contiguous nucleotides within the genomic coordinates chr16:10923219-10923239. In some embodiments, the CIITA genomic target sequence comprises at least 10 contiguous nucleotides within the genomic coordinates. In some embodiments, the CIITA genomic target sequence comprises at least 15 contiguous nucleotides within the genomic coordinates. In some embodiments, the gene editing system comprises an RNA-guided DNA-binding agent. In some embodiments, the RNA-guided DNA binding agent comprises a deaminase. In some embodiments, the RNA-guided DNA-binding agent comprises a deaminase and an RNA-guided nickase. In some embodiments, the deaminase is a APOBEC3 deaminase, such as APOBEC3A (A3A).

In some embodiments, for each given range of genomic coordinates, a range may encompass +/−10 nucleotides on either end of the specified coordinates. For each given range of genomic coordinates, the range may encompass +/−5 nucleotides on either end of the range. For example, if chr16: 10923222-10923242 is given, in some embodiments the genomic target sequence or genetic modification may fall within chr16:10923212-10923252.

In some embodiments, a given range of genomic coordinates may comprise a target sequence on both strands of the DNA (i.e., the plus (+) strand and the minus (−) strand).

Genetic modifications in the CIITA gene are described further herein. In some embodiments, a genetic modification in the CIITA gene comprises any one or more of an insertion, deletion, substitution, or deamination of at least one nucleotide in a target sequence. In some embodiments, the engineered cell which has reduced or eliminated surface expression of MHC class II relative to an unmodified cell is provided, comprising a genetic modification in the CIITA gene, wherein the genetic modification inactivates a splice site. In some embodiments, the engineered cell which has reduced or eliminated surface expression of MHC class II relative to an unmodified cell is provided, comprising a genetic modification in the CIITA gene, wherein the genetic modification comprises an insertion at a splice site nucleotide. In some embodiments, the engineered cell which has reduced or eliminated surface expression of MHC class II relative to an unmodified cell is provided, comprising a genetic modification in the CIITA gene, wherein the genetic modification comprises a deletion of a splice site nucleotide. In some embodiments, the engineered cell which has reduced or eliminated surface expression of MHC class II relative to an unmodified cell is provided, comprising a genetic modification in the CIITA gene, wherein the genetic modification comprises a substitution of a splice site nucleotide. In some embodiments, the engineered cell which has reduced or eliminated surface expression of MHC class II relative to an unmodified cell is provided, comprising a genetic modification in the CIITA gene, wherein the genetic modification comprises a deamination of a splice site nucleotide.

In some embodiments, the engineered cell which has reduced or eliminated surface expression of MHC class II relative to an unmodified cell is provided, comprising a genetic modification in the CIITA gene as described herein, and wherein the cell further has reduced or eliminated surface expression of HLA-A. In some embodiments, the engineered cell comprises a genetic modification in the HLA-A gene. In some embodiments, the engineered cell comprises a genetic modification in the HLA-A gene and wherein the cell is homozygous for HLA-B and homozygous for HLA-C. In some embodiments, the engineered cell comprises a genetic modification that eliminates expression of HLA-A protein on the surface of the engineered cell.

The engineered human cells described herein may comprise a genetic modification in any HLA-A allele of the HLA-A gene. The HLA gene is located in chromosome 6 in a genomic region referred to as the HLA superlocus; hundreds of HLA-A alleles have been reported in the art (see e.g., Shiina et al., Nature 54:15-39 (2009). Sequences for HLA-A alleles are available in the art (see e.g., IPD-IMGT/HLA database for retrieving sequences of specific HLA-A alleles https://www.ebi.ac.uk/ipd/imgt/hla/allele.html).

In any of the embodiments above, the engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16: 10902171-10923242, further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of a splice site within the genomic coordinates chr16: 10902171-10923242, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of a splice site within the genomic coordinates chr16: 10902171-10923242, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, an engineered cell which has reduced or eliminated surface expression of MHC class II relative to an unmodified cell is provided, comprising a genetic modification in the CIITA gene, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-10906507, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, chr16:10916450-10916470, chr16:10923222-10923242, chr16:10916449-10916469, and chr16:10923214-10923234, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, an engineered cell which has reduced or eliminated surface expression of MHC class II relative to an unmodified cell is provided, comprising a genetic modification in the CIITA gene, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, chr16:10916450-10916470, chr16:10923222-10923242, chr16:10916449-10916469, and chr16:10923214-10923234, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, and chr16:10916450-10916470, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, an engineered cell which has reduced or eliminated surface expression of MHC class II relative to an unmodified cell is provided, comprising a genetic modification in the CIITA gene, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, and chr16:10916450-10916470, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, an engineered cell which has reduced or eliminated surface expression of MHC class II relative to an unmodified cell is provided, comprising a genetic modification in the CIITA gene, wherein the genetic modification comprises at least one nucleotide of a splice site within the genomic coordinates chosen from: chr16:10908132-10908152, chr16:10908131-10908151, chr16:10916456-10916476, chr16:10918504-10918524, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, an engineered cell which has reduced or eliminated surface expression of MHC class II relative to an unmodified cell is provided, comprising a genetic modification in the CIITA gene, wherein the genetic modification comprises at least one nucleotide of a splice site within the genomic coordinates chosen from: chr16:10918504-10918524, chr16:10923218-10923238, chr16:10923219-10923239, and wherein the cell further comprises a genetic modification in an HLA-A gene, and wherein the genetic modification in the HLA-A gene comprises at least one nucleotide within the genomic coordinates chosen from: chr6:29942854 to chr6:29942913 and chr6:29943518 to chr6: 29943619. In some embodiments, the cell comprises a genetic modification in an HLA-A gene, and wherein the genetic modification in the HLA-A gene comprises at least one nucleotide within the genomic coordinates chosen from: chr6:29942864 to chr6: 29942903. In some embodiments, the cell comprises a genetic modification in an HLA-A gene, and wherein the genetic modification in the HLA-A gene comprises at least one nucleotide within the genomic coordinates chosen from: chr6:29943528 to chr6:29943609. In some embodiments, the cell comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B and homozygous for HLA-C.

In some embodiments, the engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10902171-10923242, and wherein the cell further has reduced or eliminated surface expression of MHC class I. In some embodiments, the engineered cell comprises a genetic modification in the beta-2-microglobulin (B2M) gene. In some embodiments, the engineered cell comprises a genetic modification that reduces expression of MHC class I protein on the surface of the engineered cell.

In some embodiments, the engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10902171-10923242, and wherein the cell further comprises an exogenous nucleic acid. In some embodiments, the exogenous nucleic acid encodes a targeting receptor that is expressed on the surface of the engineered cell. In some embodiments, the targeting receptor is a chimeric antigen receptor (CAR). In some embodiments, the targeting receptor is a universal CAR (UniCar). In some embodiments, the targeting receptor is a T cell receptor (TCR). In some embodiments, the targeting receptor is a WT1 TCR. In some embodiments, the targeting receptor is a hybrid CAR/TCR. In some embodiments, the targeting receptor comprises an antigen recognition domain (e.g., a cancer antigen recognition domain and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the exogenous nucleic acid encodes a polypeptide that is secreted by the engineered cell (i.e., a soluble polypeptide). In some embodiments, the exogenous nucleic acid encodes a therapeutic polypeptide. In some embodiments, the exogenous nucleic acid encodes an antibody. In some embodiments, the exogenous nucleic acid encodes an enzyme. In some embodiments, the exogenous nucleic acid encodes a cytokine. In some embodiments, the exogenous nucleic acid encodes a chemokine. In some embodiments, the exogenous nucleic acid encodes a fusion protein.

In some embodiments, the engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10902171-10923242, wherein the cell further has reduced or eliminated surface expression of MHC class I, and wherein the cell further comprises an exogenous nucleic acid. In some embodiments, the engineered cell comprises a genetic modification in the beta-2-microglobulin (B2M) gene. In some embodiments, the engineered cell comprises a genetic modification that reduces expression of MHC class I protein on the surface of the engineered cell. In some embodiments, the exogenous nucleic acid encodes a targeting receptor that is expressed on the surface of the engineered cell. In some embodiments, the targeting receptor is a chimeric antigen receptor (CAR). In some embodiments, the targeting receptor is a universal CAR (UniCar). In some embodiments, the targeting receptor is a T cell receptor (TCR). In some embodiments, the targeting receptor is a WT1 TCR. In some embodiments, the targeting receptor is a hybrid CAR/TCR. In some embodiments, the targeting receptor comprises an antigen recognition domain (e.g., a cancer antigen recognition domain and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the exogenous nucleic acid encodes a polypeptide that is secreted by the engineered cell (i.e., a soluble polypeptide). In some embodiments, the exogenous nucleic acid encodes a therapeutic polypeptide. In some embodiments, the exogenous nucleic acid encodes an antibody. In some embodiments, the exogenous nucleic acid encodes an enzyme. In some embodiments, the exogenous nucleic acid encodes a cytokine. In some embodiments, the exogenous nucleic acid encodes a chemokine. In some embodiments, the exogenous nucleic acid encodes a fusion protein.

In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, wherein the cell further has reduced or eliminated surface expression of HLA-A, and wherein the cell further comprises an exogenous nucleic acid. In some embodiments, the engineered cell comprises a genetic modification in the HLA-A gene. In some embodiments, the engineered cell comprises a genetic modification that reduces expression of HLA-A protein on the surface of the engineered cell. In some embodiments, the exogenous nucleic acid encodes a targeting receptor that is expressed on the surface of the engineered cell. In some embodiments, the targeting receptor is a chimeric antigen receptor (CAR). In some embodiments, the targeting receptors is a universal CAR (UniCar). In some embodiments, the targeting receptor is a T cell receptor (TCR). In some embodiments, the targeting receptor is a WT1 TCR. In some embodiments, the targeting receptor is a hybrid CAR/TCR. In some embodiments, the targeting receptor comprises an antigen recognition domain (e.g., a cancer antigen recognition domain and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the exogenous nucleic acid encodes a polypeptide that is secreted by the engineered cell (i.e., a soluble polypeptide). In some embodiments, the exogenous nucleic acid encodes a therapeutic polypeptide. In some embodiments, the exogenous nucleic acid encodes an antibody. In some embodiments, the exogenous nucleic acid encodes an enzyme. In some embodiments, the exogenous nucleic acid encodes a cytokine. In some embodiments, the exogenous nucleic acid encodes a chemokine. In some embodiments, the exogenous nucleic acid encodes a fusion protein.

In some embodiments, the engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10902171-10923242, and wherein the cell further has reduced or eliminated expression of an endogenous TCR protein relative to an unmodified cell. In some embodiments, the engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10902171-10923242, and wherein the cell further comprises an exogenous nucleic acid, and further has reduced or eliminated expression of an endogenous TCR protein relative to an unmodified cell. In some embodiments, the engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10902171-10923242, and wherein the cell further has reduced or eliminated surface expression of MHC class I, and wherein the cell further has reduced or eliminated expression of an endogenous TCR protein relative to an unmodified cell.

In some embodiments, the engineered cell which has reduced or eliminated surface expression of MHC class II relative to an unmodified cell is provided, comprising a genetic modification in the CIITA gene, wherein the modification comprises at least one nucleotide of a splice site within the genomic coordinates chr16:10902171-10923242, and wherein the cell further comprises an exogenous nucleic acid, and wherein the cell further has reduced or eliminated surface expression of MHC class I, and wherein the cell further has reduced or eliminated expression of an endogenous TCR protein relative to an unmodified cell. In some embodiments, the engineered cell has reduced or eliminated expression of a TRAC protein relative to an unmodified cell. In some embodiments, the engineered cell has reduced or eliminated expression of a TRBC protein relative to an unmodified cell. In some embodiments, the engineered cell comprises a genetic modification in the beta-2-microglobulin (B2M) gene. In some embodiments, the engineered cell comprises a genetic modification that reduces expression of MHC class I protein on the surface of the engineered cell. In some embodiments, the exogenous nucleic acid encodes a targeting receptor that is expressed on the surface of the engineered cell. In some embodiments, the targeting receptor is a chimeric antigen receptor (CAR). In some embodiments, the targeting receptors is a universal CAR (UniCar). In some embodiments, the targeting receptor is a T cell receptor (TCR). In some embodiments, the targeting receptor is a WT1 TCR. In some embodiments, the targeting receptor is a hybrid CAR/TCR. In some embodiments, the targeting receptor comprises an antigen recognition domain (e.g., a cancer antigen recognition domain and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the exogenous nucleic acid encodes a polypeptide that is secreted by the engineered cell (i.e., a soluble polypeptide). In some embodiments, the exogenous nucleic acid encodes a therapeutic polypeptide. In some embodiments, the exogenous nucleic acid encodes an antibody. In some embodiments, the exogenous nucleic acid encodes an enzyme. In some embodiments, the exogenous nucleic acid encodes a cytokine. In some embodiments, the exogenous nucleic acid encodes a chemokine. In some embodiments, the exogenous nucleic acid encodes a fusion protein.

In some embodiments, the engineered cell which has reduced or eliminated surface expression of MHC class II relative to an unmodified cell is provided, comprising a genetic modification in the CIITA gene, wherein the modification comprises at least one nucleotide of an exon within the genomic coordinates chr16: 10902662-chr16:10923285, and wherein the cell further comprises an exogenous nucleic acid, and wherein the cell further has reduced or eliminated surface expression of HLA-A, and wherein the cell further has reduced or eliminated expression of an endogenous TCR protein relative to an unmodified cell. In some embodiments, the engineered cell has reduced or eliminated expression of a TRAC protein relative to an unmodified cell. In some embodiments, the engineered cell has reduced or eliminated expression of a TRBC protein relative to an unmodified cell. In some embodiments, the engineered cell comprises a genetic modification in the HLA-A gene. In some embodiments, the engineered cell comprises a genetic modification that reduces expression of HLA-A protein on the surface of the engineered cell. In some embodiments, the exogenous nucleic acid encodes a targeting receptor that is expressed on the surface of the engineered cell. In some embodiments, the targeting receptor is a chimeric antigen receptor (CAR). In some embodiments, the targeting receptors is a universal CAR (UniCar). In some embodiments, the targeting receptor is a T cell receptor (TCR). In some embodiments, the targeting receptor is a WT1 TCR. In some embodiments, the targeting receptor is a hybrid CAR/TCR. In some embodiments, the targeting receptor comprises an antigen recognition domain (e.g., a cancer antigen recognition domain and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the exogenous nucleic acid encodes a polypeptide that is secreted by the engineered cell (i.e., a soluble polypeptide). In some embodiments, the exogenous nucleic acid encodes a therapeutic polypeptide. In some embodiments, the exogenous nucleic acid encodes an antibody. In some embodiments, the exogenous nucleic acid encodes an enzyme. In some embodiments, the exogenous nucleic acid encodes a cytokine. In some embodiments, the exogenous nucleic acid encodes a chemokine. In some embodiments, the exogenous nucleic acid encodes a fusion protein.

The engineered cell may be any of the exemplary cell types disclosed herein. In some embodiments, the engineered cell is an immune cell. In some embodiments, the engineered cell is a hematopoetic stem cell (HSC). In some embodiments, the engineered cell is an induced pluripotent stem cell (iPSC). In some embodiments, the engineered cell is a monocyte, macrophage, mast cell, dendritic cell, or granulocyte. In some embodiments, the engineered cell is monocyte. In some embodiments, the engineered cell is a macrophage. In some embodiments, the engineered cell is a mast cell. In some embodiments, the engineered cell is a dendritic cell.

In some embodiments, the engineered cell is a granulocyte. In some embodiments, the engineered cell is a lymphocyte. In some embodiments, the engineered cell is a T cell. In some embodiments, the engineered cell is a CD4+ T cell. In some embodiments, the engineered cell is a CD8+ T cell. In some embodiments, the engineered cell is a memory T cell. In some embodiments, the engineered cell is a B cell. In some embodiments, the engineered cell is a plasma B cell. In some embodiments, the engineered cell is a memory B cell.

In some embodiments, the engineered cell is homozygous for HLA-B and homozygous for HLA-C. In some embodiments, the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02.

In some embodiments, the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the engineered cell is homozygous for HLA-B and homozygous for HLA-C and the HLA-B and HLA-C alleles are selected from any one of the following HLA-B and HLA-C alleles: HLA-B*07:02 and HLA-C*07:02; HLA-B*08:01 and HLA-C*07:01; HLA-B*44:02 and HLA-C*05:01; HLA-B*35:01 and HLA-C*04:01; HLA-B*40:01 and HLA-C*03:04; HLA-B*57:01 and HLA-C*06:02; HLA-B*14:02 and HLA-C*08:02; HLA-B*15:01 and HLA-C*03:03; HLA-B*13:02 and HLA-C*06:02; HLA-B*44:03 and HLA-C*16:01; HLA-B*38:01 and HLA-C*12:03; HLA-B*18:01 and HLA-C*07:01; HLA-B*44:03 and HLA-C*04:01; HLA-B*51:01 and HLA-C*15:02; HLA-B*49:01 and HLA-C*07:01; HLA-B*15:01 and HLA-C*03:04; HLA-B*18:01 and HLA-C*12:03; HLA-B*27:05 and HLA-C*02:02; HLA-B*35:03 and HLA-C*04:01; HLA-B*18:01 and HLA-C*05:01; HLA-B*52:01 and HLA-C*12:02; HLA-B*51:01 and HLA-C*14:02; HLA-B*37:01 and HLA-C*06:02; HLA-B*53:01 and HLA-C*04:01; HLA-B*55:01 and HLA-C*03:03; HLA-B*44:02 and HLA-C*07:04; HLA-B*44:03 and HLA-C*07:01; HLA-B*35:02 and HLA-C*04:01; HLA-B*15:01 and HLA-C*04:01; and HLA-B*40:02 and HLA-C*02:02. In some embodiments, the cell is homozygous for HLA-B and homozygous for HLA-C and the HLA-B and HLA-C alleles are HLA-B*07:02 and HLA-C*07:02. In some embodiments, the cell is homozygous for HLA-B and homozygous for HLA-C and the HLA-B and HLA-C alleles are HLA-B*08:01 and HLA-C*07:01. In some embodiments, the cell is homozygous for HLA-B and homozygous for HLA-C and the HLA-B and HLA-C alleles are HLA-B*44:02 and HLA-C*05:01. In some embodiments, the cell is homozygous for HLA-B and homozygous for HLA-C and the HLA-B and HLA-C alleles are HLA-B*35:01 and HLA-C*04:01.

In some embodiments, the disclosure provides a pharmaceutical composition comprising any one of the engineered cells disclosed herein. In some embodiments, the pharmaceutical composition comprises a population of any one of the engineered cells disclosed herein. In some embodiments, the pharmaceutical composition comprises a population of engineered cells that is at least 65% negative as measured by flow cytometry. In some embodiments, the pharmaceutical composition comprises a population of engineered cells that is at least 70% negative as measured by flow cytometry. In some embodiments, the pharmaceutical composition comprises a population of engineered cells that is at least 80% negative as measured by flow cytometry. In some embodiments, the pharmaceutical composition comprises a population of engineered cells that is at least 90% negative as measured by flow cytometry. In some embodiments, the pharmaceutical composition comprises a population of engineered cells that is at least 91% negative as measured by flow cytometry. In some embodiments, the pharmaceutical composition comprises a population of engineered cells that is at least 92% negative as measured by flow cytometry. In some embodiments, the pharmaceutical composition comprises a population of engineered cells that is at least 93% negative as measured by flow cytometry. In some embodiments, the pharmaceutical composition comprises a population of engineered cells that is at least 94% negative as measured by flow cytometry. In some embodiments, the pharmaceutical composition comprises a population of engineered cells that is at least 95% endogenous TCR protein negative as measured by flow cytometry. In some embodiments, the pharmaceutical composition comprises a population of engineered cells that is at least 97% endogenous TCR protein negative as measured by flow cytometry. In some embodiments, the pharmaceutical composition comprises a population of engineered cells that is at least 98% endogenous TCR protein negative as measured by flow cytometry. In some embodiments, the pharmaceutical composition comprises a population of engineered cells that is at least 99% endogenous TCR protein negative as measured by flow cytometry.

In some embodiments, methods are provided for administering the engineered cells or pharmaceutical compositions disclosed herein to a subject in need thereof. In some embodiments, methods are provided for administering the engineered cells or pharmaceutical compositions disclosed herein to a subject as an ACT therapy. In some embodiments, methods are provided for administering the engineered cells or pharmaceutical compositions disclosed herein to a subject as a treatment for cancer. In some embodiments, methods are provided for administering the engineered cells or pharmaceutical compositions disclosed herein to a subject as a treatment for an autoimmune disease. In some embodiments, methods are provided for administering the engineered cells or pharmaceutical compositions disclosed herein to a subject as a treatment for an infectious disease.

B. Methods and Compositions for Reducing or Eliminating Surface Expression of MHC Class II

The present disclosure provides methods and compositions for reducing or eliminating surface expression of MHC class II protein on a cell relative to an unmodified cell by genetically modifying the CIITA gene. The resultant genetically modified cell may also be referred to herein as an engineered cell. In some embodiments, an already-genetically modified (or engineered) cell may be the starting cell for further genetic modification using the methods or compositions provided herein. In some embodiments, the cell is an allogeneic cell. In some embodiments, a cell with reduced MHC class II expression is useful for adoptive cell transfer therapies. In some embodiments, editing of the CIITA gene is combined with additional genetic modifications to yield a cell that is desirable for allogeneic transplant purposes.

In some embodiments, the methods comprise reducing or eliminating surface expression of MHC class II protein on the surface of a cell comprising contacting a cell with a composition comprising a CIITA guide RNA comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide, wherein the CIITA guide targets a genomic target comprising at least 10 contiguous nucleotides within the genomic coordinates chr16:10902171-10923242. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 1-101.

In some embodiments, the methods comprise making an engineered cell, which has reduced or eliminated surface expression of MHC class II protein relative to an unmodified cell, comprising contact the cell with a composition comprising a CIITA guide RNA comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide, wherein the CIITA guide targets a genomic target comprising at least 10 contiguous nucleotides within the genomic coordinates chr16:10902171-10923242. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 1-101.

In some embodiments, the methods comprise genetically modifying a cell to reduce or eliminate the surface expression of MHC class II protein comprising contacting the cell with a composition comprising a CIITA guide RNA comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide, wherein the CIITA guide targets a genomic target comprising at least 10 contiguous nucleotides within the genomic coordinates chr16:10902171-10923242. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 1-101.

In some embodiments, the methods comprise inactivating a splice site in CIITA comprising contacting a cell with a composition comprising a CIITA guide RNA comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide, wherein the CIITA guide targets a genomic target comprising at least 10 contiguous nucleotides within the genomic coordinates chr16:10902171-10923242. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 1-101.

In some embodiments, the methods comprise inducing a DSB or an single stranded break (SSB) in CIITA comprising contacting a cell with a composition comprising a CIITA guide RNA comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide, wherein the CIITA guide targets a genomic target comprising at least 10 contiguous nucleotides within the genomic coordinates chr16:10902171-10923242. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 1-101.

In some embodiments, the methods comprise reducing expression of the CIITA protein in a cell comprising delivering a composition to a cell comprising contacting a cell with a composition comprising a CIITA guide RNA comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide, wherein the CIITA guide targets a genomic target comprising at least 10 contiguous nucleotides within the genomic coordinates chr16:10902171-10923242. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the expression of MHC class II protein on the surface of the cell (i.e., engineered cell) is thereby reduced. In some embodiments, the CIITA guide RNA comprises a guide sequence selected from SEQ ID NO: 1-101.

In some embodiments, the methods of reducing expression of an MHC class II protein on the surface of a cell comprise contacting a cell with any one or more of the CIITA guide RNAs disclosed herein. In some embodiments, the CIITA guide RNA comprises a guide sequence selected from SEQ ID NO: 1-101.

In some embodiments, compositions are provided comprising a CIITA guide RNA comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide, wherein the CIITA guide targets a genomic target comprising at least 10 contiguous nucleotides within the genomic coordinates chr16:10902171-10923242. In some embodiments, the composition further comprises an RNA-guided DNA binding agent or a nucleic acid encoding an RNA-guided DNA binding agent. In some embodiments, the composition comprises an RNA-guided DNA binding agent that is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase region. In some embodiments the RNA-guided DNA binding agent comprises an APOBEC3A deaminase (A3A) and an RNA-guided nickase. In some embodiments, the CIITA guide RNA comprises a guide sequence selected from SEQ ID NO: 1-101.

In some embodiments, the composition further comprises a uracil glycosylase inhibitor (UGI). In some embodiments, the composition comprises an RNA-guided DNA binding agent that the RNA-guided DNA binding agent generates a cytosine (C) to thymine (T) conversion with the CIITA genomic target sequence. In some embodiments, the composition comprises an RNA-guided DNA binding agent that generates a adenosine (A) to guanine (G) conversion with the CIITA genomic target sequence.

In some embodiments, an engineered cell produced by the methods described herein is provided. In some embodiments, the engineered cell produced by the methods and compositions described herein is an allogeneic cell. In some embodiments, the methods produce a composition comprising an engineered cell having reduced MHC class II expression. In some embodiments, the methods produce a composition comprising an engineered cell having reduced CIITA protein expression. In some embodiments, the methods produce a composition comprising an engineered cell having reduced CIITA levels in the cell nucleus. In some embodiments, the methods produce a composition comprising an engineered cell that expresses a truncated form of the CIITA protein. In some embodiments, the methods produce a composition comprising an engineered cell that produces no detectable CIITA protein. In some embodiments, the engineered cell has reduced MHC class II expression, reduced CIITA protein, and/or reduced CIITA levels in the cell nucleus as compared to an unmodified cell. In some embodiments, the engineered cell produced by the methods disclosed herein elicits a reduced response from CD4+ T cells as compared to an unmodified cell as measured in an in vitro cell culture assay containing CD4+ T cells.

In some embodiments, an engineered cell produced by the methods or compositions disclosed herein is provided wherein the cell has reduced or eliminated surface expression of MHC class II protein and wherein the cell comprises a genetic modification comprising a modification of at least one nucleotide of a splice acceptor site. In some embodiments, an engineered cell produced by the methods or compositions disclosed herein is provided wherein the cell has reduced or eliminated surface expression of MHC class II protein and wherein the cell comprises a genetic modification comprising a modification of at least one nucleotide of a splice donor site.

In some embodiments, an engineered cell produced by the methods or compositions disclosed herein is provided wherein the cell has reduced or eliminated surface expression of MHC class II protein and wherein the cell comprises a genetic modification comprising at least 5 contiguous nucleotides within the genomic coordinates chr16: 10902171-10923242. In some embodiments, an engineered cell produced by the methods or compositions disclosed herein is provided wherein the cell has reduced or eliminated surface expression of MHC class II protein and wherein the cell comprises a genetic modification comprising at least 10 contiguous nucleotides within the genomic coordinates chr16: 10902171-10923242. In some embodiments, an engineered cell produced by the methods or compositions disclosed herein is provided wherein the cell has reduced or eliminated surface expression of MHC class II protein and wherein the cell comprises a genetic modification comprising at least one C to T substitution or at least one A to G substitution within the genomic coordinates chr16: 10902171-10923242.

In some embodiments, an engineered cell produced by the methods or compositions disclosed herein is provided wherein the cell has reduced or eliminated surface expression of MHC class II protein and wherein the cell comprises a genetic modification comprising at least one nucleotide of a splice site within the genomic coordinates chr16:10903873-chr:10923242. In some embodiments, an engineered cell produced by the methods or compositions disclosed herein is provided wherein the cell has reduced or eliminated surface expression of MHC class II protein and wherein the cell comprises a genetic modification comprising at least one nucleotide of a splice site within the genomic coordinates chr:16:10906485-chr:10923242. In some embodiments, an engineered cell produced by the methods or compositions disclosed herein is provided wherein the cell has reduced or eliminated surface expression of MHC class II protein and wherein the cell comprises a genetic modification comprising at least one nucleotide of a splice site within the genomic coordinates chr16:10908130-chr:10923242.

In some embodiments, the compositions disclosed herein further comprise a pharmaceutically acceptable carrier. In some embodiments, a cell produced by the compositions disclosed herein comprising a pharmaceutically acceptable carrier is provided. In some embodiments, compositions comprising the cells disclosed herein are provided.

1. CIITA Guide RNAs

The methods and compositions provided herein disclose CIITA guide RNAs useful for reducing the expression of MHC class II protein on the surface of a cell. In some embodiments, such guide RNAs direct an RNA-guided DNA binding agent to a CIITA genomic target sequence and may be referred to herein as “CIITA guide RNAs.” In some embodiments, the CIITA guide RNA directs an RNA-guided DNA binding agent to a human CIITA genomic target sequence. In some embodiments, the CIITA guide RNA comprises a guide sequence selected from SEQ ID NO: 1-101.

In some embodiments, the methods and compositions disclosed herein comprise a CIITA guide RNA comprising a guide sequence that targets a CIITA genomic target sequence that comprises at least one nucleotide of a splice site, wherein the CIITA guide RNA targets a CIITA genomic target sequence comprising at least 10 contiguous nucleotides within the genomic coordinates chr16:10902171-10923242. In some embodiments, the methods and compositions disclosed herein comprise a CIITA guide RNA comprising a guide sequence that targets a CIITA genomic target sequence that comprises at least one nucleotide of a splice site, wherein the CIITA guide RNA targets a CIITA genomic target sequence comprising at least one nucleotide within the genomic coordinates chr16:10903873-chr:10923242. In some embodiments, the methods and compositions disclosed herein comprise a CIITA guide RNA comprising a guide sequence that targets a CIITA genomic target sequence that comprises at least one nucleotide of a splice site, wherein the CIITA guide RNA targets a CIITA genomic target sequence comprising at least one nucleotide within the genomic coordinates chr:16:10906485-chr:10923242. In some embodiments, the methods and compositions disclosed herein comprise a CIITA guide RNA comprising a guide sequence that targets a CIITA genomic target sequence that comprises at least one nucleotide of a splice site, wherein the CIITA guide RNA targets a CIITA genomic target sequence comprising at least one nucleotide within the genomic coordinates chr16:10908130-chr:10923242.

In some embodiments, the methods and compositions disclosed herein comprise a CIITA guide RNA comprising a guide sequence that targets a CIITA genomic target sequence that comprises at least one nucleotide of a splice acceptor site. In some embodiments, the one nucleotide of the splice acceptor site is A. In some embodiments, the one nucleotide of the splice acceptor site is G. In some embodiments, the one nucleotide is the splice site boundary nucleotide of the splice acceptor site. In some embodiments, the methods and compositions disclosed herein comprise a CIITA guide RNA comprising a guide sequence that targets a CIITA genomic target sequence that comprises at least one nucleotide of a splice donor site. In some embodiments, the one nucleotide of the splice donor site is G. In some embodiments, the one nucleotide of the splice donor site is U/T. In some embodiments, the one nucleotide is the splice site boundary nucleotide of the splice donor site.

In some embodiments, the methods and compositions disclosed herein comprise a CIITA guide RNA comprising a guide sequence that directs an RNA-guided DNA binding agent to make cut in a CIITA gene that is 5 nucleotides or less from a splice site boundary nucleotide, wherein the CIITA guide RNA targets a CIITA genomic target sequence comprising at least 10 contiguous nucleotides within the genomic coordinates chr16:10902171-10923242. In some embodiments, the methods and compositions disclosed herein comprise a CIITA guide RNA comprising a guide sequence that directs an RNA-guided DNA binding agent to make cut in a CIITA gene that is 5 nucleotides or less from a splice site boundary nucleotide, wherein the CIITA guide RNA targets a CIITA genomic target sequence comprising at least one nucleotide within the genomic coordinates chr16:10903873-chr:10923242. In some embodiments, the methods and compositions disclosed herein comprise a CIITA guide RNA comprising a guide sequence that directs an RNA-guided DNA binding agent to make cut in a CIITA gene that is 5 nucleotides or less from a splice site boundary nucleotide, wherein the CIITA guide RNA targets a CIITA genomic target sequence comprising at least one nucleotide within the genomic coordinates chr:16:10906485-chr:10923242. In some embodiments, the methods and compositions disclosed herein comprise a CIITA guide RNA comprising a guide sequence that directs an RNA-guided DNA binding agent to make cut in a CIITA gene that is 5 nucleotides or less from a splice site boundary nucleotide, wherein the CIITA guide RNA targets a CIITA genomic target sequence comprising at least one nucleotide within the genomic coordinates chr16:10908130-chr:10923242.

In some embodiments, the methods and compositions disclosed herein comprise a CIITA guide RNA comprising a guide sequence that directs an RNA-guided DNA binding agent to make cut in a CIITA gene that is 5 nucleotides or less from an acceptor splice site boundary nucleotide, wherein the CIITA guide RNA targets a CIITA genomic target sequence comprising at least 10 contiguous nucleotides within the genomic coordinates chr16:10902171-10923242. In some embodiments, the methods and compositions disclosed herein comprise a CIITA guide RNA comprising a guide sequence that directs an RNA-guided DNA binding agent to make a cut in a CIITA gene that is 5 nucleotides or less from a donor splice site boundary nucleotide, wherein the CIITA guide RNA targets a CIITA genomic target sequence comprising at least 10 contiguous nucleotides within the genomic coordinates chr16:10902171-10923242.

In some embodiments, the methods and compositions disclose a CIITA guide RNA that directs an RNA-guided DNA binding agent to make a cut in a CIITA genomic target sequence that is 5 nucleotides or less from a splice site boundary nucleotide. In embodiments wherein the RNA-guided DNA cutting agent is Cas9, the cut or “cut site” occurs at the third base from the protospacer adjacent motif (PAM) sequence.

In some embodiments, the methods and compositions disclosed herein comprise a CIITA guide RNA comprising a guide sequence that directs an RNA-guided DNA binding agent to make a cut in a CIITA gene that is 5 nucleotides or less from an acceptor splice site boundary nucleotide, wherein the cut site is 3′ of the acceptor splice site boundary nucleotide. In some embodiments, the methods and compositions disclosed herein comprise a CIITA guide RNA comprising a guide sequence that directs an RNA-guided DNA binding agent to make a cut in a CIITA gene at that is 5 nucleotides or less from an acceptor splice site boundary nucleotide, wherein the cut is 5′ of the acceptor splice site boundary nucleotide.

In some embodiments, the methods and compositions disclosed herein comprise a CIITA guide RNA comprising a guide sequence that directs an RNA-guided DNA binding agent to make a cut in a CIITA gene that is 5 nucleotides or less from a donor splice site boundary nucleotide, wherein the cut is 3′ of the donor splice site boundary nucleotide. In some embodiments, the methods and compositions disclosed herein comprise a CIITA guide RNA comprising a guide sequence that directs an RNA-guided DNA binding agent to make a cut in a CIITA gene that is 5 nucleotides or less from a donor splice site boundary nucleotide, wherein the cut is 5′ of the donor splice site boundary nucleotide.

In some embodiments, the CIITA guide comprises a guide sequence that directs an RNA-guided DNA binding agent to make a cut in a CIITA gene that is 4 nucleotides or less from an acceptor splice site boundary nucleotide. In some embodiments, the CIITA guide comprises a guide sequence that directs an RNA-guided DNA binding agent to make a cut in a CIITA gene that is 3 nucleotides or less from an acceptor splice site boundary nucleotide. In some embodiments, the CIITA guide comprises a guide sequence that directs an RNA-guided DNA binding agent to make a cut in a CIITA gene that is 2 nucleotides or less from an acceptor splice site boundary nucleotide. In some embodiments, the CIITA guide comprises a guide sequence that directs an RNA-guided DNA binding agent to make a cut in a CIITA gene that is 1 nucleotide or less from an acceptor splice site boundary nucleotide. In some embodiments, the CIITA guide comprises a guide sequence that directs an RNA-guided DNA binding agent to make a cut in a CIITA gene at an acceptor splice site boundary nucleotide.

In some embodiments, the CIITA guide comprises a guide sequence that directs an RNA-guided DNA binding agent to make a cut in a CIITA gene that is 4 nucleotides or less from a donor splice site boundary nucleotide. In some embodiments, the CIITA guide comprises a guide sequence that directs an RNA-guided DNA binding agent to make a cut in a CIITA gene that is 3 nucleotides or less from a donor splice site boundary nucleotide. In some embodiments, the CIITA guide comprises a guide sequence that directs an RNA-guided DNA binding agent to make a cut in a CIITA gene that is 2 nucleotides or less from a donor splice site boundary nucleotide. In some embodiments, the CIITA guide comprises a guide sequence that directs an RNA-guided DNA binding agent to make a cut in a CIITA gene that is 1 nucleotide or less from a donor splice site boundary nucleotide. In some embodiments, the CIITA guide comprises a guide sequence that directs an RNA-guided DNA binding agent to make a cut in a CIITA gene at a donor splice site boundary nucleotide.

In some embodiments, a composition is provided comprising a CIITA guide RNA described herein and an RNA-guided DNA binding agent or a nucleic acid encoding an RNA-guided DNA binding agent.

In some embodiments, a composition is provided comprising a CIITA single-guide RNA (sgRNA) comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide, wherein the CIITA guide targets a genomic target comprising at least 10 contiguous nucleotides within the genomic coordinates chr16:10902171-10923242. In some embodiments, a composition is provided comprising a CIITA sgRNA described herein and an RNA-guided DNA binding agent or a nucleic acid encoding an RNA-guided DNA binding agent.

In some embodiments, a composition is provided comprising a CIITA dual-guide RNA (dgRNA) comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide, wherein the CIITA guide targets a genomic target comprising at least 10 contiguous nucleotides within the genomic coordinates chr16:10902171-10923242. In some embodiments, a composition is provided comprising a CIITA dgRNA described herein and an RNA-guided DNA binding agent or a nucleic acid encoding an RNA-guided DNA binding agent.

Exemplary CIITA guide sequences are shown below in Table 1 (SEQ ID NOs: 1-101 with corresponding guide RNA sequences SEQ ID NOs: 200-300 and 301-401).

TABLE 1
Exemplary CIITA guide sequences.

| | | | Exemplary Mod Sequence | | |
|---|---|---|---|---|---|
| | | | (four terminal U | Exemplary | |
| | | | residues are | Full | |
| | SEQ ID | | optional and may | Sequence | |
| | NO to the | | include 0, 1, | (SEQ ID | |
| Guide | Guide | Guide | 2, 3, 4, or more Us) | NOs: 301- | Genomic |
| ID | Sequence | Sequence | (SEQ ID NOs: 200-300) | 401) | Coordinates |
| G018021 | 1 | UCCUAC | mU*mC*mC*UACCUGUC | UCCUACC | chr16:10877360- |
| | | CUGUCA | AGAGCCCCAGUUUUAG | UGUCAGA | 10877380 |
| | | GAGCCC | AmGmCmUmAmGmAmA | GCCCCAG | |
| | | CA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018022 | 2 | GAGCCC | mG*mA*mG*CCCCAAGG | GAGCCCC | chr16:10877372- |
| | | CAAGGU | UAAAAAGGCGUUUUAG | AAGGUAA | 10877392 |
| | | AAAAAG | AmGmCmUmAmGmAmA | AAAGGCG | |
| | | GC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018023 | 3 | AGCCCC | mA*mG*mC*CCCAAGGU | AGCCCCA | chr16:10877373- |
| | | AAGGUA | AAAAAGGCCGUUUUAG | AGGUAAA | 10877393 |
| | | AAAAGG | AmGmCmUmAmGmAmA | AAGGCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018024 | 4 | UUUCCC | mU*mU*mU*CCCGGCCU | UUUCCCG | chr16:10877379- |
| | | GGCCUU | UUUUACCUUGUUUUAG | GCCUUUU | 10877399 |
| | | UUUACC | AmGmCmUmAmGmAmA | UACCUUG | |
| | | UU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018025 | 5 | CUUUCC | mC*mU*mU*UCCCGGCC | CUUUCCC | chr16:10877380- |
| | | CGGCCU | UUUUUACCUGUUUUAG | GGCCUUU | 10877400 |
| | | UUUUAC | AmGmCmUmAmGmAmA | UUACCUG | |
| | | CU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018026 | 6 | ACACUG | mA*mC*mA*CUGUGAGC | ACACUGU | chr16:10895275- |
| | | UGAGCU | UGCCUGGGAGUUUUAG | GAGCUGC | 10895295 |
| | | GCCUGG | AmGmCmUmAmGmAmA | CUGGGAG | |
| | | GA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018027 | 7 | CACACU | mC*mA*mC*ACUGUGAG | CACACUG | chr16:10895276- |
| | | GUGAGC | CUGCCUGGGGUUUUAG | UGAGCUG | 10895296 |
| | | UGCCUG | AmGmCmUmAmGmAmA | CCUGGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018028 | 8 | GUGGCA | mG*mU*mG*GCACACUG | GUGGCAC | chr16:10895280- |
| | | CACUGU | UGAGCUGCCGUUUUAG | ACUGUGA | 10895300 |
| | | GAGCUG | AmGmCmUmAmGmAmA | GCUGCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018029 | 9 | GAGAUU | mG*mA*mG*AUUGAGC | GAGAUUG | chr16:10895409- |
| | | GAGCUC | UCUACUCAGGGUUUUA | AGCUCUA | 10895429 |
| | | UACUCA | GAmGmCmUmAmGmAm | CUCAGGG | |
| | | GG | AmAmUmAmGmCAAGU | UUUUAGA | |
| | | | UAAAAUAAGGCUAGUC | GCUAGAA | |
| | | | CGUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018030 | 10 | AGAUUG | mA*mG*mA*UUGAGCUC | AGAUUGA | chr16:10895410- |
| | | AGCUCU | UACUCAGGUGUUUUAG | GCUCUAC | 10895430 |
| | | ACUCAG | AmGmCmUmAmGmAmA | UCAGGUG | |
| | | GU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018031 | 11 | CAGGUG | mC*mA*mG*GUGGGCCC | CAGGUGG | chr16:10895425- |
| | | GGCCCU | UCCUCCCUCGUUUUAG | GCCCUCC | 10895445 |
| | | CCUCCC | AmGmCmUmAmGmAmA | UCCCUCG | |
| | | UC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018032 | 12 | CGGAAG | mC*mG*mG*AAGAGACC | CGGAAGA | chr16:10895437- |
| | | AGACCA | AGAGGGAGGGUUUUAG | GACCAGA | 10895457 |
| | | GAGGGA | AmGmCmUmAmGmAmA | GGGAGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018033 | 13 | UACCGG | mU*mA*mC*CGGAAGAG | UACCGGA | chr16:10895440- |
| | | AAGAGA | ACCAGAGGGGUUUUAG | AGAGACC | 10895460 |
| | | CCAGAG | AmGmCmUmAmGmAmA | AGAGGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018034 | 14 | AGGGAG | mA*mG*mG*GAGGCUU | AGGGAGG | chr16:10895742- |
| | | GCUUAU | AUGCCAAUAUGUUUUA | CUUAUGC | 10895762 |
| | | GCCAAU | GAmGmCmUmAmGmAm | CAAUAUG | |
| | | AU | AmAmUmAmGmCAAGU | UUUUAGA | |
| | | | UAAAAUAAGGCUAGUC | GCUAGAA | |
| | | | CGUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018035 | 15 | GGCUUA | mG*mG*mC*UUAUGCCA | GGCUUAU | chr16:10895747- |
| | | UGCCAA | AUAUCGGUGGUUUUAG | GCCAAUA | 10895767 |
| | | UAUCGG | AmGmCmUmAmGmAmA | UCGGUGG | |
| | | UG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018036 | 16 | UGACUG | mU*mG*mA*CUGCGCUU | UGACUGC | chr16:10898642- |
| | | CGCUUU | UUCCUUGUCGUUUUAG | GCUUUUC | 10898662 |
| | | UCCUUG | AmGmCmUmAmGmAmA | CUUGUCG | |
| | | UC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018037 | 17 | GACUGC | mG*mA*mC*UGCGCUUU | GACUGCG | chr16:10898643- |
| | | GCUUUU | UCCUUGUCUGUUUUAG | CUUUUCC | 10898663 |
| | | CCUUGU | AmGmCmUmAmGmAmA | UUGUCUG | |
| | | CU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018038 | 18 | GCUUUU | mG*mC*mU*UUUCCUUG | GCUUUUC | chr16:10898649- |
| | | CCUUGU | UCUGGGCAGGUUUUAG | CUUGUCU | 10898669 |
| | | CUGGGC | AmGmCmUmAmGmAmA | GGGCAGG | |
| | | AG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018039 | 19 | CCAGUU | mC*mC*mA*GUUCCGCU | CCAGUUC | chr16:10898658- |
| | | CCGCUG | GCCCAGACAGUUUUAG | CGCUGCC | 10898678 |
| | | CCCAGA | AmGmCmUmAmGmAmA | CAGACAG | |
| | | CA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018040 | 20 | GACCUG | mG*mA*mC*CUGAAGCA | GACCUGA | chr16:10901536- |
| | | AAGCAC | CUGGAAGCCGUUUUAG | AGCACUG | 10901556 |
| | | UGGAAG | AmGmCmUmAmGmAmA | GAAGCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018041 | 21 | GCACUG | mG*mC*mA*CUGGAAGC | GCACUGG | chr16:10901544- |
| | | GAAGCC | CAGGUGUGCGUUUUAG | AAGCCAG | 10901564 |
| | | AGGUGU | AmGmCmUmAmGmAmA | GUGUGCG | |
| | | GC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGm UmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018042 | 22 | CACUGG | mC*mA*mC*UGGAAGCC | CACUGGA | chr16:10901545- |
| | | AAGCCA | AGGUGUGCAGUUUUAG | AGCCAGG | 10901565 |
| | | GGUGUG | AmGmCmUmAmGmAmA | UGUGCAG | |
| | | CA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018043 | 23 | GGAAGC | mG*mG*mA*AGCCAGGU | GGAAGCC | chr16:10901549- |
| | | CAGGUG | GUGCAGGGCGUUUUAG | AGGUGUG | 10901569 |
| | | UGCAGG | AmGmCmUmAmGmAmA | CAGGGCG | |
| | | GC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018044 | 24 | AGCCAG | mA*mG*mC*CAGGUGUG | AGCCAGG | chr16:10901552- |
| | | GUGUGC | CAGGGCAGGGUUUUAG | UGUGCAG | 10901572 |
| | | AGGGCA | AmGmCmUmAmGmAmA | GGCAGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018045 | 25 | GCCCAC | mG*mC*mC*CACCUGCC | GCCCACC | chr16:10901557- |
| | | CUGCCC | CUGCACACCGUUUUAG | UGCCCUG | 10901577 |
| | | UGCACA | AmGmCmUmAmGmAmA | CACACCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018046 | 26 | GGGCUC | mG*mG*mG*CUCAGCUG | GGGCUCA | chr16:10902025- |
| | | AGCUGU | UGAGGAAGUGUUUUAG | GCUGUGA | 10902045 |
| | | GAGGAA | AmGmCmUmAmGmAmA | GGAAGUG | |
| | | GU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018047 | 27 | GGGGCU | mG*mG*mG*GCUCAGCU | GGGGCUC | chr16:10902026- |
| | | CAGCUG | GUGAGGAAGGUUUUAG | AGCUGUG | 10902046 |
| | | UGAGGA | AmGmCmUmAmGmAmA | AGGAAGG | |
| | | AG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018048 | 28 | GACCAG | mG*mA*mC*CAGAUUCC | GACCAGA | chr16:10902171- |
| | | AUUCCC | CAGUAUGUUGUUUUAG | UUCCCAG | 10902191 |
| | | AGUAUG | AmGmCmUmAmGmAmA | UAUGUUG | |
| | | UU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018049 | 29 | CCAGAU | mC*mC*mA*GAUUCCCA | CCAGAUU | chr16:10902173- |
| | | UCCCAG | GUAUGUUAGGUUUUAG | CCCAGUA | 10902193 |
| | | UAUGUU | AmGmCmUmAmGmAmA | UGUUAGG | |
| | | AG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGm UmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018050 | 30 | CAGAUU | mC*mA*mG*AUUCCCAG | CAGAUUC | chr16:10902174- |
| | | CCCAGU | UAUGUUAGGGUUUUAG | CCAGUAU | 10902194 |
| | | AUGUUA | AmGmCmUmAmGmAmA | GUUAGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018051 | 31 | UCCCAG | mU*mC*mC*CAGUAUGU | UCCCAGU | chr16:10902179- |
| | | UAUGUU | UAGGGGGCUGUUUUAG | AUGUUAG | 10902199 |
| | | AGGGGG | AmGmCmUmAmGmAmA | GGGGCUG | |
| | | CU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018052 | 32 | UCCAAG | mU*mC*mC*AAGCCCCC | UCCAAGC | chr16:10902183- |
| | | CCCCCU | UAACAUACUGUUUUAG | CCCCUAA | 10902203 |
| | | AACAUA | AmGmCmUmAmGmAmA | CAUACUG | |
| | | CU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018053 | 33 | CUCCAA | mC*mU*mC*CAAGCCCC | CUCCAAG | chr16:10902184- |
| | | GCCCCC | CUAACAUACGUUUUAG | CCCCCUA | 10902204 |
| | | UAACAU | AmGmCmUmAmGmAmA | ACAUACG | |
| | | AC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018054 | 34 | AAAGGC | mA*mA*mA*GGCACUGC | AAAGGCA | chr16:10902644- |
| | | ACUGCA | AAGAGACAAGUUUUAG | CUGCAAG | 10902664 |
| | | AGAGAC | AmGmCmUmAmGmAmA | AGACAAG | |
| | | AA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018055 | 35 | UCCAGU | mU*mC*mC*AGUAUAUU | UCCAGUA | chr16:10902779- |
| | | AUAUUC | CAUCUACCAGUUUUAG | UAUUCAU | 10902799 |
| | | AUCUAC | AmGmCmUmAmGmAmA | CUACCAG | |
| | | CA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018056 | 36 | UUCAUC | mU*mU*mC*AUCUACCA | UUCAUCU | chr16:10902788- |
| | | UACCAU | UGGUGAGUGGUUUUAG | ACCAUGG | 10902808 |
| | | GGUGAG | AmGmCmUmAmGmAmA | UGAGUGG | |
| | | UG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018057 | 37 | UCAUCU | mU*mC*mA*UCUACCAU | UCAUCUA | chr16:10902789- |
| | | ACCAUG | GGUGAGUGCGUUUUAG | CCAUGGU | 10902809 |
| | | GUGAGU | AmGmCmUmAmGmAmA | GAGUGCG | |
| | | GC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018058 | 38 | CAUCUA | mC*mA*mU*CUACCAUG | CAUCUAC | chr16:10902790- |
| | | CCAUGG | GUGAGUGCGGUUUUAG | CAUGGUG | 10902810 |
| | | UGAGUG | AmGmCmUmAmGmAmA | AGUGCGG | |
| | | CG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018059 | 39 | ACCAUG | mA*mC*mC*AUGGUGAG | ACCAUGG | chr16:10902795- |
| | | GUGAGU | UGCGGGGCCGUUUUAG | UGAGUGC | 10902815 |
| | | GCGGGG | AmGmCmUmAmGmAmA | GGGGCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018060 | 40 | GCCAGG | mG*mC*mC*AGGCCCCG | GCCAGGC | chr16:10902799- |
| | | CCCCGC | CACUCACCAGUUUUAG | CCCGCAC | 10902819 |
| | | ACUCAC | AmGmCmUmAmGmAmA | UCACCAG | |
| | | CA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018061 | 41 | CCACUC | mC*mC*mA*CUCUCCAC | CCACUCU | chr16:10903708- |
| | | UCCACC | CCCCAAUGUGUUUUAG | CCACCCC | 10903728 |
| | | CCCAAU | AmGmCmUmAmGmAmA | CAAUGUG | |
| | | GU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018062 | 42 | CUCCAC | mC*mU*mC*CACCCCCA | CUCCACC | chr16:10903713- |
| | | CCCCAA | AUGUAGGUGGUUUUAG | CCCAAUG | 10903733 |
| | | UGUAGG | AmGmCmUmAmGmAmA | UAGGUGG | |
| | | UG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGm UmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018063 | 43 | CACCUC | mC*mA*mC*CUCACCUA | CACCUCA | chr16:10903718- |
| | | ACCUAC | CAUUGGGGGGUUUUAG | CCUACAU | 10903738 |
| | | AUUGGG | AmGmCmUmAmGmAmA | UGGGGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018064 | 44 | GGGCAC | mG*mG*mG*CACCUCAC | GGGCACC | chr16:10903721- |
| | | CUCACC | CUACAUUGGGUUUUAG | UCACCUA | 10903741 |
| | | UACAUU | AmGmCmUmAmGmAmA | CAUUGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018065 | 45 | UGGGGC | mU*mG*mG*GGCACCUC | UGGGGCA | chr16:10903723- |
| | | ACCUCA | ACCUACAUUGUUUUAG | CCUCACC | 10903743 |
| | | CCUACA | AmGmCmUmAmGmAmA | UACAUUG | |
| | | UU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018066 | 46 | CUGGGG | mC*mU*mG*GGGCACCU | CUGGGGC | chr16:10903724- |
| | | CACCUC | CACCUACAUGUUUUAG | ACCUCAC | 10903744 |
| | | ACCUAC | AmGmCmUmAmGmAmA | CUACAUG | |
| | | AU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGm UmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018067 | 47 | ACCUCC | mA*mC*mC*UCCCGAGC | ACCUCCC | chr16:10903873- |
| | | CGAGCA | AAACAUGACGUUUUAG | GAGCAAA | 10903893 |
| | | AACAUG | AmGmCmUmAmGmAmA | CAUGACG | |
| | | AC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018068 | 48 | CCGAGC | mC*mC*mG*AGCAAACA | CCGAGCA | chr16:10903878- |
| | | AAACAU | UGACAGGUAGUUUUAG | AACAUGA | 10903898 |
| | | GACAGG | AmGmCmUmAmGmAmA | CAGGUAG | |
| | | UA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018069 | 49 | GUACCU | mG*mU*mA*CCUCUCAC | GUACCUC | chr16:10903905- |
| | | CUCACA | AGGCCCUAAGUUUUAG | UCACAGG | 10903925 |
| | | GGCCCU | AmGmCmUmAmGmAmA | CCCUAAG | |
| | | AA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018070 | 50 | AGUACC | mA*mG*mU*ACCUCUCA | AGUACCU | chr16:10903906- |
| | | UCUCAC | CAGGCCCUAGUUUUAG | CUCACAG | 10903926 |
| | | AGGCCC | AmGmCmUmAmGmAmA | GCCCUAG | |
| | | UA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018071 | 51 | GACGUC | mG*mA*mC*GUCUUGUG | GACGUCU | chr16:10904736- |
| | | UUGUGC | CUCUGGAGAGUUUUAG | UGUGCUC | 10904756 |
| | | UCUGGA | AmGmCmUmAmGmAmA | UGGAGAG | |
| | | GA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGm UmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018072 | 52 | AACAAG | mA*mA*mC*AAGCUUCC | AACAAGC | chr16:10904790- |
| | | CUUCCA | AAAAUGGCCGUUUUAG | UUCCAAA | 10904810 |
| | | AAAUGG | AmGmCmUmAmGmAmA | AUGGCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018073 | 53 | GAGAUC | mG*mA*mG*AUCCCGCA | GAGAUCC | chr16:10904811- |
| | | CCGCAU | UCACUCACCGUUUUAG | CGCAUCA | 10904831 |
| | | CACUCA | AmGmCmUmAmGmAmA | CUCACCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018074 | 54 | GCCCCU | mG*mC*mC*CCUGGCCU | GCCCCUG | chr16:10906481- |
| | | GGCCUU | UUGCAGAGCGUUUUAG | GCCUUUG | 10906501 |
| | | UGCAGA | AmGmCmUmAmGmAmA | CAGAGCG | |
| | | GC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGm UmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018075 | 55 | ACCGGC | mA*mC*mC*GGCUCUGC | ACCGGCU | chr16:10906485- |
| | | UCUGCA | AAAGGCCAGGUUUUAG | CUGCAAA | 10906505 |
| | | AAGGCC | AmGmCmUmAmGmAmA | GGCCAGG | |
| | | AG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018076 | 56 | CACCGG | mC*mA*mC*CGGCUCUG | CACCGGC | chr16:10906486- |
| | | CUCUGC | CAAAGGCCAGUUUUAG | UCUGCAA | 10906506 |
| | | AAAGGC | AmGmCmUmAmGmAmA | AGGCCAG | |
| | | CA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018077 | 57 | CCACCG | mC*mC*mA*CCGGCUCU | CCACCGG | chr16:10906487- |
| | | GCUCUG | GCAAAGGCCGUUUUAG | CUCUGCA | 10906507 |
| | | CAAAGG | AmGmCmUmAmGmAmA | AAGGCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018078 | 58 | CUGCUC | mC*mU*mG*CUCCACCG | CUGCUCC | chr16:10906492- |
| | | CACCGG | GCUCUGCAAGUUUUAG | ACCGGCU | 10906512 |
| | | CUCUGC | AmGmCmUmAmGmAmA | CUGCAAG | |
| | | AA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018079 | 59 | UCAGCU | mU*mC*mA*GCUGUGUC | UCAGCUG | chr16:10908127- |
| | | GUGUCA | ACCCGUUUCGUUUUAG | UGUCACC | 10908147 |
| | | CCCGUU | AmGmCmUmAmGmAmA | CGUUUCG | |
| | | UC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018080 | 60 | GCUGUG | mG*mC*mU*GUGUCACC | GCUGUGU | chr16:10908130- |
| | | UCACCC | CGUUUCAGGGUUUUAG | CACCCGU | 10908150 |
| | | GUUUCA | AmGmCmUmAmGmAmA | UUCAGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018081 | 61 | CUGUGU | mC*mU*mG*UGUCACCC | CUGUGUC | chr16:10908131- |
| | | CACCCG | GUUUCAGGUGUUUUAG | ACCCGUU | 10908151 |
| | | UUUCAG | AmGmCmUmAmGmAmA | UCAGGUG | |
| | | GU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018082 | 62 | UGUGUC | mU*mG*mU*GUCACCCG | UGUGUCA | chr16:10908132- |
| | | ACCCGU | UUUCAGGUGGUUUUAG | CCCGUUU | 10908152 |
| | | UUCAGG | AmGmCmUmAmGmAmA | CAGGUGG | |
| | | UG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018083 | 63 | CACCCG | mC*mA*mC*CCGUUUCA | CACCCGU | chr16:10908137- |
| | | UUUCAG | GGUGGGGUGGUUUUAG | UUCAGGU | 10908157 |
| | | GUGGGG | AmGmCmUmAmGmAmA | GGGGUGG | |
| | | UG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018084 | 64 | ACCCGU | mA*mC*mC*CGUUUCAG | ACCCGUU | chr16:10908138- |
| | | UUCAGG | GUGGGGUGAGUUUUAG | UCAGGUG | 10908158 |
| | | UGGGGU | AmGmCmUmAmGmAmA | GGGUGAG | |
| | | GA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018085 | 65 | CCCGUU | mC*mC*mC*GUUUCAGG | CCCGUUU | chr16:10908139- |
| | | UCAGGU | UGGGGUGAGGUUUUAG | CAGGUGG | 10908159 |
| | | GGGGUG | AmGmCmUmAmGmAmA | GGUGAGG | |
| | | AG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018086 | 66 | GUCUGA | mG*mU*mC*UGAGGCCC | GUCUGAG | chr16:10909006- |
| | | GGCCCU | UCCCUCCACGUUUUAG | GCCCUCC | 10909026 |
| | | CCCUCC | AmGmCmUmAmGmAmA | CUCCACG | |
| | | AC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018087 | 67 | UCUGAG | mU*mC*mU*GAGGCCCU | UCUGAGG | chr16:10909007- |
| | | GCCCUC | CCCUCCACAGUUUUAG | CCCUCCC | 10909027 |
| | | CCUCCA | AmGmCmUmAmGmAmA | UCCACAG | |
| | | CA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018088 | 68 | CAAGGC | mC*mA*mA*GGCAGCCC | CAAGGCA | chr16:10909018- |
| | | AGCCCU | UGUGGAGGGGUUUUAG | GCCCUGU | 10909038 |
| | | GUGGAG | AmGmCmUmAmGmAmA | GGAGGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018089 | 69 | GCUCAA | mG*mC*mU*CAAGGCAG | GCUCAAG | chr16:10909021- |
| | | GGCAGC | CCCUGUGGAGUUUUAG | GCAGCCC | 10909041 |
| | | CCUGUG | AmGmCmUmAmGmAmA | UGUGGAG | |
| | | GA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018090 | 70 | CGCUCA | mC*mG*mC*UCAAGGCA | CGCUCAA | chr16:10909022- |
| | | AGGCAG | GCCCUGUGGGUUUUAG | GGCAGCC | 10909042 |
| | | CCCUGU | AmGmCmUmAmGmAmA | CUGUGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018091 | 71 | UGUGCA | mU*mG*mU*GCAGACUC | UGUGCAG | chr16:10909172- |
| | | GACUCA | AGAGGUGAGGUUUUAG | ACUCAGA | 10909192 |
| | | GAGGUG | AmGmCmUmAmGmAmA | GGUGAGG | |
| | | AG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGm UmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018092 | 72 | UAACAU | mU*mA*mA*CAUUGCCU | UAACAUU | chr16:10910165- |
| | | UGCCUG | GUUCUCUCCGUUUUAG | GCCUGUU | 10910185 |
| | | UUCUCU | AmGmCmUmAmGmAmA | CUCUCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018093 | 73 | CUUCUC | mC*mU*mU*CUCGUCCU | CUUCUCG | chr16:10910176- |
| | | GUCCUG | GGAGAGAACGUUUUAG | UCCUGGA | 10910196 |
| | | GAGAGA | AmGmCmUmAmGmAmA | GAGAACG | |
| | | AC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018094 | 74 | UUCCGA | mU*mU*mC*CGAGGAAC | UUCCGAG | chr16:10910186- |
| | | GGAACU | UUCUCGUCCGUUUUAG | GAACUUC | 10910206 |
| | | UCUCGU | AmGmCmUmAmGmAmA | UCGUCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018095 | 75 | ACCCUU | mA*mC*mC*CUUGCUCU | ACCCUUG | chr16:10915547- |
| | | GCUCUU | UUGCCUCCUGUUUUAG | CUCUUUG | 10915567 |
| | | UGCCUC | AmGmCmUmAmGmAmA | CCUCCUG | |
| | | CU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018096 | 76 | UUGCUC | mU*mU*mG*CUCUUUGC | UUGCUCU | chr16:10915551- |
| | | UUUGCC | CUCCUAGGCGUUUUAG | UUGCCUC | 10915571 |
| | | UCCUAG | AmGmCmUmAmGmAmA | CUAGGCG | |
| | | GC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018097 | 77 | UGCUCU | mU*mG*mC*UCUUUGCC | UGCUCUU | chr16:10915552- |
| | | UUGCCU | UCCUAGGCUGUUUUAG | UGCCUCC | 10915572 |
| | | CCUAGG | AmGmCmUmAmGmAmA | UAGGCUG | |
| | | CU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018098 | 78 | CCUGAG | mC*mC*mU*GAGACAGG | CCUGAGA | chr16:10915567- |
| | | ACAGGG | GCCCAGCCUGUUUUAG | CAGGGCC | 10915587 |
| | | CCCAGC | AmGmCmUmAmGmAmA | CAGCCUG | |
| | | CU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018099 | 79 | AUCUGA | mA*mU*mC*UGAUUCCA | AUCUGAU | chr16:10916348- |
| | | UUCCAC | CCUGCAGCCGUUUUAG | UCCACCU | 10916368 |
| | | CUGCAG | AmGmCmUmAmGmAmA | GCAGCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018100 | 80 | CAGCGC | mC*mA*mG*CGCAUCCA | CAGCGCA | chr16:10916359- |
| | | AUCCAG | GGCUGCAGGGUUUUAG | UCCAGGC | 10916379 |
| | | GCUGCA | AmGmCmUmAmGmAmA | UGCAGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018101 | 81 | ACUCAG | mA*mC*mU*CAGCGCAU | ACUCAGC | chr16:10916362- |
| | | CGCAUC | CCAGGCUGCGUUUUAG | GCAUCCA | 10916382 |
| | | CAGGCU | AmGmCmUmAmGmAmA | GGCUGCG | |
| | | GC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018102 | 82 | AAACCC | mA*mA*mA*CCCUCAAG | AAACCCU | chr16:10916449- |
| | | UCAAGU | UGAGUGAGCGUUUUAG | CAAGUGA | 10916469 |
| | | GAGUGA | AmGmCmUmAmGmAmA | GUGAGCG | |
| | | GC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018103 | 83 | AACCCU | mA*mA*mC*CCUCAAGU | AACCCUC | chr16:10916450- |
| | | CAAGUG | GAGUGAGCUGUUUUAG | AAGUGAG | 10916470 |
| | | AGUGAG | AmGmCmUmAmGmAmA | UGAGCUG | |
| | | CU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018104 | 84 | GGCCCA | mG*mG*mC*CCAGCUCA | GGCCCAG | chr16:10916455- |
| | | GCUCAC | CUCACUUGAGUUUUAG | CUCACUC | 10916475 |
| | | UCACUU | AmGmCmUmAmGmAmA | ACUUGAG | |
| | | GA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018105 | 85 | AGGCCC | mA*mG*mG*CCCAGCUC | AGGCCCA | chr16:10916456- |
| | | AGCUCA | ACUCACUUGGUUUUAG | GCUCACU | 10916476 |
| | | CUCACU | AmGmCmUmAmGmAmA | CACUUGG | |
| | | UG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGm UmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018106 | 86 | CAGACU | mC*mA*mG*ACUGCGGG | CAGACUG | chr16:10918423- |
| | | GCGGGG | GACACAGUGGUUUUAG | CGGGGAC | 10918443 |
| | | ACACAG | AmGmCmUmAmGmAmA | ACAGUGG | |
| | | UG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018107 | 87 | CUGCAU | mC*mU*mG*CAUCCCUG | CUGCAUC | chr16:10918504- |
| | | CCCUGC | CUCAGGCUAGUUUUAG | CCUGCUC | 10918524 |
| | | UCAGGC | AmGmCmUmAmGmAmA | AGGCUAG | |
| | | UA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018108 | 88 | CCUGCU | mC*mC*mU*GCUCAGGC | CCUGCUC | chr16:10918511- |
| | | CAGGCU | UAAGGUGAGGUUUUAG | AGGCUAA | 10918531 |
| | | AAGGUG | AmGmCmUmAmGmAmA | GGUGAGG | |
| | | AG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018109 | 89 | CUGCUC | mC*mU*mG*CUCAGGCU | CUGCUCA | chr16:10918512- |
| | | AGGCUA | AAGGUGAGUGUUUUAG | GGCUAAG | 10918532 |
| | | AGGUGA | AmGmCmUmAmGmAmA | GUGAGUG | |
| | | GU | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018110 | 90 | CAGCAC | mC*mA*mG*CACCUGAC | CAGCACC | chr16:10918539- |
| | | CUGACC | CGGUAUCCGGUUUUAG | UGACCGG | 10918559 |
| | | GGUAUC | AmGmCmUmAmGmAmA | UAUCCGG | |
| | | CG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018111 | 91 | GUACAA | mG*mU*mA*CAAGCUGU | GUACAAG | chr16:10922153- |
| | | GCUGUC | CGGAAACAGGUUUUAG | CUGUCGG | 10922173 |
| | | GGAAAC | AmGmCmUmAmGmAmA | AAACAGG | |
| | | AG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018112 | 92 | GGAGAC | mG*mG*mA*GACGCUGG | GGAGACG | chr16:10922478- |
| | | GCUGGC | CGUAAGUCCGUUUUAG | CUGGCGU | 10922498 |
| | | GUAAGU | AmGmCmUmAmGmAmA | AAGUCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGm UmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018113 | 93 | GGCGUA | mG*mG*mC*GUAAGUCC | GGCGUAA | chr16:10922487- |
| | | AGUCCA | AGGCAACCCGUUUUAG | GUCCAGG | 10922507 |
| | | GGCAAC | AmGmCmUmAmGmAmA | CAACCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018114 | 94 | CUCCAC | mC*mU*mC*CACCCACC | CUCCACC | chr16:10922499- |
| | | CCACCA | AGGGUUGCCGUUUUAG | CACCAGG | 10922519 |
| | | GGGUUG | AmGmCmUmAmGmAmA | GUUGCCG | |
| | | CC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018115 | 95 | UGAGUC | mU*mG*mA*GUCCCAUC | UGAGUCC | chr16:10923205- |
| | | CCAUCC | CCCCCUUGCGUUUUAG | CAUCCCC | 10923225 |
| | | CCCCUU | AmGmCmUmAmGmAmA | CCUUGCG | |
| | | GC | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018116 | 96 | CCACAU | mC*mC*mA*CAUCCUGC | CCACAUC | chr16:10923214- |
| | | CCUGCA | AAGGGGGGAGUUUUAG | CUGCAAG | 10923234 |
| | | AGGGGG | AmGmCmUmAmGmAmA | GGGGGAG | |
| | | GA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGm UmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018117 | 97 | GCGUCC | mG*mC*mG*UCCACAUC | GCGUCCA | chr16:10923218- |
| | | ACAUCC | CUGCAAGGGGUUUUAG | CAUCCUG | 10923238 |
| | | UGCAAG | AmGmCmUmAmGmAmA | CAAGGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018118 | 98 | GGCGUC | mG*mG*mC*GUCCACAU | GGCGUCC | chr16:10923219- |
| | | CACAUC | CCUGCAAGGGUUUUAG | ACAUCCU | 10923239 |
| | | CUGCAA | AmGmCmUmAmGmAmA | GCAAGGG | |
| | | GG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018119 | 99 | GGGCGU | mG*mG*mG*CGUCCACA | GGGCGUC | chr16:10923220- |
| | | CCACAU | UCCUGCAAGGUUUUAG | CACAUCC | 10923240 |
| | | CCUGCA | AmGmCmUmAmGmAmA | UGCAAGG | |
| | | AG | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018120 | 100 | UGGGCG | mU*mG*mG*GCGUCCAC | UGGGCGU | chr16:10923221- |
| | | UCCACA | AUCCUGCAAGUUUUAG | CCACAUC | 10923241 |
| | | UCCUGC | AmGmCmUmAmGmAmA | CUGCAAG | |
| | | AA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |
| G018121 | 101 | GUGGGC | mG*mU*mG*GGCGUCCA | GUGGGCG | chr16:10923222- |
| | | GUCCAC | CAUCCUGCAGUUUUAG | UCCACAU | 10923242 |
| | | AUCCUG | AmGmCmUmAmGmAmA | CCUGCAG | |
| | | CA | mAmUmAmGmCAAGUU | UUUUAGA | |
| | | | AAAAUAAGGCUAGUCC | GCUAGAA | |
| | | | GUUAUCAmAmCmUmU | AUAGCAA | |
| | | | mGmAmAmAmAmAmGm | GUUAAAA | |
| | | | UmGmGmCmAmCmCmG | UAAGGCU | |
| | | | mAmGmUmCmGmGmUm | AGUCCGU | |
| | | | GmCmU*mU*mU*mU | UAUCAAC | |
| | | | | UUGAAAA | |
| | | | | AGUGGCA | |
| | | | | CCGAGUC | |
| | | | | GGUGCUU | |
| | | | | UU | |

The terms “mA,” “mC,” “mU,” or “mG” may be used to denote a nucleotide that has been modified with 2′-O-Me.

In some embodiments, the CIITA guide RNA comprises a guide sequence selected from SEQ TD NOs: 1-101. In some embodiments, the CIITA guide RNA comprises a guide sequence that is at least 17, 18, 19, or 20 contiguous nucleotides of a sequence selected from SEQ ID NOs: 1-101. In some embodiments, the CIITA guide RNA comprises a guide sequence that is at least 95%, 90%, or 85% identical to a sequence selected from SEQ ID NOs: 1-101. In some embodiments, the CIITA guide RNA comprises a guide sequence that is at least 95% identical to a sequence selected from SEQ ID NOs: 1-101.

In some embodiments, the CIITA guide RNA comprises a guide sequence that comprises at least 10 contiguous nucleotides±10 nucleotides of a genomic coordinate listed in Table 1. As used herein, at least 10 contiguous nucleotides±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Table 1. For example, a CIITA guide RNA may comprise 10 contiguous nucleotides within the genomic coordinates chr16:10877360-10877380 or within chr16:10877350-10877390, including the boundary nucleotides of these ranges. In some embodiments, the CIITA guide RNA comprises a guide sequence that is at least 17, 18, 19, or 20 contiguous nucleotides of a sequence that comprises 10 contiguous nucleotides±10 nucleotides of a genomic coordinate listed in Table 1. In some embodiments, the CIITA guide RNA comprises a guide sequence that is at least 95%, 90%, or 85% identical to a sequence selected from a sequence that is 17, 18, 19, or 20 contiguous nucleotides of a sequence that comprises 10 contiguous nucleotides±10 nucleotides of a genomic coordinate listed in Table 1.

In some embodiments, the CIITA guide RNA comprises a guide sequence that comprises at least 15 contiguous nucleotides±10 nucleotides of a genomic coordinate listed in Table 1. In some embodiments, the CIITA guide RNA comprises a guide sequence that comprises at least 20 contiguous nucleotides±10 nucleotides of a genomic coordinate listed in Table 1.

In some embodiments, the CIITA guide RNA comprises SEQ ID NO: 1. In some embodiments, the CIITA guide RNA comprises SEQ ID NO: 2. In some embodiments, the CIITA guide RNA comprises SEQ ID NO: 3. In some embodiments, the CIITA guide RNA comprises SEQ ID NO: 4. In some embodiments, the CIITA guide RNA comprises SEQ ID NO: 5. In some embodiments, the CIITA guide RNA comprises SEQ ID NO: 6. In some embodiments, the CIITA guide RNA comprises SEQ ID NO: 7. In some embodiments, the CIITA guide RNA comprises SEQ ID NO: 8. In some embodiments, the CIITA guide RNA comprises SEQ ID NO: 9. In some embodiments, the CIITA guide RNA comprises SEQ ID NO: 10. In some embodiments, the CIITA guide RNA comprises SEQ ID NO: 11. In some embodiments, the CIITA guide RNA comprises SEQ ID NO: 12. In some embodiments, the CIITA guide RNA comprises SEQ ID NO: 13. In some embodiments, the CIITA guide RNA comprises SEQ ID NO: 14. In some embodiments, the CIITA guide RNA comprises SEQ ID NO: 15. In some embodiments, the CIITA guide RNA comprises SEQ ID NO: 16. In some embodiments, the CIITA guide RNA comprises SEQ ID NO: 17. In some embodiments, the CIITA guide RNA comprises SEQ ID NO: 18. In some embodiments, the CIITA guide RNA comprises SEQ ID NO: 19. In some embodiments, the CIITA guide RNA comprises SEQ ID NO: 20. In some embodiments, the CIITA guide RNA comprises SEQ ID NO: 21. In some embodiments, the CIITA guide RNA comprises SEQ ID NO: 22. In some embodiments, the CIITA guide RNA comprises SEQ ID NO: 23. In some embodiments, the CIITA guide RNA comprises SEQ ID NO: 24. In some embodiments, the CIITA guide RNA comprises SEQ ID NO: 25. In some embodiments, the CIITA guide RNA comprises SEQ ID NO: 26. In some embodiments, the CIITA guide RNA comprises SEQ ID NO: 27. In some embodiments, the CIITA guide RNA comprises SEQ ID NO: 28. In some embodiments, the CIITA guide RNA comprises SEQ ID NO: 29. In some embodiments, the CIITA guide RNA comprises SEQ ID NO: 30. In some embodiments, the CIITA guide RNA comprises SEQ ID NO: 31. In some embodiments, the CIITA guide RNA comprises SEQ ID NO: 32. In some embodiments, the CIITA guide RNA comprises SEQ ID NO: 33. In some embodiments, the CIITA guide RNA comprises SEQ ID NO: 34. In some embodiments, the CIITA guide RNA comprises SEQ ID NO: 35. In some embodiments, the CIITA guide RNA comprises SEQ ID NO: 36. In some embodiments, the CIITA guide RNA comprises SEQ ID NO: 37. In some embodiments, the CIITA guide RNA comprises SEQ ID NO: 38. In some embodiments, the CIITA guide RNA comprises SEQ ID NO: 39. In some embodiments, the CIITA guide RNA comprises SEQ ID NO: 40. In some embodiments, the CIITA guide RNA comprises SEQ ID NO: 41. In some embodiments, the CIITA guide RNA comprises SEQ ID NO: 42. In some embodiments, the CIITA guide RNA comprises SEQ ID NO: 43. In some embodiments, the CIITA guide RNA comprises SEQ ID NO: 44. In some embodiments, the CIITA guide RNA comprises SEQ ID NO: 45. In some embodiments, the CIITA guide RNA comprises SEQ ID NO: 46. In some embodiments, the CIITA guide RNA comprises SEQ ID NO: 47. In some embodiments, the CIITA guide RNA comprises SEQ ID NO: 48. In some embodiments, the CIITA guide RNA comprises SEQ ID NO: 49. In some embodiments, the CIITA guide RNA comprises SEQ ID NO: 50. In some embodiments, the CIITA guide RNA comprises SEQ ID NO:51. In some embodiments, the CIITA guide RNA comprises SEQ ID NO: 52. In some embodiments, the CIITA guide RNA comprises SEQ ID NO: 53. In some embodiments, the CIITA guide RNA comprises SEQ ID NO: 54. In some embodiments, the CIITA guide RNA comprises SEQ ID NO: 55. In some embodiments, the CIITA guide RNA comprises SEQ ID NO: 56. In some embodiments, the CIITA guide RNA comprises SEQ ID NO: 57. In some embodiments, the CIITA guide RNA comprises SEQ ID NO: 58. In some embodiments, the CIITA guide RNA comprises SEQ ID NO: 59. In some embodiments, the CIITA guide RNA comprises SEQ ID NO: 60. In some embodiments, the CIITA guide RNA comprises SEQ ID NO: 61. In some embodiments, the CIITA guide RNA comprises SEQ ID NO: 62. In some embodiments, the CIITA guide RNA comprises SEQ ID NO: 63. In some embodiments, the CIITA guide RNA comprises SEQ ID NO: 64. In some embodiments, the CIITA guide RNA comprises SEQ ID NO: 65. In some embodiments, the CIITA guide RNA comprises SEQ ID NO: 66. In some embodiments, the CIITA guide RNA comprises SEQ ID NO: 67. In some embodiments, the CIITA guide RNA comprises SEQ ID NO: 68. In some embodiments, the CIITA guide RNA comprises SEQ ID NO: 69. In some embodiments, the CIITA guide RNA comprises SEQ ID NO: 70. In some embodiments, the CIITA guide RNA comprises SEQ ID NO: 71. In some embodiments, the CIITA guide RNA comprises SEQ ID NO: 72. In some embodiments, the CIITA guide RNA comprises SEQ ID NO: 73. In some embodiments, the CIITA guide RNA comprises SEQ ID NO: 74. In some embodiments, the CIITA guide RNA comprises SEQ ID NO: 75. In some embodiments, the CIITA guide RNA comprises SEQ ID NO: 76. In some embodiments, the CIITA guide RNA comprises SEQ ID NO: 77. In some embodiments, the CIITA guide RNA comprises SEQ ID NO: 78. In some embodiments, the CIITA guide RNA comprises SEQ ID NO: 79. In some embodiments, the CIITA guide RNA comprises SEQ ID NO: 80. In some embodiments, the CIITA guide RNA comprises SEQ ID NO: 81. In some embodiments, the CIITA guide RNA comprises SEQ ID NO: 82. In some embodiments, the CIITA guide RNA comprises SEQ ID NO: 83. In some embodiments, the CIITA guide RNA comprises SEQ ID NO: 84. In some embodiments, the CIITA guide RNA comprises SEQ ID NO: 85. In some embodiments, the CIITA guide RNA comprises SEQ ID NO: 86. In some embodiments, the CIITA guide RNA comprises SEQ ID NO: 87. In some embodiments, the CIITA guide RNA comprises SEQ ID NO: 88. In some embodiments, the CIITA guide RNA comprises SEQ ID NO: 89. In some embodiments, the CIITA guide RNA comprises SEQ ID NO: 90. In some embodiments, the CIITA guide RNA comprises SEQ ID NO: 91. In some embodiments, the CIITA guide RNA comprises SEQ ID NO: 92. In some embodiments, the CIITA guide RNA comprises SEQ ID NO: 93. In some embodiments, the CIITA guide RNA comprises SEQ ID NO: 94. In some embodiments, the CIITA guide RNA comprises SEQ ID NO: 95. In some embodiments, the CIITA guide RNA comprises SEQ ID NO: 96. In some embodiments, the CIITA guide RNA comprises SEQ ID NO: 97. In some embodiments, the CIITA guide RNA comprises SEQ ID NO: 98. In some embodiments, the CIITA guide RNA comprises SEQ ID NO: 99. In some embodiments, the CIITA guide RNA comprises SEQ ID NO: 100. In some embodiments, the CIITA guide RNA comprises SEQ ID NO: 101.

In some embodiments, the CIITA guide RNA comprises a nucleotide chosen from: SEQ ID NO: 47, SEQ ID NO: 55, SEQ ID NO: 71, SEQ ID NO: 80, SEQ ID NO: 82, SEQ ID NO: 83, SEQ ID NO: 87, SEQ ID NO: 91, SEQ ID NO: 96, SEQ ID NO: 97, SEQ ID NO: 98, SEQ ID NO: 100, and SEQ ID NO: 101.

Additional embodiments of CIITA guide RNAs are provided herein, including e.g., exemplary modifications to the guide RNA.

2. Genetic Modifications to CIITA

In some embodiments, the methods and compositions disclosed herein genetically modify at least one nucleotide of a splice site in the CIITA gene in a cell. Because CIITA protein regulates expression of MHC class II, in some embodiments, the genetic modification to CIITA alters the production of CIITA protein, and thereby reduces the expression of MHC class II protein on the surface of the genetically modified cell (or engineered cell). Genetic modifications encompass the population of modifications that results from contact with a gene editing system (e.g., the population of edits that result from Cas9 and a CIITA guide RNA, or the population of edits that result from BC22 and a CIITA guide RNA).

In some embodiments, the genetic modification comprises at least one nucleotide of a splice site within the genomic coordinates chr16:10902171-10923242. In some embodiments, the genetic modification comprises at least one nucleotide of a splice site within the genomic coordinates chr16:10903873-chr:10923242. In some embodiments, the genetic modification comprises at least one nucleotide of a splice site within the genomic coordinates chr:16:10906485-chr:10923242. In some embodiments, the genetic modification comprises at least one nucleotide of a splice site within the genomic coordinates chr16:10908130-chr:10923242.

In some embodiments, the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150.

In some embodiments, the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, and chr16:10922478-10922498.

In some embodiments, the genetic modification comprises at least one nucleotide of a splice site within the genomic coordinates chosen from: chr16:10908132-10908152, chr16:10908131-10908151, chr16:10916456-10916476, and chr16:10918504-10918524. In some embodiments, the genetic modification comprises at least one nucleotide of a splice site within the genomic coordinates chr16:10908132-10908152. In some embodiments, the genetic modification comprises at least one nucleotide of a splice site within the genomic coordinates chr16:10908131-10908151. In some embodiments, the genetic modification comprises at least one nucleotide of a splice site within the genomic coordinates chr16:10916456-10916476. In some embodiments, the genetic modification comprises at least one nucleotide of a splice site within the genomic coordinates chr16:10918504-10918524.

In some embodiments, the genetic modification comprises at least one nucleotide of a splice site within the genomic coordinates chosen from chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, chr16:10906487-10906507.

In some embodiments, the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, and chr16:10922153-10922173.

In some embodiments, the genetic modification comprises at least one nucleotide of a splice site within the genomic coordinates chosen from: chr16:10918504-10918524, chr16:10923218-10923238, and chr16:10923219-10923239. In some embodiments, the genetic modification comprises at least one nucleotide of a splice site within the genomic coordinates chr16:10918504-10918524. In some embodiments, the genetic modification comprises at least one nucleotide of a splice site within the genomic coordinates, chr16:10923218-10923238. In some embodiments, the genetic modification comprises at least one nucleotide of a splice site within the genomic coordinates chr16:10923219-10923239.

In some embodiments, the genetic modification comprises at least 5 contiguous nucleotides within the genomic coordinates chr16: 10902171-10923242. In some embodiments, the genetic modification comprises at least 10 contiguous nucleotides within the genomic coordinates chr16: 10902171-10923242. In some embodiments, the genetic modification comprises at least one C to T substitution or at least one A to G substitution within the genomic coordinates chr16: 10902171-10923242.

In some embodiments, the modification to CIITA comprises any one or more of an insertion, deletion, substitution or deamination of at least one nucleotide in a target sequence. In some embodiments, the modification to CIITA comprises an insertion of 1, 2, 3, 4 or 5 or more nucleotides in a target sequence. In some embodiments, the modification to CIITA comprises a deletion of 1, 2, 3, 4 or 5 or more nucleotides in a target sequence. In other embodiments, the modification to CIITA comprises an insertion of 1, 2, 3, 4, 5, 6, 7, 8, 9, 10, 15, 20 or 25 or more nucleotides in a target sequence. In other embodiments, the modification to CIITA comprises a deletion of 1, 2, 3, 4, 5, 6, 7, 8, 9, 10, 15, 20 or 25 or more nucleotides in a target sequence. In some embodiments, the modification to CIITA comprises an indel, which is generally defined in the art as an insertion or deletion of less than 1000 base pairs (bp). In some embodiments, the modification to CIITA comprises an indel which results in a frameshift mutation in a target sequence. In some embodiments, the modification to CIITA comprises a substitution of 1, 2, 3, 4, 5, 6, 7, 8, 9, 10, 15, 20 or 25 or more nucleotides in a target sequence. In some embodiments, the modification to CIITA comprises one or more of an insertion, deletion, or substitution of nucleotides resulting from the incorporation of a template nucleic acid. In some embodiments, the modification to CIITA comprises an insertion of a donor nucleic acid in a target sequence. In some embodiments, the modification to CIITA is not transient.

In some embodiments, at least one nucleotide of a splice site is modified. In some embodiments, at least one nucleotide of a splice acceptor site is modified. In some embodiments, at least one nucleotide of a splice donor site is modified. In some embodiments, a acceptor splice site boundary nucleotide is modified. In some embodiments, a donor splice site boundary nucleotide is modified. In some embodiments, one of the conserved nucleotides of a splice acceptor site is modified. In some embodiments, the conserved nucleotide of a splice acceptor site, A, is modified. In some embodiments, the conserved nucleotide of a splice acceptor site, G, is modified. In some embodiments, one of the conserved nucleotides of a splice donor site is modified. In some embodiments, the conserved nucleotide of a splice donor site, G, is modified. In some embodiments, the conserved nucleotide of a splice donor site, T, is modified.

In some embodiments, a nucleotide that is located 5 nucleotides or less from an acceptor splice site boundary nucleotide is modified. In some embodiments, a nucleotide that is located 4 nucleotides or less from an acceptor splice site boundary nucleotide is modified. In some embodiments, a nucleotide that is located 3 nucleotides or less from an acceptor splice site boundary nucleotide is modified. In some embodiments, a nucleotide that is located 2 nucleotides or less from an acceptor splice site boundary nucleotide is modified. In some embodiments, a nucleotide that is located 1 nucleotide or less from an acceptor splice site boundary nucleotide is modified.

In some embodiments, a nucleotide that is located 5 nucleotides or less from a donor splice site boundary is modified. In some embodiments, a nucleotide that is located 4 nucleotides or less from a donor splice site boundary nucleotide is modified. In some embodiments, a nucleotide that is located 3 nucleotides or less from a donor splice site boundary nucleotide is modified. In some embodiments, a nucleotide that is located 2 nucleotides or less from a donor splice site boundary nucleotide is modified. In some embodiments, a nucleotide that is located 1 nucleotide or less from a donor splice site boundary nucleotide is modified.

In some embodiments, the methods and compositions disclosed herein modify a splice site of CIITA in a cell using an RNA-guided DNA binding agent (e.g., a Cas enzyme). In some embodiments, the RNA-guided DNA binding agent is Cas9. In some embodiments, the RNA-guided DNA binding agent cuts CIITA 5 nucleotides or less from a splice site boundary nucleotide, wherein the CIITA guide RNA targets a CIITA genomic target sequence comprising at least 10 contiguous nucleotides within the genomic coordinates chr16:10902171-10923242. In some embodiments, the RNA-guided DNA binding agent cuts a CIITA 4 nucleotides or less from a splice site boundary nucleotide, wherein the CIITA guide RNA targets a CIITA genomic target sequence comprising at least 10 contiguous nucleotides within the genomic coordinates chr16:10902171-10923242. In some embodiments, the RNA-guided DNA binding agent cuts CIITA 3 nucleotides or less from a splice site boundary nucleotide, wherein the CIITA guide RNA targets a CIITA genomic target sequence comprising at least 10 contiguous nucleotides within the genomic coordinates chr16:10902171-10923242. In some embodiments, the RNA-guided DNA binding agent cuts CIITA 2 nucleotides or less from a splice site boundary nucleotide, wherein the CIITA guide RNA targets a CIITA genomic target sequence comprising at least 10 contiguous nucleotides within the genomic coordinates chr16:10902171-10923242. In some embodiments, the RNA-guided DNA binding agent cuts CIITA 1 nucleotide or less from a splice site boundary nucleotide, wherein the CIITA guide RNA targets a CIITA genomic target sequence comprising at least 10 contiguous nucleotides within the genomic coordinates chr16:10902171-10923242. In some embodiments, the RNA-guided DNA binding agent cuts CIITA at a splice site boundary nucleotide, wherein the CIITA guide RNA targets a CIITA genomic target sequence comprising at least 10 contiguous nucleotides within the genomic coordinates chr16:10902171-10923242. In some embodiments, the splice site boundary nucleotide is an acceptor splice site boundary nucleotide. In some embodiments, the splice site boundary nucleotide is a donor splice site boundary nucleotide.

In some embodiments, the genetic modification to CIITA inactivates the splice site, i.e., splicing does not occur at the modified splice site. In some embodiments, the genetic modification to CIITA inactivates a splice acceptor site. In some embodiments, the genetic modification to CIITA inactivates a splice donor site.

In some embodiments, the genetic modification to the splice site of CIITA removes all three nucleotides of a splice site. In some embodiments, the genetic modification removes 2 nucleotides of a splice site. In some embodiments, the genetic modification removes 1 nucleotide of a splice site. In some embodiments, the genetic modification to the splice site of CIITA removes 1 or 2 nucleotides of the splice acceptor site. In some embodiments, the genetic modification to the splice site of CIITA removes 1 or 2 nucleotides of the splice donor site. In some embodiments, at least 1 nucleotide of a splice site is deleted. In some embodiments, at least 2 nucleotides of a splice site are deleted. In some embodiments, the acceptor splice site boundary nucleotide is deleted. In some embodiments, the donor splice site boundary nucleotide is deleted.

In some embodiments, the genetic modification to CIITA results in utilization of an out-of-frame stop codon. In some embodiments, the genetic modification to CIITA results in exon skipping during splicing. In some embodiments, the genetic modification to CIITA results in reduced CIITA protein expression by the cell. In some embodiments, the genetic modification to CIITA results in reduced CIITA in the cell nucleus. In some embodiments, the modification to the splice site of CIITA results in reduced MHC class II protein expression on the surface of the cell.

In some embodiments, the genetic modification to CIITA results in a truncated form of the CIITA protein. In some embodiments, the truncated CIITA protein does not bind to GTP. In some embodiments, the truncated CIITA protein does not localize to the nucleus. In some embodiments, the CIITA protein (e.g., a truncated form of the CIITA protein) has impaired activity as compared to the wildtype CIITA protein's activity relating to regulating MHC class II expression. In some embodiments, MHC class II expression on the surface of a cell is reduced as a result of impaired CIITA protein activity. In some embodiments, MHC class II expression on the surface of a cell is absent as a result of impaired CIITA protein activity.

3. Efficacy of CIITA Guide RNAs

The efficacy of a CIITA guide RNA may be determined by techniques available in the art that assess the editing efficiency of a guide RNA, the levels of CIITA protein and/or mRNA, and/or the levels of MHC class II in a target cell. In some embodiments, the reduction or elimination of HLA-A protein on the surface of a cell may be determined by comparison to an unmodified cell (or “relative to an unmodified cell”). An engineered cell or cell population may also be compared to a population of unmodified cells.

In some embodiments, the efficacy of a CIITA guide RNA is determined by measuring levels of CIITA protein in a cell. The levels of CIITA protein may be detected by, e.g., cell lysate and western blot with an anti-CIITA antibody. In some embodiments, the efficacy of a CIITA guide RNA is determined by measuring levels of CIITA protein in the cell nucleus. In some embodiments, the efficacy of a CIITA guide RNA is determined by measuring levels of CIITA mRNA in a cell. The levels of CIITA mRNA may be detected by e.g., RT-PCR. In some embodiments, a decrease in the levels CIITA protein and/or CIITA mRNA in the target cell as compared to an unmodified cell is indicative of an effective splice site CIITA guide RNA.

An “unmodified cell” (or “unmodified cells”) refers to a control cell (or cells) of the same type of cell in an experiment or test, wherein the “unmodified” control cell has not been contacted with a CIITA guide (i.e., a non-engineered cell). Therefore, an unmodified cell (or cells) may be a cell that has not been contacted with a guide RNA, or a cell that has been contacted with a guide RNA that does not target CIITA.

In some embodiments, the efficacy of a CIITA guide RNA is determined by measuring the reduction or elimination of MHC class II protein expression by the target cells. The CIITA protein functions as a transactivator, activating the MHC class II promoter, and is essential for the expression of MHC class II protein. In some embodiments, MHC class II protein expression may be detected on the surface of the target cells. In some embodiments, MHC class II protein expression is measured by flow cytometry. In some embodiments, an antibody against MHC class II protein (e.g., anti-HLA-DR, -DQ, -DP) may be used to detect MHC class II protein expression e.g., by flow cytometry. In some embodiments, a reduction or elimination in MHC class II protein on the surface of a cell (or population of cells) as compared to an unmodified cell (or population of unmodified cells) is indicative of an effective CIITA guide RNA. In some embodiments, a cell (or population of cells) that has been contacted with a particular CIITA guide RNA and RNA-guided DNA binding agent that is negative for MHC class II protein by flow cytometry is indicative of an effective CIITA guide RNA.

In some embodiments, the MHC class II protein expression is reduced or eliminated in a population of cells using the methods and compositions disclosed herein. In some embodiments, the population of cells is enriched (e.g., by FACS or MACS) and is at least 65%, 70%, 80%, 90%, 91%, 92%, 93%, or 94% MHC class II negative as measured by flow cytometry relative to a population of unmodified cells. In some embodiments, the population of cells is not enriched (e.g., by FACS or MACS) and is at least 65%, 70%, 80%, 90%, 91%, 92%, 93%, or 94% MHC class II negative as measured by flow cytometry relative to a population of unmodified cells.

In some embodiments, the population of cells is at least 65% MHC class II negative as measured by flow cytometry relative to a population of unmodified cells. In some embodiments, the population of cells is at least 70% MHC class II negative as measured by flow cytometry relative to a population of unmodified cells. In some embodiments, the population of cells is at least 80% MHC class II negative as measured by flow cytometry relative to a population of unmodified cells. In some embodiments, the population of cells is at least 90% MHC class II negative as measured by flow cytometry relative to a population of unmodified cells. In some embodiments, the population of cells is at least 91% MHC class II negative as measured by flow cytometry relative to a population of unmodified cells. In some embodiments, the population of cells is at least 92% MHC class II negative as measured by flow cytometry relative to a population of unmodified cells. In some embodiments, the population of cells is at least 93% MHC class II negative as measured by flow cytometry relative to a population of unmodified cells. In some embodiments, the population of cells is at least 94% MHC class II negative as measured by flow cytometry relative to a population of unmodified cells.

In some embodiments, the efficacy of a CIITA guide RNA (i.e., the potency of the guide RNA) is related to the distance between the cut site in the genomic target sequence (e.g., generated by Cas9) relative to a splice site boundary nucleotide in CIITA. In some embodiments, there is a correlation between distance (calculated as the number of nucleotides between the cut site and a splice site boundary nucleotide) and the loss of MHC class II expression. In some embodiments, the shorter the distance between the splice site boundary nucleotide and the cut site (e.g., generated by Cas9), the greater the reduction in MHC class II expression by the target cell. In some embodiments, the distance between the splice site boundary nucleotide in CIITA and the cut site in the genomic target sequence is 5 nucleotides or less, 4 nucleotides or less, 3 nucleotides or less, 2 nucleotides or less, or 1 nucleotide or less. In some embodiments, the cut site is 5′ of the splice site boundary nucleotide. In some embodiments, the cut site is 3′ of the splice site boundary nucleotide. In some embodiments, the CIITA splice site boundary is an acceptor splice site boundary nucleotide. In some embodiments, the CIITA splice site boundary is a donor splice site boundary nucleotide.

In some embodiments, an effective CIITA guide RNA may be determined by measuring the response of immune cells in vitro or in vivo (e.g., CD4+ T cells) to the genetically modified target cell. A CD4+ T cell response may be evaluated by an assay that measures the activation response of CD4+ T cells e.g., CD4+ T cell proliferation, expression of activation markers, and/or cytokine production (IL-2, IL-12, IFN-7) (e.g., flow cytometry, ELISA). The response of CD4+ T cells may be evaluated in in vitro cell culture assays in which the genetically modified cell is co-cultured with cells comprising CD4+ T cells. For example, the genetically modified cell may be co-cultured e.g., with PBMCs, purified CD3+ T cells comprising CD4+ T cells, purified CD4+ T cells, or a CD4+ T cell line. The CD4+ T cell response elicited from the genetically modified cell may be compared to the response elicited from an unmodified cell. A reduced response from CD4+ T cells is indicative of an effective CIITA guide RNA.

The efficacy of a CIITA guide RNA may also be assessed by the survival of the cell post-editing. In some embodiments, the cell survives post editing for at least one week to six weeks. In some embodiments, the cell survives post editing for at least one week to twelve weeks. In some embodiments, the cell survives post editing for at least two weeks. In some embodiments, the cell survives post editing for at least three weeks. In some embodiments, the cell survives post editing for at least four weeks. In some embodiments, the cell survives post editing for at least five weeks. In some embodiments, the cell survives post editing for at least six weeks. The viability of a genetically modified cell may be measured using standard techniques, including e.g., by measures of cell death, by flow cytometry live/dead staining, or cell proliferation.

C. Methods and Compositions for Reducing or Elimination MHC Class II and Additional Modifications

1. MHC Class I Knock Out

In some embodiments, methods for reducing or eliminating expression of MHC class II protein on the surface of a cell by genetically modifying CIITA as disclosed herein are provided, wherein the methods further provide for reducing or eliminating expression of MHC class I protein on the surface of the cell relative to an unmodified cell. In one approach, MHC class I protein expression is reduced or eliminated by genetically modifying the B2M gene. In some embodiments, MHC class I protein expression is reduced or eliminated by contacting the cell with a B2M guide RNA. In another approach, expression of the MHC class I protein HLA-A reduced or eliminated by genetically modifying HLA-A thereby reducing or eliminating the surface expression of HLA-A in a human cell, wherein the human cell is homozygous for HLA-B and homozygous for HLA-C. Therefore, in some embodiments, HLA-A protein expression is reduced or eliminated by contacting a human cell with an HLA-A guide RNA, wherein the human cell is homozygous for HLA-B and homozygous for HLA-C. In some embodiments, the resulting cell is an allogeneic cell.

In some embodiments, the methods comprise reducing expression of MHC class II protein on the surface of a cell comprising genetically modifying CIITA comprising contacting the cell with a composition comprising a CIITA guide RNA disclosed herein, the method further comprising contacting the cell with a B2M guide RNA. In some embodiments, the method further comprises contacting the cell with an RNA-guided DNA binding agent. In some embodiments, the method further comprises inducing a DSB or an SSB in the B2M target sequence. In some embodiments, B2M expression is thereby reduced by the cell. In some embodiments, MHC class I protein expression is thereby reduced by the cell.

In some embodiments, the methods comprise inactivating a splice site in CIITA comprising contacting the cell with a composition comprising a CIITA guide RNA disclosed herein, the method further comprising contacting the cell with a B2M guide RNA. In some embodiments, the method further comprises contacting the cell with an RNA-guided DNA binding agent. In some embodiments, the method further comprises inducing a DSB or an SSB in the B2M target sequence. In some embodiments, B2M expression is thereby reduced by the cell. In some embodiments, MHC class I protein expression is thereby reduced by the cell.

In some embodiments, the methods comprise inducing a DSB or an SSB in CIITA comprising contacting the cell with a composition comprising a CIITA guide RNA disclosed herein, the method further comprising contacting the cell with a B2M guide RNA. In some embodiments, the method further comprises contacting the cell with an RNA-guided DNA binding agent. In some embodiments, the method further comprises inducing a DSB or an SSB in the B2M target sequence. In some embodiments, B2M expression is thereby reduced by the cell. In some embodiments, MHC class I protein expression is thereby reduced by the cell.

In some embodiments, the methods comprise reducing expression of the CIITA protein in a cell comprising delivering a composition to a cell comprising a CIITA guide RNA disclosed herein, the method further comprising contacting the cell with a B2M guide RNA. In some embodiments, the method further comprises contacting the cell with an RNA-guided DNA binding agent. In some embodiments, the method further comprises inducing a DSB or an SSB in the B2M target sequence. In some embodiments, B2M expression is thereby reduced by the cell. In some embodiments, MHC class I protein expression is thereby reduced by the cell.

In some embodiments, the B2M guide RNA targets the human B2M gene.

In some embodiments, the B2M guide RNA comprises SEQ ID NO: 701. In some embodiments, the B2M guide RNA comprises a guide sequence that is at least 17, 18, 19, or 20 contiguous nucleotides of SEQ ID NO: 701. In some embodiments, the B2M guide RNA comprises a guide sequence that is at least 95%, 90%, or 85% identical to SEQ ID NO: 701.

Additional embodiments of B2M guide RNAs are provided herein, including e.g., exemplary modifications to the guide RNA.

In some embodiments, the efficacy of a B2M guide RNA is determined by measuring levels of B2M protein in a cell relative to an unmodified cell. In some embodiments, the efficacy of a B2M guide RNA is determined by measuring levels of B2M protein expressed by the cell. In some embodiments, an antibody against B2M protein (e.g., anti-B2M) may be used to detect the level of B2M protein by e.g., flow cytometry. In some embodiments, the efficacy of a B2M guide RNA is determined by measuring levels of B2M mRNA in a cell e.g., by RT-PCR. In some embodiments, reduction or elimination in the levels of B2M protein or B2M mRNA is indicative of an effective B2M guide RNA as compared to the levels of B2M protein in an unmodified cell. In some embodiments, a cell (or population of cells) that is negative for B2M protein by flow cytometry as compared to an unmodified cell (or population of unmodified cells) is indicative of an effective B2M guide RNA. In some embodiments, a cell (or population of cells) that has been contacted with a particular B2M guide RNA and RNA-guided DNA binding agent that is negative for MHC class I protein by flow cytometry is indicative of an effective B2M guide RNA.

In some embodiments, the efficacy of a B2M guide RNA is determined by measuring levels of MHC class I protein on the surface of a cell. In some embodiments, MHC class I protein levels are measured by flow cytometry (e.g., with an antibody against HLA-A, HLA-B, or HLA-C). In some embodiments, the population of cells is at least 65% MHC I negative as measured by flow cytometry relative to a population of unmodified cells. In some embodiments, the population of cells is at least 70% MHC I negative as measured by flow cytometry relative to a population of unmodified cells. In some embodiments, the population of cells is at least 80% MHC class I negative as measured by flow cytometry relative to a population of unmodified cells. In some embodiments, the population of cells is at least 90% MHC I negative as measured by flow cytometry relative to a population of unmodified cells. In some embodiments, the population of cells is at least 95% MHC I negative as measured by flow cytometry relative to a population of unmodified cells. In some embodiments, the population of cells is at least 100% MHC class I negative as measured by flow cytometry relative to a population of unmodified cells.

In some embodiments, the methods comprise reducing or eliminating surface expression of MHC class II protein in an engineered cell relative to an unmodified cell comprising contacting the cell with a composition comprising a CIITA guide RNA disclosed herein, the method further comprising reducing or eliminating the HLA-A expression of the cell by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the methods comprise reducing or eliminating surface expression of MHC class II protein in an engineered cell relative to an unmodified cell comprising contacting the cell with a composition comprising a CIITA guide RNA disclosed herein, the method further comprising reducing or eliminating the HLA-A expression of the cell by a gene editing system that binds to an HLA-A genomic target sequence comprising at least 10 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A genomic coordinates are chosen from chr6:29942864-29942884. In some embodiments, the HLA-A genomic coordinates are chosen from chr6:29942868-29942888. In some embodiments, the HLA-A genomic coordinates are chosen from chr6:29942876-29942896. In some embodiments, the HLA-A genomic coordinates are chosen from chr6:29942877-29942897. In some embodiments, the HLA-A genomic coordinates are chosen from chr6:29942883-29942903. In some embodiments, the HLA-A genomic coordinates are chosen from chr6:29943126-29943146. In some embodiments, the HLA-A genomic coordinates are chosen from chr6:29943528-29943548. In some embodiments, the HLA-A genomic coordinates are chosen from chr6:29943529-29943549. In some embodiments, the HLA-A genomic coordinates are chosen from chr6:29943530-29943550. In some embodiments, the HLA-A genomic coordinates are chosen from chr6:29943537-29943557. In some embodiments, the HLA-A genomic coordinates are chosen from chr6:29943549-29943569. In some embodiments, the HLA-A genomic coordinates are chosen from chr6:29943589-29943609. In some embodiments, the HLA-A genomic coordinates are chosen from chr6:29944026-29944046. In some embodiments, the gene editing system comprises an RNA-guided DNA-binding agent. In some embodiments, the RNA-guided DNA-binding agent comprises a Cas9 protein, such as an S. pyogenes Cas9. In some embodiments, the cell is homozygous for HLA-B and homozygous for HLA-C.

In some embodiments, the methods comprise reducing or eliminating surface expression of MHC class II protein in an engineered cell relative to an unmodified cell comprising contacting the cell with a composition comprising a CIITA guide RNA disclosed herein, the method further comprising contacting the cell with an HLA-A guide RNA. In some embodiments the HLA-A guide RNA comprises a guide sequence selected from SEQ ID NOs: 2001-2095 (with corresponding guide RNA sequences SEQ ID NOs: 1811-1905 and 1906-2000) (see Table 2 below). In some embodiments, the method further comprises contacting the cell with an RNA-guided DNA binding agent. In some embodiments, the RNA-guided DNA-binding agent comprises a Cas9 protein, such as an S. pyogenes Cas9. In some embodiments, the cell is homozygous for HLA-B and homozygous for HLA-C.

In some embodiments, methods are provided for making an engineered cell which has reduced or eliminated surface expression of MHC class II protein relative to an unmodified cell, comprising: a. contacting the cell with a CIITA guide RNA, wherein the guide RNA comprises a guide sequence selected from SEQ ID NOs: 1-117; and b. contacting the cell with an HLA-A guide RNA, wherein the HLA-A guide RNA comprises a guide sequence selected from any one of SEQ ID NOs: 2001-2095 (with corresponding guide RNA sequences SEQ ID NOs: 1811-1905 and 1906-2000) (see Table 2 below); and c. optionally contacting the cell with an RNA-guided DNA binding agent or nucleic acid encoding an RNA-guided DNA binding agent; wherein the cell has reduced or eliminated surface expression of HLA-A in the cell relative to an unmodified cell. In some embodiments, the method comprises contacting the cell with an RNA-guided DNA binding agent or nucleic acid encoding an RNA-guided DNA binding agent. In some embodiments, the RNA-guided DNA binding agent comprises an S. pyogenes Cas9. In some embodiments, the cell is homozygous for HLA-B and homozygous for HLA-C.

Exemplary HLA-A guide RNAs are provided in Table 2 (guide sequences SEQ ID NOs: 2001-2095 (with corresponding guide RNA sequences SEQ ID NOs: 1811-1905 and 1906-2000).

TABLE 2
Exemplary HLA-A guide RNAs

| | | | | Exemplary Mod | |
|---|---|---|---|---|---|
| | | | | Sequence | |
| | | | | (four terminal U | |
| | | | | residues are | |
| | | | | optional and may | |
| | SEQ ID | | Exemplary | include 0, 1, 2, | |
| | NO to the | | Full Sequence | 3, 4, or more | |
| Guide | Guide | Guide | (SEQ ID NOs: | Us) (SEQ ID | Genomic |
| ID | Sequence | Sequence | 1811-1905) | NOs: 1906-2000) | Coordinates |
| G018983 | 2001 | UGGAGGGCC | UGGAGGGCC | mU*mG*mG*AGG | chr6:29945290- |
| | | UGAUGUGUG | UGAUGUGUG | GCCUGAUGUGUG | 29945310 |
| | | UU | UUGUUUUAG | UUGUUUUAGAmG | (mismatch to |
| | | | AGCUAGAAA | mCmUmAmGmAm | hg38 = 2) |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018984 | 2002 | GCCUGAUGU | GCCUGAUGU | mG*mC*mC*UGAU | chr6:29945296- |
| | | GUGUUGGGU | GUGUUGGGU | GUGUGUUGGGUG | 29945316 |
| | | GU | GUGUUUUAG | UGUUUUAGAmGm | (mismatch to |
| | | | AGCUAGAAA | CmUmAmGmAmA | hg38 = 2) |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018985 | 2003 | CCUGAUGUG | CCUGAUGUG | mC*mC*mU*GAUG | chr6:29945297- |
| | | UGUUGGGUG | UGUUGGGUG | UGUGUUGGGUGU | 29945317 |
| | | UU | UUGUUUUAG | UGUUUUAGAmGm | (mismatch to |
| | | | AGCUAGAAA | CmUmAmGmAmA | hg38 = 1) |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018986 | 2004 | CCCAACACCC | CCCAACACCC | mC*mC*mC*AACA | chr6:29945300- |
| | | AACACACAUC | AACACACAUC | CCCAACACACAU | 29945320 |
| | | | GUUUUAGAG | CGUUUUAGAmGm | (mismatch to |
| | | | CUAGAAAUA | CmUmAmGmAmA | hg38 = 1) |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018965 | 2005 | UCAGGAAAC | UCAGGAAAC | mU*mC*mA*GGA | chr6:29890117- |
| | | AUGAAGAAA | AUGAAGAAA | AACAUGAAGAAA | 29890137 |
| | | GC | GCGUUUUAG | GCGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G019018 | 2006 | AGGCGCCUG | AGGCGCCUG | mA*mG*mG*CGCC | chr6:29927058- |
| | | GGCCUCUCCC | GGCCUCUCCC | UGGGCCUCUCCC | 29927078 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018937 | 2007 | CGGGCUGGCC | CGGGCUGGCC | mC*mG*mG*GCUG | chr6:29934330- |
| | | UCCCACAAGG | UCCCACAAGG | GCCUCCCACAAG | 29934350 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018990 | 2008 | ACGGCCAUCC | ACGGCCAUCC | mA*mC*mG*GCCA | chr6:29942541- |
| | | UCGGCGUCU | UCGGCGUCU | UCCUCGGCGUCU | 29942561 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018991 | 2009 | GACGGCCAUC | GACGGCCAUC | mG*mA*mC*GGCC | chr6:29942542- |
| | | CUCGGCGUCU | CUCGGCGUCU | AUCCUCGGCGUC | 29942562 |
| | | | GUUUUAGAG | UGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018992 | 2010 | GACGCCGAG | GACGCCGAG | mG*mA*mC*GCCG | chr6:29942543- |
| | | GAUGGCCGU | GAUGGCCGU | AGGAUGGCCGUC | 29942563 |
| | | CA | CAGUUUUAG | AGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018993 | 2011 | UGACGGCCA | UGACGGCCA | mU*mG*mA*CGGC | chr6:29942543- |
| | | UCCUCGGCGU | UCCUCGGCGU | CAUCCUCGGCGU | 29942563 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018994 | 2012 | GGCGCCAUG | GGCGCCAUG | mG*mG*mC*GCCA | chr6:29942550- |
| | | ACGGCCAUCC | ACGGCCAUCC | UGACGGCCAUCC | 29942570 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018995 | 2013 | ACAGCGACGC | ACAGCGACGC | mA*mC*mA*GCGA | chr6:29942864- |
| | | CGCGAGCCAG | CGCGAGCCAG | CGCCGCGAGCCA | 29942884 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018996 | 2014 | CGACGCCGCG | CGACGCCGCG | mC*mG*mA*CGCC | chr6:29942868- |
| | | AGCCAGAGG | AGCCAGAGG | GCGAGCCAGAGG | 29942888 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018997 | 2015 | CGAGCCAGA | CGAGCCAGA | mC*mG*mA*GCCA | chr6:29942876- |
| | | GGAUGGAGC | GGAUGGAGC | GAGGAUGGAGCC | 29942896 |
| | | CG | CGGUUUUAG | GGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018998 | 2016 | CGGCUCCAUC | CGGCUCCAUC | mC*mG*mG*CUCC | chr6:29942876- |
| | | CUCUGGCUCG | CUCUGGCUCG | AUCCUCUGGCUC | 29942896 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018999 | 2017 | GAGCCAGAG | GAGCCAGAG | mG*mA*mG*CCAG | chr6:29942877- |
| | | GAUGGAGCC | GAUGGAGCC | AGGAUGGAGCCG | 29942897 |
| | | GC | GCGUUUUAG | CGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G019000 | 2018 | GCGCCCGCGG | GCGCCCGCGG | mG*mC*mG*CCCG | chr6:29942883- |
| | | CUCCAUCCUC | CUCCAUCCUC | CGGCUCCAUCCU | 29942903 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019001 | 2019 | GCCCGUCCGU | GCCCGUCCGU | mG*mC*mC*CGUC | chr6:29943062- |
| | | GGGGGAUGA | GGGGGAUGA | CGUGGGGGAUGA | 29943082 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019002 | 2020 | UCAUCCCCCA | UCAUCCCCCA | mU*mC*mA*UCCC | chr6:29943063- |
| | | CGGACGGGCC | CGGACGGGCC | CCACGGACGGGC | 29943083 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019003 | 2021 | AUCUCGGACC | AUCUCGGACC | mA*mU*mC*UCGG | chr6:29943092- |
| | | CGGAGACUG | CGGAGACUG | ACCCGGAGACUG | 29943112 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019004 | 2022 | GGGGUCCCGC | GGGGUCCCGC | mG*mG*mG*GUCC | chr6:29943115- |
| | | GGCUUCGGG | GGCUUCGGG | CGCGGCUUCGGG | 29943135 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019005 | 2023 | CUCGGGGUCC | CUCGGGGUCC | mC*mU*mC*GGGG | chr6:29943118- |
| | | CGCGGCUUCG | CGCGGCUUCG | UCCCGCGGCUUC | 29943138 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019006 | 2024 | UCUCGGGGU | UCUCGGGGU | mU*mC*mU*CGGG | chr6:29943119- |
| | | CCCGCGGCUU | CCCGCGGCUU | GUCCCGCGGCUU | 29943139 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019007 | 2025 | GUCUCGGGG | GUCUCGGGG | mG*mU*mC*UCGG | chr6:29943120- |
| | | UCCCGCGGCU | UCCCGCGGCU | GGUCCCGCGGCU | 29943140 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019008 | 2026 | GCAAGGGUC | GCAAGGGUC | mG*mC*mA*AGG | chr6:29943126- |
| | | UCGGGGUCCC | UCGGGGUCCC | GUCUCGGGGUCC | 29943146 |
| | | G | GGUUUUAGA | CGGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019009 | 2027 | GGACCCCGAG | GGACCCCGAG | mG*mG*mA*CCCC | chr6:29943128- |
| | | ACCCUUGCCC | ACCCUUGCCC | GAGACCCUUGCC | 29943148 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019010 | 2028 | GACCCCGAGA | GACCCCGAGA | mG*mA*mC*CCCG | chr6:29943129- |
| | | CCCUUGCCCC | CCCUUGCCCC | AGACCCUUGCCC | 29943149 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019011 | 2029 | CGAGACCCUU | CGAGACCCUU | mC*mG*mA*GACC | chr6:29943134- |
| | | GCCCCGGGAG | GCCCCGGGAG | CUUGCCCCGGGA | 29943154 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019012 | 2030 | CUCCCGGGGC | CUCCCGGGGC | mC*mU*mC*CCGG | chr6:29943134- |
| | | AAGGGUCUC | AAGGGUCUC | GGCAAGGGUCUC | 29943154 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019013 | 2031 | UCUCCCGGGG | UCUCCCGGGG | mU*mC*mU*CCCG | chr6:29943135- |
| | | CAAGGGUCU | CAAGGGUCU | GGGCAAGGGUCU | 29943155 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019014 | 2032 | CUCUCCCGGG | CUCUCCCGGG | mC*mU*mC*UCCC | chr6:29943136- |
| | | GCAAGGGUC | GCAAGGGUC | GGGGCAAGGGUC | 29943156 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019015 | 2033 | CCUUGCCCCG | CCUUGCCCCG | mC*mC*mU*UGCC | chr6:29943140- |
| | | GGAGAGGCC | GGAGAGGCC | CCGGGAGAGGCC | 29943160 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019016 | 2034 | CUGGGCCUCU | CUGGGCCUCU | mC*mU*mG*GGCC | chr6:29943142- |
| | | CCCGGGGCAA | CCCGGGGCAA | UCUCCCGGGGCA | 29943162 |
| | | | GUUUUAGAG | AGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019017 | 2035 | CCUGGGCCUC | CCUGGGCCUC | mC*mC*mU*GGGC | chr6:29943143- |
| | | UCCCGGGGCA | UCCCGGGGCA | CUCUCCCGGGGC | 29943163 |
| | | | GUUUUAGAG | AGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019019 | 2036 | UUUAGGCCA | UUUAGGCCA | mU*mU*mU*AGG | chr6:29943188- |
| | | AAAAUCCCCC | AAAAUCCCCC | CCAAAAAUCCCC | 29943208 |
| | | C | CGUUUUAGA | CCGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021208 | 2037 | CGCUGCAGCG | CGCUGCAGCG | mC*mG*mC*UGCA | chr6:29943528- |
| | | CACGGGUACC | CACGGGUACC | GCGCACGGGUAC | 29943548 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021209 | 2038 | GCUGCAGCGC | GCUGCAGCGC | mG*mC*mU*GCAG | chr6:29943529- |
| | | ACGGGUACC | ACGGGUACC | CGCACGGGUACC | 29943549 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021210 | 2039 | CUGCAGCGCA | CUGCAGCGCA | mC*mU*mG*CAGC | chr6:29943530- |
| | | CGGGUACCA | CGGGUACCA | GCACGGGUACCA | 29943550 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018932 | 2040 | CGCACGGGU | CGCACGGGU | mC*mG*mC*ACGG | chr6:29943536- |
| | | ACCAGGGGCC | ACCAGGGGCC | GUACCAGGGGCC | 29943556 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018933 | 2041 | GCACGGGUA | GCACGGGUA | mG*mC*mA*CGGG | chr6:29943537- |
| | | CCAGGGGCCA | CCAGGGGCCA | UACCAGGGGCCA | 29943557 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018934 | 2042 | CACGGGUACC | CACGGGUACC | mC*mA*mC*GGGU | chr6:29943538- |
| | | AGGGGCCAC | AGGGGCCAC | ACCAGGGGCCAC | 29943558 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018935 | 2043 | GGGAGGCGC | GGGAGGCGC | mG*mG*mG*AGG | chr6:29943549- |
| | | CCCGUGGCCC | CCCGUGGCCC | CGCCCCGUGGCC | 29943569 |
| | | C | CGUUUUAGA | CCGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018936 | 2044 | GCGAUCAGG | GCGAUCAGG | mG*mC*mG*AUCA | chr6:29943556- |
| | | GAGGCGCCCC | GAGGCGCCCC | GGGAGGCGCCCC | 29943576 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021211 | 2045 | UCCUUGUGG | UCCUUGUGG | mU*mC*mC*UUGU | chr6:29943589- |
| | | GAGGCCAGCC | GAGGCCAGCC | GGGAGGCCAGCC | 29943609 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018938 | 2046 | CUCCUUGUG | CUCCUUGUG | mC*mU*mC*CUUG | chr6:29943590- |
| | | GGAGGCCAG | GGAGGCCAG | UGGGAGGCCAGC | 29943610 |
| | | CC | CCGUUUUAG | CGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018939 | 2047 | GGCUGGCCUC | GGCUGGCCUC | mG*mG*mC*UGGC | chr6:29943590- |
| | | CCACAAGGA | CCACAAGGA | CUCCCACAAGGA | 29943610 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018940 | 2048 | UUGUCUCCCC | UUGUCUCCCC | mU*mU*mG*UCUC | chr6:29943599- |
| | | UCCUUGUGG | UCCUUGUGG | CCCUCCUUGUGG | 29943619 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018941 | 2049 | CCACAAGGA | CCACAAGGA | mC*mC*mA*CAAG | chr6:29943600- |
| | | GGGGAGACA | GGGGAGACA | GAGGGGAGACAA | 29943620 |
| | | AU | AUGUUUUAG | UGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018942 | 2050 | CACAAGGAG | CACAAGGAG | mC*mA*mC*AAGG | chr6:29943601- |
| | | GGGAGACAA | GGGAGACAA | AGGGGAGACAAU | 29943621 |
| | | UU | UUGUUUUAG | UGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018943 | 2051 | CAAUUGUCU | CAAUUGUCU | mC*mA*mA*UUG | chr6:29943602- |
| | | CCCCUCCUUG | CCCCUCCUUG | UCUCCCCUCCUU | 29943622 |
| | | U | UGUUUUAGA | GUGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018944 | 2052 | CCAAUUGUC | CCAAUUGUC | mC*mC*mA*AUUG | chr6:29943603- |
| | | UCCCCUCCUU | UCCCCUCCUU | UCUCCCCUCCUU | 29943623 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018945 | 2053 | AUCCCUCGAA | AUCCCUCGAA | mA*mU*mC*CCUC | chr6:29943774- |
| | | UACUGAUGA | UACUGAUGA | GAAUACUGAUGA | 29943794 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018946 | 2054 | AACCACUCAU | AACCACUCAU | mA*mA*mC*CACU | chr6:29943779- |
| | | CAGUAUUCG | CAGUAUUCG | CAUCAGUAUUCG | 29943799 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018947 | 2055 | GAACCACUCA | GAACCACUCA | mG*mA*mA*CCAC | chr6:29943780- |
| | | UCAGUAUUC | UCAGUAUUC | UCAUCAGUAUUC | 29943800 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018948 | 2056 | GAGGAAAAG | GAGGAAAAG | mG*mA*mG*GAA | chr6:29943822- |
| | | UCACGGGCCC | UCACGGGCCC | AAGUCACGGGCC | 29943842 |
| | | A | AGUUUUAGA | CAGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018949 | 2057 | GGCCCGUGAC | GGCCCGUGAC | mG*mG*mC*CCGU | chr6:29943824- |
| | | UUUUCCUCUC | UUUUCCUCUC | GACUUUUCCUCU | 29943844 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018950 | 2058 | UGCUUCACAC | UGCUUCACAC | mU*mG*mC*UUCA | chr6:29943857- |
| | | UCAAUGUGU | UCAAUGUGU | CACUCAAUGUGU | 29943877 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018951 | 2059 | GCUUCACACU | GCUUCACACU | mG*mC*mU*UCAC | chr6:29943858- |
| | | CAAUGUGUG | CAAUGUGUG | ACUCAAUGUGUG | 29943878 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018952 | 2060 | CUUCACACUC | CUUCACACUC | mC*mU*mU*CACA | chr6:29943859- |
| | | AAUGUGUGU | AAUGUGUGU | CUCAAUGUGUGU | 29943879 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018953 | 2061 | UUCACACUCA | UUCACACUCA | mU*mU*mC*ACAC | chr6:29943860- |
| | | AUGUGUGUG | AUGUGUGUG | UCAAUGUGUGUG | 29943880 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018954 | 2062 | UUGAGAAUG | UUGAGAAUG | mU*mU*mG*AGA | chr6:29944026- |
| | | GACAGGACA | GACAGGACA | AUGGACAGGACA | 29944046 |
| | | CC | CCGUUUUAG | CCGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021205 | 2063 | AGGCAUUUU | AGGCAUUUU | mA*mG*mG*CAU | chr6:29944077- |
| | | GCAUCUGUC | GCAUCUGUC | UUUGCAUCUGUC | 29944097 |
| | | AU | AUGUUUUAG | AUGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021206 | 2064 | CAGGCAUUU | CAGGCAUUU | mC*mA*mG*GCAU | chr6:29944078- |
| | | UGCAUCUGU | UGCAUCUGU | UUUGCAUCUGUC | 29944098 |
| | | CA | CAGUUUUAG | AGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018955 | 2065 | AGGGGCCCU | AGGGGCCCU | mA*mG*mG*GGCC | chr6:29944458- |
| | | GACCCUGCUA | GACCCUGCUA | CUGACCCUGCUA | 29944478 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018956 | 2066 | UGGGAAAAG | UGGGAAAAG | mU*mG*mG*GAA | chr6:29944478- |
| | | AGGGGAAGG | AGGGGAAGG | AAGAGGGGAAGG | 29944498 |
| | | UG | UGGUUUUAG | UGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018957 | 2067 | UGGAGGAGG | UGGAGGAGG | mU*mG*mG*AGG | chr6:29944597- |
| | | AAGAGCUCA | AAGAGCUCA | AGGAAGAGCUCA | 29944617 |
| | | GG | GGGUUUUAG | GGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018958 | 2068 | UGAGAUUUC | UGAGAUUUC | mU*mG*mA*GAU | chr6:29944642- |
| | | UUGUCUCAC | UUGUCUCAC | UUCUUGUCUCAC | 29944662 |
| | | UG | UGGUUUUAG | UGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018959 | 2069 | GAGAUUUCU | GAGAUUUCU | mG*mA*mG*AUU | chr6:29944643- |
| | | UGUCUCACU | UGUCUCACU | UCUUGUCUCACU | 29944663 |
| | | GA | GAGUUUUAG | GAGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018960 | 2070 | UAAAGCACC | UAAAGCACC | mU*mA*mA*AGC | chr6:29944772- |
| | | UGUUAAAAU | UGUUAAAAU | ACCUGUUAAAAU | 29944792 |
| | | GA | GAGUUUUAG | GAGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018961 | 2071 | AAUCUGUCC | AAUCUGUCC | mA*mA*mU*CUG | chr6:29944782- |
| | | UUCAUUUUA | UUCAUUUUA | UCCUUCAUUUUA | 29944802 |
| | | AC | ACGUUUUAG | ACGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018962 | 2072 | GUCACAGGG | GUCACAGGG | mG*mU*mC*ACAG | chr6:29944850- |
| | | GAAGGUCCC | GAAGGUCCC | GGGAAGGUCCCU | 29944870 |
| | | UG | UGGUUUUAG | GGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018964 | 2073 | AAACAUGAA | AAACAUGAA | mA*mA*mA*CAU | chr6:29944907- |
| | | GAAAGCAGG | GAAAGCAGG | GAAGAAAGCAGG | 29944927 |
| | | UG | UGGUUUUAG | UGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018966 | 2074 | UGUCCUGUG | UGUCCUGUG | mU*mG*mU*CCUG | chr6:29945024- |
| | | AGAUACCAG | AGAUACCAG | UGAGAUACCAGA | 29945044 |
| | | AA | AAGUUUUAG | AGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018967 | 2075 | AUGAAGGAG | AUGAAGGAG | mA*mU*mG*AAG | chr6:29945097- |
| | | GCUGAUGCC | GCUGAUGCC | GAGGCUGAUGCC | 29945117 |
| | | UG | UGGUUUUAG | UGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018968 | 2076 | AGGCUGAUG | AGGCUGAUG | mA*mG*mG*CUG | chr6:29945104- |
| | | CCUGAGGUCC | CCUGAGGUCC | AUGCCUGAGGUC | 29945124 |
| | | U | UGUUUUAGA | CUGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018969 | 2077 | GGCUGAUGC | GGCUGAUGC | mG*mG*mC*UGA | chr6:29945105- |
| | | CUGAGGUCC | CUGAGGUCC | UGCCUGAGGUCC | 29945125 |
| | | UU | UUGUUUUAG | UUGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018970 | 2078 | CACAAUAUCC | CACAAUAUCC | mC*mA*mC*AAUA | chr6:29945116- |
| | | CAAGGACCUC | CAAGGACCUC | UCCCAAGGACCU | 29945136 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018971 | 2079 | GGUCCUUGG | GGUCCUUGG | mG*mG*mU*CCUU | chr6:29945118- |
| | | GAUAUUGUG | GAUAUUGUG | GGGAUAUUGUGU | 29945138 |
| | | UU | UUGUUUUAG | UGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018972 | 2080 | GUCCUUGGG | GUCCUUGGG | mG*mU*mC*CUUG | chr6:29945119- |
| | | AUAUUGUGU | AUAUUGUGU | GGAUAUUGUGUU | 29945139 |
| | | UU | UUGUUUUAG | UGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018973 | 2081 | CUCCCAAACA | CUCCCAAACA | mC*mU*mC*CCAA | chr6:29945124- |
| | | CAAUAUCCCA | CAAUAUCCCA | ACACAAUAUCCC | 29945144 |
| | | | GUUUUAGAG | AGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018974 | 2082 | UCCUCUAGCC | UCCUCUAGCC | mU*mC*mC*UCUA | chr6:29945176- |
| | | ACAUCUUCU | ACAUCUUCU | GCCACAUCUUCU | 29945196 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018975 | 2083 | ACAGAAGAU | ACAGAAGAU | mA*mC*mA*GAA | chr6:29945177- |
| | | GUGGCUAGA | GUGGCUAGA | GAUGUGGCUAGA | 29945197 |
| | | GG | GGGUUUUAG | GGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018976 | 2084 | CCUCUAGCCA | CCUCUAGCCA | mC*mC*mU*CUAG | chr6:29945177- |
| | | CAUCUUCUG | CAUCUUCUG | CCACAUCUUCUG | 29945197 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018977 | 2085 | CCCACAGAAG | CCCACAGAAG | mC*mC*mC*ACAG | chr6:29945180- |
| | | AUGUGGCUA | AUGUGGCUA | AAGAUGUGGCUA | 29945200 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018978 | 2086 | GUCAGAUCCC | GUCAGAUCCC | mG*mU*mC*AGA | chr6:29945187- |
| | | ACAGAAGAU | ACAGAAGAU | UCCCACAGAAGA | 29945207 |
| | | G | GGUUUUAGA | UGGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018979 | 2087 | AUCUUCUGU | AUCUUCUGU | mA*mU*mC*UUCU | chr6:29945188- |
| | | GGGAUCUGA | GGGAUCUGA | GUGGGAUCUGAC | 29945208 |
| | | CC | CCGUUUUAG | CGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018980 | 2088 | CCCAGGCAGU | CCCAGGCAGU | mC*mC*mC*AGGC | chr6:29945228- |
| | | GACAGUGCCC | GACAGUGCCC | AGUGACAGUGCC | 29945248 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018981 | 2089 | CUGGGCACU | CUGGGCACU | mC*mU*mG*GGCA | chr6:29945230- |
| | | GUCACUGCCU | GUCACUGCCU | CUGUCACUGCCU | 29945250 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018982 | 2090 | CCUGGGCACU | CCUGGGCACU | mC*mC*mU*GGGC | chr6:29945231- |
| | | GUCACUGCCU | GUCACUGCCU | ACUGUCACUGCC | 29945251 |
| | | | GUUUUAGAG | UGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021207 | 2091 | CCCUGGGCAC | CCCUGGGCAC | mC*mC*mC*UGGG | chr6:29945232- |
| | | UGUCACUGCC | UGUCACUGCC | CACUGUCACUGC | 29945252 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018987 | 2092 | UUGGGUGUU | UUGGGUGUU | mU*mU*mG*GGU | chr6:29945308- |
| | | GGGCGGAAC | GGGCGGAAC | GUUGGGCGGAAC | 29945328 |
| | | AG | AGGUUUUAG | AGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018988 | 2093 | UGGAUGUAU | UGGAUGUAU | mU*mG*mG*AUG | chr6:29945361- |
| | | UGAGCAUGC | UGAGCAUGC | UAUUGAGCAUGC | 29945381 |
| | | GA | GAGUUUUAG | GAGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018989 | 2094 | GGAUGUAUU | GGAUGUAUU | mG*mG*mA*UGU | chr6:29945362- |
| | | GAGCAUGCG | GAGCAUGCG | AUUGAGCAUGCG | 29945382 |
| | | AU | AUGUUUUAG | AUGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018963 | 2095 | AACAUGAAG | AACAUGAAG | mA*mA*mC*AUG | chr6:31382543- |
| | | AAAGCAGGU | AAAGCAGGU | AAGAAAGCAGGU | 31382563 |
| | | GU | GUGUUUUAG | GUGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |

In some embodiments, the efficacy of an HLA-A guide RNA is determined by measuring levels of HLA-A protein on the surface of a cell. In some embodiments, HLA-A protein levels are measured by flow cytometry (e.g., with an antibody against HLA-A2 and/or HLA-A3). In some embodiments, the population of cells is at least 65% HLA-A negative as measured by flow cytometry relative to a population of unmodified cells. In some embodiments, the population of cells is at least 70% HLA-A negative as measured by flow cytometry relative to a population of unmodified cells. In some embodiments, the population of cells is at least 80% HLA-A negative as measured by flow cytometry relative to a population of unmodified cells. In some embodiments, the population of cells is at least 90% HLA-A negative as measured by flow cytometry relative to a population of unmodified cells. In some embodiments, the population of cells is at least 95% HLA-A negative as measured by flow cytometry relative to a population of unmodified cells. In some embodiments, the population of cells is at least 100% HLA-A negative as measured by flow cytometry relative to a population of unmodified cells.

In some embodiments, the efficacy of a B2M guide RNA or an HLA-A guide may be determined by measuring the response of immune cells in vitro or in vivo (e.g., CD8+ T cells) to the genetically modified target cell as compared to an unmodified cell. For example, a reduced response from CD8+ T cells is indicative of an effective B2M guide RNA or an HLA-A. A CD8+ T cell response may be evaluated by an assay that measures CD8+ T cell activation responses, e.g., CD8+ T cell proliferation, expression of activation markers, and/or cytokine production (IL-2, IFN-γ, TNF-α) (e.g., flow cytometry, ELISA). The CD8+ T cell response may be assessed in vitro or in vivo. In some embodiments, the CD8+ T cell response may be evaluated by co-culturing the genetically modified cell with CD8+ T cells in vitro. In some embodiments, CD8+ T cell activity may be evaluated in an in vivo model, e.g., a rodent model. In an in vivo model, e.g., genetically modified cells may be administered with CD8+ T cell; survival of the genetically modified cells is indicative of the ability to avoid CD8+ T cell lysis. In some embodiments, the methods produce a composition comprising a cell that survives in vivo in the presence of CD8+ T cells for greater than 1, 2, 3, 4, 5, or 6 weeks or more. In some embodiments, the methods produce a composition comprising a cell that survives in vivo in the presence of CD8+ T cells for at least one week to six weeks. In some embodiments, the methods produce a composition comprising a cell that survives in vivo in the presence of CD8+ T cells for at least two to four weeks. In some embodiments, the methods produce a composition comprising a cell that survives in vivo in the presence of CD8+ T cells for at least four to six weeks. In some embodiments, the methods produce a composition comprising a cell that survives in vivo in the presence of CD8+ T cells for more than six weeks.

In some embodiments, the methods produce a composition comprising a cell having reduced or eliminated MHC class II expression and reduced or eliminated MHC class I expression relative to an unmodified cell. In some embodiments, the methods produce a composition comprising a cell having reduced or eliminated MHC class II protein expression, reduced or eliminated CIITA protein expression, and/or reduced or eliminated CIITA levels in the cell nucleus, and or eliminated reduced MHC class I protein expression. In some embodiments, the methods produce a composition comprising a cell having reduced or eliminated MHC class II protein expression, reduced or eliminated CIITA protein expression, and/or reduced or eliminated CIITA levels in the cell nucleus, and or eliminated reduced B2M protein expression. In some embodiments, the methods produce a composition comprising a cell having reduced or eliminated MHC class II protein expression, reduced or eliminated CIITA protein expression, and/or reduced or eliminated CIITA levels in the cell nucleus, and reduced or eliminated B2M mRNA levels. In some embodiments, the cell elicits a reduced or eliminated response from CD8+ T cells.

In some embodiments, the methods produce a composition comprising a cell having reduced or eliminated MHC class II expression and reduced or eliminated HLA-A expression relative to an unmodified cell, wherein the cell is homozygous for HLA-B and homozygous for HLA-C. In some embodiments, the methods produce a composition comprising a cell having reduced or eliminated MHC class II protein expression, reduced or eliminated CIITA protein expression, and/or reduced or eliminated CIITA levels in the cell nucleus, and or eliminated reduced HLA-A protein expression. In some embodiments, the methods produce a composition comprising a cell having reduced or eliminated MHC class II protein expression, reduced or eliminated CIITA protein expression, and/or reduced or eliminated CIITA levels in the cell nucleus, and/or eliminated or reduced HLA-A protein expression. In some embodiments, the cell elicits a reduced or eliminated response from CD8+ T cells.

In some embodiments, an engineered cell is provided wherein the cell has reduced or eliminated expression of MHC class II and MHC class I protein on the cell surface, wherein the cell comprises a genetic modification in CIITA, and wherein the cell comprises a modification in B2M. In some embodiments, the allogeneic cell elicits a reduced response from CD4+ T cells and elicits a reduced response from CD8+ T cells.

In some embodiments, an engineered cell is provided wherein the cell has reduced or eliminated expression of MHC class II and HLA-A protein on the cell surface, wherein the cell comprises a genetic modification in CIITA, and wherein the cell comprises a genetic modification in the HLA-A gene, wherein the cell is homozygous for HLA-B and homozygous for HLA-C. In some embodiments, an engineered cell is provided wherein the cell has reduced or eliminated expression of MHC class II and HLA-A protein on the cell surface, wherein the cell comprises a genetic modification in CIITA, and wherein the cell comprises a genetic modification in the HLA-A gene. In some embodiments, the cell is homozygous for HLA-B and HLAC. In some embodiments, the cell elicits a reduced response from CD4+ T cells and elicits a reduced response from CD8+ T cells.

2. Exogenous Nucleic Acids

In some embodiments, the present disclosure provides methods and compositions for reducing or eliminating expression of MHC class II protein on the surface of a cell by genetically modifying CIITA as disclosed herein, wherein the methods and compositions further provide for expression of an exogenous nucleic acid by the engineered cell.

a) NK Cell Inhibitor Knock-In

In some embodiments, the present disclosure provides methods for reducing or eliminating expression of MHC class II protein on the surface of a cell by genetically modifying CIITA as disclosed herein, wherein the methods further provide for expression of an exogenous nucleic acid by the cell, wherein the exogenous nucleic acid encodes an NK cell inhibitor molecule. In some embodiments, the NK cell inhibitor molecule is expressed on the surface of the cell, thereby avoiding the activity of NK cells (e.g., lysis of the cell by the NK cell). In some embodiments, the ability of the genetically modified cell to avoid NK cell lysis makes the cell amenable to adoptive cell transfer therapies. In some embodiments, the cell is an allogeneic cell.

In some embodiments, the methods comprise reducing or eliminating expression of MHC class II protein on the surface of a cell comprising genetically modifying CIITA comprising contacting the cell with a composition comprising a CIITA guide RNA disclosed herein, the method further comprising contacting the cell with a nucleic acid encoding an NK cell inhibitor molecule. In some embodiments, the methods comprise reducing or eliminating expression of MHC class II protein on the surface of a cell comprising modifying CIITA comprising contacting the cell with a composition comprising a CIITA guide RNA disclosed, the method further comprising contacting the cell with a nucleic acid encoding an NK cell inhibitor molecule, and a B2M guide RNA, thereby reducing or eliminating expression of MHC class I protein on the surface of the cell. In some embodiments, the method further comprises contacting the cell with an RNA-guided DNA binding agent.

In some embodiments, the methods comprise reducing or eliminating expression of MHC class II protein on the surface of a cell, comprising genetically modifying the cell with one or more compositions comprising a CIITA guide RNA disclosed herein, a B2M guide RNA, a nucleic acid encoding an NK cell inhibitor molecule, and an RNA-guided DNA binding agent or a nucleic acid encoding an RNA-guided DNA binding agent.

In some embodiments, the methods comprise inactivating a splice site in CIITA comprising contacting the cell with a composition comprising a CIITA guide RNA disclosed herein, the method further comprising contacting the cell with a nucleic acid encoding an NK cell inhibitor molecule. In some embodiments, the methods comprise inactivating a splice site in CIITA comprising contacting the cell with a composition comprising a CIITA guide RNA disclosed herein, the method further comprising contacting the cell with a nucleic acid encoding an NK cell inhibitor molecule, and a B2M guide RNA, thereby reducing expression of MHC class I protein on the surface of the cell. In some embodiments, the method further comprises contacting the cell with an RNA-guided DNA binding agent.

In some embodiments, the methods comprise inducing a DSB or an SSB in CIITA comprising contacting the cell with a composition comprising a CIITA guide RNA disclosed herein, the method further comprising contacting the cell with a nucleic acid encoding an NK cell inhibitor molecule. In some embodiments, the methods comprise inducing a DSB or an SSB in CIITA comprising contacting the cell with a composition comprising a CIITA guide RNA disclosed herein, the method further comprising contacting the cell with a nucleic acid encoding an NK cell inhibitor molecule, and a B2M guide RNA, thereby reducing expression of MHC class I protein on the surface of the cell. In some embodiments, the method further comprises contacting the cell with an RNA-guided DNA binding agent.

In some embodiments, the methods comprise reducing or eliminating expression of the CIITA protein in a cell comprising delivering a composition comprising a CIITA guide RNA disclosed herein, the method further comprising contacting the cell with a nucleic acid encoding an NK cell inhibitor molecule. In some embodiments, the methods comprise reducing expression of the CIITA protein in a cell comprising delivering a composition comprising a CIITA guide RNA disclosed herein, the method further comprising contacting the cell with a nucleic acid encoding an NK cell inhibitor molecule, and a B2M guide RNA, thereby reducing expression of MHC class I protein on the surface of the cell. In some embodiments, the method further comprises contacting the cell with an RNA-guided DNA binding agent.

In some embodiments, the NK cell inhibitor molecule binds to an inhibitory receptor on an NK cell. In some embodiments, the NK cell inhibitor molecule binds to an inhibitory receptor specific for MHC class I. In some embodiments, the NK cell inhibitor molecule binds to an inhibitory receptor that is not specific for MHC class I. NK cell inhibitory receptors include e.g., KIR (human), CD94-NKG2A heterodimer (human/mouse), Ly49 (mouse), 2B4, SLAMF6, NKFP-B, TIGIT, KIR2DL4.

In some embodiments, the NK cell inhibitor molecule binds to NKG2A.

In some embodiments, the NK cell inhibitor molecule is an MHC class I molecule. In some embodiments, the NK cell inhibitor molecule is a classical MHC class I molecule. In some embodiments, the NK cell inhibitor molecule is a non-classical MHC class I molecule. In some embodiments, the NK cell inhibitor molecule is an HLA molecule. NK cell inhibitor molecules include e.g., HLA-C, HLA-E, HLA-G, Cd1, CD48, SLAMF6, Clr-b, and CD155.

In some embodiments, the NK cell inhibitor molecule is HLA-E.

In some embodiments, the NK cell inhibitor molecule is a fusion protein. In some embodiments, the NK cell inhibitor molecule is a fusion protein comprising HLA-E. In some embodiments, the NK cell inhibitor molecule comprising B2M. In some embodiments, the NK cell inhibitor molecule comprising HLA-E and B2M. In some embodiments, the fusion protein includes a linker. In some embodiments, the HLA-E construct is provided in a vector. In some embodiments, a vector comprising the HLA-E construct is a lentiviral vector. In some embodiments, the HLA-E construct is delivered to the cell via lentiviral transduction.

In some embodiments, the NK cell inhibitor molecule is inserted into the genome of the target cell. In some embodiments, the NK cell inhibitor molecule is integrated into the genome of the target cell. In some embodiments, the NK cell inhibitor molecule is integrated into the genome of the target cell by homologous recombination (HR). In some embodiments, the NK cell inhibitor molecule is integrated into the genome of the target cell by blunt end insertion. In some embodiments, the NK cell inhibitor molecule is integrated into the genome of the target cell by non-homologous end joining. In some embodiments, the NK cell inhibitor molecule is integrated into a safe harbor locus in the genome of the cell. In some embodiments, the NK cell inhibitor molecule is integrated into one of the TRAC locus, B2M locus, AAVS1 locus, and/or CIITA locus. In some embodiments, the NK cell inhibitor molecule is provided to the cell in a lipid nucleic acid assembly composition. In some embodiments, the lipid nucleic acid assembly composition is a lipid nanoparticle (LNP).

In some embodiments, the methods produce an engineered cell that elicits a reduced response from NK cells. The NK cell response may be assessed in vitro or in vivo. In some embodiments, NK cell activity may be evaluated by co-culturing the genetically modified cell with NK cells in vitro. In some embodiments, NK cell activity may be evaluated in an in vivo model, e.g., a rodent model. In an in vivo model, e.g., genetically modified cells may be administered with NK cells; survival of the genetically modified cells is indicative of the ability to avoid NK cell lysis. In some embodiments, the methods produce a composition comprising a cell that survives in vivo in the presence of NK cells for greater than 1, 2, 3, 4, 5, or 6 weeks or more. In some embodiments, the methods produce a composition comprising a cell that survives in vivo in the presence of NK cells for at least one week to six weeks. In some embodiments, the methods produce a composition comprising a cell that survives in vivo in the presence of NK cells for at least two to four weeks. In some embodiments, the methods produce a composition comprising a cell that survives in vivo in the presence of NK cells for at least four to six week. In some embodiments, the methods produce a composition comprising a cell that survives in vivo in the presence of NK cells for more than six weeks.

In some embodiments, the methods produce a composition comprising an engineered cell having reduced or eliminated MHC class II expression and comprising a nucleic acid encoding an NK cell inhibitor molecule. In some embodiments, the methods produce a composition comprising an engineered cell having reduced or eliminated MHC class II expression and expression of an NK cell inhibitor molecule on the cell surface. In some embodiments, the methods produce a composition comprising a cell having reduced or eliminated MHC class II expression and eliciting a reduced response from NK cells. In some embodiments, the methods produce a composition comprising a cell having reduced or eliminated MHC class II protein expression, reduced or eliminated CIITA protein expression, and/or reduced or eliminated CIITA levels in the cell nucleus, and eliciting a reduced response from NK cells, and having reduced or eliminated MHC class I protein expression. In some embodiments, the cell elicits a reduced response from CD4+ T cells, CD8+ T cells, and/or NK cells.

In some embodiments, an allogeneic cell is provided wherein the cell has reduced or eliminated expression of MHC class II and MHC class I protein on the cell surface, wherein the cell comprises a modification in CIITA as disclosed herein, wherein the cell comprises a modification in B2M, and wherein the cell comprises a nucleic acid encoding an NK cell inhibitor molecule. In some embodiments, the allogeneic cell elicits a reduced response from CD4+ T cells, CD8+ T cells, and/or NK cells.

b) Targeting Receptors and Other Cell-Surface Expressed Polypeptides; Secreted Polypeptides

In some embodiments, the present disclosure provides methods for reducing or eliminating expression of MHC class II protein on the surface of a cell by genetically modifying CIITA as disclosed herein, wherein the methods further provide for expression of one or more exogenous nucleic acids (e.g., an antibody, chimeric antigen receptor (CAR), T cell receptor (TCR), cytokine or cytokine receptor, chemokine or chemokine receptor, enzyme, fusion protein, or other type of cell-surface bound or soluble polypeptide). In some embodiments, the exogenous nucleic acid encodes a protein that is expressed on the cell surface. For example, in some embodiments, the exogenous nucleic acid encodes a targeting receptor expressed on the cell surface (described further herein). In some embodiments, the genetically modified cell may function as a “cell factory” for the expression of a secreted polypeptide encoded by an exogenous nucleic acid, including e.g., as a source for continuous production of a polypeptide in vivo (as described further herein). In some embodiments, the cell is an allogeneic cell.

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with an exogenous nucleic acid. In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with an exogenous nucleic acid, and a B2M guide RNA, thereby reducing or eliminating expression of MHC class I protein on the surface of the cell. In some embodiments, the methods comprise reducing or eliminating expression of MHC class II protein on the surface of a cell comprising genetically modifying a splice site of the CIITA gene comprising contacting the cell with a composition comprising a CIITA guide RNA as disclosed herein, the method further comprising contacting the cell with an exogenous nucleic acid, a cell-surface expressed (e.g. targeting receptor) or soluble (e.g. secreted) polypeptide, and a B2M guide RNA, thereby reducing or eliminating expression of MHC class I protein on the surface of the cell. In some embodiments, the methods comprise contacting the cell with more than one exogenous nucleic acid. In some embodiments, the method further comprises contacting the cell with an RNA-guided DNA binding agent.

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with an exogenous nucleic acid. In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with an exogenous nucleic acid, and an HLA-A guide RNA, thereby reducing or eliminating expression of HLA-A protein on the surface of the cell. In some embodiments, the methods comprise reducing or eliminating expression of HLA-A protein on the surface of a cell comprising genetically modifying a splice site of the CIITA gene comprising contacting the cell with a composition comprising a CIITA guide RNA as disclosed herein, the method further comprising contacting the cell with an exogenous nucleic acid, a cell-surface expressed (e.g. targeting receptor) or soluble (e.g. secreted) polypeptide, and an HLA-A guide RNA, thereby reducing or eliminating expression of HLA-A protein on the surface of the cell. In some embodiments, the methods comprise contacting the cell with more than one exogenous nucleic acid. In some embodiments, the method further comprises contacting the cell with an RNA-guided DNA binding agent.

In some embodiments, the methods comprise reducing or eliminating expression of MHC class II protein on the surface of a cell, comprising genetically modifying the cell with one or more compositions comprising a CIITA guide RNA as disclosed herein, a B2M guide RNA, an exogenous nucleic acid encoding an NK cell inhibitor molecule, an exogenous nucleic acid encoding a polypeptide (e.g., a targeting receptor), and an RNA-guided DNA binding agent or a nucleic acid encoding an RNA-guided DNA binding agent.

In some embodiments, the methods comprise reducing or eliminating expression of MHC class II protein and MHC class I protein on the surface of a cell, comprising genetically modifying the cell with one or more compositions comprising a CIITA guide RNA as disclosed herein, a B2M guide RNA, an exogenous nucleic acid encoding an NK cell inhibitor molecule, an exogenous nucleic acid encoding a polypeptide (e.g., a targeting receptor), and an RNA-guided DNA binding agent or a nucleic acid encoding an RNA-guided DNA binding agent.

In some embodiments, the methods comprise inactivating a splice site in CIITA comprising contacting the cell with a composition comprising a CIITA guide RNA as disclosed herein, the method further comprising contacting the cell with an exogenous nucleic acid. In some embodiments, the methods comprise inactivating a splice site in CIITA comprising contacting the cell with a composition comprising a CIITA guide RNA that as disclosed herein, the method further comprising contacting the cell with an exogenous nucleic acid, and a B2M guide, thereby reducing expression of MHC class I protein on the surface of the cell. In some embodiments, the methods comprise inactivating a splice site in CIITA comprising contacting the cell with a composition comprising a CIITA guide RNA as disclosed herein, the method further comprising contacting the cell with an exogenous nucleic acid, a nucleic acid encoding an NK cell inhibitor, and a B2M guide RNA, thereby reducing expression of MHC class I protein on the surface of the cell. In some embodiments, the methods comprise inactivating a splice site in CIITA comprising contacting the cell with a composition comprising a CIITA guide RNA that as disclosed herein, the method further comprising contacting the cell with an exogenous nucleic acid, and a nucleic acid encoding an NK cell inhibitor. In some embodiments, the methods comprise contacting the cell with more than one exogenous nucleic acid. In some embodiments, the method further comprises contacting the cell with an RNA-guided DNA binding agent.

In some embodiments, the methods comprise inactivating a splice site in CIITA comprising contacting the cell with a composition comprising a CIITA guide RNA as disclosed herein, the method further comprising contacting the cell with an exogenous nucleic acid. In some embodiments, the methods comprise inactivating a splice site in CIITA comprising contacting the cell with a composition comprising a CIITA guide RNA that as disclosed herein, the method further comprising contacting the cell with an exogenous nucleic acid, and an HLA-A guide, thereby reducing expression of HLA-A protein on the surface of the cell. In some embodiments, the methods comprise contacting the cell with more than one exogenous nucleic acid. In some embodiments, the method further comprises contacting the cell with an RNA-guided DNA binding agent.

In some embodiments, the exogenous nucleic acid encodes a polypeptide that is expressed on the surface of the cell. In some embodiments, the exogenous nucleic acid encodes a soluble polypeptide. As used herein, “soluble” polypeptide refers to a polypeptide that is secreted by the cell. In some embodiments, the soluble polypeptide is a therapeutic polypeptide. In some embodiments, the soluble polypeptide is an antibody. In some embodiments, the soluble polypeptide is an enzyme. In some embodiments, the soluble polypeptide is a cytokine. In some embodiments, the soluble polypeptide is a chemokine. In some embodiments, the soluble polypeptide is a fusion protein.

In some embodiments, the exogenous nucleic acid encodes an antibody. In some embodiments, the exogenous nucleic acid encodes an antibody fragment (e.g., Fab, Fab2). In some embodiments, the exogenous nucleic acid encodes is a full-length antibody. In some embodiments, the exogenous nucleic acid encodes is a single-chain antibody (e.g., scFv). In some embodiments, the antibody is an IgG, IgM, IgD, IgA, or IgE. In some embodiments, the antibody is an IgG antibody. In some embodiments, the antibody is an IgG1 antibody. In some embodiments, the antibody is an IgG4 antibody. In some embodiments, the heavy chain constant region contains mutations known to reduce effector functions. In some embodiments, the heavy chain constant region contains mutations known to enhance effector functions. In some embodiments, the antibody is a bispecific antibody. In some embodiments, the antibody is a single-domain antibody (e.g., VH domain-only antibody).

In some embodiments, the exogenous nucleic acid encodes a neutralizing antibody. A neutralizing antibody neutralizes the activity of its target antigen. In some embodiments, the antibody is a neutralizing antibody against a virus antigen. In some embodiments, the antibody neutralizes a target viral antigen, blocking the ability of the virus to infect a cell. In some embodiments, a cell-based neutralization assay may be used to measure the neutralizing activity of an antibody. The particular cells and readout will depend on the target antigen of the neutralizing antibody. The half maximal effective concentration (EC₅₀) of the antibody can be measured in a cell-based neutralization assay, wherein a lower EC₅₀ is indicative of more potent neutralizing antibody.

In some embodiments, the exogenous nucleic acid encodes an antibody that binds to an antigen associated with a disease or disorder (see e.g., diseases and disorders described in Section IV).

In some embodiments, the exogenous nucleic acid encodes a polypeptide that is expressed on the surface of the cell (i.e., a cell-surface bound protein). In some embodiments, the exogenous nucleic acid encodes a targeting receptor. A “targeting receptor” is a receptor present on the surface of a cell, e.g., a T cell, to permit binding of the cell to a target site, e.g., a specific cell or tissue in an organism. In some embodiments, the targeting receptor is a CAR. In some embodiments, the targeting receptor is a universal CAR (UniCAR). In some embodiments, the targeting receptor is a TCR. In some embodiments, the targeting receptor is a TRuC. In some embodiments, the targeting receptor is a B cell receptor (BCR) (e.g., expressed on a B cell). In some embodiments, the targeting receptor is chemokine receptor. In some embodiments, the targeting receptor is a cytokine receptor.

In some embodiments, targeting receptors include a chimeric antigen receptor (CAR), a T-cell receptor (TCR), and a receptor for a cell surface molecule operably linked through at least a transmembrane domain in an internal signaling domain capable of activating a T cell upon binding of the extracellular receptor portion. In some embodiments, a CAR refers to an extracellular antigen recognition domain, e.g., an scFv, VHH, nanobody; operably linked to an intracellular signaling domain, which activates the T cell when an antigen is bound. CARs are composed of four regions: an antigen recognition domain, an extracellular hinge region, a transmembrane domain, and an intracellular T-cell signaling domain. Such receptors are well known in the art (see, e.g., WO2020092057, WO2019191114, WO2019147805, WO2018208837). A universal CAR (UniCAR) for recognizing various antigens (see, e.g., EP 2 990 416 A1) and a reversed universal CAR (RevCAR) that promotes binding of an immune cell to a target cell through an adaptor molecule (see, e.g., WO2019238722) are also contemplated. CARs can be targeted to any antigen to which an antibody can be developed and are typically directed to molecules displayed on the surface of a cell or tissue to be targeted. In some embodiments, the targeting receptor comprises an antigen recognition domain (e.g., a cancer antigen recognition domain and a subunit of a TCR (e.g., a TRuC). (See Baeuerle et al. Nature Communications 2087 (2019).)

In some embodiments, the exogenous nucleic acid encodes a TCR. In some embodiments, the exogenous nucleic acid encodes a genetically modified TCR. In some embodiments, the exogenous nucleic acid encodes is a genetically modified TCR with specificity for a polypeptide expressed by cancer cells. In some embodiments, the exogenous nucleic acid encodes a targeting receptor specific for Wilms' tumor gene (WT1) antigen. In some embodiments, the exogenous nucleic acid encodes the WT1-specific TCR (see e.g., WO2020/081613A1).

In some embodiments, an exogenous nucleic acid is inserted into the genome of the target cell. In some embodiments, the exogenous nucleic acid is integrated into the genome of the target cell. In some embodiments, the exogenous nucleic acid is integrated into the genome of the target cell by homologous recombination (HR). In some embodiments, the exogenous nucleic acid is integrated into the genome of the target cell by blunt end insertion. In some embodiments, the exogenous nucleic acid is integrated into the genome of the target cell by non-homologous end joining. In some embodiments, the exogenous nucleic acid is integrated into a safe harbor locus in the genome of the cell. In some embodiments, the exogenous nucleic acid is integrated into one of the TRAC locus, B2M locus, AAVS1 locus, and/or CIITA locus. In some embodiments, the exogenous nucleic acid is provided to the cell in a lipid nucleic acid assembly composition. In some embodiments, the lipid nucleic acid assembly composition is a lipid nanoparticle (LNP).

In some embodiments, the methods produce a composition comprising an engineered cell having reduced or eliminated MHC class II expression and comprising an exogenous nucleic acid. In some embodiments, the methods produce a composition comprising an engineered cell having reduced or eliminated MHC class II expression and that secretes and/or expresses a polypeptide encoded by an exogenous nucleic acid integrated into the genome of the cell. In some embodiments, the methods produce a composition comprising an engineered cell having reduced or eliminated MHC class II protein expression, reduced or eliminated CIITA protein expression, and/or reduced or eliminated CIITA levels in the cell nucleus, and eliciting a reduced response from NK cells, and having reduced MHC class I protein expression, and secreting and/or expressing a polypeptide encoded by an exogenous nucleic acid integrated into the genome of the cell. In some embodiments, the engineered cell elicits a reduced response from CD4+ T cells, and/or CD8+ T cells.

In some embodiments, an engineered cell is provided wherein the cell has reduced or eliminated expression of MHC class II and MHC class I protein on the cell surface, wherein the cell comprises a modification in CIITA as disclosed herein, wherein the cell comprises a modification in B2M, wherein the cell comprises an exogenous nucleic acid encoding an NK cell inhibitor molecule, and wherein the cell further comprises an exogenous nucleic acid encoding a polypeptide (e.g., a targeting receptor). In some embodiments, the engineered cell elicits a reduced response from CD4+ T cells, and/or CD8+ T cells.

In embodiments, an engineered cell is provided wherein the cell has reduced or eliminated expression of MHC class II and HLA-A protein on the cell surface, wherein the cell comprises a modification in CIITA as disclosed herein, wherein the cell comprises a modification in the HLA-A gene, wherein the cell further comprises an exogenous nucleic acid encoding a polypeptide (e.g., a targeting receptor). In some embodiments, the engineered cell elicits a reduced response from CD4+ T cells, and/or CD8+ T cells.

In some embodiments, the present disclosure provides methods for reducing or eliminating expression of MHC class II protein on the surface of a cell by genetically modifying CIITA as disclosed herein, wherein the methods further provide for reducing expression of one or more additional target genes (e.g., TRAC, TRBC). In some embodiments, the additional genetic modifications provide further advantages for use of the genetically modified cells for adoptive cell transfer applications. In some embodiments, the cell is an allogeneic cell.

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene (e.g., a gene other than CIITA or B2M or HLA-A), thereby reducing or eliminating expression of the other gene. In some embodiments, the methods comprise reducing expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, and a B2M guide RNA, thereby reducing or eliminating expression of MHC class I protein on the surface of the cell. In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing or eliminating expression of the other gene, and an exogenous nucleic acid encoding a polypeptide (e.g., a targeting receptor).

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing expression of the other gene, a B2M guide RNA, thereby reducing expression of MHC class I protein on the surface of the cell, and an exogenous nucleic acid encoding an NK cell inhibitor.

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing expression of the other gene, and an HLA-A guide RNA, thereby reducing expression of HLA-A protein on the surface of the cell.

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing or eliminating expression of the other gene, a B2M guide RNA, thereby reducing or eliminating expression of MHC class I protein on the surface of the cell, and an exogenous nucleic acid encoding a polypeptide (e.g., a targeting receptor).

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, an exogenous nucleic acid encoding an NK cell inhibitor molecule, and an exogenous nucleic acid encoding a polypeptide (e.g., a targeting receptor).

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing expression of the other gene, and an HLA-A guide RNA, thereby reducing expression of HLA-A protein on the surface of the cell, and an exogenous nucleic acid encoding a polypeptide (e.g., a targeting receptor).

In some embodiments, the methods comprise reducing or eliminating expression of MHC class II protein on the surface of a cell comprising genetically modifying CIITA comprising contacting the cell with a composition comprising a CIITA guide RNA as disclosed herein, the method further comprising contacting the cell with a guide RNA that directs an RNA-guided DNA binding agent to a target sequence located in an another gene, thereby reducing or eliminating expression of the additional gene, a B2M guide RNA that directs an RNA-guided DNA binding agent to a target sequence located in an another gene, thereby reducing or eliminating expression of MHC class I protein on the surface of the cell, an exogenous nucleic acid encoding an NK cell inhibitor molecule, and an exogenous nucleic acid encoding a polypeptide (e.g., a targeting receptor). In some embodiments, the method further comprises contacting the cell with an RNA-guided DNA binding agent.

In some embodiments, the methods comprise reducing or eliminating expression of MHC class II protein on the surface of a cell, comprising genetically modifying the cell with one or more compositions comprising a CIITA guide RNA as disclosed herein, a B2M guide RNA, an exogenous nucleic acid encoding an NK cell inhibitor molecule, an exogenous nucleic acid encoding polypeptide (e.g., a targeting receptor), a guide RNA that directs an RNA-guided DNA binding agent to a target sequence located in an another gene, thereby reducing or eliminating expression of the other gene, and an RNA-guided DNA binding agent or a nucleic acid encoding an RNA-guided DNA binding agent.

In some embodiments, the methods comprise reducing or eliminating expression of MHC class II protein on the surface of a cell, comprising genetically modifying the cell with one or more compositions comprising a CIITA guide RNA as disclosed herein, an HLA-A guide RNA, an exogenous nucleic acid encoding polypeptide (e.g., a targeting receptor), a guide RNA that directs an RNA-guided DNA binding agent to a target sequence located in an another gene, thereby reducing or eliminating expression of the other gene, and an RNA-guided DNA binding agent or a nucleic acid encoding an RNA-guided DNA binding agent.

In some embodiments, the additional target gene is TRAC. In some embodiments, the additional target gene is TRBC.

D. Exemplary Cell Types

In some embodiments, methods and compositions disclosed herein genetically modify a cell. In some embodiments, the cell is an allogeneic cell. In some embodiments the cell is a human cell. In some embodiments the genetically modified cell is referred to as an engineered cell. An engineered cell refers to a cell (or progeny of a cell) comprising an engineered genetic modification, e.g. that has been contacted with a gene editing system and genetically modified by the gene editing system. The terms “engineered cell” and “genetically modified cell” are used interchangeably throughout. The engineered cell may be any of the exemplary cell types disclosed herein.

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3⁺, CD4⁺ and CD8⁺ T cells, regulatory T cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T cell. In some embodiments, the cell is a B cell. In some embodiments, the cell is a NK cell. In some embodiments, the lymphocyte is allogeneic.

As used herein, a T cell can be defined as a cell that expresses a T cell receptor (“TCR” or “αβ TCR” or “γδ TCR”), however in some embodiments, the TCR of a T cell may be genetically modified to reduce its expression (e.g., by genetic modification to the TRAC or TRBC genes), therefore expression of the protein CD3 may be used as a marker to identify a T cell by standard flow cytometry methods. CD3 is a multi-subunit signaling complex that associates with the TCR. Thus, a T cell may be referred to as CD3+. In some embodiments, a T cell is a cell that expresses a CD3+ marker and either a CD4+ or CD8+ marker. In some embodiments, the T cell is allogeneic.

In some embodiments, the T cell expresses the glycoprotein CD8 and therefore is CD8+ by standard flow cytometry methods and may be referred to as a “cytotoxic” T cell. In some embodiments, the T cell expresses the glycoprotein CD4 and therefore is CD4+ by standard flow cytometry methods and may be referred to as a “helper” T cell. CD4+ T cells can differentiate into subsets and may be referred to as a Th1 cell, Th2 cell, Th9 cell, Th17 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T cell may be isolated from a subject by CD4+ or CD8+ selection methods.

In some embodiments, the T cell is a memory T cell. In the body, a memory T cell has encountered antigen. A memory T cell can be located in the secondary lymphoid organs (central memory T cells) or in recently infected tissue (effector memory T cells). A memory T cell may be a CD8+ T cell. A memory T cell may be a CD4+ T cell.

As used herein, a “central memory T cell” can be defined as an antigen-experienced T cell, and for example, may expresses CD62L and CD45RO. A central memory T cell may be detected as CD62L+ and CD45RO+ by Central memory T cells also express CCR7, therefore may be detected as CCR7+ by standard flow cytometry methods.

As used herein, an “early stem-cell memory T cell” (or “Tscm”) can be defined as a T cell that expresses CD27 and CD45RA, and therefore is CD27+ and CD45RA+ by standard flow cytometry methods. A Tscm does not express the CD45 isoform CD45RO, therefore a Tscm will further be CD45RO− if stained for this isoform by standard flow cytometry methods. A CD45RO-CD27+ cell is therefore also an early stem-cell memory T cell. Tscm cells further express CD62L and CCR7, therefore may be detected as CD62L+ and CCR7+ by standard flow cytometry methods. Early stem-cell memory T cells have been shown to correlate with increased persistence and therapeutic efficacy of cell therapy products.

In some embodiments, the cell is a B cell. As used herein, a “B cell” can be defined as a cell that expresses CD19 and/or CD20, and/or B cell mature antigen (“BCMA”), and therefore a B cell is CD19+, and/or CD20+, and/or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+, or has a class-switched B cell receptor (e.g., IgG+, or IgA+). In some embodiments, the B cell is allogeneic.

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”). In some embodiments, the mononuclear cell is allogeneic.

Cells used in ACT and/or tissue regenerative therapy are included, such as stem cells, progenitor cells, and primary cells. Stem cells, for example, include pluripotent stem cells (PSCs); induced pluripotent stem cells (iPSCs); embryonic stem cells (ESCs); mesenchymal stem cells (MSCs, e.g., isolated from bone marrow (BM), peripheral blood (PB), placenta, umbilical cord (UC) or adipose); hematopoietic stem cells (HSCs; e.g. isolated from BM or UC); neural stem cells (NSCs); tissue specific progenitor stem cells (TSPSCs); and limbal stem cells (LSCs). Progenitor and primary cells include mononuclear cells (MNCs, e.g., isolated from BM or PB); endothelial progenitor cells (EPCs, e.g. isolated from BM, PB, and UC); neural progenitor cells (NPCs); and tissue-specific primary cells or cells derived therefrom (TSCs) including chondrocytes, myocytes, and keratinocytes. Cells for organ or tissue transplantations such as islet cells, cardiomyocytes, thyroid cells, thymocytes, neuronal cells, skin cells, and retinal cells are also included.

In some embodiments, the cell is a human cell, such as a cell isolated from a human subject. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the methods are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

In some embodiments, when the cell is homozygous for HLA-B, the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02.

In some embodiments, when the cell is homozygous for HLA-C, the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the cell is homozygous for HLA-B and homozygous for HLA-C and the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the cell is homozygous for HLA-B and homozygous for HLA-C and the HLA-B and HLA-C alleles are selected from any one of the following HLA-B and HLA-C alleles: HLA-B*07:02 and HLA-C*07:02; HLA-B*08:01 and HLA-C*07:01; HLA-B*44:02 and HLA-C*05:01; HLA-B*35:01 and HLA-C*04:01; HLA-B*40:01 and HLA-C*03:04; HLA-B*57:01 and HLA-C*06:02; HLA-B*14:02 and HLA-C*08:02; HLA-B*15:01 and HLA-C*03:03; HLA-B*13:02 and HLA-C*06:02; HLA-B*44:03 and HLA-C*16:01; HLA-B*38:01 and HLA-C*12:03; HLA-B*18:01 and HLA-C*07:01; HLA-B*44:03 and HLA-C*04:01; HLA-B*51:01 and HLA-C*15:02; HLA-B*49:01 and HLA-C*07:01; HLA-B*15:01 and HLA-C*03:04; HLA-B*18:01 and HLA-C*12:03; HLA-B*27:05 and HLA-C*02:02; HLA-B*35:03 and HLA-C*04:01; HLA-B*18:01 and HLA-C*05:01; HLA-B*52:01 and HLA-C*12:02; HLA-B*51:01 and HLA-C*14:02; HLA-B*37:01 and HLA-C*06:02; HLA-B*53:01 and HLA-C*04:01; HLA-B*55:01 and HLA-C*03:03; HLA-B*44:02 and HLA-C*07:04; HLA-B*44:03 and HLA-C*07:01; HLA-B*35:02 and HLA-C*04:01; HLA-B*15:01 and HLA-C*04:01; and HLA-B*40:02 and HLA-C*02:02. In some embodiments, the cell is homozygous for HLA-B and homozygous for HLA-C and the HLA-B and HLA-C alleles are HLA-B*07:02 and HLA-C*07:02. In some embodiments, the cell is homozygous for HLA-B and homozygous for HLA-C and the HLA-B and HLA-C alleles are HLA-B*08:01 and HLA-C*07:01. In some embodiments, the cell is homozygous for HLA-B and homozygous for HLA-C and the HLA-B and HLA-C alleles are HLA-B*44:02 and HLA-C*05:01. In some embodiments, the cell is homozygous for HLA-B and homozygous for HLA-C and the HLA-B and HLA-C alleles are HLA-B*35:01 and HLA-C*04:01.

### III. Details of the Gene Editing Systems

Various suitable gene editing systems may be used to make the engineered cells disclosed herein, including but not limited to the CRISPR/Cas system; zinc finger nuclease (ZFN) system; and the transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507(7491): 258-261), which also may have the potential for uses in gene editing and gene therapy.

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing in situ, a technique known as gene editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

In some embodiments, the gene editing system is a CRISPR/Cas system, including e.g., a CRISPR guide RNA comprising a guide sequence and RNA-guided DNA binding agent, and described further herein.

A. CRISPR Guide RNA

Provided herein are guide sequences useful for modifying a target sequence, e.g., using a guide RNA comprising a disclosed guide sequence with an RNA-guided DNA binding agent (e.g., a CRISPR/Cas system).

Each of the guide sequences disclosed herein may further comprise additional nucleotides to form a crRNA, e.g., with the following exemplary nucleotide sequence following the guide sequence at its 3′ end: GUUUUAGAGCUAUGCUGUUUUG (SEQ ID NO: 170) in 5′ to 3′ orientation. In the case of a sgRNA, the above guide sequences may further comprise additional nucleotides (scaffold sequence) to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUUGA AAAAGUGGCACCGAGUCGGUGCUUUU (SEQ ID NO: 171) or GUUUUAGAGCUAGAAAUAGCAAGUUAAAAUAAGGCUAGUCCGUUAUCAACUUGA AAAAGUGGCACCGAGUCGGUGC (SEQ ID NO: 172, which is SEQ ID NO: 171 without the four terminal U's) in 5′ to 3′ orientation. In some embodiments, the four terminal U's of SEQ ID NO: 171 are not present. In some embodiments, only 1, 2, or 3 of the four terminal U's of SEQ ID NO: 171 are present.

In some embodiments, the sgRNA comprises any one of the guide sequences of SEQ ID Nos: 1-101 and additional nucleotides to form a crRNA, e.g., with the following exemplary scaffold nucleotide sequence following the guide sequence at its 3′ end: GUUUUAGAGCUAGAAAUAGCAAGUUAAAAUAAGGCUAGUCCGUUAUCAACUUGG CACCGAGUCGGUGC (SEQ ID NO: 173) in 5′ to 3′ orientation. SEQ ID NO: 173 lacks 8 nucleotides with reference to a wild-type guide RNA conserved sequence: GUUUUAGAGCUAGAAAUAGCAAGUUAAAAUAAGGCUAGUCCGUUAUCAACUUGA AAAAGUGGCACCGAGUCGGUGC (SEQ ID NO: 172).

Other exemplary scaffold nucleotide sequences are provided in Table 23. In some embodiments, the sgRNA comprises any one of the guide sequences of SEQ ID Nos: 1-101 and additional guide scaffold sequences, in 5′ to 3′ orientation, in Table 23 including modified versions of the scaffold sequences, as shown.

The guide RNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, a crRNA and/or trRNA sequence may be referred to as a “scaffold” or “conserved portion” of a guide RNA.

In each of the compositions, use, and method embodiments described herein, the guide RNA may comprise two RNA molecules as a “dual guide RNA” or “dgRNA.” The dgRNA comprises a first RNA molecule comprising a crRNA comprising, e.g., a guide sequence shown in Table 1, and a second RNA molecule comprising a trRNA. The first and second RNA molecules may not be covalently linked, but may form an RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Table 1, covalently linked to a trRNA. The sgRNA may comprise 17, 18, 19, or 20 contiguous nucleotides of a guide sequence shown in Table 1. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, a composition comprising one or more guide RNAs comprising a guide sequence of any one in Table 1 is provided. In some embodiments, a composition comprising one or more guide RNAs comprising a guide sequence of any one in Table 1 is provided, wherein the nucleotides of SEQ ID NO: 170, 171, 172, or 173 follow the guide sequence at its 3′ end. In some embodiments, the one or more guide RNAs comprising a guide sequence of any one in Table 1, wherein the nucleotides of SEQ ID NO: 170, 171, 172, or 173 follow the guide sequence at its 3′ end, is modified according to the modification pattern of SEQ ID NO: 300.

In some embodiments, a composition comprising one or more guide RNAs comprising a guide sequence of any one in Table 1 is provided. In one aspect, a composition comprising one or more gRNAs is provided, comprising a guide sequence that is at least 99%, 98%, 97%, 96%, 95%, 94%, 93%, 92%, 91%, or 90% identical to any of the nucleic acids of SEQ ID NOs: 1-101.

In other embodiments, a composition is provided that comprises at least one, e.g., at least two gRNA's comprising guide sequences selected from any two or more of the guide sequences shown in Table 1. In some embodiments, the composition comprises at least two gRNA's that each comprise a guide sequence at least 99%, 98%, 97%, 96%, 95%, 94%, 93%, 92%, 91%, or 90% identical to any of the guide sequences shown in Table 1.

In some embodiments, the guide RNA compositions of the present invention are designed to recognize (e.g., hybridize to) a target sequence in CIITA. For example, the CIITA target sequence may be recognized and cleaved by a provided Cas cleavase comprising a guide RNA. In some embodiments, an RNA-guided DNA binding agent, such as a Cas cleavase, may be directed by a guide RNA to a target sequence in CIITA, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within CIITA. In some embodiments, the compositions comprising one or more guide sequences comprise a guide sequence that is complementary to the corresponding genomic region shown in Table 1, according to coordinates from human reference genome hg38. Guide sequences of further embodiments may be complementary to sequences in the close vicinity of the genomic coordinate listed in any of the Table 1 within CIITA. For example, guide sequences of further embodiments may be complementary to sequences that comprise 10 contiguous nucleotides±10 nucleotides of a genomic coordinate listed in Table 1.

Without being bound by any particular theory, modifications (e.g., frameshift mutations resulting from indels occurring as a result of a nuclease-mediated DSB) in certain regions of the target gene may be less tolerable than mutations in other regions, thus the location of a DSB is an important factor in the amount or type of protein knockdown that may result. In some embodiments, a gRNA complementary or having complementarity to a target sequence within the target gene used to direct an RNA-guided DNA binding agent to a particular location in the target gene.

In some embodiments, the guide sequence is at least 99%, 98%, 97%, 96%, 95%, 94%, 93%, 92%, 91%, 90%, 85%, or 80% identical to a target sequence present in the target gene. In some embodiments, the guide sequence is at least 99%, 98%, 97%, 96%, 95%, 94%, 93%, 92%, 91%, 90%, 85%, or 80% identical to a target sequence present in the human CIJTA gene.

In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

B. Modifications of gRNAs

In some embodiments, the gRNA (e.g., sgRNA, short-sgRNA, dgRNA, or crRNA) is modified. The term “modified” or “modification” in the context of a gRNA described herein includes, the modifications described above, including, for example, (a) end modifications, e.g., 5′ end modifications or 3′ end modifications, including 5′ or 3′ protective end modifications, (b) nucleobase (or “base”) modifications, including replacement or removal of bases, (c) sugar modifications, including modifications at the 2′, 3′, and/or 4′ positions, (d) internucleoside linkage modifications, and (e) backbone modifications, which can include modification or replacement of the phosphodiester linkages and/or the ribose sugar. A modification of a nucleotide at a given position includes a modification or replacement of the phosphodiester linkage immediately 3′ of the sugar of the nucleotide. Thus, for example, a nucleic acid comprising a phosphorothioate between the first and second sugars from the 5′ end is considered to comprise a modification at position 1. The term “modified gRNA” generally refers to a gRNA having a modification to the chemical structure of one or more of the base, the sugar, and the phosphodiester linkage or backbone portions, including nucleotide phosphates, all as detailed and exemplified herein.

Further description and exemplary patterns of modifications are provided in in Table 1 of WO2019/237069 published Dec. 12, 2019, the entire contents of which are incorporated herein by reference.

In some embodiments, a gRNA comprises modifications at 1, 2, 3, 4, 5, 6, 7, 8, 9, 10, 11, 12, 13, 14, 15, 16, or more YA sites. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, 2′-H, inosine, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

In some embodiments, the guide sequence (or guide region) of a gRNA comprises 1, 2, 3, 4, 5, or more YA sites (“guide region YA sites”) that may comprise YA modifications. In some embodiments, one or more YA sites located at 5-end, 6-end, 7-end, 8-end, 9-end, or 10-end from the 5′ end of the 5′ terminus (where “5-end”, etc., refers to position 5 to the 3′ end of the guide region, i.e., the most 3′ nucleotide in the guide region) comprise YA modifications.. A modified guide region YA site comprises a YA modification.

In some embodiments, a modified guide region YA site is within 20, 19, 18, 17, 16, 15, 14, 13, 12, 11, 10, or 9 nucleotides of the 3′ terminal nucleotide of the guide region. For example, if a modified guide region YA site is within 10 nucleotides of the 3′ terminal nucleotide of the guide region and the guide region is 20 nucleotides long, then the modified nucleotide of the modified guide region YA site is located at any of positions 11-20. In some embodiments, a modified guide region YA site is at or after nucleotide 4, 5, 6, 7, 8, 9, 10, or 11 from the 5′ end of the 5′ terminus.

In some embodiments, a modified guide region YA site is other than a 5′ end modification. For example, a sgRNA can comprise a 5′ end modification as described herein and further comprise a modified guide region YA site. Alternatively, a sgRNA can comprise an unmodified 5′ end and a modified guide region YA site. Alternatively, a short-sgRNA can comprise a modified 5′ end and an unmodified guide region YA site.

In some embodiments, a modified guide region YA site comprises a modification that at least one nucleotide located 5′ of the guide region YA site does not comprise. For example, if nucleotides 1-3 comprise phosphorothioates, nucleotide 4 comprises only a 2′-OMe modification, and nucleotide 5 is the pyrimidine of a YA site and comprises a phosphorothioate, then the modified guide region YA site comprises a modification (phosphorothioate) that at least one nucleotide located 5′ of the guide region YA site (nucleotide 4) does not comprise. In another example, if nucleotides 1-3 comprise phosphorothioates, and nucleotide 4 is the pyrimidine of a YA site and comprises a 2′-OMe, then the modified guide region YA site comprises a modification (2′-OMe) that at least one nucleotide located 5′ of the guide region YA site (any of nucleotides 1-3) does not comprise. This condition is also always satisfied if an unmodified nucleotide is located 5′ of the modified guide region YA site.

In some embodiments, the modified guide region YA sites comprise modifications as described for YA sites above. The guide region of a gRNA may be modified according to any embodiment comprising a modified guide region set forth herein. Any embodiments set forth elsewhere in this disclosure may be combined to the extent feasible with any of the foregoing embodiments.

In some embodiments, the 5′ and/or 3′ terminus regions of a gRNA are modified.

In some embodiments, the terminal (i.e., last) 1, 2, 3, 4, 5, 6, or 7 nucleotides in the 3′ terminus region are modified. Throughout, this modification may be referred to as a “3′ end modification”. In some embodiments, the terminal (i.e., last) 1, 2, 3, 4, 5, 6, or 7 nucleotides in the 3′ terminus region comprise more than one modification. In some embodiments, the 3′ end modification comprises or further comprises any one or more of the following: a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof. In some embodiments, the 3′ end modification comprises or further comprises modifications of 1, 2, 3, 4, 5, 6, or 7 nucleotides at the 3′ end of the gRNA. In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides. In some embodiments, the 3′ end modification comprises or further comprises PS linkages between any one or more of the last 2, 3, 4, 5, 6, or 7 nucleotides. In some embodiments, the gRNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified. In some embodiments, a gRNA is provided comprising a 3′ protective end modification. In some embodiments, the 3′ tail comprises between 1 and about 20 nucleotides, between 1 and about 15 nucleotides, between 1 and about 10 nucleotides, between 1 and about 5 nucleotides, between 1 and about 4 nucleotides, between 1 and about 3 nucleotides, and between 1 and about 2 nucleotides. In some embodiments, the gRNA does not comprise a 3′ tail.

In some embodiments, the 5′ terminus region is modified, for example, the first 1, 2, 3, 4, 5, 6, or 7 nucleotides of the gRNA are modified. Throughout, this modification may be referred to as a “5′ end modification”. In some embodiments, the first 1, 2, 3, 4, 5, 6, or 7 nucleotides of the 5′ terminus region comprise more than one modification. In some embodiments, at least one of the terminal (i.e., first) 1, 2, 3, 4, 5, 6, or 7 nucleotides at the 5′ end are modified. In some embodiments, both the 5′ and 3′ terminus regions (e.g., ends) of the gRNA are modified. In some embodiments, only the 5′ terminus region of the gRNA is modified. In some embodiments, only the 3′ terminus region (plus or minus a 3′ tail) of the conserved portion of a gRNA is modified. In some embodiments, the gRNA comprises modifications at 1, 2, 3, 4, 5, 6, or 7 of the first 7 nucleotides at a 5′ terminus region of the gRNA. In some embodiments, the gRNA comprises modifications at 1, 2, 3, 4, 5, 6, or 7 of the 7 terminal nucleotides at a 3′ terminus region. In some embodiments, 2, 3, or 4 of the first 4 nucleotides at the 5′ terminus region, and/or 2, 3, or 4 of the terminal 4 nucleotides at the 3′ terminus region are modified. In some embodiments, 2, 3, or 4 of the first 4 nucleotides at the 5′ terminus region are linked with phosphorothioate (PS) bonds. In some embodiments, the modification to the 5′ terminus and/or 3′ terminus comprises a 2′-O-methyl (2′-O-Me) or 2′-O-(2-methoxyethyl) (2′-O-moe) modification. In some embodiments, the modification comprises a 2′-fluoro (2′-F) modification to a nucleotide. In some embodiments, the modification comprises a phosphorothioate (PS) linkage between nucleotides. In some embodiments, the modification comprises an inverted abasic nucleotide. In some embodiments, the modification comprises a protective end modification. In some embodiments, the modification comprises a more than one modification selected from protective end modification, 2′-O-Me, 2′-O-moe, 2′-fluoro (2′-F), a phosphorothioate (PS) linkage between nucleotides, and an inverted abasic nucleotide. In some embodiments, an equivalent modification is encompassed.

In some embodiments, a gRNA is provided comprising a 5′ end modification and a 3′ end modification. In some embodiments, the gRNA comprises modified nucleotides that are not at the 5′ or 3′ ends.

In some embodiments, a sgRNA is provided comprising an upper stem modification, wherein the upper stem modification comprises a modification to any one or more of US1-US12 in the upper stem region. In some embodiments, a sgRNA is provided comprising an upper stem modification, wherein the upper stem modification comprises a modification of at least 1, 2, 3, 4, 5, 6, 7, 8, 9, 10, 11, or all 12 nucleotides in the upper stem region. In some embodiments, an sgRNA is provided comprising an upper stem modification, wherein the upper stem modification comprises 1, 2, 3, 4, or 5 YA modifications in a YA site. In some embodiments, the upper stem modification comprises a 2′-OMe modified nucleotide, a 2′-O-moe modified nucleotide, a 2′-F modified nucleotide, and/or combinations thereof. Other modifications described herein, such as a 5′ end modification and/or a 3′ end modification may be combined with an upper stem modification.

In some embodiments, the sgRNA comprises a modification in the hairpin region. In some embodiments, the hairpin region modification comprises at least one modified nucleotide selected from a 2′-O-methyl (2′-OMe) modified nucleotide, a 2′-fluoro (2′-F) modified nucleotide, and/or combinations thereof. In some embodiments, the hairpin region modification is in the hairpin 1 region. In some embodiments, the hairpin region modification is in the hairpin 2 region. In some embodiments, the hairpin modification comprises 1, 2, or 3 YA modifications in a YA site. In some embodiments, the hairpin modification comprises at least 1, 2, 3, 4, 5, or 6 YA modifications. Other modifications described herein, such as an upper stem modification, a 5′ end modification, and/or a 3′ end modification may be combined with a modification in the hairpin region.

In some embodiments, a gRNA comprises a substituted and optionally shortened hairpin 1 region, wherein at least one of the following pairs of nucleotides are substituted in the substituted and optionally shortened hairpin 1 with Watson-Crick pairing nucleotides: H1-1 and H1-12, H1-2 and H1-11, H1-3 and H1-10, and/or H1-4 and H1-9. “Watson-Crick pairing nucleotides” include any pair capable of forming a Watson-Crick base pair, including A-T, A-U, T-A, U-A, C-G, and G-C pairs, and pairs including modified versions of any of the foregoing nucleotides that have the same base pairing preference. In some embodiments, the hairpin 1 region lacks any one or two of H1-5 through H1-8. In some embodiments, the hairpin 1 region lacks one, two, or three of the following pairs of nucleotides: H1-1 and H1-12, H1-2 and H1-11, H1-3 and H1-10 and/or H1-4 and H1-9. In some embodiments, the hairpin 1 region lacks 1-8 nucleotides of the hairpin 1 region. In any of the foregoing embodiments, the lacking nucleotides may be such that the one or more nucleotide pairs substituted with Watson-Crick pairing nucleotides (H1-1 and H1-12, H1-2 and H1-11, H1-3 and H1-10, and/or H1-4 and H1-9) form a base pair in the gRNA.

In some embodiments, the gRNA further comprises an upper stem region lacking at least 1 nucleotide, e.g., any of the shortened upper stem regions indicated in Table 7 of U.S. Application No. 62/946,905, the contents of which are hereby incorporated by reference in its entirety, or described elsewhere herein, which may be combined with any of the shortened or substituted hairpin 1 regions described herein.

In some embodiments, the gRNA described herein further comprises a nexus region, wherein the nexus region lacks at least one nucleotide.

In some embodiments, an sgRNA provided herein is a short-single guide RNAs (short-sgRNAs), e.g., comprising a conserved portion of an sgRNA comprising a hairpin region, wherein the hairpin region lacks at least 5-10 nucleotides or 6-10 nucleotides. In some embodiments, the 5-10 nucleotides or 6-10 nucleotides are consecutive.

In some embodiments, a short-sgRNA lacks at least nucleotides 54-58 (AAAAA) of the conserved portion of a spyCas9 sgRNA. In some embodiments, a short-sgRNA is a non-spyCas9 sgRNA that lacks nucleotides corresponding to nucleotides 54-58 (AAAAA) of the conserved portion of a spyCas9 as determined, for example, by pairwise or structural alignment.

In some embodiments, the short-sgRNA described herein comprises a conserved portion comprising a hairpin region, wherein the hairpin region lacks 5, 6, 7, 8, 9, 10, 11, or 12 nucleotides. In some embodiments, the lacking nucleotides are 5-10 lacking nucleotides or 6-10 lacking nucleotides. In some embodiments, the lacking nucleotides are consecutive. In some embodiments, the lacking nucleotides span at least a portion of hairpin 1 and a portion of hairpin 2. In some embodiments, the 5-10 lacking nucleotides comprise or consist of nucleotides 54-58, 54-61, or 53-60 of SEQ ID NO: 171.

In some embodiments, the short-sgRNA described herein further comprises a nexus region, wherein the nexus region lacks at least one nucleotide (e.g., 1, 2, 3, 4, 5, 6, 7, 8, 9, or 10 nucleotides in the nexus region). In some embodiments, the short-sgRNA lacks each nucleotide in the nexus region.

In some embodiments, the SpyCas9 short-sgRNA described herein comprises a sequence of NNNNNNNNNNNNNNNNNNNNGUUUUAGAGCUAGAAAUAGCAAGUUAAAAUAAGG CUAGUCCGUUAUCACGAAAGGGCACCGAGUCGGUGCU (SEQ ID NO: 976).

In some embodiments, the short-sgRNA described herein comprises a modification pattern as shown in SEQ ID NO: 977: mN*mN*mN*NNNNNNNNNNNNNNNNNGUUUUAGAmGmCmUmAmGmAmAmAmUmA mGmCAAGUUAAAAUAAGGCUAGUCCGUUAUCACGAAAGGGCACCGAGUCGGmUm GmC*mU (SEQ ID NO: 977), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides.

In certain embodiments, using SEQ ID NO: 172 (“Exemplary SpyCas9 sgRNA-1”) as an example, the Exemplary SpyCas9 sgRNA-1 further includes one or more of:
A. a shortened hairpin 1 region, or a substituted and optionally shortened hairpin 1 region, wherein
   1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 6-8 nucleotides, preferably 6 nucleotides; and
      a. one or more of positions H1-1, H1-2, or H1-3 is deleted or substituted relative to Exemplary SpyCas9 sgRNA-1 (SEQ ID NO: 172) or
      b. one or more of positions H1-6 through H1-10 is substituted relative to Exemplary SpyCas9 sgRNA-1 (SEQ ID NO: 172); or
   3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 172); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 172); or
C. a substitution relative to Exemplary SpyCas9 sgRNA-1 (SEQ ID NO: 172) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. Exemplary SpyCas9 sgRNA-1 (SEQ ID NO: 172) with an upper stem region, wherein the upper stem modification comprises a modification to any one or more of US1-US12 in the upper stem region, wherein
   1. the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof, or
   2. the modified nucleotide optionally includes a 2′-OMe modified nucleotide.

In certain embodiments, Exemplary SpyCas9 sgRNA-1, or an sgRNA, such as an sgRNA comprising Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide, or a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. Modified nucleosides and nucleotides can include one or more of. (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20. In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges. In some embodiments, the 2′ hydroxyl group modification can included “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a gRNA molecule are deoxynucleotides.

In some embodiments, the gRNAs disclosed herein comprise one of the modification patterns disclosed in WO2018/107028 A1, published Jun. 14, 2018 the contents of which are hereby incorporated by reference in their entirety.

The terms “mA,” “mC,” “mU,” or “mG” may be used to denote a nucleotide that has been modified with 2′-O-Me. The terms “fA,” “fC,” “fU,” or “fG” may be used to denote a nucleotide that has been substituted with 2′-F. A “*” may be used to depict a PS modification. The terms A*, C*, U*, or G* may be used to denote a nucleotide that is linked to the next (e.g., 3′) nucleotide with a PS bond. The terms “mA*,” “mC*,” “mU*,” or “mG*” may be used to denote a nucleotide that has been substituted with 2′-O-Me and that is linked to the next (e.g., 3′) nucleotide with a PS bond.

Exemplary spyCas9 sgRNA-1 (SEQ ID NO: 172)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B2-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| Nexus | | | | | | | | | | H1-1 through H1-12 | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

C. Ribonucleoprotein Complex

In some embodiments, the disclosure provides compositions comprising one or more gRNAs comprising one or more guide sequences from Table 1 and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes, S. aureus, and other prokaryotes (see e.g., the list in the next paragraph), and modified (e.g., engineered or mutant) versions thereof. See e.g., US2016/0312198 A1; US 2016/0312199 A1. Other examples of Cas nucleases include a Csm or Cmr complex of a type III CRISPR system or the Cas10, Csm1, or Cmr2 subunit thereof, and a Cascade complex of a type I CRISPR system, or the Cas3 subunit thereof. In some embodiments, the Cas nuclease may be from a Type-IIA, Type-JIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015). In some embodiments, the RNA-guided DNA-binding agent comprises a Cas nickase. In some embodiments, the RNA-guided nickase is modified or derived from a Cas protein, such as a Class 2 Cas nuclease (which may be, e.g., a Cas nuclease of Type II, V, or VI). Class 2 Cas nuclease include, for example, Cas9, Cpf1, C2c1, C2c2, and C2c3 proteins and modifications thereof.

Non-limiting exemplary species that the Cas nuclease or Cas nickase can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonfex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the Cas nickase is derived from the Cas9 nuclease from Streptococcus pyogenes. In some embodiments, the Cas nickase is derived from the Cas9 nuclease from Streptococcus thermophilus. In some embodiments, the Cas nickase is a nickase form of the Cas9 nuclease from Neisseria meningitidis. See e.g., WO/2020081568, describing an Nme2Cas9 D16A nickase fusion protein.. In some embodiments, the Cas nickase is derived from the Cas9 nuclease is from Staphylococcus aureus. In some embodiments, the Cas nickase is derived from the Cpf1 nuclease from Francisella novicida. In some embodiments, the Cas nickase is derived from the Cpf1 nuclease from Acidaminococcus sp. In some embodiments, the Cas nickase is derived from the Cpf1 nuclease from Lachnospiraceae bacterium ND2006. In further embodiments, the Cas nickase is derived from the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nickase is derived from a Cpf1 nuclease from an Acidaminococcus or Lachnospiraceae. As discussed elsewhere, a nickase may be derived from a nuclease by inactivating one of the two catalytic domains, e.g., by mutating an active site residue essential for nucleolysis, such as D10, H840, of N863 in Spy Cas9. One skilled in the art will be familiar with techniques for easily identifying corresponding residues in other Cas proteins, such as sequence alignment and structural alignment, which is discussed in detail below.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease or Cas nickase may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—AOQ7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA binding agent comprises one or more heterologous functional domains (e.g., is or comprises a fusion polypeptide).

In some embodiments, the RNA-guided DNA binding agent comprises a APOBEC3 deaminase. In some embodiments, a APOBEC3 deaminase is a APOBEC3A (A3A). In some embodiments, the A3A is a human A3A. In some embodiments, the A3A is a wild-type A3A.

In some embodiments, the RNA-guided DNA binding agent comprises a deaminase and an RNA-guided nickase. In some embodiments, the mRNA further comprises a linker to link the sequencing encoding A3A to the sequence sequencing encoding RNA-guided nickase. In some embodiments, the linker is an organic molecule, group, polymer, or chemical moiety. In some embodiments, the linker is a peptide linker. In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 900), SGSETPGTSESA (SEQ ID NO: 901), or SGSETPGTSESATPEGGSGGS (SEQ ID NO: 902). In some embodiments, the peptide linker comprises one or more sequences selected from SEQ ID NOs: 903-913.

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be fused at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two NLS sequences (e.g., SV40) fused at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 600) or PKKKRRV (SEQ ID NO: 601). In some embodiments, the NLS may be a bipartite sequence, such as the NLS of nucleoplasmin, KRPAATKKAGQAKKKK (SEQ ID NO: 602). In a specific embodiment, a single PKKKRKV (SEQ ID NO: 600) NLS may be fused at the C-terminus of the RNA-guided DNA-binding agent. One or more linkers are optionally included at the fusion site.

In some embodiments, the RNA-guided DNA binding agent comprises an editor. An exemplary editor is BC22n which includes a H. sapiens APOBEC3A fused to S. pyogenes-D10A Cas9 nickase by an XTEN linker, and mRNA encoding BC22n. An mRNA encoding BC22n is provided (SEQ ID NO:804).

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub 1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyani, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6xHis, 8xHis, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain such as an editor domain. When the RNA-guided DNA-binding agent is directed to its target sequence, e.g., when a Cas nuclease is directed to a target sequence by a gRNA, the effector such as an editor domain may modify or affect the target sequence. In some embodiments, the effector such as an editor domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA.

D. Determination of Efficacy of Guide RNAs

In some embodiments, the efficacy of a guide RNA is determined when delivered or expressed together with other components (e.g., an RNA-guided DNA binding agent) forming an RNP. In some embodiments, the guide RNA is expressed together with an RNA-guided DNA binding agent, such as a Cas protein, e.g., Cas9. In some embodiments, the guide RNA is delivered to or expressed in a cell line that already stably expresses an RNA-guided DNA nuclease, such as a Cas nuclease or nickase, e.g., Cas9 nuclease or nickase. In some embodiments the guide RNA is delivered to a cell as part of a RNP. In some embodiments, the guide RNA is delivered to a cell along with a mRNA encoding an RNA-guided DNA nuclease, such as a Cas nuclease or nickase, e.g., Cas9 nuclease or nickase.

As described herein, use of an RNA-guided DNA nuclease and a guide RNA disclosed herein can lead to DSBs, SSBs, and/or site-specific binding that results in nucleic acid modification in the DNA or pre-mRNA which can produce errors in the form of insertion/deletion (indel) mutations upon repair by cellular machinery. Many mutations due to indels alter the reading frame, introduce premature stop codons, or induce exon skipping and, therefore, produce a non-functional protein.

In some embodiments, the efficacy of particular guide RNAs is determined based on in vitro models. In some embodiments, the in vitro model is T cell line. In some embodiments, the in vitro model is HEK293 T cells. In some embodiments, the in vitro model is HEK293 cells stably expressing Cas9 (HEK293_Cas9). In some embodiments, the in vitro model is a lymphoblastoid cell line. In some embodiments, the in vitro model is primary human T cells. In some embodiments, the in vitro model is primary human B cells. In some embodiments, the in vitro model is primary human peripheral blood lymphocytes. In some embodiments, the in vitro model is primary human peripheral blood mononuclear cells.

In some embodiments, the number of off-target sites at which a deletion or insertion occurs in an in vitro model is determined, e.g., by analyzing genomic DNA from the cells transfected in vitro with Cas9 mRNA and the guide RNA. In some embodiments, such a determination comprises analyzing genomic DNA from cells transfected in vitro with Cas9 mRNA, the guide RNA, and a donor oligonucleotide. Exemplary procedures for such determinations are provided in the working examples below.

In some embodiments, the efficacy of particular gRNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of particular guide RNAs is determined based on in vivo models. In some embodiments, the in vivo model is a rodent model. In some embodiments, the rodent model is a mouse which expresses the target gene. In some embodiments, the rodent model is a mouse which expresses a CIITA gene. In some embodiments, the rodent model is a mouse which expresses a human CIITA gene. In some embodiments, the rodent model is a mouse which expresses a B2M gene. In some embodiments, the rodent model is a mouse which expresses a human B2M gene. In some embodiments, the in vivo model is a non-human primate, for example cynomolgus monkey.

In some embodiments, the efficacy of a guide RNA is evaluated by on target cleavage efficiency. In some embodiments, the efficacy of a guide RNA is measured by percent editing at the target location, e.g., CIITA, or B2M. In some embodiments, deep sequencing may be utilized to identify the presence of modifications (e.g., insertions, deletions) introduced by gene editing. Indel percentage can be calculated from next generation sequencing “NGS.”

In some embodiments, the efficacy of a guide RNA is measured by the number and/or frequency of indels at off-target sequences within the genome of the target cell type. In some embodiments, efficacious guide RNAs are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., T cells or B cells), or which produce a frequency of off-target indel formation of <5% in a cell population and/or relative to the frequency of indel creation at the target site. In some embodiments, the disclosure provides guide RNAs which do not exhibit any off target indel formation in the target cell type (e.g., T cells or B cells). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., T cells or B cells) genome.

In some embodiments, linear amplification is used to detect gene editing events, such as the formation of insertion/deletion (“indel”) mutations, translocations, and homology directed repair (HDR) events in target DNA. For example, linear amplification with a unique sequence-tagged primer and isolating the tagged amplification products (herein after referred to as “UnIT,” or “Unique Identifier Tagmentation” method) may be used.

In some embodiments, the efficacy of a guide RNA is measured by the number of chromosomal rearrangements within the target cell type. Kromatid dGH assay may used to detect chromosomal rearrangements, including e.g., translocations, reciprocal translocations, translocations to off-target chromosomes, deletions (i.e., chromosomal rearrangements where fragments were lost during the cell replication cycle due to the editing event). In some embodiments, the target cell type has less than 10, less than 8, less than 5, less than 4, less than 3, less than 2, or less than 1 chromosomal rearrangement. In some embodiments, the target cell type has no chromosomal rearrangements.

E. Delivery of gRNA Compositions

Lipid nanoparticles (LNPs) are a well-known means for delivery of nucleotide and protein cargo and may be used for delivery of the guide RNAs, compositions, or pharmaceutical formulations disclosed herein. In some embodiments, the LNPs deliver nucleic acid, protein, or nucleic acid together with protein.

In some embodiments, the invention comprises a method for delivering any one of the gRNAs disclosed herein to a subject, wherein the gRNA is formulated as an LNP. In some embodiments, the LNP comprises the gRNA and a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO/2017/173054 and references described therein. In some embodiments, the LNPs comprise molar ratios of a cationic lipid amine to RNA phosphate (N:P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, the gRNAs disclosed herein are formulated as LNPs for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is formulated as an LNP or not formulated as an LNP. In some embodiments, the LNP comprises the gRNA and a Cas9 or an mRNA encoding Cas9.

In some embodiments, the guide RNA compositions described herein, alone or encoded on one or more vectors, are formulated in or administered via a lipid nanoparticle; see e.g., WO/2017/173054 and WO 2019/067992, the contents of which are hereby incorporated by reference in their entirety.

In certain embodiments, the invention comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

### IV. Therapeutic Methods and Uses

Any of the engineered cells and compositions described herein can be used in a method of treating a variety of diseases and disorders, as described herein. In some embodiments, the genetically modified cell (engineered cell) and/or population of genetically modified cells (engineered cells) and compositions may be used in methods of treating a variety of diseases and disorders. In some embodiments, a method of treating any one of the diseases or disorders described herein is encompassed, comprising administering any one or more composition described herein.

In some embodiments, the methods and compositions described herein may be used to treat diseases or disorders in need of delivery of a therapeutic agent. In some embodiments, the invention provides a method of providing an immunotherapy in a subject, the method including administering to the subject an effective amount of an engineered cell (or population of engineered cells) as described herein, for example, a cell of any of the aforementioned cell aspects and embodiments.

In some embodiments, the methods comprise administering to a subject a composition comprising an engineered cell described herein as an adoptive cell transfer therapy. In some embodiments, the engineered cell is an allogeneic cell.

In some embodiments, the methods comprise administering to a subject a composition comprising an engineered cell described herein, wherein the cell produces, secretes, and/or expresses a polypeptide (e.g., a targeting receptor) useful for treatment of a disease or disorder in a subject. In some embodiments, the cell acts as a cell factory to produce a soluble polypeptide. In some embodiments, the cell acts as a cell factory to produce an antibody. In some embodiments, the cell continuously secretes the polypeptide in vivo. In some embodiments, the cell continuously secretes the polypeptide following transplantation in vivo for at least 1, 2, 3, 4, 5, or 6 weeks. In some embodiments, the cell continuously secretes the polypeptide following transplantation in vivo for more than 6 weeks. In some embodiments, the soluble polypeptide (e.g., an antibody) is produced by the cell at a concentration of at least 10², 10³, 10⁴, 10⁵, 10⁶, 10⁷, or 10⁸ copies per day. In some embodiments, the polypeptide is an antibody and is produced by the cell at a concentration of at least 10⁸ copies per day.

In some embodiments of the methods, the method includes administering a lymphodepleting agent or immunosuppressant prior to administering to the subject an effective amount of the engineered cell (or engineered cells) as described herein, for example, a cell of any of the aforementioned cell aspects and embodiments. In another aspect, the invention provides a method of preparing engineered cells (e.g., a population of engineered cells).

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer cells, cytotoxic T lymphocytes (CTLs), T helper cells, B cells, or their progenitors such as hematopoietic stem cells (HSC) or induced pluripotent stem cells (iPSC) can be programmed to act in response to abnormal antigens expressed on the surface of tumor cells. Thus, cancer immunotherapy allows components of the immune system to destroy tumors or other cancerous cells. Cell-based immunotherapies have also been demonstrated to be effective in the treatment of autoimmune diseases or transplant rejection. Immune effector cells such as regulatory T cells (Tregs) or mesenchymal stem cells can be programmed to act in response to autoantigens or transplant antigens expressed on the surface of normal tissues.

In some embodiments, the invention provides a method of preparing engineered cells (e.g., a population of engineered cells). The population of engineered cells may be used for immunotherapy.

In some embodiments, the invention provides a method of treating a subject in need thereof that includes administering engineered cells prepared by a method of preparing cells described herein, for example, a method of any of the aforementioned aspects and embodiments of methods of preparing cells.

In some embodiments, the engineered cells can be used to treat cancer, infectious diseases, inflammatory diseases, autoimmune diseases, cardiovascular diseases, neurological diseases, ophthalmologic diseases, renal diseases, liver diseases, musculoskeletal diseases, red blood cell diseases, or transplant rejections. In some embodiments, the engineered cells can be used in cell transplant, e.g., to the heart, liver, lung, kidney, pancreas, skin, or brain. (See e.g., Deuse et al., Nature Biotechnology 37:252-258 (2019).)

In some embodiments, the engineered cells can be used as a cell therapy comprising an allogeneic stem cell therapy. In some embodiments, the cell therapy comprises induced pluripotent stem cells (iPSCs). iPSCs may be induced to differentiate into other cell types including e.g., beta islet cells, neurons, and blood cells. In some embodiments, the cell therapy comprises hematopoietic stem cells. In some embodiments, the stem cells comprise mesenchymal stem cells that can develop into bone, cartilage, muscle, and fat cells. In some embodiments, the stem cells comprise ocular stem cells. In some embodiments, the allogeneic stem cell transplant comprises allogeneic bone marrow transplant. In some embodiments, the stem cells comprise pluripotent stem cells (PSCs). In some embodiments, the stem cells comprise induced embryonic stem cells (ESCs).

The engineered cells disclosed herein are suitable for further engineering, e.g., by introduction of further edited, or modified genes or alleles. In some embodiments, the polypeptide is a wild-type or variant TCR. Cells of the invention may also be suitable for further engineering by introduction of an exogenous nucleic acid encoding e.g., a targeting receptor, e.g., a TCR, CAR, UniCAR. CARs are also known as chimeric immunoreceptors, chimeric T cell receptors or artificial T cell receptors.

In some embodiments, the cell therapy is a transgenic T cell therapy. In some embodiments, the cell therapy comprises a Wilms' Tumor 1 (WT1) targeting transgenic T cell. In some embodiments, the cell therapy comprises a targeting receptor or a donor nucleic acid encoding a targeting receptor of a commercially available T cell therapy, such as a CAR T cell therapy. There are number of targeting receptors currently approved for cell therapy. The cells and methods provided herein can be used with these known constructs. Commercially approved cell products that include targeting receptor constructs for use as cell therapies include e.g., Kymriah® (tisagenlecleucel); Yescarta® (axicabtagene ciloleucel); Tecartus™ (brexucabtagene autoleucel); Tabelecleucel (Tab-cel®); Viralym-M (ALVR105); and Viralym-C.

In some embodiments, the methods provide for administering the engineered cells to a subject, wherein the administration is an injection. In some embodiments, the methods provide for administering the engineered cells to a subject, wherein the administration is an intravascular injection or infusion. In some embodiments, the methods provide for administering the engineered cells to a subject, wherein the administration is a single dose.

In some embodiments, the methods provide for reducing a sign or symptom associated of a subject's disease treated with a composition disclosed herein. In some embodiments, the subject has a response to treatment with a composition disclosed herein that lasts more than one week. In some embodiments, the subject has a response to treatment with a composition disclosed herein that lasts more than two weeks. In some embodiments, the subject has a response to treatment with a composition disclosed herein that lasts more than three weeks. In some embodiments, the subject has a response to treatment with a composition disclosed herein that lasts more than one month.

In some embodiments, the methods provide for administering the engineered cells to a subject, and wherein the subject has a response to the administered cell that comprises a reduction in a sign or symptom associated with the disease treated by the cell therapy. In some embodiments, the subject has a response that lasts more than one week. In some embodiments, the subject has a response that lasts more than one month. In some embodiments, the subject has a response that lasts for at least 1-6 weeks.

### EXAMPLES

The following examples are provided to illustrate certain disclosed embodiments and are not to be construed as limiting the scope of this disclosure in any way.

### Example 1. General Methods

### 1.1. Next-Generation Sequencing (“NGS”) and Analysis for On-Target Cleavage Efficiency.

Genomic DNA was extracted using QuickExtract™ DNA Extraction Solution (Lucigen, Cat. No. QE09050) according to manufacturer's protocol.

To quantitatively determine the efficiency of editing at the target location in the genome, deep sequencing was utilized to identify the presence of insertions and deletions introduced by gene editing. PCR primers were designed around the target site within the gene of interest (e.g., CIITA) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. Reads that overlapped the target region of interest were re-aligned to the local genome sequence to improve the alignment. Then the number of wild type reads versus the number of reads which contain C-to-T mutations, C-to-A/G mutations or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### 1.2. T Cell Culture Media Preparation.

T cell culture media compositions used below are described here and in Table 3. “X-VIVO Base Media” consists of X-VIVO™ 15 Media, 1% Penstrep, 50 μM Beta-Mercaptoethanol, 10 mM NAC. In addition to above mentioned components, other variable media components used were: 1. Serum (Fetal Bovine Serum (FBS)); and 2. Cytokines (IL-2, IL-7, IL-15), also described in Table 3. T cell media components are described in Table 3 below.

TABLE 3
T cell media.

| | | | Media |
|---|---|---|---|
| Base Media | Serum | Cytokines | Number |
| XVIVO ™ 15 (Lonza, BE02-060Q) | 5% Human Serum | 5 ng/mL rh-IL2 | 1 |
| 1% Pen-Strep | AB | 5 ng/mL rh-IL7 | |
| 50 μM Beta-mercaptoethanol | (Gemini Bio | 200 Units/mL rh IL-15 | |
| 10 mM N-Acetyl L-Cystine (NAC) | Products, 100- | 5 ng/mL rh-IL2 | 2 |
| | 512) | 200 Units/mL rh IL-15 | |
| RPMI (Corning) | 10% Fetal Bovine | 5 ng/mL rh-IL2 | 11 |
| 2 mM L-glutamine | Serum | | |
| 1% Pen-Strep | | | |
| 100 μM Non-essential amino acids | | | |
| 1 mM Sodium pyruvate | | | |
| 10 mM HEPES | | | |
| 55 μM Beta-mercaptoethanol | | | |
| RPMI 1640 (Corning) | 10% Fetal Bovine | Cytokine free | 18 |
| 1X Glutamax | Serum | 100 U/mL rhIL-2 | 19 |
| 1% Pen-Strep, | | 2.5 ng/ml rIL-7 | |
| 100 uM Non-Essential Amino | | 2.5 ng/ml rIL-15 | |
| Acids | | | |
| 1 mM Sodium Pyruvate | | | |
| 10 mM HEPES Buffer | | | |
| 55 uM Beta-Mercaptoethanol | | | |
| RPMI 1640 (Corning) | 10% Fetal Bovine | 100 U/mL rhIL-2 | 20 |
| 22 mM Glutamax | Serum | | |
| 1% Pen-Strep, | | | |
| 100 uM Non-Essential Amino | | | |
| Acids | | | |
| 1 mM Sodium Pyruvate | | | |
| 10 mM HEPES Buffer | | | |
| 22 uM Beta-Mercaptoethanol | | | |

### 1.3. Preparation of Lipid Nanoparticles.

The lipid components were dissolved in 10000 ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL.

The lipid nucleic acid assemblies contained ionizable Lipid A ((9Z,12Z)-3-((4,4-bis(octyloxy)butanoyl)oxy)-2-((((3-(di ethyl amino)propoxy)carbonyl)oxy)methyl)propyl octadeca-9,12-dienoate, also called 3-((4,4-bis(octyloxy)butanoyl)oxy)-2-((((3-(diethyl amino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. The lipid nucleic acid assemblies were formulated with a lipid amine to RNA phosphate (N:P) molar ratio of about 6, and a ratio of gRNA to mRNA of 1:1 by weight.

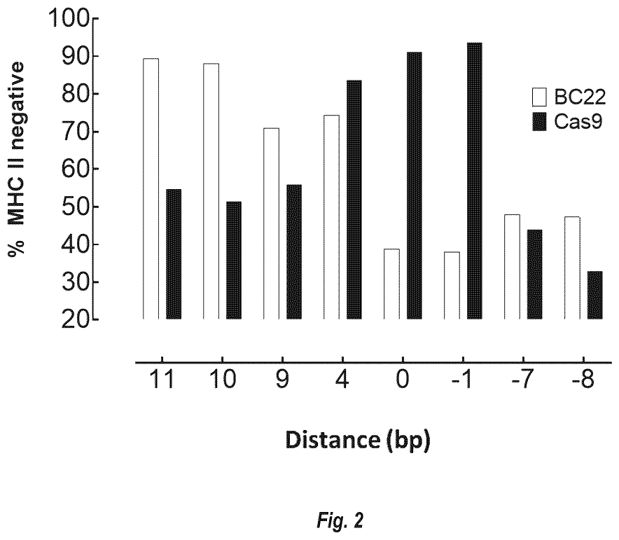

Lipid nanoparticles (LNPs) were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2.). The LNPs were held for 1 hour at room temperature (RT), and further diluted with water (approximately 1:1 v/v). LNPs were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 m sterile filter. The final LNP was stored at 4° C. or −80° C. until further use.

1.4. In Vitro Transcription (“IVT”) of Nuclease mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation region region was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID Nos: 801-803 (see sequences in Table 19). BC22n mRNA was generated from plasmid DNA encoding an open reading frame according to SEQ ID Nos: 804-805. BC22 mRNA was generated from plasmid DNA encoding an open reading frame according to SEQ ID No: 806. UGI mRNA was generated from plasmid DNA encoding an open reading frame according to SEQ ID Nos: 807-808. When SEQ ID NOs: 801-808 are referred to below with respect to RNAs, it is understood that Ts should be replaced with Us (which were N1-methyl pseudouridines as described above). Messenger RNAs used in the Examples include a 5′ cap and a 3′ polyadenylation region, e.g., up to 100 nts, and are identified by SEQ ID NOs: 801-808 in Table 19 below.

### Example 2. Screening of CITA Guide RNAs

CIITA guide RNAs were screened for efficacy in T cells by assessing loss of MHC class II cell surface expression. The percentage of T cells negative for MHC class II protein (“% MHC II negative”) was assayed following CIITA editing by electroporation with RNP.

### 2.1. RNP Electroporation of T Cells.

Cas9 editing activity was assessed using electroporation of Cas9 ribonucleoprotein (RNP). Upon thaw, Pan CD3+ T cells (StemCell, HLA-A*02.01/A*03.01) were plated at a density of 0.5×10⁶ cells/mL in T cell RPMI media composed of RPMI 1640 (Invitrogen, Cat. 22400-089) containing 5% (v/v) of fetal bovine serum, 1× Glutamax (Gibco, Cat. 35050-061), 50 μM of 2-Mercaptoethanol, 100 uM non-essential amino acids (Invitrogen, Cat. 11140-050), 1 mM sodium pyruvate, 10 mM HEPES buffer, 1% of Penicillin-Streptomycin, and 100 U/mL of recombinant human interleukin-2 (Peprotech, Cat. 200-02). T cells were activated with TransAct™ (1:100 dilution, Miltenyi Biotec). Cells were expanded in T cell RPMI media for 72 hours prior to RNP transfection.

CIITA targeting sgRNAs were removed from their storage plates and denatured for 2 minutes at 95° C. before cooling at room temperature for 10 minutes. RNP mixture of 20 uM sgRNA and 10 uM recombinant Cas9-NLS protein (SEQ ID NO: 800) is prepared and incubated at 25° C. for 10 minutes. Five μL of RNP mixture was combined with 100,000 cells in 20 μL P3 electroporation Buffer (Lonza). 22 μL of RNP/cell mix was transferred to the corresponding wells of a Lonza shuttle 96-well electroporation plate. Cells were electroporated in duplicate with the manufacturer's pulse code. T cell RPMI media was added to the cells immediately post electroporation. Electroporated T cells were subsequently cultured and collected for NGS sequencing as described in Example 1 at 2 days post edit.

### 2.2. Flow Cytometry.

On day 10 post-edit, T cells were phenotyped by flow cytometry to determine MHC class II protein expression. Briefly, T cells were incubated in cocktails of antibodies targeting HLA-DR, DQ, DP-PE (BioLegend® Cat. No. 361704) and Isotype Control-AF647 (BioLegend® Cat. No. 400234). Cells were subsequently washed, processed on a Cytoflex flow cytometer (Beckman Coulter) and analyzed using the FlowJo software package. T cells were gated based on size, shape, viability, and MHC class II expression. DNA samples were subjected to PCR and subsequent NGS analysis, as described in Example 1.

### 2.3. Results of CIITA Guide RNA Screening.

Table 4A shows the mean percentage of T cells negative for cell surface expression of MHC class II. Control 1 and Control 2 target B2M and TRAC respectively for comparative expression of MHC class I. For each guide, the genomic coordinate of the cut site with spCas9 is shown, as well as the distance (# of nucleotides) between the acceptor splice site boundary nucleotide or the donor splice site boundary nucleotide and the cut site (referred to in Table 4A as Distance from Cut Site). Numerical values without parentheses show the number of nucleotides in the 5′ directed between a splice site boundary nucleotide and cut site, whereas the numerical values in parentheses show the number of nucleotides in the 3′ direction between a splice site boundary nucleotide and cut site. Table 4B shows the results of the NGS analysis.

TABLE 4A
CIITA guide RNA screen.

| | | Mean % | | | | Distance | | |
|---|---|---|---|---|---|---|---|---|
| | SEQ | MHC | | | | from Cut | | |
| | ID | Class II | | | | Site | Genomic | |
| Guide | NO | negative | SD | Target Seq | Cut Site | (bp) | Location | Genomic Coord. |
| G018021 | 1 | 35.20 | 3.11 | UCCUACCUGUCAGAG | 10,877,378 | 4 | Exon 1 | chr16:10877360- |
| | | | | CCCCA | | | end | 10877380 |
| G018022 | 2 | 26.50 | 2.97 | GAGCCCCAAGGUAAA | 10,877,390 | (8) | Exon 1 | chr16:10877372- |
| | | | | AAGGC | | | end | 10877392 |
| G018023 | 3 | 27.15 | 5.16 | AGCCCCAAGGUAAAA | 10,877,391 | (9) | Exon 1 | chr16:10877373- |
| | | | | AGGCC | | | end | 10877393 |
| G018024 | 4 | 30.40 | 2.55 | UUUCCCGGCCUUUUU | 10,877,382 | — | Exon 1 | chr16:10877379- |
| | | | | ACCUU | | | end | 10877399 |
| G018025 | 5 | 30.40 | 1.84 | CUUUCCCGGCCUUUU | 10,877,383 | (1) | Exon 1 | chr16:10877380- |
| | | | | UACCU | | | end | 10877400 |
| G018026 | 6 | 39.50 | 5.09 | ACACUGUGAGCUGCC | 10,895,278 | 4 | Exon 2 | chr16:10895275- |
| | | | | UGGGA | | | start | 10895295 |
| G018027 | 7 | 38.70 | 0.28 | CACACUGUGAGCUGC | 10,895,279 | 3 | Exon 2 | chr16:10895276- |
| | | | | CUGGG | | | start | 10895296 |
| G018028 | 8 | 40.45 | 2.48 | GUGGCACACUGUGAG | 10,895,283 | (1) | Exon 2 | chr16:10895280- |
| | | | | CUGCC | | | start | 10895300 |
| G018029 | 9 | 36.70 | 2.83 | GAGAUUGAGCUCUAC | 10,895,427 | 1 | Exon 2 | chr16:10895409- |
| | | | | UCAGG | | | end | 10895429 |
| G018030 | 10 | 67.35 | 4.60 | AGAUUGAGCUCUACU | 10,895,428 | — | Exon 2 | chr16:10895410- |
| | | | | CAGGU | | | end | 10895430 |
| G018031 | 11 | 26.85 | 5.45 | CAGGUGGGCCCUCCU | 10,895,443 | (15) | Exon 2 | chr16:10895425- |
| | | | | CCCUC | | | end | 10895445 |
| G018032 | 12 | 12.30 | 2.26 | CGGAAGAGACCAGAG | 10,895,440 | (12) | Exon 2 | chr16:10895437- |
| | | | | GGAGG | | | end | 10895457 |
| G018033 | 13 | 31.45 | 2.33 | UACCGGAAGAGACCA | 10,895,443 | (15) | Exon 2 | chr16:10895440- |
| | | | | GAGGG | | | end | 10895460 |
| G018034 | 14 | 85.75 | 4.74 | AGGGAGGCUUAUGCC | 10,895,760 | 4 | Exon 3 | chr16:10895742- |
| | | | | AAUAU | | | end | 10895762 |
| G018035 | 15 | 80.00 | 2.97 | GGCUUAUGCCAAUAU | 10,895,765 | (1) | Exon 3 | chr16:10895747- |
| | | | | CGGUG | | | end | 10895767 |
| G018036 | 16 | 40.75 | 8.56 | UGACUGCGCUUUUCC | 10,898,660 | 10 | Exon 4 | chr16:10898642- |
| | | | | UUGUC | | | start | 10898662 |
| G018037 | 17 | 46.15 | 0.50 | GACUGCGCUUUUCCU | 10,898,661 | 9 | Exon 4 | chr16:10898643- |
| | | | | UGUCU | | | start | 10898663 |
| G018038 | 18 | 62.95 | 7.00 | GCUUUUCCUUGUCUG | 10,898,667 | 3 | Exon 4 | chr16:10898649- |
| | | | | GGCAG | | | start | 10898669 |
| G018039 | 19 | 43.85 | 3.04 | CCAGUUCCGCUGCCC | 10,898,661 | 9 | Exon 4 | chr16:10898658- |
| | | | | AGACA | | | start | 10898678 |
| G018040 | 20 | 29.65 | 3.47 | GACCUGAAGCACUGG | 10,901,554 | 4 | Exon 6 | chr16:10901536- |
| | | | | AAGCC | | | end | 10901556 |
| G018041 | 21 | 31.00 | 2.83 | GCACUGGAAGCCAGG | 10,901,562 | (4) | Exon 6 | chr16:10901544- |
| | | | | UGUGC | | | end | 10901564 |
| G018042 | 22 | 41.40 | 2.83 | CACUGGAAGCCAGGU | 10,901,563 | (5) | Exon 6 | chr16:10901545- |
| | | | | GUGCA | | | end | 10901565 |
| G018043 | 23 | 30.30 | 0.71 | GGAAGCCAGGUGUGC | 10,901,567 | (9) | Exon 6 | chr16:10901549- |
| | | | | AGGGC | | | end | 10901569 |
| G018044 | 24 | 25.90 | 0.28 | AGCCAGGUGUGCAGG | 10,901,570 | (12) | Exon 6 | chr16:10901552- |
| | | | | GCAGG | | | end | 10901572 |
| G018045 | 25 | 37.30 | 3.39 | GCCCACCUGCCCUGC | 10,901,560 | (2) | Exon 6 | chr16:10901557- |
| | | | | ACACC | | | end | 10901577 |
| G018046 | 26 | 44.30 | 8.77 | GGGCUCAGCUGUGAG | 10,902,028 | 10 | Exon 7 | chr16:10902025- |
| | | | | GAAGU | | | start | 10902045 |
| G018047 | 27 | 42.15 | 5.73 | GGGGCUCAGCUGUGA | 10,902,029 | 9 | Exon 7 | chr16:10902026- |
| | | | | GGAAG | | | start | 10902046 |
| G018048 | 28 | 37.15 | 6.58 | GACCAGAUUCCCAGU | 10,902,189 | (5) | Exon 7 | chr16:10902171- |
| | | | | AUGUU | | | end | 10902191 |
| G018049 | 29 | 34.30 | 5.66 | CCAGAUUCCCAGUAU | 10,902,191 | (7) | Exon 7 | chr16:10902173- |
| | | | | GUUAG | | | end | 10902193 |
| G018050 | 30 | 31.90 | 8.63 | CAGAUUCCCAGUAUG | 10,902,192 | (8) | Exon 7 | chr16:10902174- |
| | | | | UUAGG | | | end | 10902194 |
| G018051 | 31 | 30.50 | 5.66 | UCCCAGUAUGUUAGG | 10,902,197 | (13) | Exon 7 | chr16:10902179- |
| | | | | GGGCU | | | end | 10902199 |
| G018052 | 32 | 38.25 | 3.75 | UCCAAGCCCCCUAAC | 10,902,186 | (2) | Exon 7 | chr16:10902183- |
| | | | | AUACU | | | end | 10902203 |
| G018053 | 33 | 34.20 | 6.08 | CUCCAAGCCCCCUAA | 10,902,187 | (3) | Exon 7 | chr16:10902184- |
| | | | | CAUAC | | | end | 10902204 |
| G018054 | 34 | 37.20 | 3.82 | AAAGGCACUGCAAGA | 10,902,647 | 11 | Exon 8 | chr16:10902644- |
| | | | | GACAA | | | start | 10902664 |
| G018055 | 35 | 54.80 | 4.24 | UCCAGUAUAUUCAUC | 10,902,797 | 4 | Exon 8 | chr16:10902779- |
| | | | | UACCA | | | end | 10902799 |
| G018056 | 36 | 35.30 | 3.11 | UUCAUCUACCAUGGU | 10,902,806 | (5) | Exon 8 | chr16:10902788- |
| | | | | GAGUG | | | end | 10902808 |
| G018057 | 37 | 35.35 | 4.17 | UCAUCUACCAUGGUG | 10,902,807 | (6) | Exon 8 | chr16:10902789- |
| | | | | AGUGC | | | end | 10902809 |
| G018058 | 38 | 29.30 | 4.38 | CAUCUACCAUGGUGA | 10,902,808 | (7) | Exon 8 | chr16:10902790- |
| | | | | GUGCG | | | end | 10902810 |
| G018059 | 39 | 24.75 | 1.06 | ACCAUGGUGAGUGCG | 10,902,813 | (12) | Exon 8 | chr16:10902795- |
| | | | | GGGCC | | | end | 10902815 |
| G018060 | 40 | 34.75 | 5.02 | GCCAGGCCCCGCACU | 10,902,802 | (1) | Exon 8 | chr16:10902799- |
| | | | | CACCA | | | end | 10902819 |
| G018061 | 41 | 33.95 | 0.64 | CCACUCUCCACCCCC | 10,903,726 | 5 | Exon 9 | chr16:10903708- |
| | | | | AAUGU | | | start | 10903728 |
| G018062 | 42 | 60.40 | 3.54 | CUCCACCCCCAAUGU | 10,903,731 | — | Exon 9 | chr16:10903713- |
| | | | | AGGUG | | | start | 10903733 |
| G018063 | 43 | 33.05 | 8.56 | CACCUCACCUACAUU | 10,903,721 | 10 | Exon 9 | chr16:10903718- |
| | | | | GGGGG | | | start | 10903738 |
| G018064 | 44 | 35.55 | 7.00 | GGGCACCUCACCUAC | 10,903,724 | 7 | Exon 9 | chr16:10903721- |
| | | | | AUUGG | | | start | 10903741 |
| G018065 | 45 | 46.45 | 2.19 | UGGGGCACCUCACCU | 10,903,726 | 5 | Exon 9 | chr16:10903723- |
| | | | | ACAUU | | | start | 10903743 |
| G018066 | 46 | 50.95 | 2.19 | CUGGGGCACCUCACC | 10,903,727 | 4 | Exon 9 | chr16:10903724- |
| | | | | UACAU | | | start | 10903744 |
| G018067 | 47 | 40.75 | 2.19 | ACCUCCCGAGCAAAC | 10,903,891 | 4 | Exon 9 | chr16:10903873- |
| | | | | AUGAC | | | end | 10903893 |
| G018068 | 48 | 33.90 | 2.83 | CCGAGCAAACAUGAC | 10,903,896 | (1) | Exon 9 | chr16:10903878- |
| | | | | AGGUA | | | end | 10903898 |
| G018069 | 49 | 29.25 | 1.91 | GUACCUCUCACAGGC | 10,903,908 | (13) | Exon 9 | chr16:10903905- |
| | | | | CCUAA | | | end | 10903925 |
| G018070 | 50 | 20.85 | 1.48 | AGUACCUCUCACAGG | 10,903,909 | (14) | Exon 9 | chr16:10903906- |
| | | | | CCCUA | | | end | 10903926 |
| G018071 | 51 | 24.30 | 6.08 | GACGUCUUGUGCUCU | 10,904,739 | 5 | Exon 10 | chr16:10904736- |
| | | | | GGAGA | | | start | 10904756 |
| G018072 | 52 | 33.90 | 5.37 | AACAAGCUUCCAAAA | 10,904,808 | 4 | Exon 10 | chr16:10904790- |
| | | | | UGGCC | | | end | 10904810 |
| G018073 | 53 | 23.45 | 2.62 | GAGAUCCCGCAUCAC | 10,904,814 | (2) | Exon 10 | chr16:10904811- |
| | | | | UCACC | | | end | 10904831 |
| G018074 | 54 | 68.40 | 1.56 | GCCCCUGGCCUUUGC | 10,906,499 | — | Exon 11 | chr16:10906481- |
| | | | | AGAGC | | | start | 10906501 |
| G018075 | 55 | 54.70 | 3.54 | ACCGGCUCUGCAAAG | 10,906,488 | 11 | Exon 11 | chr16:10906485- |
| | | | | GCCAG | | | start | 10906505 |
| G018076 | 56 | 51.45 | 6.29 | CACCGGCUCUGCAAA | 10,906,489 | 10 | Exon 11 | chr16:10906486- |
| | | | | GGCCA | | | start | 10906506 |
| G018077 | 57 | 55.90 | 0.14 | CCACCGGCUCUGCAA | 10,906,490 | 9 | Exon 11 | chr16:10906487- |
| | | | | AGGCC | | | start | 10906507 |
| G018078 | 58 | 83.65 | 3.04 | CUGCUCCACCGGCUC | 10,906,495 | 4 | Exon 11 | chr16:10906492- |
| | | | | UGCAA | | | start | 10906512 |
| G018079 | 59 | 19.25 | 2.62 | UCAGCUGUGUCACCC | 10,908,145 | 4 | Exon 11 | chr16:10908127- |
| | | | | GUUUC | | | end | 10908147 |
| G018080 | 60 | 63.90 | 4.53 | GCUGUGUCACCCGUU | 10,908,148 | 1 | Exon 11 | chr16:10908130- |
| | | | | UCAGG | | | end | 10908150 |
| G018081 | 61 | 91.10 | 0.42 | CUGUGUCACCCGUUU | 10,908,149 | — | Exon 11 | chr16:10908131- |
| | | | | CAGGU | | | end | 10908151 |
| G018082 | 62 | 93.60 | 2.26 | UGUGUCACCCGUUUC | 10,908,150 | (1) | Exon 11 | chr16:10908132- |
| | | | | AGGUG | | | end | 10908152 |
| G018083 | 63 | 21.50 | 2.97 | CACCCGUUUCAGGUG | 10,908,155 | (6) | Exon 11 | chr16:10908137- |
| | | | | GGGUG | | | end | 10908157 |
| G018084 | 64 | 44.00 | 3.39 | ACCCGUUUCAGGUGG | 10,908,156 | (7) | Exon 11 | chr16:10908138- |
| | | | | GGUGA | | | end | 10908158 |
| G018085 | 65 | 32.95 | 0.92 | CCCGUUUCAGGUGGG | 10,908,157 | (8) | Exon 11 | chr16:10908139- |
| | | | | GUGAG | | | end | 10908159 |
| G018086 | 66 | 83.30 | 4.38 | GUCUGAGGCCCUCCC | 10,909,024 | 5 | Exon 12 | chr16:10909006- |
| | | | | UCCAC | | | start | 10909026 |
| G018087 | 67 | 67.95 | 2.76 | UCUGAGGCCCUCCCU | 10,909,025 | 4 | Exon 12 | chr16:10909007- |
| | | | | CCACA | | | start | 10909027 |
| G018088 | 68 | 61.70 | 0.14 | CAAGGCAGCCCUGUG | 10,909,021 | 8 | Exon 12 | chr16:10909018- |
| | | | | GAGGG | | | start | 10909038 |
| G018089 | 69 | 26.10 | 0.28 | GCUCAAGGCAGCCCU | 10,909,024 | 5 | Exon 12 | chr16:10909021- |
| | | | | GUGGA | | | start | 10909041 |
| G018090 | 70 | 86.75 | 0.07 | CGCUCAAGGCAGCCC | 10,909,025 | 4 | Exon 12 | chr16:10909022- |
| | | | | UGUGG | | | start | 10909042 |
| G018091 | 71 | 84.20 | 1.56 | UGUGCAGACUCAGAG | 10,909,190 | (3) | Exon 12 | chr16:10909172- |
| | | | | GUGAG | | | end | 10909192 |
| G018092 | 72 | 39.65 | 8.98 | UAACAUUGCCUGUUC | 10,910,183 | 5 | Exon 13 | chr16:10910165- |
| | | | | UCUCC | | | start | 10910185 |
| G018093 | 73 | 29.55 | 5.59 | CUUCUCGUCCUGGAG | 10,910,179 | 9 | Exon 13 | chr16:10910176- |
| | | | | AGAAC | | | start | 10910196 |
| G018094 | 74 | 70.65 | 11.24 | UUCCGAGGAACUUCU | 10,910,189 | (1) | Exon 13 | chr16:10910186- |
| | | | | CGUCC | | | start | 10910206 |
| G018095 | 75 | 41.25 | 0.50 | ACCCUUGCUCUUUGC | 10,915,565 | 5 | Exon 14 | chr16:10915547- |
| | | | | CUCCU | | | start | 10915567 |
| G018096 | 76 | 24.35 | 4.03 | UUGCUCUUUGCCUCC | 10,915,569 | 1 | Exon 14 | chr16:10915551- |
| | | | | UAGGC | | | start | 10915571 |
| G018097 | 77 | 55.05 | 8.98 | UGCUCUUUGCCUCCU | 10,915,570 | — | Exon 14 | chr16:10915552- |
| | | | | AGGCU | | | start | 10915572 |
| G018098 | 78 | 33.60 | 2.55 | CCUGAGACAGGGCCC | 10,915,570 | — | Exon 14 | chr16:10915567- |
| | | | | AGCCU | | | start | 10915587 |
| G018099 | 79 | 72.60 | 1.41 | AUCUGAUUCCACCUG | 10,916,366 | 1 | Exon 15 | chr16:10916348- |
| | | | | CAGCC | | | start | 10916368 |
| G018100 | 80 | 62.90 | 5.37 | CAGCGCAUCCAGGCU | 10,916,362 | 5 | Exon 15 | chr16:10916359- |
| | | | | GCAGG | | | start | 10916379 |
| G018101 | 81 | 85.70 | 4.38 | ACUCAGCGCAUCCAG | 10,916,365 | 2 | Exon 15 | chr16:10916362- |
| | | | | GCUGC | | | start | 10916382 |
| G018102 | 82 | 55.75 | 5.59 | AAACCCUCAAGUGAG | 10,916,467 | (8) | Exon 15 | chr16:10916449- |
| | | | | UGAGC | | | end | 10916469 |
| G018103 | 83 | 28.00 | 0.57 | AACCCUCAAGUGAGU | 10,916,468 | (9) | Exon 15 | chr16:10916450- |
| | | | | GAGCU | | | end | 10916470 |
| G018104 | 84 | 85.30 | 8.34 | GGCCCAGCUCACUCA | 10,916,458 | 1 | Exon 15 | chr16:10916455- |
| | | | | CUUGA | | | end | 10916475 |
| G018105 | 85 | 89.90 | 1.70 | AGGCCCAGCUCACUC | 10,916,459 | — | Exon 15 | chr16:10916456- |
| | | | | ACUUG | | | end | 10916476 |
| G018106 | 86 | 30.25 | 2.05 | CAGACUGCGGGGACA | 10,918,426 | 14 | Exon 16 | chr16:10918423- |
| | | | | CAGUG | | | start | 10918443 |
| G018107 | 87 | 89.90 | 0.28 | CUGCAUCCCUGCUCA | 10,918,522 | 4 | Exon 16 | chr16:10918504- |
| | | | | GGCUA | | | end | 10918524 |
| G018108 | 88 | 86.10 | 6.22 | CCUGCUCAGGCUAAG | 10,918,529 | (3) | Exon 16 | chr16:10918511- |
| | | | | GUGAG | | | end | 10918531 |
| G018109 | 89 | 86.55 | 2.19 | CUGCUCAGGCUAAGG | 10,918,530 | (4) | Exon 16 | chr16:10918512- |
| | | | | UGAGU | | | end | 10918532 |
| G018110 | 90 | 31.30 | 5.52 | CAGCACCUGACCGGU | 10,918,542 | (16) | Exon 16 | chr16:10918539- |
| | | | | AUCCG | | | end | 10918559 |
| G018111 | 91 | 26.65 | 2.76 | GUACAAGCUGUCGGA | 10,922,156 | 11 | Exon 17 | chr16:10922153- |
| | | | | AACAG | | | start | 10922173 |
| G018112 | 92 | 81.95 | 2.33 | GGAGACGCUGGCGUA | 10,922,496 | (6) | Exon 18 | chr16:10922478- |
| | | | | AGUCC | | | end | 10922498 |
| G018113 | 93 | 22.50 | 0.85 | GGCGUAAGUCCAGGC | 10,922,505 | (15) | Exon 18 | chr16:10922487- |
| | | | | AACCC | | | end | 10922507 |
| G018114 | 94 | 36.05 | 4.74 | CUCCACCCACCAGGG | 10,922,502 | (12) | Exon 18 | chr16:10922499- |
| | | | | UUGCC | | | end | 10922519 |
| G018115 | 95 | 31.40 | 4.67 | UGAGUCCCAUCCCCC | 10,923,223 | 5 | Exon 19 | chr16:10923205- |
| | | | | CUUGC | | | start | 10923225 |
| G018116 | 96 | 22.80 | 1.84 | CCACAUCCUGCAAGG | 10,923,217 | 11 | Exon 19 | chr16:10923214- |
| | | | | GGGGA | | | start | 10923234 |
| G018117 | 97 | 34.50 | 3.82 | GCGUCCACAUCCUGC | 10,923,221 | 7 | Exon 19 | chr16:10923218- |
| | | | | AAGGG | | | start | 10923238 |
| G018118 | 98 | 43.30 | 3.82 | GGCGUCCACAUCCUG | 10,923,222 | 6 | Exon 19 | chr16:10923219- |
| | | | | CAAGG | | | start | 10923239 |
| G018119 | 99 | 25.35 | 1.77 | GGGCGUCCACAUCCU | 10,923,223 | 5 | Exon 19 | chr16:10923220- |
| | | | | GCAAG | | | start | 10923240 |
| G018120 | 100 | 62.60 | 4.24 | UGGGCGUCCACAUCC | 10,923,224 | 4 | Exon 19 | chr16:10923221- |
| | | | | UGCAA | | | start | 10923241 |
| G018121 | 101 | 35.35 | 4.46 | GUGGGCGUCCACAUC | 10,923,225 | 3 | Exon 19 | chr16:10923222- |
| | | | | CUGCA | | | start | 10923242 |
| No Guide | 105 | 22.95 | 1.20 | | | | | |
| (Cells only) | | | | | | | | |
| G000529 | 700 | 26.40 | 2.69 | GGCCACGGAGCGAGA | | | | chr15:44711544- |
| (Control 1) | | | | CAUCU | | | | 44711564 |
| G010036 | 107 | 20.00 | 2.97 | ACACAAAUACCAGUC | | | | chr18:50681533- |
| (Control 2) | | | | CAGCG | | | | 50681553 |

TABLE 4B
CIITA NGS Analysis.

| | Seq ID | Primer Set 1 | | Primer Set 2 | |
|---|---|---|---|---|---|
| Guide ID | NO | Mean % Edit | SD % Edit | Mean % Edit | SD % Edit |
| G018021 | 1 | 56.80 | 14.28 | 48.55 | 7.85 |
| G018022 | 2 | 43.45 | 2.76 | 29.85 | 5.30 |
| G018023 | 3 | 68.55 | 5.44 | 32.30 | 0.85 |
| G018024 | 4 | | | 26.65 | 2.05 |
| G018025 | 5 | 57.00 | 2.55 | 31.75 | 1.06 |
| G018026 | 6 | 50.40 | 0.42 | 38.65 | 6.72 |
| G018027 | 7 | 39.00 | 14.00 | 25.40 | 0.14 |
| G018028 | 8 | 28.45 | 8.84 | 29.75 | 5.44 |
| G018029 | 9 | | | 9.35 | 1.20 |
| G018030 | 10 | | | 21.65 | 1.48 |
| G018031 | 11 | | | 18.65 | 3.46 |
| G018032 | 12 | | | 9.65 | 2.62 |
| G018033 | 13 | 35.80 | | 30.40 | 1.27 |
| G018034 | 14 | 46.05 | 23.69 | 57.05 | 2.90 |
| G018035 | 15 | 48.15 | 14.21 | 48.55 | 8.41 |
| G018036 | 16 | 46.75 | 19.02 | | |
| G018037 | 17 | | | 50.15 | 13.08 |
| G018038 | 18 | | | 44.40 | 21.92 |
| G018039 | 19 | | | 45.00 | 21.92 |
| G018040 | 20 | 7.95 | 5.16 | 5.10 | 3.54 |
| G018041 | 21 | 23.85 | 13.51 | 9.60 | 6.51 |
| G018042 | 22 | 52.10 | 18.67 | 38.75 | 13.79 |
| G018043 | 23 | 41.10 | 5.09 | 16.35 | 7.57 |
| G018044 | 24 | 72.45 | 4.74 | 19.15 | 12.80 |
| G018045 | 25 | 25.70 | 11.46 | 27.70 | 6.65 |
| G018046 | 26 | 39.50 | 24.61 | 30.65 | 13.36 |
| G018047 | 27 | 36.10 | 22.63 | 23.25 | 8.27 |
| G018048 | 28 | | | 32.75 | 15.34 |
| G018049 | 29 | | | 38.05 | 6.15 |
| G018050 | 30 | | | 35.95 | 0.35 |
| G018051 | 31 | | | 28.60 | 3.82 |
| G018052 | 32 | | | 40.45 | 4.03 |
| G018053 | 33 | | | 38.20 | 8.06 |
| G018054 | 34 | 48.30 | 12.30 | 39.40 | 5.37 |
| G018055 | 35 | 59.20 | | 46.50 | 7.64 |
| G018056 | 36 | 66.50 | | 36.15 | 15.63 |
| G018057 | 37 | 37.20 | | 32.80 | 12.02 |
| G018058 | 38 | | | 56.00 | 12.30 |
| G018059 | 39 | | | 6.35 | 2.47 |
| G018060 | 40 | | | 48.35 | 9.26 |
| G018061 | 41 | 45.15 | 20.29 | 50.65 | 6.86 |
| G018062 | 42 | 45.75 | 11.24 | 37.90 | 9.76 |
| G018063 | 43 | 40.00 | 9.76 | 34.65 | 1.20 |
| G018064 | 44 | 58.30 | 0.99 | 50.75 | 0.92 |
| G018065 | 45 | 52.95 | 7.99 | 49.75 | 0.21 |
| G018066 | 46 | 51.55 | 15.77 | 47.65 | 2.05 |
| G018067 | 47 | 63.30 | | 32.40 | 13.01 |
| G018068 | 48 | 45.75 | 7.14 | 40.75 | 1.63 |
| G018069 | 49 | 51.20 | 3.96 | 45.40 | 2.69 |
| G018070 | 50 | 37.85 | 13.79 | 49.00 | 4.95 |
| G018071 | 51 | 24.20 | | 9.50 | 13.44 |
| G018072 | 52 | 41.30 | | | |
| G018073 | 53 | 31.05 | 2.19 | 43.20 | 2.40 |
| G018074 | 54 | 29.05 | 12.09 | 29.00 | 3.39 |
| G018075 | 55 | 31.30 | | 30.75 | 7.85 |
| G018076 | 56 | 34.30 | 3.54 | 32.40 | 2.97 |
| G018077 | 57 | 32.00 | 10.89 | 33.55 | 1.34 |
| G018078 | 58 | 38.05 | 7.42 | 38.30 | 9.48 |
| G018079 | 59 | 0.10 | 0.00 | 0.10 | 0.00 |
| G018080 | 60 | 25.30 | 2.12 | 29.75 | 1.34 |
| G018081 | 61 | 24.80 | 19.23 | 15.95 | 2.47 |
| G018082 | 62 | 27.05 | 20.44 | 18.55 | 7.28 |
| G018083 | 63 | | | 0.35 | 0.07 |
| G018084 | 64 | 30.30 | 17.39 | 20.35 | 1.77 |
| G018085 | 65 | 37.80 | 26.16 | 22.70 | 8.49 |
| G018086 | 66 | 36.90 | 26.02 | 39.35 | 6.01 |
| G018087 | 67 | 46.95 | 19.87 | 41.15 | 2.47 |
| G018088 | 68 | 27.50 | 0.57 | 34.40 | 0.00 |
| G018089 | 69 | 70.75 | 23.55 | 60.70 | 6.08 |
| G018090 | 70 | 49.85 | 29.63 | 35.05 | 4.17 |
| G018091 | 71 | 62.70 | 23.76 | 0.00 | |
| G018092 | 72 | 11.25 | 0.49 | | |
| G018093 | 73 | 12.15 | 4.03 | | |
| G018094 | 74 | 18.40 | 15.84 | | |
| G018095 | 75 | 6.35 | 8.98 | | |
| G018096 | 76 | 1.55 | 0.35 | | |
| G018097 | 77 | 15.60 | 1.27 | | |
| G018098 | 78 | 12.30 | 3.11 | | |
| G018099 | 79 | 36.60 | 8.34 | | |
| G018100 | 80 | | | | |
| G018101 | 81 | 0.00 | 0.00 | | |
| G018102 | 82 | 4.00 | 1.84 | | |
| G018103 | 83 | 6.45 | 0.78 | | |
| G018104 | 84 | 27.40 | 0.28 | | |
| G018105 | 85 | 37.30 | 25.88 | | |
| G018106 | 86 | 14.65 | 1.48 | | |
| G018107 | 87 | 22.00 | 19.66 | | |
| G018108 | 88 | 10.65 | 5.73 | | |
| G018109 | 89 | 63.50 | 8.91 | | |
| G018110 | 90 | 33.45 | 16.62 | | |
| G018111 | 91 | 19.50 | 5.66 | | |
| G018112 | 92 | 30.85 | 0.21 | | |
| G018113 | 93 | | | | |
| G018114 | 94 | 6.70 | 5.09 | | |
| G018115 | 95 | 0.00 | 0.00 | | |
| G018116 | 96 | | | | |
| G018117 | 97 | | | | |
| G018118 | 98 | | | | |
| G018119 | 99 | | | | |
| G018120 | 100 | | | | |
| G018121 | 101 | | | | |
| G000529 | 700 | 43.30 | | | |
| G010036 | 107 | | | | |

### Example 3. Screening of CITA Guide RNAs Using BC22

CIITA guide RNAs were screened using BC22, a base conversion editor nuclease that includes a fusion of Cas9D10A nickase, human APOBEC3A deaminase and uracil glycosylase inhibitor. The characteristic edit of this construct is a cytosine to thymine conversion, rather than the indel typical of Cas9 cleavase. The efficacy in T cells was assessed by loss of MHC class II cell surface expression. The percentage of T cells negative for MHC class II protein was assayed following CIITA editing by electroporation with mRNA and guide.

3.1. mRNA Electroporation of T Cells.

Upon thaw, Pan CD3+ T cells isolated from a commercially obtained leukopak (StemCell) were plated at a density of 0.5×10⁶ cells/mL in Media 20 from Table 3. T cells were activated with Dynabeads® Human T-Activator CD3/CD28 (ThermoFisher). Cells were expanded in T cell for 72 hours prior to mRNA transfection.

CIITA sgRNAs (Table 4A) were removed from their storage plates and denatured for 2 minutes at 95° C. before cooling at room temperature for 10 minutes. Fifty microliter of the electroporation mix was prepared with 100,00 T cells in P3 buffer (Lonza) and 10 ng/uL mRNA encoding UGI (SEQ ID No. 807), 10 ng/uL mRNA encoding BC22 (SEQ ID No. 806) and 2 μM sgRNA. This mix was transferred to the corresponding wells of a Lonza shuttle 96-well electroporation plate. Cells were electroporated in duplicate wells using Lonza shuttle 96w using manufacturer's pulse code. Media 20 was added to the cells immediately post electroporation. Electroporated T cells were subsequently cultured and collected for NGS sequencing and flow cytometry 10 days post edit. Flow cytometry was performed as described in Example 2. DNA samples were subjected to PCR and subsequent NGS analysis, as described in Example 1.

Table 5 shows the mean percentage of T cells negative for cell surface expression of MHC class II as well as the mean percent editing.

TABLE 5
CIITA guide screen using BC22.

| | | Mean % | | Mean % | |
|---|---|---|---|---|---|
| | SEQ | MHC Class II | | C -to- T | |
| Guide | ID NO | Negative | SD | Editing | SD |
| G018021 | 1 | 38.65 | 0.21 | 87.55 | 0.21 |
| G018022 | 2 | 23.05 | 0.64 | 88.15 | 0.64 |
| G018023 | 3 | 25.00 | 0.71 | 84.05 | 0.78 |
| G018024 | 4 | 24.85 | 1.63 | 87.20 | 0.57 |
| G018025 | 5 | 23.05 | 2.76 | 84.95 | 0.92 |
| G018026 | 6 | 35.65 | 2.90 | 71.00 | 0.71 |
| G018027 | 7 | 30.65 | 1.06 | 75.00 | 0.85 |
| G018028 | 8 | 25.20 | 3.11 | 75.50 | 0.71 |
| G018029 | 9 | 24.00 | 0.28 | 5.70 | 0.57 |
| G018030 | 10 | 24.60 | 0.00 | 38.65 | 1.77 |
| G018031 | 11 | 22.85 | 0.07 | 59.70 | 4.38 |
| G018032 | 12 | 20.15 | 1.63 | 21.75 | 0.21 |
| G018033 | 13 | 21.05 | 1.34 | 64.85 | 1.06 |
| G018034 | 14 | 72.90 | 2.69 | 74.00 | 1.13 |
| G018035 | 15 | 47.00 | 0.28 | 73.70 | 0.28 |
| G018036 | 16 | 22.90 | 0.28 | 36.25 | 0.78 |
| G018037 | 17 | 21.90 | 2.26 | 49.30 | 2.26 |
| G018038 | 18 | 20.90 | 1.84 | 58.00 | 6.65 |
| G018039 | 19 | 62.55 | 4.60 | 67.45 | 2.05 |
| G018040 | 20 | 20.15 | 2.62 | 11.60 | 0.14 |
| G018041 | 21 | 22.25 | 2.33 | 17.15 | 0.49 |
| G018042 | 22 | 34.80 | 0.28 | 47.25 | 0.78 |
| G018043 | 23 | 31.20 | 2.97 | 36.15 | 0.78 |
| G018044 | 24 | 33.15 | 1.34 | 31.65 | 0.92 |
| G018045 | 25 | 22.75 | 1.20 | 66.35 | 1.20 |
| G018046 | 26 | 31.65 | 5.44 | 79.10 | 1.70 |
| G018047 | 27 | 28.20 | 4.38 | 75.55 | 0.35 |
| G018048 | 28 | 41.00 | 2.83 | 64.75 | 1.34 |
| G018049 | 29 | 28.95 | 4.17 | 53.25 | 1.48 |
| G018050 | 30 | 31.00 | 3.11 | 75.35 | 0.78 |
| G018051 | 31 | 27.90 | 4.10 | 50.80 | |
| G018052 | 32 | 20.90 | 1.27 | 74.25 | 0.78 |
| G018053 | 33 | 21.75 | 0.92 | 52.90 | 1.41 |
| G018054 | 34 | 39.95 | 0.21 | 82.50 | 0.57 |
| G018055 | 35 | 23.50 | 2.97 | 71.25 | 0.92 |
| G018056 | 36 | 24.85 | 2.05 | 77.20 | 3.11 |
| G018057 | 37 | 23.30 | 0.99 | 68.55 | 1.06 |
| G018058 | 38 | 25.45 | 0.64 | 67.15 | 0.78 |
| G018059 | 39 | 24.55 | 0.49 | 4.80 | |
| G018060 | 40 | 22.50 | 2.26 | 91.65 | 0.64 |
| G018061 | 41 | 22.50 | 0.71 | 73.60 | 0.57 |
| G018062 | 42 | 24.65 | 0.07 | 72.75 | 0.78 |
| G018063 | 43 | 34.80 | 0.14 | 68.85 | 0.49 |
| G018064 | 44 | 24.95 | 2.33 | 76.75 | 2.62 |
| G018065 | 45 | 26.60 | 0.71 | 70.35 | 0.07 |
| G018066 | 46 | 24.35 | 4.88 | 70.65 | 0.35 |
| G018067 | 47 | 86.65 | 1.20 | 83.80 | 0.42 |
| G018068 | 48 | 50.50 | 2.55 | 82.10 | 0.14 |
| G018069 | 49 | 24.50 | 1.98 | 83.95 | 0.21 |
| G018070 | 50 | 25.55 | 0.64 | 82.35 | 0.49 |
| G018071 | 51 | 32.25 | 0.49 | 70.40 | 0.28 |
| G018072 | 52 | 23.65 | 3.32 | 74.45 | 1.63 |
| G018073 | 53 | 22.65 | 2.76 | 75.90 | 1.84 |
| G018074 | 54 | 21.60 | 2.97 | 50.50 | 0.99 |
| G018075 | 55 | 88.30 | 1.56 | 87.05 | 0.07 |
| G018076 | 56 | 88.45 | 0.64 | 87.45 | 0.35 |
| G018077 | 57 | 69.15 | 2.47 | 78.65 | 1.48 |
| G018078 | 58 | 71.35 | 4.17 | 88.35 | 0.64 |
| G018079 | 59 | 39.15 | 8.27 | 73.75 | 1.34 |
| G018080 | 60 | 32.85 | 0.07 | 57.45 | 1.77 |
| G018081 | 61 | 37.90 | 1.27 | 77.30 | 3.82 |
| G018082 | 62 | 36.75 | 1.91 | 77.05 | 3.18 |
| G018083 | 63 | 37.65 | 1.06 | 49.50 | 0.71 |
| G018084 | 64 | 45.25 | 3.75 | 77.35 | 0.49 |
| G018085 | 65 | 45.75 | 2.19 | 75.00 | 1.13 |
| G018086 | 66 | 18.85 | 1.06 | 77.85 | 0.21 |
| G018087 | 67 | 18.45 | 2.05 | 77.00 | 1.56 |
| G018088 | 68 | 42.20 | 4.38 | 90.10 | 0.28 |
| G018089 | 69 | 34.30 | 3.25 | 88.70 | 1.41 |
| G018090 | 70 | 39.70 | 3.39 | 90.20 | 0.28 |
| G018091 | 71 | 85.60 | 1.56 | 82.30 | 1.13 |
| G018092 | 72 | 26.70 | 3.96 | 56.65 | 0.78 |
| G018093 | 73 | 74.05 | 1.48 | 80.95 | 1.63 |
| G018094 | 74 | 36.10 | 1.41 | 72.25 | 0.92 |
| G018095 | 75 | 25.75 | 3.46 | 91.70 | 1.27 |
| G018096 | 76 | 27.15 | 3.32 | 16.60 | 1.13 |
| G018097 | 77 | 28.90 | 1.70 | 49.40 | 0.28 |
| G018098 | 78 | 28.40 | 0.57 | 16.65 | 2.76 |
| G018099 | 79 | 26.75 | 0.64 | 84.50 | 1.13 |
| G018100 | 80 | 87.80 | 0.00 | 85.05 | 0.49 |
| G018101 | 81 | 81.10 | 2.26 | 85.50 | 4.53 |
| G018102 | 82 | 72.25 | 0.35 | 64.50 | 0.71 |
| G018103 | 83 | 80.90 | 1.56 | 71.50 | 0.14 |
| G018104 | 84 | 26.00 | 4.53 | 82.95 | 1.77 |
| G018105 | 85 | 24.85 | 1.48 | 86.30 | 0.14 |
| G018106 | 86 | 85.25 | 1.20 | 81.40 | 2.26 |
| G018107 | 87 | 94.33 | 0.06 | 87.10 | 0.99 |
| G018108 | 88 | 55.25 | 0.64 | 85.75 | 1.63 |
| G018109 | 89 | 44.40 | 0.71 | 84.85 | 0.64 |
| G018110 | 90 | 26.05 | 0.49 | 76.15 | 2.19 |
| G018111 | 91 | 81.55 | 0.07 | 83.35 | 0.49 |
| G018112 | 92 | 33.05 | 1.91 | 75.30 | 1.41 |
| G018113 | 93 | 32.85 | 1.06 | 80.25 | 3.32 |
| G018114 | 94 | 33.60 | 3.54 | 64.40 | 1.84 |
| G018115 | 95 | 23.95 | 2.05 | 55.45 | 2.33 |
| G018116 | 96 | 68.10 | 2.12 | 53.30 | 2.26 |
| G018117 | 97 | 93.25 | 0.20 | 86.10 | 0.71 |
| G018118 | 98 | 92.96 | 0.42 | 84.00 | 0.14 |
| G018119 | 99 | 57.80 | 2.12 | 44.60 | 2.69 |
| G018120 | 100 | 89.40 | 0.28 | 84.15 | 1.91 |
| G018121 | 101 | 75.85 | 0.49 | 75.00 | 0.99 |

3.4. Guide Position Vs. MHC Class II Protein Knockdown

Table 6 and FIG. 2 show the percent knockout of MHC class II using Cas9 and BC22 in relation to the distance from the cut site to the splice site boundary nucleotide.

For each guide, the genomic coordinate of the cut site with spCas9 is shown, as well as the distance (# of nucleotides) between the acceptor splice site boundary nucleotide or the donor splice site boundary nucleotide and the cut site. Positive numerical values show the number of nucleotides in the 5′ direction between a splice site boundary nucleotide and cut site, whereas the negative numerical values show the number of nucleotides in the 3′ direction between a splice site boundary nucleotide and cut site.

TABLE 6
Guide position vs. protein knockdown efficiency

| | |
|---|---|
| BC22 | Cas9 |

| | | Mean % | | Mean % | |
|---|---|---|---|---|---|
| | Distance | MHC | | MHC | |
| | from Cut | Class II | | Class II | |
| Guide | Site (bp) | negative | SD | negative | SD |
| G018075 | 11 | 88.3 | 1.56 | 54.7 | 3.54 |
| G018076 | 10 | 88.45 | 0.64 | 51.45 | 6.29 |
| G018077 | 9 | 69.15 | 2.48 | 55.9 | 0.14 |
| G018078 | 4 | 71.35 | 4.17 | 83.65 | 3.04 |
| G018081 | 0 | 37.9 | 1.27 | 91.1 | 0.42 |
| G018082 | −1 | 36.75 | 1.91 | 93.6 | 2.26 |
| G018084 | −7 | 45.25 | 3.75 | 44 | 3.39 |
| G018085 | −8 | 45.75 | 2.19 | 32.95 | 0.92 |

### Example 4. Editing in T Cells with UGI in Trans

### 4.1 Editing in T Cells

T cells were edited at the CIITA locus with UGI in trans and either BC22n or Cas9 to assess the impact on editing type on MHC class II antigens. BC22n is a base conversion editor nuclease that includes a fusion of Cas9D10A nickase, human APOBEC3A deaminase. The characteristic edit of this construct is a cytosine to thymine conversion, rather than the indel typical of Cas9 cleavase.

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in a 1:1 formulation of Cryostor® CS10 (StemCell Technologies Cat. No. 07930) and Plasmalyte A (Baxter Cat. No. 2B2522X) for future use. Upon thaw, T cells were plated at a density of 1.0×10⁶ cells/mL in T cell basal media composed of X-VIVO 15™ serum-free hematopoietic cell medium (Lonza Bioscience) containing 5% (v/v) of fetal bovine serum, 50 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 μg/mL of recombinant human interleukin-7 and 5 μg/mL of recombinant human interleukin-15). T-cells were activated with TransAct™ (1:100 dilution, Miltenyi Biotec). Cells were expanded in T cell basal media containing TransAct™ for 72 hours prior to electroporation.

### 4.2. Electroporation of T Cells

Solutions containing mRNAs encoding Cas9, BC22n (SEQ ID NO:804) or UGI (SEQ ID NO: 807) were prepared in sterile water. 50 μM CIITA sgRNAs (G018076 and G018117) (SEQ ID NOs: 56 and 97, respectively) were removed from their storage plates and denatured for 2 minutes at 95° C. before cooling on ice. Seventy-two hours post activation, T cells were harvested, centrifuged, and resuspended at a concentration of 12.5×10⁶ T cells/mL in P3 electroporation buffer (Lonza). For each well to be electroporated, 1×10⁵ T cells were mixed with 200 ng of editor mRNA, 200 ng of UGI mRNA and 20 pmols of sgRNA in a final volume of 20 uL of P3 electroporation buffer. This mix was transferred in triplicate to a 96-well Nucleofector™ plate and electroporated using the manufacturer's pulse code. Electroporated T cells were immediately rested in 80 μL of T cell basal media without cytokines for 10 minutes before being transferred to a new flat-bottom 96-well plate containing an additional 100 μL of T cell basal media supplemented with 2× cytokines. The resulting plate was incubated at 37° C. for 4 days. After 96 hours, T cells were diluted 1:3 into fresh T cell basal media with 1× cytokines. Electroporated T cells were subsequently cultured for 3 additional days and were collected for flow cytometry analysis, NGS sequencing and transcriptomics. NGS analysis was performed as described in Example 1.

### 4.3. Flow Cytometry and NGS Sequencing.

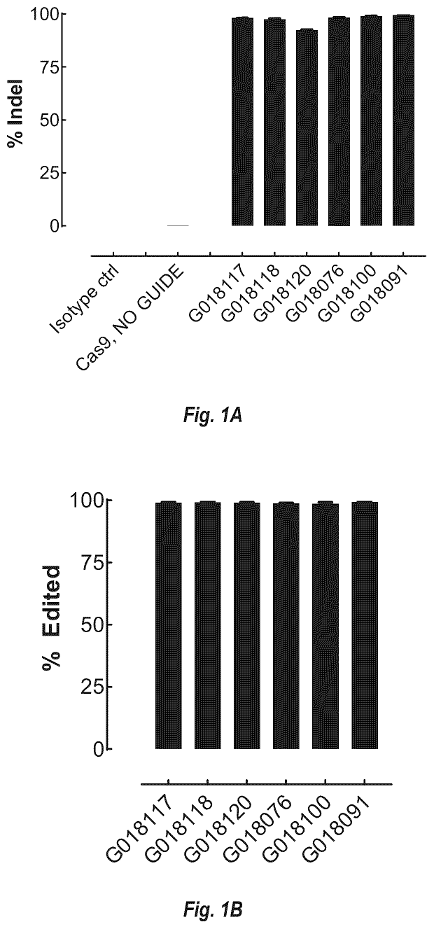

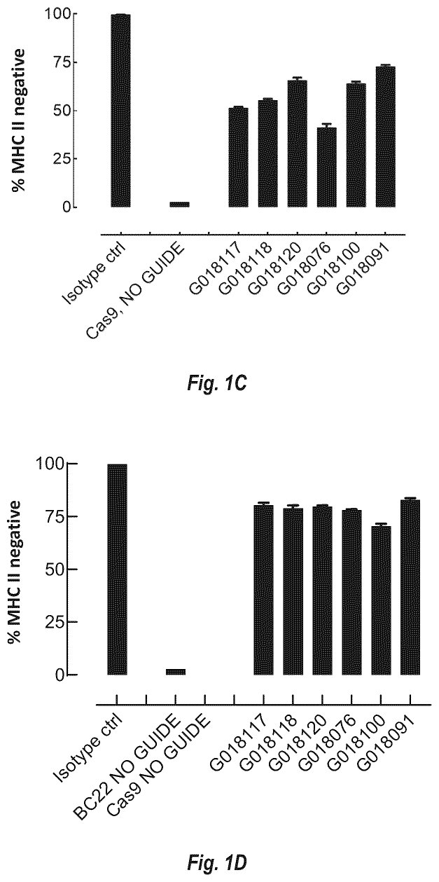

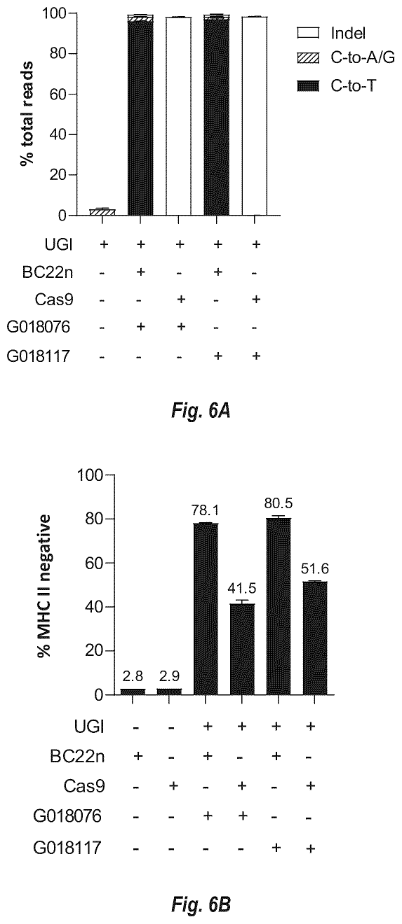

On day 7 post-editing, T cells were phenotyped by flow cytometry to determine MHC class II protein expression. Briefly, T cells were incubated in a cocktail of antibodies targeting HLA-DR, DQ, DP-PE (BioLegend® Cat. No. 361704) and Isotype Control-PE (BioLegend® Cat. No. 400234). Cells were subsequently washed, processed on a Cytoflex flow cytometer (Beckman Coulter) and analyzed using the FlowJo software package. T cells were gated based on size, shape, viability, and MHC class II expression. DNA samples were subjected to PCR and subsequent NGS analysis, as described in Example 1. Table 7 and FIGS. 1A, 1B, and 6A show CIITA gene editing. For both Cas9 and BC22n conditions, total editing went to near completion, above 9500. Table 7 and FIGS. 1C, 1D, and 6B show mean percentage of MHC class II negative cells following electroporation with UGI mRNA combined with Cas9 or BC22n mRNA.

TABLE 7
CIITA editing in T cells presented at a percentage of total NGS reads;
Flow cytometry assessment of the mean percentage of MHC class II negative.

| | | | | | | | | % MHC | | |
|---|---|---|---|---|---|---|---|---|---|---|
| | | % C-to-T | | % C-to-A/G | | % Indel | | class II neg | | |
| Editor | Guide | Mean | SD | Mean | SD | Mean | SD | Mean | SD | N |
| Cas9 | G018117 | 0.0 | 0.0 | 0.1 | 0.0 | 98.0 | 0.2 | 70.3 | 1.5 | 3 |
| | G018118 | 0.0 | 0.0 | 0.1 | 0.1 | 97.5 | 0.5 | 73.7 | 0.5 | 3 |
| | G018120 | 0.1 | 0.1 | 0.2 | 0.0 | 92.3 | 0.3 | 83.1 | 1.1 | 3 |
| | G018076 | 0.0 | 0.0 | 0.1 | 0.0 | 98.3 | 0.1 | 62.8 | 1.1 | 3 |
| | G018100 | 0.0 | 0.0 | 0.1 | 0.0 | 98.9 | 0.2 | 84.1 | 1.3 | 3 |
| | G018091 | 0.0 | 0.0 | 0.0 | 0.0 | 99.4 | 0.2 | 96.2 | 0.4 | 3 |
| | No guide | not reported | | | | | | 10.5 | 0.0 | 1 |
| BC22n | G018117 | 95.7 | 0.2 | 2.3 | 0.1 | 1.1 | 0.1 | 99.2 | 0.1 | 3 |
| | G018118 | 95.8 | 0.3 | 2.3 | 0.1 | 1.2 | 0.3 | 99.0 | 0.1 | 3 |
| | G018120 | 95.7 | 0.5 | 2.6 | 0.4 | 0.7 | 0.1 | 99.0 | 0.1 | 3 |
| | G018076 | 96.5 | 0.4 | 1.2 | 0.2 | 1.1 | 0.3 | 98.5 | 0.2 | 3 |
| | G018100 | 90.7 | 0.3 | 2.8 | 0.3 | 5.1 | 0.7 | 95.7 | 0.6 | 3 |
| | G018091 | 95.4 | 0.6 | 2.8 | 0.1 | 1.1 | 0.3 | 98.7 | 0.2 | 3 |

| | | | | |
|---|---|---|---|---|
| No guide | not reported | 10.8 | 0.0 | 1 |

### Example 5. Gene Expression Analysis in T Cells

### 5.1. Whole Transcriptome Sequencing.

On day 7 post-editing, T cells treated with G018117 (SEQ TD NO: 97) and G018078 (SEQ ID NO: 58) in Example 4 were harvested and preserved at −80C for future processing. Total RNA was extracted from samples in TRIzol™ reagent using the Direct-zol RNA microprep kit (Zymo Research, Cat No. R2062) following the manufacturer's protocol. Purified RNA samples were quantified in a NanoDrop™ 8000 spectrophotometer (Thermo Fisher Scientific) and diluted to 41.67 ng/uL using nuclease-free water. From each experimental triplicate shown in FIG. 1, two samples per group were randomly chosen for transcriptomic analysis. 500 ng (12 uL) of purified total RNA were depleted of ribosomal RNA (rRNA) components using the NEBNext® rRNA Depletion Kit (New England Biolabs, Cat. No. E6350L) according to the manufacturer's instructions. rRNA-depleted samples were converted into double-stranded DNA libraries using NEBNext® Ultra™ II Directional RNA Library Prep Kit for Illumina® (New England Biolabs, Cat No. E7765S) following the manufacturer's protocol. Amplified libraries were quantified in a Qubit 4 fluorometer and the average fragment size of each library was obtained by capillary electrophoresis. Libraries were pooled at an equimolar concentration of 4 nM and pair-end sequenced using a high-output 300-cycle kit (Illumina, Cat No. 20024908) in a NextSeq550 sequencing platform (Illumina).

### 5.2. Data Processing for Differential Gene Expression Analysis.

Sequencing reads in FASTQ format were generated and demultiplexed using the bcl2fastq program (Illumina, v2.20). Reads were assigned to a sample if the Hamming distance (Hamming, R.W. Bell Syst. Tech. J. 29, 147-160) between each index read and the sample indexes was less than or equal to one. The sequencing quality was examined with FastQC program (v0.11.9) (Andrews S. Babraham Inst.). Ribosomal RNA reads were identified by aligning all reads to human rRNA sequences (GenBank U13369.1) with Bowtie2 (v2.3.5.1) (Langmead, B. and Salzberg, S.L. Nat. Methods 9, 357-359). Transcriptome quantification was performed using Salmon (v0.14.1) (Patro R., et al. Nat. Methods 14, 417-419) with non-ribosomal RNA reads. Differential gene expression analysis was carried out using DESeq2 (v1.26.0) (Love, M. I., et al. Genome Biol. 15, 550) on the outputs of Salmon. Genes or transcripts with Benjamini-Hochberg adjusted p-value less than 0.05 were determined to be differentially expressed. Lists of differentially expressed genes were analyzed in terms of gene ontology using Metascape (Zhou, Y., et al. Nat. Comm. 10, 1523). Protein-protein interactions were determined using the BioGrid, InWeb_IM and OmniPath8 databases (Li, T., et al. Nat. Methods 14, 61-64; Stark, C., et al. Nucleic Acids Res. 34, 535-539; Türei, D., et al. Nat. Methods 13, 966-967). Densely connected networks were identified using the molecular complex detection (MCODE) algorithm (Bader, G. D., et al. BMC Bioinformatics 4, 1-27) and the three best-scoring terms by p-value were retained as the functional description of the corresponding network components.

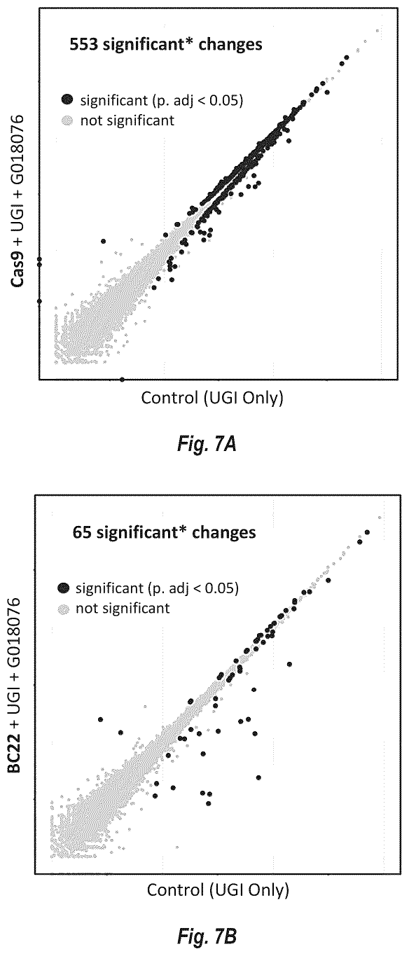

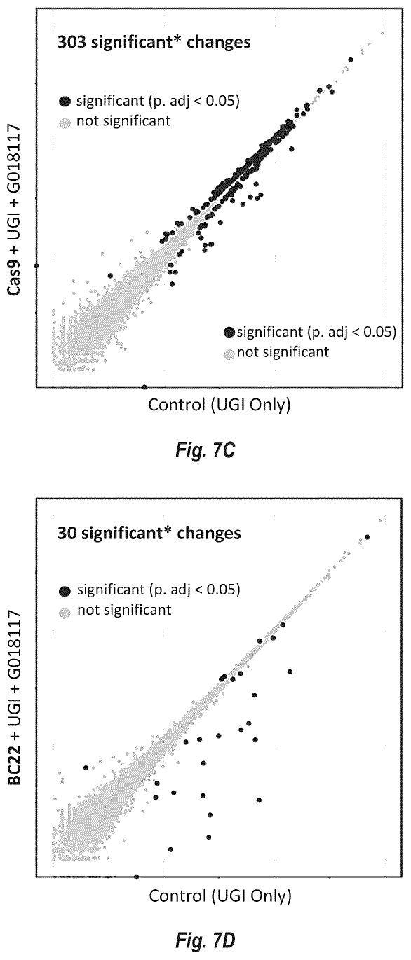

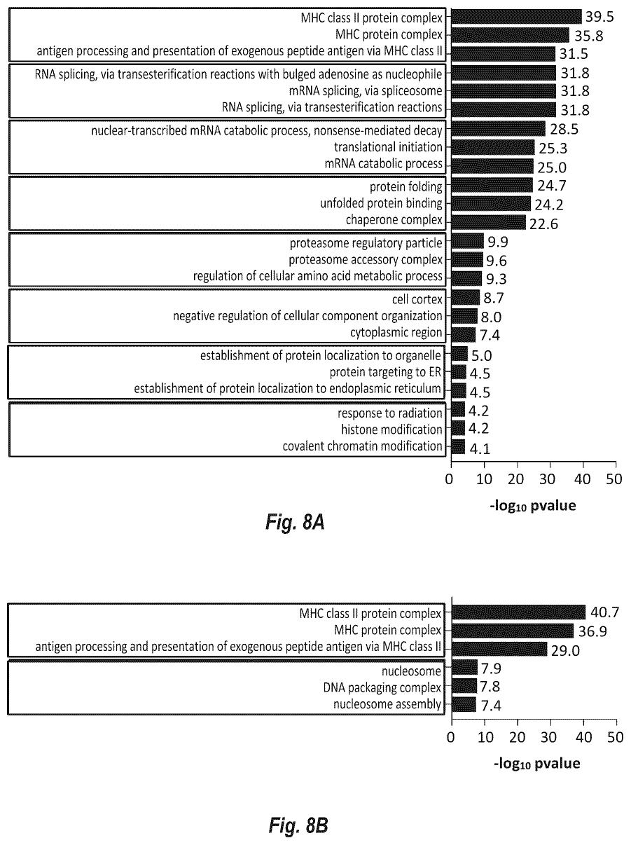

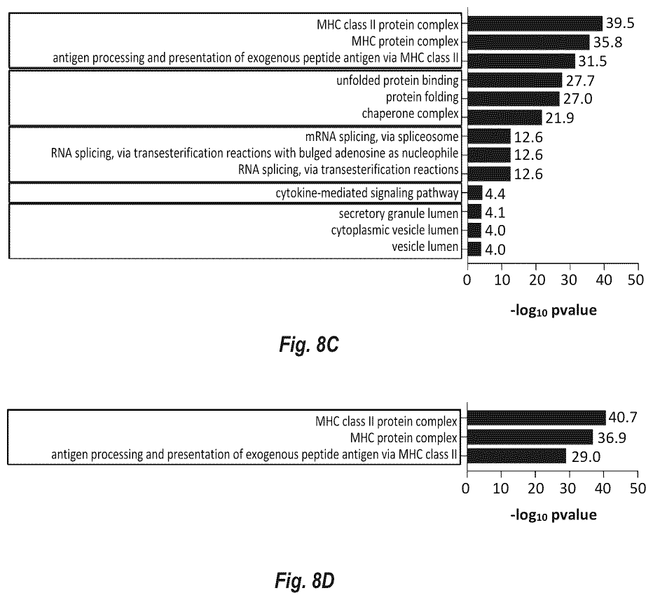

Compared to samples treated with mRNA encoding Cas9 (SEQ ID NO: 809), T cells electroporated with BC22n mRNA (SEQ ID NO: 806) displayed a significantly stronger downregulation of MHC class II genes and the HLA-associated CD74 gene (Table 8 and Table 9). Minimal effects on class I MHC genes were observed (Table 10 and Table 11). In terms of transcriptome-wide differential gene expression events, treatment with BC22n mRNA led to fewer differentially expressed genes (p. adjusted <0.05) when compared to Cas9 mRNA. In T cells electroporated with sgRNA G018076 (SEQ ID NO: 56), a total of 553 and 65 differential gene expression events were observed for Cas9 and BC22n mRNA treatments, respectively (FIGS. 7A and 7B). A similar trend was observed in T cells electroporated with sgRNA G018117 (SEQ ID NO: 97), which displayed 303 and 30 differential gene expression events when treated with Cas9 and BC22n mRNA, respectively (FIGS. 7C and 7D). Fewer protein-protein interaction networks were identified among the list of differentially expressed genes in T cells treated with BC22n mRNA when compared to those treated with Cas9 mRNA with sgRNA G018076 (SEQ ID NO: 56) (FIGS. 8A (Cas9) and 8B (BC22n)) and with sgRNA G018117 (SEQ ID NO: 97) (FIGS. 8C (Cas9) and 8D (BC22n)).

TABLE 8
Differential gene expression of MHC class II genes in T cells.

| | G018076 sgRNA | | | | G018117 sgRNA | | | |
|---|---|---|---|---|---|---|---|---|
| | Cas9 vs | | BC22n vs | | Cas9 vs | | BC22n vs | |
| | control | | control | | control | | control | |
| | Fold | p. | Fold | p. | Fold | p. | Fold | p. |
| Gene | change | adj. | change | adj. | change | adj. | change | adj. |
| CD74 | 0.446 | *** | 0.116 | *** | 0.360 | *** | 0.100 | *** |
| HLA-DMA | 0.457 | *** | 0.150 | *** | 0.356 | *** | 0.146 | *** |
| HLA-DMB | 0.363 | *** | 0.113 | *** | 0.267 | *** | 0.091 | *** |
| HLA-DOA | 0.450 | *** | 0.299 | *** | 0.420 | *** | 0.280 | *** |
| HLA-DPA1 | 0.474 | *** | 0.181 | *** | 0.385 | *** | 0.170 | *** |
| HLA-DPB1 | 0.381 | *** | 0.087 | *** | 0.300 | *** | 0.073 | *** |
| HLA-DQA1 | 0.316 | *** | 0.017 | *** | 0.214 | *** | 0.009 | *** |
| HLA-DQA2 | 0.215 | *** | 0.007 | *** | 0.221 | *** | 0.011 | *** |
| HLA-DQB1 | 0.383 | *** | 0.069 | *** | 0.290 | *** | 0.069 | *** |
| HLA-DQB1- | 0.323 | *** | 0.102 | *** | 0.311 | *** | 0.097 | *** |
| AS1 | | | | | | | | |
| HLA-DRA | 0.288 | *** | 0.004 | *** | 0.205 | *** | 0.002 | *** |
| HLA-DRB1 | 0.287 | *** | 0.029 | *** | 0.207 | *** | 0.027 | *** |
| HLA-DRB3 | 0.268 | *** | 0.012 | *** | 0.219 | *** | 0.004 | *** |
| HLA-DRB4 | 0.282 | *** | 0.024 | *** | 0.224 | *** | 0.026 | *** |

(ns = not significant,
* = p. adj. < 0.05,
** = p. adj. < 0.01,
*** = p. adj. < 0.001).
For transcript quantification data, refer to Table 9.

TABLE 9
Transcript quantification of the expression
of MHC class II genes in T cells.

| | | G0188117 | | G018076 | |
|---|---|---|---|---|---|
| Guide | none | Cas9 | BC22n | Cas9 | BC22n |
| mRNAs | UGI | UGI | UGI | UGI | UGI |
| CD74 | 385 | 154 | 38 | 174 | 45 |
| HLA-DMA | 27 | 11 | 4 | 12 | 4 |
| HLA-DMB | 11 | 3 | 1 | 4 | 1 |
| HLA-DOA | 3 | 2 | 1 | 2 | 1 |
| HLA-DPA1 | 70 | 30 | 12 | 34 | 13 |
| HLA-DPB1 | 16 | 5 | 1 | 6 | 1 |
| HLA-DQA1 | 12 | 3 | 0 | 4 | 0 |
| HLA-DQA2 | 3 | 1 | 0 | 1 | 0 |
| HLA-DQB1 | 60 | 19 | 4 | 23 | 4 |
| HLA-DQB1-AS1 | 9 | 3 | 1 | 3 | 1 |
| HLA-DRA | 120 | 27 | 0 | 35 | 1 |
| HLA-DRB1 | 117 | 27 | 3 | 34 | 3 |
| HLA-DRB3 | 17 | 4 | 0 | 5 | 0 |
| HLA-DRB4 | 13 | 3 | 0 | 4 | 0 |

Each square contains the average number of transcripts from a given gene per one million of mRNA molecules. For statistical significance, please refer to Table 8.

TABLE 10
Differential gene expression of class I HLA genes in T cells harvested
7 days post-treatment with different mRNA combinations and CIITA
sgRNAs.

| | G018076 sgRNA | | | | G018117 sgRNA | | | |
|---|---|---|---|---|---|---|---|---|
| | Cas9 vs | | BC22n vs | | Cas9 vs | | BC22n vs | |
| | control | | control | | control | | control | |
| | Fold | p. | Fold | p. | Fold | p. | Fold | p. |
| Gene | change | adj. | change | adj. | change | adj. | change | adj. |
| HLA-A | 0.995 | ns | 0.910 | * | 0.969 | ns | 0.926 | ns |
| HLA-B | 1.001 | ns | 0.881 | ** | 1.043 | ns | 0.913 | ns |
| HLA-C | 1.013 | ns | 0.917 | ns | 0.995 | ns | 0.922 | ns |
| HLA-E | 0.925 | ns | 0.919 | ns | 0.870 | *** | 0.949 | ns |
| HLA-F | 0.897 | ns | 0.812 | ** | 0.930 | ns | 0.891 | ns |

(ns = not significant,
* = p. adj. < 0.05,
** = p. adj. < 0.01,
*** = p. adj. < 0.001).
For transcript quantification data, refer to Table 11.

TABLE 11
Transcript quantification of the expression
of class I HLA genes in T cells.

| | |
|---|---|
| G0188117 | G018076 |

| Guide | none | Cas9 | BC22n | Cas9 | BC22n |
|---|---|---|---|---|---|
| mRNAs | UGI | UGI | UGI | UGI | UGI |
| HLA-A | 880 | 947 | 797 | 887 | 814 |
| HLA-B | 457 | 528 | 407 | 463 | 409 |
| HLA-C | 479 | 528 | 430 | 490 | 445 |
| HLA-E | 166 | 161 | 154 | 156 | 155 |
| HLA-F | 68 | 70 | 59 | 61 | 56 |

Each square contains the average number of transcripts from a given gene per one million of mRNA molecules. For statistical significance, please refer to Table 12.

### Example 6. LNP Titration in T Cells with Fixed Ratio of BC22n:UGI

Using LNP delivery to activated human T cells, the potency of single-target and multi-target editing was assessed with either Cas9 or BC22n.

### 6.1. T Cell Preparation.

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in a 1:1 formulation of Cryostor® CS10 (StemCell Technologies Cat. No. 07930) and Plasmalyte A (Baxter Cat. No. 2B2522X) for future use. Upon thaw, T cells were plated at a density of 1.0×10⁶ cells/mL in T cell basal media composed of X-VIVO 15™ serum-free hematopoietic cell medium (Lonza Bioscience) containing 5% (v/v) of fetal bovine serum, 50 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 ng/mL of recombinant human interleukin-7 and 5 ng/mL of recombinant human interleukin-15). T cells were activated with TransAct™ (1:100 dilution, Miltenyi Biotec). Cells were expanded in T cell basal media for 72 hours prior to LNP transfection.

### 6.2. T Cell Editing.

Each RNA species, i.e. UGI mRNA, sgRNA or editor mRNA, was formulated separately in an LNP as described in Example 1. Editor mRNAs encoded either BC22n (SEQ ID NO: 805) or Cas9. Guides targeting B2M (G015995) (SEQ ID NO: 704), TRAC (G016017) (SEQ ID NO: 705), TRBC1/2 (G016206) (SEQ ID NO: 706) and CIITA (G018117) (SEQ ID NO: 97) were used either singly or in combination. Messenger RNA encoding UGI (SEQ ID NO: 807) is delivered in both Cas9 and BC22n arms of the experiment to normalize lipid amounts. Previous experiments have established UGI mRNA does not impact total editing or editing profile when used with Cas9 mRNA. LNPs were mixed to fixed total mRNA weight ratios of 6:3:2 for editor mRNA, guide RNA, and UGI mRNA respectively as described in Table 12. In the 4-guide experiment described in Table 12, individual guides are diluted 4-fold to maintain the overall 6:3 editor mRNA: guide weight ratio and to allow comparison to individual guide potency based on total lipid delivery. LNP mixtures were incubated for 5 minutes at 37° C. in T cell basal media substituting 6% cynomolgus monkey serum (Bioreclamation IVT, Cat. CYN220760) for fetal bovine serum.

Seventy-two hours post activation, T cells were washed and suspended in basal T cell media. Pre-incubated LNP mix was added to the each well with 1×10e5 Tcells/well. T cells were incubated at 37° C. with 5% C02 for the duration of the experiment. T cell media was changed 6 days and 8 days after activation and on tenth day post activation, cells were harvested for analysis by NGS and flow cytometry. NGS was performed as in Example 1.

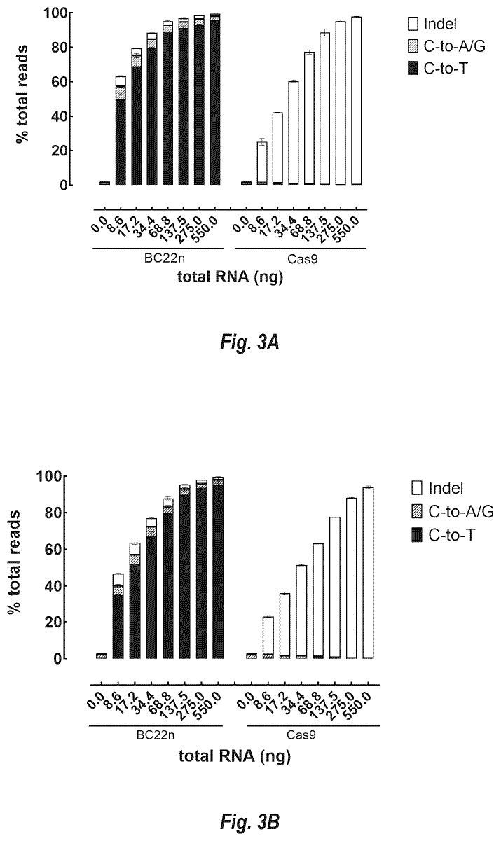

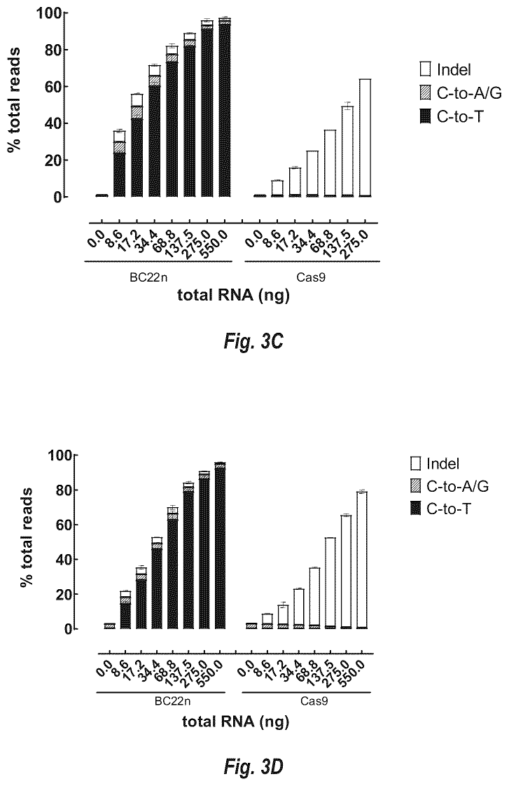

Table 12 and FIGS. 3A-D describe the editing profile of T cells when an individual guide was used for editing. Total editing and C to T editing showed direct, dose responsive relationships to increasing amounts of BC22n mRNA, UGI mRNA and guide across all guides tested. Indel and C conversions to A or G are in an inverse relationship with dose where lower doses resulted in a higher percentage of these mutations. In samples edited with Cas9, total editing and indel activity increase with the total RNA dose.

TABLE 12
Editing as a percent of total reads—single guide delivery.

| | | Total | | | | | | | |
|---|---|---|---|---|---|---|---|---|---|
| | | RNA | C-to-T % | | C-to-A/G % | | Indel % | | |
| Guide | Editor | (ng) | mean | SD | mean | SD | mean | SD | N |
| G015995 | BC22n | 0.0 | 0.3 | 0.0 | 1.5 | 0.1 | 0.2 | 0.0 | 2 |
| B2M | | 8.6 | 49.5 | 3.5 | 7.7 | 0.6 | 6.0 | 0.4 | 2 |
| | | 17.2 | 68.5 | 1.7 | 6.7 | 1.3 | 4.3 | 0.1 | 2 |
| | | 34.4 | 79.0 | 0.9 | 5.7 | 0.3 | 3.8 | 0.0 | 2 |
| | | 68.8 | 88.2 | 0.8 | 4.6 | 0.0 | 2.5 | 0.2 | 2 |
| | | 137.5 | 90.6 | 1.8 | 4.1 | 0.4 | 2.2 | 0.5 | 2 |
| | | 275.0 | 92.6 | 0.8 | 3.7 | 0.3 | 2.2 | 0.3 | 2 |
| | | 550.0 | 95.2 | 0.4 | 2.8 | 0.0 | 1.6 | 0.2 | 2 |
| | Cas9 | 0.0 | 0.3 | 0.0 | 1.5 | 0.2 | 0.2 | 0.0 | 2 |
| | | 8.6 | 0.3 | 0.0 | 1.2 | 0.1 | 23.7 | 2.1 | 2 |
| | | 17.2 | 0.3 | 0.0 | 0.9 | 0.1 | 41.1 | 0.2 | 2 |
| | | 34.4 | 0.3 | 0.0 | 0.6 | 0.0 | 59.4 | 0.6 | 2 |
| | | 68.8 | 0.2 | 0.1 | 0.4 | 0.0 | 76.8 | 1.2 | 2 |
| | | 137.5 | 0.1 | 0.1 | 0.2 | 0.0 | 88.2 | 2.0 | 2 |
| | | 275.0 | 0.1 | 0.0 | 0.1 | 0.1 | 95.1 | 0.5 | 2 |
| | | 550.0 | 0.1 | 0.0 | 0.1 | 0.0 | 97.5 | 0.3 | 2 |
| G016017 | BC22n | 0.0 | 0.2 | 0.0 | 2.2 | 0.1 | 0.2 | 0.1 | 2 |
| TRAC | | 8.6 | 34.6 | 1.1 | 5.6 | 0.8 | 6.6 | 0.2 | 2 |
| | | 17.2 | 51.3 | 0.8 | 5.7 | 0.1 | 6.7 | 1.0 | 2 |
| | | 34.4 | 66.9 | 2.6 | 5.4 | 0.2 | 4.7 | 0.4 | 2 |
| | | 68.8 | 79.0 | 0.6 | 4.4 | 0.7 | 4.5 | 0.9 | 2 |
| | | 137.5 | 89.2 | 0.4 | 3.6 | 0.9 | 2.5 | 0.2 | 2 |
| | | 275.0 | 92.8 | 0.9 | 2.9 | 0.0 | 2.3 | 0.0 | 2 |
| | | 550.0 | 94.5 | 1.3 | 3.4 | 1.0 | 1.6 | 0.2 | 2 |
| | Cas9 | 0.0 | 0.2 | 0.0 | 2.3 | 0.1 | 0.1 | 0.0 | 2 |
| | | 8.6 | 0.2 | 0.0 | 2.1 | 0.2 | 20.7 | 0.5 | 2 |
| | | 17.2 | 0.1 | 0.0 | 1.4 | 0.0 | 34.6 | 0.7 | 2 |
| | | 34.4 | 0.1 | 0.0 | 1.5 | 0.4 | 49.8 | 0.4 | 2 |
| | | 68.8 | 0.1 | 0.0 | 1.0 | 0.0 | 62.3 | 0.1 | 2 |
| | | 137.5 | 0.1 | 0.0 | 0.6 | 0.1 | 77.0 | 0.1 | 2 |
| | | 275.0 | 0.0 | 0.0 | 0.3 | 0.0 | 87.8 | 0.2 | 2 |
| | | 550.0 | 0.0 | 0.0 | 0.2 | 0.0 | 93.8 | 0.6 | 2 |
| G016206 | BC22n | 0.0 | 0.4 | 0.1 | 0.6 | 0.1 | 0.1 | 0.1 | 2 |
| TRBC1/2 | | 8.6 | 23.7 | 1.3 | 6.1 | 0.0 | 6.1 | 0.8 | 2 |
| | | 17.2 | 42.4 | 2.2 | 6.8 | 0.1 | 6.8 | 0.3 | 2 |
| | | 34.4 | 60.1 | 2.2 | 5.7 | 0.3 | 5.9 | 0.7 | 2 |
| | | 68.8 | 73.2 | 4.2 | 4.3 | 0.1 | 4.7 | 1.1 | 2 |
| | | 137.5 | 81.7 | 0.8 | 3.6 | 0.2 | 3.7 | 0.4 | 2 |
| | | 275.0 | 91.0 | 1.7 | 2.3 | 0.1 | 2.8 | 0.8 | 2 |
| | | 550.0 | 93.6 | 1.9 | 2.0 | 0.2 | 1.7 | 0.6 | 2 |
| | Cas9 | 0.0 | 0.3 | 0.0 | 0.5 | 0.0 | 0.1 | 0.0 | 1 |
| | | 8.6 | 0.3 | 0.2 | 0.5 | 0.1 | 8.1 | 0.2 | 2 |
| | | 17.2 | 0.3 | 0.1 | 0.7 | 0.1 | 14.9 | 0.6 | 2 |
| | | 34.4 | 0.2 | 0.0 | 0.8 | 0.0 | 24.1 | 0.0 | 1 |
| | | 68.8 | 0.2 | 0.0 | 0.4 | 0.0 | 35.9 | 0.0 | 1 |
| | | 137.5 | 0.2 | 0.0 | 0.5 | 0.0 | 48.6 | 2.1 | 2 |
| | | 275.0 | 0.1 | 0.0 | 0.4 | 0.0 | 63.8 | 0.0 | 1 |
| | | 550.0 | Not assayed | | | | | | |
| G018117 | BC22n | 0.0 | 0.3 | 0.0 | 2.7 | 0.1 | 0.3 | 0.0 | 2 |
| CIITA | | 8.6 | 14.5 | 1.5 | 3.8 | 0.3 | 3.5 | 0.3 | 2 |
| | | 17.2 | 28.1 | 0.6 | 3.5 | 0.3 | 3.9 | 1.0 | 2 |
| | | 34.4 | 45.9 | 0.4 | 3.3 | 0.4 | 3.6 | 0.0 | 2 |
| | | 68.8 | 62.8 | 5.3 | 3.6 | 0.1 | 3.7 | 1.2 | 2 |
| | | 137.5 | 78.9 | 1.3 | 2.7 | 0.1 | 2.7 | 0.7 | 2 |
| | | 275.0 | 86.3 | 1.8 | 2.6 | 0.1 | 2.0 | 0.1 | 2 |
| | | 550.0 | 92.3 | 1.2 | 2.6 | 0.2 | 1.1 | 0.2 | 2 |
| | Cas9 | 0.0 | 0.2 | 0.0 | 2.8 | 0.1 | 0.3 | 0.0 | 2 |
| | | 8.6 | 0.3 | 0.0 | 2.5 | 0.0 | 6.0 | 0.2 | 2 |
| | | 17.2 | 0.2 | 0.0 | 2.4 | 0.1 | 11.2 | 1.6 | 2 |
| | | 34.4 | 0.2 | 0.0 | 2.1 | 0.0 | 20.8 | 0.3 | 2 |
| | | 68.8 | 0.2 | 0.0 | 1.9 | 0.1 | 33.2 | 0.4 | 2 |
| | | 137.5 | 0.1 | 0.0 | 1.3 | 0.1 | 51.2 | 0.0 | 2 |
| | | 275.0 | 0.1 | 0.0 | 0.9 | 0.2 | 64.5 | 0.9 | 2 |
| | | 550.0 | 0.1 | 0.0 | 0.6 | 0.0 | 78.4 | 1.1 | 2 |

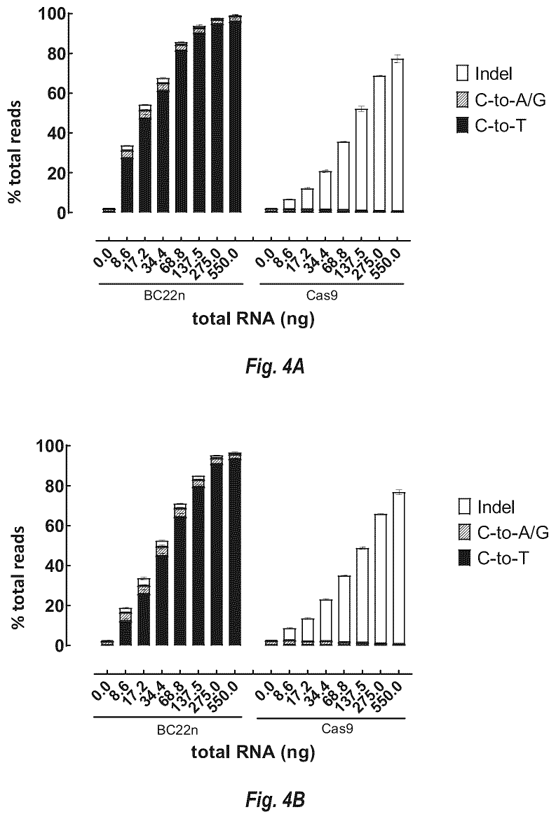

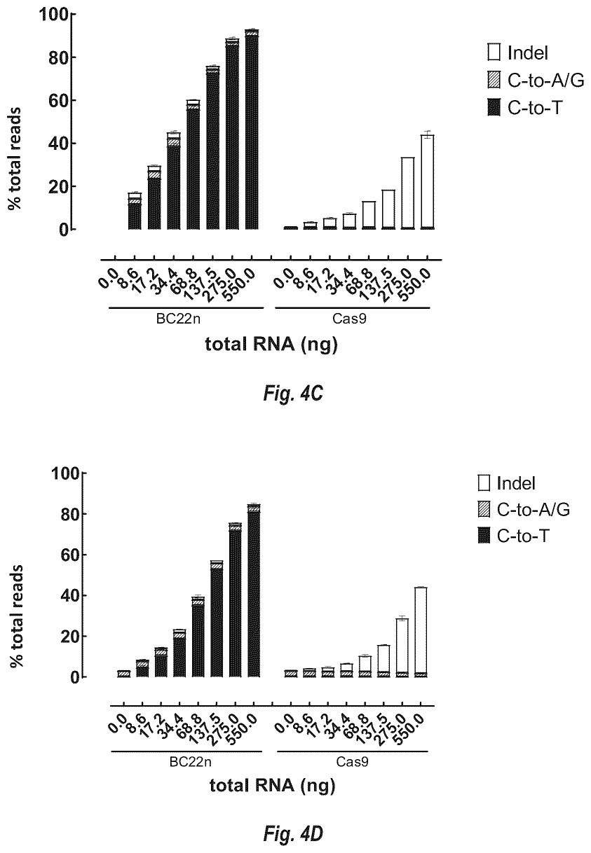

Table 13 and FIGS. 4A-D describe the editing profile for T cells in percent of total reads when four guides were used simultaneously for editing. In this arm of the experiment, each guide is used at 25% the concentration compared to the single guide editing experiment. In total, T cells were exposed to 6 different LNPs simultaneously (editor mRNA, UGI mRNA, 4 guides). Editing with BC22n and trans UGI lead to higher percentages of maximum total editing for each locus compared to editing with Cas9.

TABLE 13
Editing as a percentage of total reads—multiple guide delivery.

| | | Total | | | C-to-A/G | | | | |
|---|---|---|---|---|---|---|---|---|---|
| Locus | | RNA | C-to-T % | | % | | Indel % | | |
| Assayed | Editor | (ng) | mean | SD | mean | SD | mean | SD | N |
| G015995 | BC22n | 0.0 | 0.3 | 0.0 | 1.5 | 0.2 | 0.2 | 0.0 | 2 |
| B2M | | 8.6 | 27.3 | 0.2 | 3.8 | 0.1 | 2.6 | 0.1 | 2 |
| | | 17.2 | 47.2 | 2.2 | 4.1 | 0.4 | 3.0 | 0.1 | 2 |
| | | 34.4 | 61.2 | 3.0 | 3.9 | 0.1 | 2.6 | 0.3 | 2 |
| | | 68.8 | 81.4 | 0.1 | 2.9 | 0.1 | 1.4 | 0.1 | 2 |
| | | 137.5 | 90.0 | 1.1 | 2.6 | 0.3 | 1.3 | 0.5 | 2 |
| | | 275.0 | 94.7 | 0.1 | 2.2 | 0.1 | 0.8 | 0.0 | 2 |
| | | 550.0 | 95.9 | 0.9 | 2.9 | 1.0 | 0.4 | 0.3 | 2 |
| | Cas9 | 0.0 | 0.3 | 0.0 | 1.4 | 0.1 | 0.2 | 0.0 | 2 |
| | | 8.6 | 0.3 | 0.0 | 1.4 | 0.0 | 5.0 | 0.1 | 2 |
| | | 17.2 | 0.3 | 0.0 | 1.3 | 0.0 | 10.5 | 0.4 | 2 |
| | | 34.4 | 0.3 | 0.0 | 1.1 | 0.0 | 19.3 | 0.6 | 2 |
| | | 68.8 | 0.3 | 0.0 | 0.9 | 0.0 | 34.4 | 0.1 | 2 |
| | | 137.5 | 0.2 | 0.0 | 0.7 | 0.0 | 51.1 | 1.3 | 2 |
| | | 275.0 | 0.2 | 0.1 | 0.5 | 0.0 | 68.0 | 0.1 | 2 |
| | | 550.0 | 0.3 | 0.1 | 0.4 | 0.1 | 76.7 | 2.0 | 2 |
| G016017 | BC22n | 0.0 | 0.1 | 0.1 | 1.9 | 0.6 | 0.2 | 0.0 | 2 |
| TRAC | | 8.6 | 12.1 | 1.3 | 4.3 | 0.2 | 2.4 | 0.2 | 2 |
| | | 17.2 | 25.7 | 2.2 | 4.2 | 0.5 | 3.8 | 0.7 | 2 |
| | | 34.4 | 44.7 | 1.4 | 4.7 | 1.0 | 3.0 | 0.3 | 2 |
| | | 68.8 | 64.2 | 1.9 | 4.4 | 0.6 | 2.5 | 0.1 | 2 |
| | | 137.5 | 79.3 | 1.1 | 3.6 | 0.4 | 2.1 | 0.1 | 2 |
| | | 275.0 | 90.7 | 0.0 | 3.0 | 0.1 | 1.5 | 0.0 | 2 |
| | | 550.0 | 93.3 | 0.6 | 2.4 | 0.1 | 0.9 | 0.4 | 2 |
| | Cas9 | 0.0 | 0.1 | 0.1 | 2.1 | 0.2 | 0.1 | 0.0 | 2 |
| | | 8.6 | 0.2 | 0.1 | 2.3 | 0.2 | 6.1 | 0.2 | 2 |
| | | 17.2 | 0.1 | 0.0 | 1.8 | 0.2 | 11.5 | 0.5 | 2 |
| | | 34.4 | 0.1 | 0.0 | 2.0 | 0.4 | 21.0 | 0.4 | 2 |
| | | 68.8 | 0.1 | 0.0 | 1.4 | 0.0 | 33.5 | 0.1 | 2 |
| | | 137.5 | 0.1 | 0.0 | 1.2 | 0.1 | 47.5 | 0.5 | 2 |
| | | 275.0 | 0.1 | 0.0 | 0.9 | 0.1 | 64.8 | 0.2 | 2 |
| | | 550.0 | 0.1 | 0.0 | 0.6 | 0.1 | 76.1 | 1.3 | 2 |
| G016206 | BC22n | 0.0 | No data | | | | | | |
| TRBC1/2 | | 8.6 | 11.6 | 0.3 | 2.6 | 0.2 | 2.8 | 0.3 | 2 |
| | | 17.2 | 23.4 | 0.4 | 3.6 | 0.3 | 2.6 | 0.5 | 2 |
| | | 34.4 | 38.5 | 1.4 | 3.7 | 0.2 | 2.9 | 0.7 | 2 |
| | | 68.8 | 55.6 | 1.7 | 2.3 | 0.4 | 2.4 | 0.0 | 2 |
| | | 137.5 | 72.4 | 1.2 | 1.8 | 0.5 | 1.7 | 0.5 | 2 |
| | | 275.0 | 85.1 | 1.0 | 1.9 | 0.5 | 1.7 | 0.6 | 2 |
| | | 550.0 | 89.8 | 2.8 | 2.2 | 0.1 | 0.9 | 0.3 | 2 |
| | Cas9 | 0.0 | 0.2 | 0.0 | 0.6 | 0.0 | 0.1 | 0.0 | 1 |
| | | 8.6 | 0.2 | 0.1 | 0.7 | 0.1 | 2.3 | 0.3 | 2 |
| | | 17.2 | 0.3 | 0.0 | 0.7 | 0.3 | 4.2 | 0.4 | 2 |
| | | 34.4 | 0.1 | 0.0 | 0.5 | 0.1 | 6.6 | 0.5 | 2 |
| | | 68.8 | 0.4 | 0.0 | 0.5 | 0.0 | 12.3 | 0.0 | 1 |
| | | 137.5 | 0.2 | 0.0 | 0.5 | 0.0 | 17.8 | 0.0 | 1 |
| | | 275.0 | 0.1 | 0.0 | 0.5 | 0.0 | 33.0 | 0.0 | 1 |
| | | 550.0 | 0.3 | 0.2 | 0.3 | 0.0 | 43.3 | 1.7 | 2 |
| G018117 | BC22n | 0.0 | 0.2 | 0.0 | 2.6 | 0.1 | 0.3 | 0.0 | 2 |
| CIITA | | 8.6 | 4.6 | 0.9 | 3.1 | 0.2 | 0.8 | 0.2 | 2 |
| | | 17.2 | 10.5 | 0.2 | 2.9 | 0.1 | 1.1 | 0.2 | 2 |
| | | 34.4 | 18.8 | 0.3 | 2.9 | 0.2 | 1.6 | 0.2 | 2 |
| | | 68.8 | 35.1 | 0.6 | 2.7 | 0.2 | 1.6 | 0.7 | 2 |
| | | 137.5 | 52.9 | 0.2 | 2.9 | 0.3 | 1.5 | 0.0 | 2 |
| | | 275.0 | 71.9 | 2.4 | 2.5 | 0.3 | 1.3 | 0.1 | 2 |
| | | 550.0 | 81.1 | 1.9 | 2.6 | 0.1 | 1.1 | 0.6 | 2 |
| | Cas9 | 0.0 | 0.3 | 0.0 | 2.7 | 0.1 | 0.3 | 0.0 | 2 |
| | | 8.6 | 0.2 | 0.0 | 2.6 | 0.2 | 1.4 | 0.0 | 2 |
| | | 17.2 | 0.2 | 0.0 | 2.5 | 0.0 | 2.1 | 0.3 | 2 |
| | | 34.4 | 0.3 | 0.0 | 2.5 | 0.0 | 3.9 | 0.1 | 2 |
| | | 68.8 | 0.2 | 0.0 | 2.5 | 0.2 | 7.7 | 0.6 | 2 |
| | | 137.5 | 0.2 | 0.0 | 2.2 | 0.1 | 13.3 | 0.2 | 2 |
| | | 275.0 | 0.1 | 0.0 | 1.9 | 0.0 | 26.7 | 1.3 | 2 |
| | | 550.0 | 0.1 | 0.0 | 1.7 | 0.1 | 42.3 | 0.3 | 2 |

On day 10 post-activation, T cells were phenotyped by flow cytometry to determine if editing resulted in loss of cell surface proteins. Briefly, T cells were incubated in a mix of the following antibodies: B2M-FITC (BioLegend, Cat. 316304), CD3-AF700 (BioLegend, Cat. 317322), HLA DR DQ DP-PE (BioLegend, Cat 361704) and DAPI (BioLegend, Cat 422801). A subset of unedited cells was incubated with Isotype Control-PE (BioLegend® Cat. No. 400234). Cells were subsequently washed, processed on a Cytoflex instrument (Beckman Coulter) and analyzed using the FlowJo software package. T cells were gated based on size, shape, viability, and antigen expression.

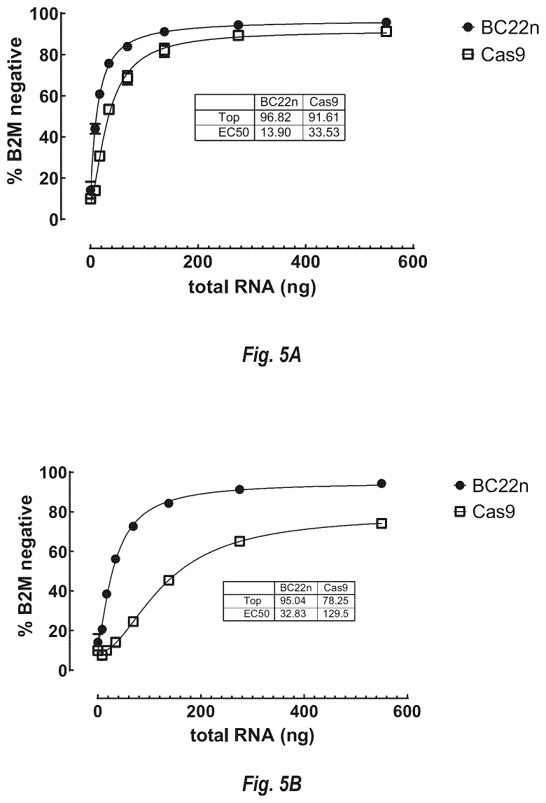

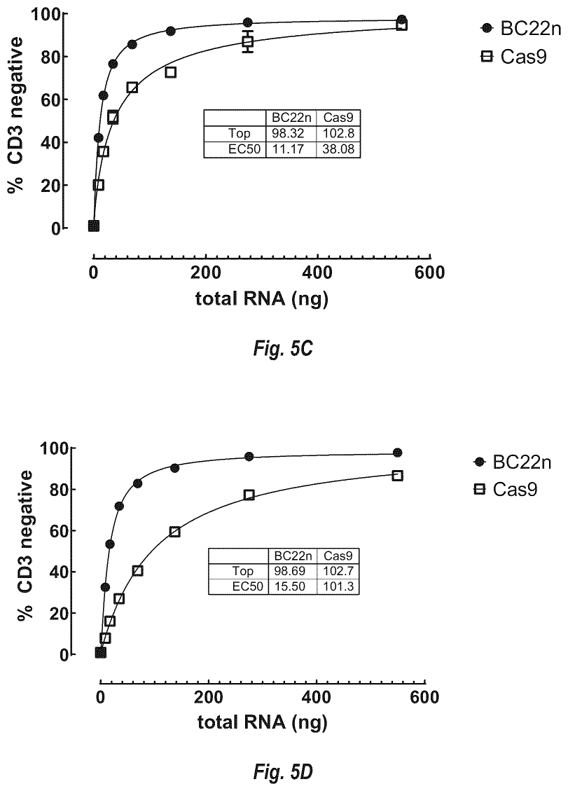

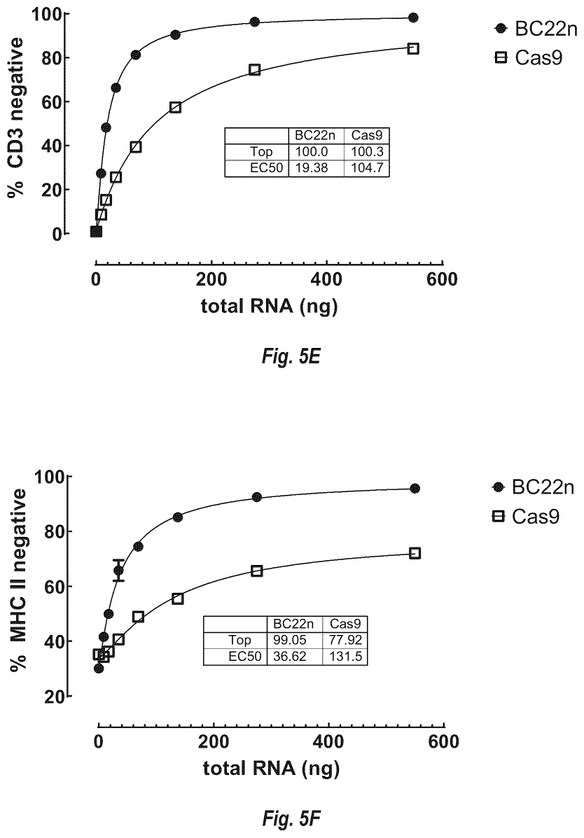

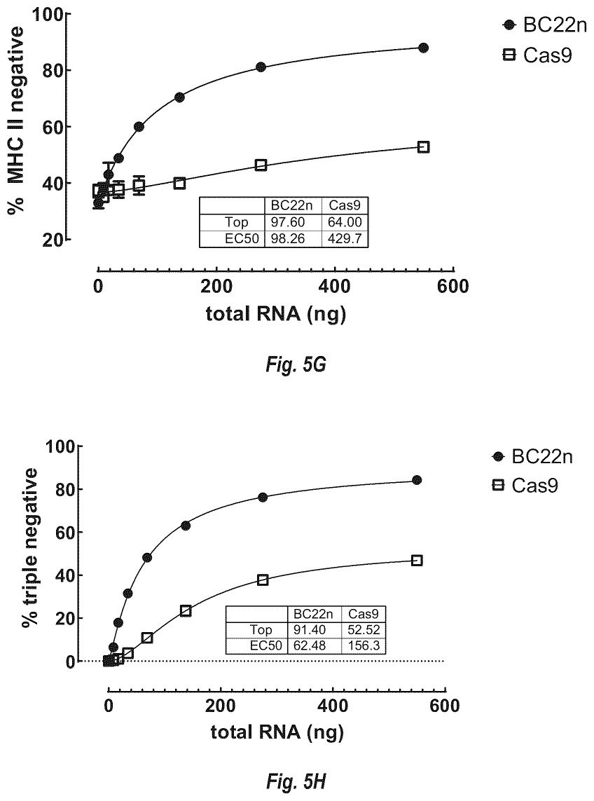

Table 14 and FIGS. 5A-H report phenotyping results as percent of cells negative for antibody binding. The percentage of antigen negative cells increased in a dose responsive manner with increasing total RNA for both BC22n and Cas9 samples. Cells edited with BC22n showed comparable or higher protein knockout compared to cells edited with Cas9 for all guides tested. In multi-edited cells, BC22n with trans UGI showed substantially higher percentages of antigen negative cells than Cas9 with trans UGI. For example, BC22n edited samples at the highest total RNA dose of 550 ng showed 84.2% of cells lacking all three antigens, while Cas9 editing led to only 46.8% such triple knockout cells. For samples treated with one guide only, the correlation between DNA editing and antigen reduction was robust. BC22n had an R square measurement of 0.93 when comparing C to T conversions to antigen knockout. Cas9 had an R square measurement of 0.95 when comparing indels to antigen knockout.

TABLE 14
Flow cytometry data—percent cells negative for antigen (n = 2).

| | | Total | BC22n | | Cas9 | |
|---|---|---|---|---|---|---|
| | | RNA | Mean | | Mean | |
| Guide(s) | Phenotype | (ng) | % | SD | % | SD |
| G015995 | B2M neg | 550.0 | 95.7 | 0.1 | 91.3 | 0.6 |
| B2M | | 275.0 | 94.4 | 0.4 | 89.3 | 0.1 |
| | | 137.5 | 91.2 | 0.1 | 82.1 | 3.3 |
| | | 68.8 | 83.9 | 0.4 | 68.7 | 3.3 |
| | | 34.4 | 75.7 | 1.4 | 53.4 | 0.2 |
| | | 17.2 | 60.8 | 2.0 | 30.7 | 1.3 |
| | | 8.6 | 44.0 | 2.3 | 13.9 | 2.0 |
| | | 0.0 | 14.1 | 4.1 | 9.9 | 1.9 |
| G015995 | B2M neg | 550.0 | 94.4 | 0.1 | 74.2 | 0.4 |
| G016017 | | 275.0 | 91.3 | 0.1 | 65.2 | 0.1 |
| G016206 | | 137.5 | 84.3 | 0.2 | 45.4 | 1.9 |
| G018117 | | 68.8 | 72.7 | 0.4 | 24.5 | 0.8 |
| | | 34.4 | 56.2 | 1.2 | 14.1 | 2.3 |
| | | 17.2 | 38.5 | 0.2 | 9.9 | 0.8 |
| | | 8.6 | 20.6 | 0.7 | 7.6 | 2.4 |
| | | 0.0 | 14.1 | 4.1 | 9.9 | 1.9 |
| G016017 | CD3 neg | 550.0 | 97.3 | 0.3 | 94.8 | 0.4 |
| TRAC | | 275.0 | 96.0 | 0.2 | 87.0 | 4.9 |
| | | 137.5 | 91.9 | 0.2 | 72.7 | 0.9 |
| | | 68.8 | 85.7 | 0.5 | 65.6 | 0.1 |
| | | 34.4 | 76.6 | 0.8 | 51.7 | 3.0 |
| | | 17.2 | 61.8 | 1.7 | 35.7 | 1.1 |
| | | 8.6 | 42.1 | 0.7 | 20.1 | 1.5 |
| | | 0.0 | 1.0 | 0.1 | 0.9 | 0.1 |
| G016206 | CD3 neg | 550.0 | 97.9 | 0.1 | 86.6 | 0.3 |
| TRBC1/2 | | 275.0 | 96.0 | 0.1 | 77.3 | 0.1 |
| | | 137.5 | 90.4 | 0.8 | 59.4 | 0.4 |
| | | 68.8 | 82.9 | 0.1 | 40.6 | 1.2 |
| | | 34.4 | 71.9 | 1.5 | 27.0 | 1.6 |
| | | 17.2 | 53.4 | 0.3 | 16.1 | 0.1 |
| | | 8.6 | 32.6 | 0.6 | 7.9 | 0.4 |
| | | 0.0 | 0.8 | 0.0 | 0.9 | 0.4 |
| G015995 | CD3 neg | 550.0 | 98.3 | 0.2 | 84.2 | 0.1 |
| G016017 | | 275.0 | 96.3 | 0.1 | 74.6 | 0.5 |
| G016206 | | 137.5 | 90.4 | 0.3 | 57.4 | 1.0 |
| G018117 | | 68.8 | 81.3 | 0.3 | 39.4 | 0.1 |
| | | 34.4 | 66.3 | 1.6 | 25.6 | 0.8 |
| | | 17.2 | 48.2 | 1.0 | 15.3 | 0.5 |
| | | 8.6 | 27.3 | 0.7 | 8.6 | 0.5 |
| | | 0.0 | 0.9 | 0.1 | 0.9 | 0.2 |
| G018117 | HLA DR | 550.0 | 95.7 | 0.4 | 72.0 | 0.1 |
| CIITA | DP DQ | 275.0 | 92.5 | 1.1 | 65.6 | 0.4 |
| | neg | 137.5 | 85.2 | 0.6 | 55.5 | 0.6 |
| | | 68.8 | 74.5 | 1.1 | 48.9 | 0.0 |
| | | 34.4 | 65.8 | 3.7 | 40.7 | 0.6 |
| | | 17.2 | 49.9 | 0.1 | 36.2 | 0.6 |
| | | 8.6 | 41.6 | 0.8 | 34.2 | 1.3 |
| | | 0.0 | 30.1 | 1.6 | 35.2 | 0.4 |
| G015995 | HLA DR | 550.0 | 88.0 | 0.2 | 52.8 | 1.1 |
| G016017 | DP DQ | 275.0 | 81.2 | 0.2 | 46.4 | 0.4 |
| G016206 | neg | 137.5 | 70.4 | 1.3 | 39.9 | 1.8 |
| G018117 | | 68.8 | 60.0 | 0.4 | 39.1 | 3.3 |
| | | 34.4 | 48.8 | 0.6 | 37.7 | 2.9 |
| | | 17.2 | 43.0 | 4.2 | 37.5 | 0.6 |
| | | 8.6 | 37.8 | 2.1 | 35.0 | 0.0 |
| | | 0.0 | 33.0 | 1.9 | 37.3 | 2.1 |
| G015995 | B2M neg | 550.0 | 84.2 | 0.0 | 46.8 | 1.1 |
| G016017 | CD3 neg | 275.0 | 76.2 | 0.0 | 37.8 | 0.2 |
| G016206 | HLA DR | 137.5 | 63.0 | 1.3 | 23.4 | 2.4 |
| G018117 | DP DQ | 68.8 | 48.2 | 0.2 | 10.8 | 0.9 |
| | neg | 34.4 | 31.5 | 1.1 | 3.6 | 0.9 |
| | | 17.2 | 17.8 | 1.7 | 1.1 | 0.2 |
| | | 8.6 | 6.4 | 0.0 | 0.4 | 0.1 |
| | | 0.0 | 0.1 | 0.0 | 0.1 | 0.0 |

### Example 7: HLA-E Protection of B2M Knockout T Cells in an NK Cell In Vivo Killing Mouse Model

Female NOG-hIL-15 mice were engrafted with 1.5×10⁶ primary NK cells followed by the injection of either wild-type T cells or B2M knockout T cells containing luciferase+/−HILA-E to test for HILA-E protection from in vivo NK cell killing of injected T cells.

7.1. Sequential Infection with Luciferase and HLA-E Lentivirus.

This example explains the production of wild type or B2M^{-/-} T cells containing luciferase+/−HLA-E. Electroporated wild type and B2M^{-/-} T-cells were first infected with luciferase lentivirus (Imanis Life Sciences; Cat #LV050L). B2M^{-/-} T cells were later sequentially infected with HLA-E lentivirus (LVP112). Luciferase infection was performed by infecting 1×10⁶ cells in 150 ul of luciferase lentivirus supplemented with 350 ul of Media Number 18 and centrifuged at 1000×G for 60 mins at 37° C. Prior to second round of infection with HLA-E lentivirus for B2M^{-/-} HLA-E, cells were rested in Media Number 2 for 2 hours after which 1×10⁶ cells were infected with 60 ul of HLA-E virus in 440 ul of Media Number 18. WT luciferase⁺ T cells and B2M^{-/-} luciferase⁺ cells group did not receive any virus but were spun in Media Number 18 only to keep conditions similar across different groups. After infection, cells were re-suspended and combined into their designated groups in a 24 well G-Rex plate and brought up to 7 mL of Media Number 19, as described in Table 3. Fresh cytokines were added every 2 days in culture.

### 7.2. Preparation of Wildtype and B2M^{-/-} T Cells Containing Luciferase+/−HLA-E.

Fresh healthy human peripheral blood leukapheresis pack was received from Stemcell Technologies, and cells were resuspended in PBS and washed once. Cell pellet was then re-suspended in Ammonium Chloride RBC lysis buffer (Stemcell Technologies; Cat #07800) for 15 mins followed by washing with PBS. PBMC count was determined post lysis and T cell isolation was performed using EasySep Human T cell isolation kit (Stemcell Technologies, Cat #17951) according to manufacturer's protocol. Isolated CD3+ T cells were re-suspended in Cryostor CS10 media (Stemcell Technologies, Cat #07930) and frozen down in liquid nitrogen until further use.

7.3. sgRNA Electroporation in T Cells.

RNP's were formulated for performing B2M knockout using guide G000193 (100 μM), recombinant Cas9-NLS protein (50 μM) and Reaction Buffer (1×). Single guide G000193 was first denatured at 95° C. for 2 mins followed by cooling down on ice for 10 mins. 6 μl of 50 μM Cas9 was mixed with 18 μl of 1× Reaction buffer and 6 μl of denatured G000193 guide in a PCR tube to make final volume of 30 μl. RNP's were formulated by incubating at 25° C. for 10 mins followed by leaving on ice until further use. Upon thaw, CD3+ T-cells were plated at a density of 0.5×10⁶ cells/ml in Media Number 19 containing IL-2 (100 U/ml)(Peprotech; Cat #200-02), IL-7 (2.5 ng/ml) (Stemcell; Cat #78053.1) and IL-15 (2.5 ng/ml)(Stemcell; Cat #78031.1). Cells were stimulated with Transact (1:100 Dilution, Miltenyi Biotec; Cat #130-111-160) for 48 hours. Post stimulation 10×10⁶ cells were centrifuged and re-suspended in 80 ul P3 electroporation buffer (Lonza; Cat #V4XP-3024) followed by adding 25 μl of RNP and electroporated in cuvettes using Lonza electroporator with pulse code EO-115. Wild type T cells went through a similar process for mock electroporation in P3 buffer only.

### 7.4. Preparation of Purified Luciferase+, HLA-E+, MHC Class I-T Cells.

On day 7 post infection, cells were collected and washed with FACS Buffer and blocked using Human Tru Stain Fc Block (Biolegend) for 5 mins followed by staining with anti-human MHC-I APC antibody (Clone #W6/, Biolegend) alone or co-stained with anti-human HLA-E BV421 antibody (Clone #3D12, Biolegend) for 30 mins. Cells were sorted using BD FACS Aria by gating on GFP⁺ only, GFP⁺/MHC-I⁻, GFP⁺/MHC-I⁻/HLA-E⁺ for WT luciferase⁺ T cells, B2M^{-/-} luciferase⁺ T cells, and B2M^{-/-} HLA-E⁺ luciferase⁺ T cells respectively. Collected cells were washed and resuspended in Media Number 19 and transferred to a 6-well G-Rex. Cells were stimulated with another round of Transact at 1:100 dilution for 48 hours. Transact was washed out of stimulated T cells post 48 hours of stimulation and resuspended in Media Number 19 and cultured in G-Rex plate with fresh cytokines added every 2 days.

### 7.5. Preparation of T Cells for Injection.

T cells were injected 10 days post second stimulation after washing in PBS and resuspending in HBSS solution for injection into NOG-IL15 mice.

### 7.6. Protective Effect of HLA-E on B2M Knockout T Cells In Vivo.

NK cells were washed with HBSS (Gibco, Cat. No. 14025-092) and resuspended at 10×10⁶ cells/mL for injection in 150 μL. Mice were separated into two groups initially: (1) Non-injected NOG/IL-15 mice, control group (n=1); and (2) 1.5 million primary NK cells (n=2). An additional six groups received the following treatments: (3) Wild-type T cells only (n=4); (4) B2M^{-/-} T cells only (n=4); (5) B2M^{-/-} HLA-E T cells only (n=4), (6) 1.5 million primary NK cells + wild-type T cells (n=5); (7) 1.5 million primary NK cells +B2M^{-/-} T cells (n=5); and (8) 1.5 million primary NK cells +B2M^{-/-} HLA-E T cells (n=5). NK cell solution was injected via the tail vein with a 27-gauge needle.

Mice were inoculated with wild-type, B2M^{-/-}, or B2M^{-/-} +HLA-E T cells 28 days post NK cell injection. Cells were prepared at a concentration of 6×10⁶ cells/150 μL volume.

IVIS imaging was performed to identify luciferase-positive T cells by IVIS spectrum. IVIS imagine was done at 6 hours, 24 hours, 48 hours, 4 days, 6 days, 8 days, 11 days, 18 days, 25 days, 29 days, 33 days, 50 days, 55 days, 61 days, 74 days, and 90 days after T cell injection. Mice were prepared for imaging with an injection of D-luciferin i.p. at 10 μL/g body weight per the manufacturer's recommendation, about 150 μL per animal. Animals were anesthetized and then placed in the IVIS imaging unit. The visualization was performed with the exposure time set to auto, field of view D, medium binning, and F/stop set to 1.

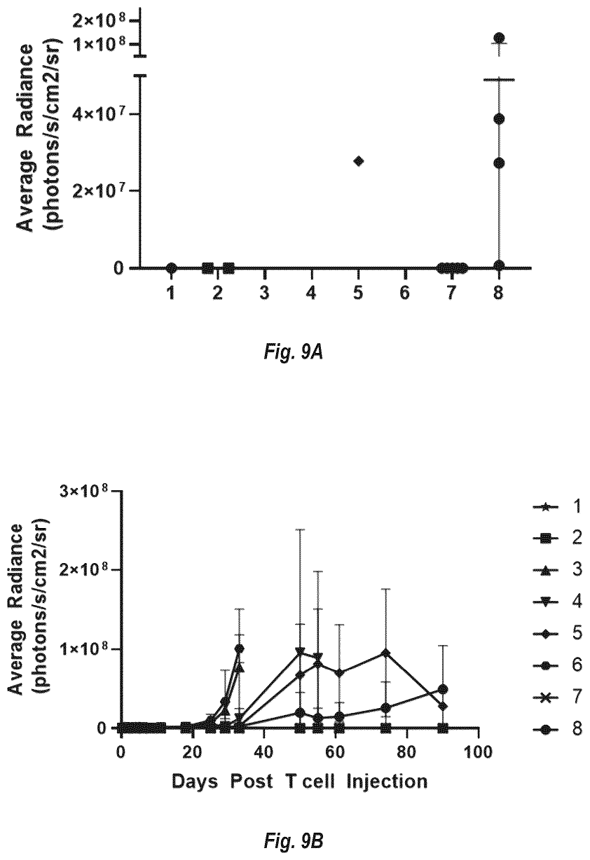

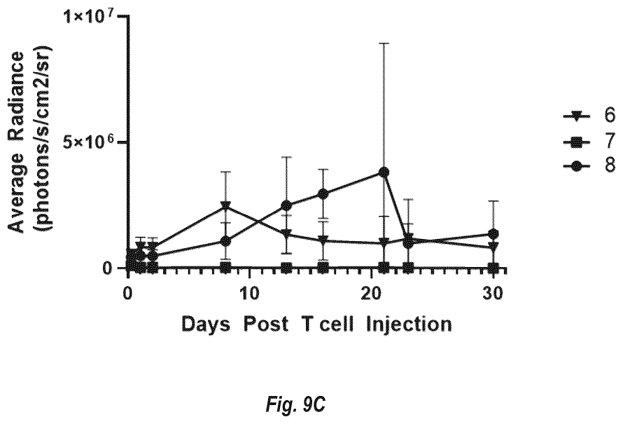

FIG. 9A shows the HLA-E protection of B2M KO T cells from NK cell lysis 90 days post T cell injection. FIG. 9B shows the HLA-E protection of B2M KO T cells from NK cells over the 90-day time course post T cell injection. FIG. 9C shows the protective effect of HLA-E over the 30-day time course of a replicate study.

### Example 8: CIITA Editing in Lymphoblastoid Cell Lines

Lipid nanoparticles (LNPs) comprising a CIITA guide RNA are used to edit two lymphoblastoid cell lines (LCLs). LCLs are developed by infecting peripheral blood lymphocytes (PBLs) from human donors with Epstein Barr Virus (EBV). This process has been demonstrated to immortalize human resting B cells in vitro giving rise to an actively proliferating B cell population positive for B cell marker CD19 and negative for T cell marker CD3 as well as for NK cell marker CD56 (Neitzel H. A routine method for the establishment of permanent growing lymphoblastoid cell lines. Hum Genet. 1986; 73(4):320-6).

Lymphoblastoid cell lines GM26200 and GM20340 are obtained from the Coriell Institute for Medical Research (Camden, NJ, USA). LCLs are grown in RPMI-1640 with L-glutamine and 15% FBS. At the time of LNP contact, cells are activated with 4 ng/ml IL-4 (R&D System Cat. No. 204-IL-010), 1 ng/mL IL-40 (R&D System Cat. No. 6245-CL-050), 25 ng/ml BAFF (R&D System Cat. No. 2149-BF-010). The LNPs targeting B2M are formulated at a ratio of 50/10/38.5/1.5 ionizable Lipid B, cholesterol, DSPC, and PEG2k-DMG as described in Example 1.3. LNPs targeting ATTR were formulated with lipid A, cholesterol, DSPC, and PEG2k-DMG in a 50:38.5:9:3 molar ratio, respectively. The lipid nucleic acid assemblies were formulated with a lipid amine to RNA phosphate (N:P) molar ratio of about 6, and a ratio of gRNA to mRNA of 1:2 by weight. LNPs formulated with Cas9 mRNA and a CIITA gRNA are pre-incubated at 37° C. for about 5 minutes with M. fascicularis (cynomolgus monkey) serum (BioReclamationIVT, Cat. No. CYN197452) at 6% (v/v) are delivered to lymphoblastoid cells. Six days post LNP treatment, half of the cells are collected for NGS sequencing and a day later the other half of the cells for flow cytometry analyses. NGS analysis is performed according to the following using genomic DNA that was extracted using QuickExtract™ DNA Extraction Solution (Lucigen, Cat. No. QE09050) according to manufacturer's protocol.

NGS analysis is performed as in Example 1.1.

Flow cytometry is performed. For flow cytometry analysis, cells are washed in FACS buffer (PBS+2% FBS+2 mM EDTA). Then the cells are blocked with Human TruStain FcX (Biolegend® Cat. No. 422302) at room temperature (RT) for 5 minutes and incubated with APC- or PE-conjugated antibody at 1:200 dilution for 30 mins at 4° C. After the incubation, the cells are washed and resuspended buffer containing live-dead marker 7AAD (1:1000 dilution; Biolegend© Cat. No. 420404). The cells are processed by flow cytometry, for example using a Beckman Coulter CytoflexS™, and are analyzed using the FlowJo™ software package.

### Example 9. Directional Genomic Hybridization Analysis for Chromosomal Translocation Following Gene Editing of CIITA, B2M, and TRAC

T cells treated with electroporation or lipid nanoparticles (LNPs) to deliver Cas9 mRNA and sgRNAs were analyzed for chromosomal structural variations including translocations by directional Genomic Hybridization (dGH™) by KromaTiD (Longmont, CO).

### 9.1. Electroporation Treatments.

For the electroporation treatment, T cells were isolated and cryopreserved as follows: T cells were either obtained commercially (e.g. Human Peripheral Blood CD4⁺CD45RA⁺ T Cells, Frozen, Stem Cell Technology, Cat. 70029) or prepared internally from a leukopak. For internal preparation, T cells were isolated by negative selection using the EasySep Human T cell Isolation Kit (Stem Cell Technology, Cat. 17951) following the manufacturers protocol. T cells were cryopreserved in Cryostor® CS10 freezing media (Cat. No. 07930) for future use. Cryopreserved T cells were thawed and rested overnight in Media Number 1, as described in Table 3.

Rested T cells were electroporated to deliver ribonucleoprotein (RNP) complexes containing guides G013674 (SEQ ID NO: 702) or G000529 (SEQ ID NO: 701), targeting CIITA and B2M genes respectively. Briefly stock RNPs were prepared by incubating recombinant Cas9-NLS protein (50 μM stock) with sgRNA (100 uM) to a final concentration of 20 μM Cas9 with 40 μM sgRNA (1:2 Cas9 protein to guide ratio). Cultured T cells were harvested at 1×10⁶ cells resuspended in 100 μL Buffer P3 (Lonza, Cat. No. V4SP-3960) and incubated with 12.5 μL of RNPs to a final concentration of 2 μM each. T cells were subsequently electroporated using the Lonza 4D-Nucleofector™5. Electroporated cells were collected and rested for 48 hours in Media Number 1, as described in Table 3. Subsequently, T cells were harvested, resuspended to a density of 1×10⁶ cells/mL in Media Number 1, as described in Table 3 and activated with T cell TransAct™ reagent (Miltenyi Biotec, Cat. No. 130-111-160) at a 1/100 dilution. Forty-eight hours after T cell activation, T cells were electroporated as described above with Cas9-RNPs including G012086 (SEQ ID NO: 703) targeting TRAC. Triple edited T cells was transferred back to Media Number 1, as described in Table 3 and expanded for future analysis.

After expansion, the cells were passed through the Magnetic-Activated Cell Sorting (MACS) depletion process for selecting the triple knockout cells using the Anti-Biotin microbeads (Miltenyi Biotec, Cat. No. 130-090-485) protocol for MHC Class I (Miltenyi Biotec, Cat. No. 130-120-431), MHC Class II (Miltenyi Biotec, 130-104-823) and CD3-biotin (Miltenyi Biotec, Cat. No. 130-098-612) as per the manufacturer's protocol. The negatively selected cells were collected for flow cytometry analysis and NGS analysis. The protocols described in Examples 3.3 and 1.1 were used for these analyses.

### 9.2. Sequential and Simultaneous LNP Treatment.

For the LNP treatment, T cells were isolated and cryopreserved as in Example 3.1. Upon thaw, T cells were activated with T cell TransAct™ (Miltenyi Biotec, Cat. No. 130-111-160) as recommended by the manufacturer's protocol and cultured at 37° C. for 24-72 hours as specified below.

For the simultaneous LNP treatment, T cells were treated 72 hours post activation with three LNPs delivering mRNA encoding Cas9 (SEQ ID NO: 809) and sgRNAs G000529 (SEQ ID NO: 701), G012086 (SEQ ID NO: 703), and G013674 (SEQ ID NO: 702), targeting B2M, TRAC and CIITA respectively. LNPs were pre-incubated with cyano serum at 37° C. for 5 mins and dosed at 100 ng of total RNA cargo per 100,000 T cells. After 24 hours LNP exposure, the cells were washed and resuspended in Media Number 11, as described in Table 3, and cultured at 37° C. for 5 days.

For sequential LNP treatment, T cells were treated 24 hours post activation with a single LNP delivering mRNA encoding Cas9 (SEQ ID NO: 809) and G000529 (SEQ ID NO: 701) targeting B2M as described for simultaneous LNP treatment above. Following wash and resuspension, a single LNP delivering mRNA encoding Cas9 (SEQ ID NO: 809) and G013674 (SEQ ID NO: 702) targeting CIITA was added at 48 hours post activation. Lastly, following wash and resuspension, a single LNP delivering mRNA encoding Cas9 (SEQ ID NO: 809) and G012086 (SEQ ID NO: 703) targeting TRAC was added at 72 hours post activation. After 24 hours exposure to the final LNP, cells were washed and resuspended in Media Number 11, as described in Table 3, and cultured at 37° C. for 5 days.

LNP treated T cells were passed through the MACS triple negative selection process and further flow cytometry analysis (as described in Example 3.2) and NGS analysis (as described in Example 1.1) were performed on these samples as described for electroporated cells.

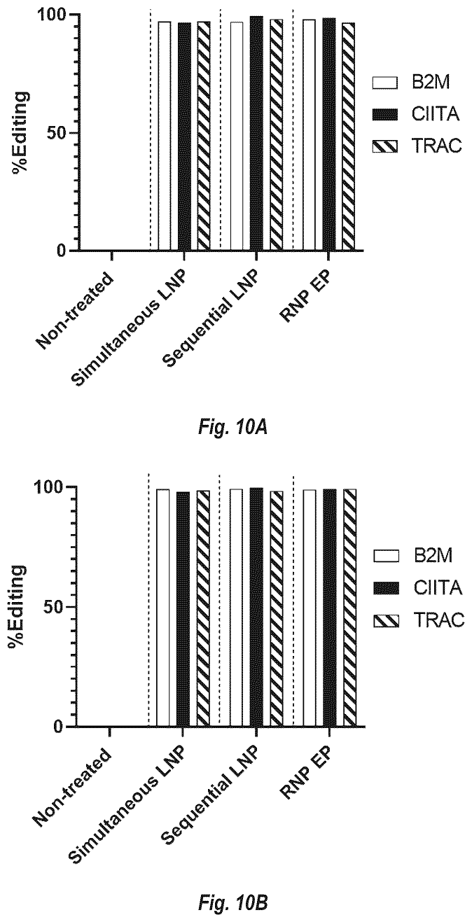

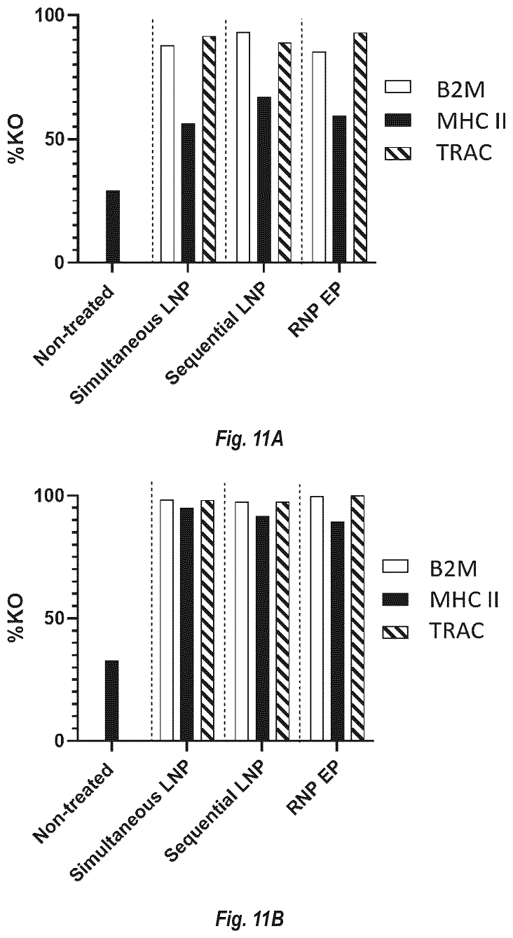

Treated and non-treated cells were assayed for percent editing by NGS (as described in Example 1.1) and protein expression by flow cytometry (as described in Example 3.3) both before and after MACS processing. The following flow cytometry reagents were used as phenotypic readouts of gene editing for B2M, CIITA and TRAC, respectively: FITC anti-human 02-microglobulin Antibody (Biolegend®, Cat. No. 316304), APC anti-human CD3 Antibody (Biolegend®, Cat. No. 300412), PE anti-human HLA-DR, DP, DQ Antibody (Biolegend®, Cat. No. 361716). NGS editing results are shown in Table 15 and FIG. 10A (before MACs), FIG. 10B (after MACs). Flow cytometry results are shown in Table 16 and FIG. 11A (before MACs), FIG. 11B (after MACs).

TABLE 15
Editing analysis by NGS.

| | Condition | | | | | |
|---|---|---|---|---|---|---|
| | B2M % edit | | CIITA % edit | | TRAC % edit | |
| MACS | Before | After | Before | After | Before | After |
| Non-treated | 0.1 | 0.1 | 0.2 | 0.2 | 0.2 | 0.1 |
| Simultaneous LNP | 97.3 | 99.3 | 96.5 | 98.2 | 97.3 | 98.6 |
| Sequential LNP | 97.0 | 99.4 | 99.6 | 99.8 | 98.2 | 98.5 |
| RNP EP | 98.0 | 99.1 | 98.7 | 99.4 | 96.7 | 99.4 |

TABLE 16
Flow cytometry analysis.

| | Condition | | | | | |
|---|---|---|---|---|---|---|
| | B2M % | | MHC class II % | | CD3 % | |
| | negative | | negative | | negative | |
| MACS | Before | After | Before | After | Before | After |
| Non-treated | 0.2 | 0.2 | 29.4 | 32.8 | 0.3 | 0.2 |
| Simultaneous LNP | 87.9 | 98.4 | 56.5 | 95.0 | 91.7 | 98.3 |
| Sequential LNP | 93.2 | 97.6 | 67.0 | 91.8 | 89.0 | 97.6 |
| RNP EP | 85.4 | 99.9 | 59.6 | 89.4 | 93.1 | 100 |

9.3. Kromatid dGH™ Analysis for Chromosomal Structural Rearrangements.

Engineered T cells were prepared for the dGH procedure according to the KromaTiD's protocol. Briefly, T cells were cultured for 17 hours with the addition of 5 μM BrdU and 1 μM BrdC as provided by KromaTiD. Colcemid was added at a concentration of 10 μl/ml for an additional 4 hours. Cells were harvested by centrifugation, incubated in 75 mM KCl hypotonic solution for 30 minutes at room temperature, and fixed in a 3:1 methanol to acetic acid solution.

Three sets of fluorescence in situ hybridization (FISH) probes were designed to bracket the genomic target sites of the guides used to engineer these T cells, which are located on separate chromosomes. KromaTiD imaged 200 metaphase spreads per sample using their proprietary dGH FISH and scored the spreads for chromosomal structural rearrangements. Cells without chromosomal structural rearrangements showed 3 matched-color, adjacent pairs of FISH signals. “Deletions” were scored when zero FISH signals for a target site were identified in the cell, indicating chromosomal rearrangement where fragments were lost during the cell replication cycle due to the editing event occurring. “Reciprocal translocations” were scored for each pair of adjacent, color-mismatched FISH signals, indicating a translocation between two Cas9 targeted cleavages (e.g. between B2M and TRAC target sites). “Translocations to off-target chromosomes” showed a single FISH signal, indicating a fusion between a Cas9-targeted cleavage site and unlabeled chromosomal site.

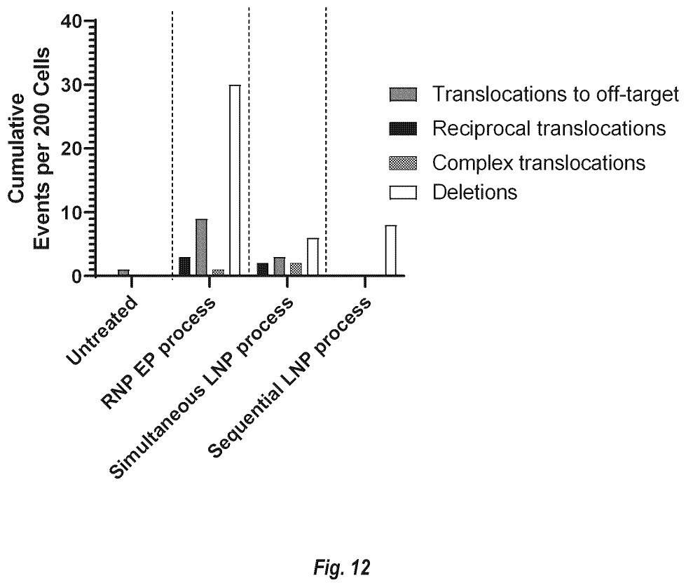

“Complex translocations” denote FISH signals not included in reciprocal translocations and translocations to off-target sites. Total translocations were calculated as a sum total of the reciprocal translocations, translocations to off-target chromosomes/sites in the genome and complex translocations. Table 17 and FIG. 12 show the chromosomal rearrangements identified by this method for each condition.

TABLE 17
Translocations analysis by Kromatid dGH assay.

| Chromosomal | | | | |
|---|---|---|---|---|
| rearrangements | | Sequential | Simultaneous | RNP |
| events: | Untreated | LNP | LNP | EP |
| Total Translocations | 1 | 0 | 7 | 13 |
| Reciprocal | 0 | 0 | 2 | 3 |
| translocations | | | | |
| Translocations to | 1 | 0 | 3 | 9 |
| off-target | | | | |
| chromosomes | | | | |
| Complex | 0 | 0 | 2 | 1 |
| Translocations | | | | |
| Deletions | 0 | 8 | 6 | 30 |

### Example 10: Off Target Analysis

### 10.1. Biochemical Off-Target Analysis.

A biochemical method (See, e.g., Cameron et al., Nature Methods. 6, 600-606; 2017) was used to determine potential off-target genomic sites cleaved by Cas9 using specific guides targeting CIITA. In this experiment, two sgRNAs targeting human CIITA were screened using genomic DNA purified from lymphoblast cell line NA24385 (Coriell Institute) alongside three control guides with known off-target profiles. The number of potential off-target sites detected using a guide concentration of 192 nM and 64 nM Cas9 protein in the biochemical assay are shown in Table 18.

TABLE 18
Biochemical Off-Target Analysis.

| SEQ ID | | | Number of |
|---|---|---|---|
| NO: | | Target | Sites |
| 71 | G018091 | CIITA | 603 |
| 80 | G018100 | CIITA | 291 |
| 100 | G018120 | CIITA | 203 |
| 707 | G000644 | EMX1 | 440 |
| 708 | G000645 | VEGFA | 6384 |
| 709 | G000646 | RAG1B | 144 |
| 14 | G018034 | CIITA | 14 |
| 15 | G018035 | CIITA | 3 |
| 58 | G018078 | CIITA | 49 |
| 61 | G018081 | CIITA | 9 |
| 62 | G018082 | CIITA | 23 |
| 66 | G018086 | CIITA | 353 |
| 81 | G018101 | CIITA | 13 |
| 84 | G018104 | CIITA | 38 |
| 85 | G018105 | CIITA | 93 |
| 87 | G018107 | CIITA | 36 |
| 88 | G018108 | CIITA | 56 |
| 89 | G018109 | CIITA | 73 |
| 707 | G000644 | EMX1 | 276 |
| 708 | G000645 | VEGFA | 3259 |
| 709 | G000646 | RAGIB | 32 |

### 10.2. Targeted Sequencing for Validating Potential Off-Target Sites.

Potential off-target sites predicted by detection assays such as the biochemical method used above may be assessed using targeted sequencing of the identified potential off-target sites to determine whether off-target cleavage at that site is detected.

In one approach, Cas9 and a sgRNA of interest (e.g., a sgRNA having potential off-target sites for evaluation) are introduced to primary T cells. The T cells are then lysed and primers flanking the potential off-target site(s) are used to generate an amplicon for NGS analysis. Identification of indels at a certain level may validate potential off-target site, whereas the lack of indels found at the potential off-target site may indicate a false positive from the off-target predictive assay that was utilized.

### Example 11: CIITA Guide RNA Screening in T Cells with BC22n

Different sgRNAs were screened for their potency in knocking out the CIITA gene in human T cells using C to T base editing. The percentage of T cells negative for MHC class II and/or CD74 protein expression was assayed following CIITA editing following electroporation with mRNA and different sgRNAs.

### 11.1 T Cell Preparation

Healthy human donor apheresis was obtained commercially (Hemacare), and cells were washed and resuspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTimizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/mL recombinant human interleukin-7 (Peprotech, Cat. 200-07), and 5 ng/mL recombinant human interleukin-15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to electroporation.

11.2 T Cell Editing with RNA Electroporation

Solutions containing mRNA encoding BC22n (SEQ ID NO: 804 or 805) and UGI (SEQ ID NO: 807 or 808) were prepared in P3 buffer. One hundred μM of CIITA-targeting sgRNAs were removed from their storage plates and denatured for 2 minutes at 95° C. and incubated at room temperature for 5 minutes. Forty-eight hours post activation, T cells were harvested, centrifuged, and resuspended at a concentration of 12.5×10\hat{ }6 T cells/mL in P3 electroporation buffer (Lonza). For electroporation, 1×10\hat{ }5 T cells were mixed with 20 ng/μL of BC22n mRNAs, 20 ng/μL of UGI mRNA, and 20 pmols of sgRNA as described in Table 1 in a final volume of 20 μL of P3 electroporation buffer. This mix was transferred in duplicate to a 96-well Nucleofector™ plate and electroporated using the manufacturer's pulse code. Electroporated T cells were immediately rested in 80 μL of CTS Optimizer T cell growth media without cytokines for 15 minutes before being transferred to new flat-bottom 96-well plates containing an additional 80 μL of CTS Optimizer T cell growth media supplemented with 2× cytokines. The resulting plates were incubated at 37° C. for 10 days. On day 4 post-electroporation, cells were split 1:2 in 2 U-bottom plates. One plate was collected for NGS sequencing, while the other plate was replenished with CTS Optimizer fresh media with 1× cytokines. This plate was used for flow cytometry on Day 7.

### 11.3 Flow Cytometry and NGS Sequencing

On day 7 post-editing, T cells were assayed by flow cytometry to determine the surface expression of CD74 and HLA-DR, DP, DQ. Briefly, T cells were incubated for 30 minutes at 4° C. with a mixture of antibodies diluted in cell staining buffer (BioLegend, Cat. No. 420201). Antibodies against CD3 (BioLegend, Cat. No. 317336), CD4 (BioLegend, Cat. No. 317434), CD8 (BioLegend, Cat. No. 301046), and Viakrome (Beckman Coulter, Cat. No. C36628) were diluted at 1:100, and antibodies against HLA II-DR (BioLegend, Cat. No. 327018), HLA II-DP (BD Biosciences Cat No. 750872), HLA II-DQ (BioLegend, Cat. No. 561504), and CD74 (BioLegend, Cat. No. 326808) were diluted at 1:50. Cells were subsequently washed, resuspended in 100 μL of cell staining buffer and processed on a Cytoflex flow cytometer (Beckman Coulter). Flow cytometry data was analyzed using the FlowJo software package. T cells were gated based on size, shape, viability, CD8, HLA II-DP, HLA II-DQ, HLA II-DR, and CD74 expression.

TABLE 19
Percentage of cells negative for surface protein following
genomic editing of CIITA with BC22n. (n = 2)

| Guide | % HLA II-DP- | | % HLA II-DQ- | | % HLA II-DR- | | % CD74- | |
|---|---|---|---|---|---|---|---|---|
| ID | Mean | SD | Mean | SD | Mean | SD | Mean | SD |
| G000502 | 68.70 | 3.54 | 76.30 | 4.24 | 76.70 | 3.96 | 66.25 | 5.87 |
| G018021 | 76.60 | 0.99 | 84.00 | 0.57 | 84.65 | 0.49 | 76.85 | 0.07 |
| G018022 | 60.00 | 3.39 | 72.20 | 1.13 | 73.25 | 1.34 | 60.35 | 2.62 |
| G018023 | 64.80 | 2.97 | 74.35 | 0.07 | 75.85 | 0.35 | 63.85 | 2.33 |
| G018024 | 64.10 | 1.70 | 74.80 | 1.84 | 74.90 | 1.98 | 63.75 | 4.03 |
| G018025 | 63.85 | 6.15 | 73.50 | 6.08 | 74.90 | 6.36 | 63.75 | 6.72 |
| G018026 | 72.15 | 4.17 | 81.35 | 0.92 | 82.55 | 1.48 | 73.00 | 0.71 |
| G018027 | 82.40 | 0.99 | 81.45 | 1.91 | 82.40 | 2.26 | 72.35 | 3.32 |
| G018028 | 78.25 | 0.49 | 78.40 | 0.00 | 80.05 | 0.49 | 67.90 | 2.40 |
| G018029 | 75.60 | 3.11 | 74.80 | 0.14 | 76.95 | 0.07 | 64.15 | 2.05 |
| G018030 | 76.70 | 1.56 | 76.35 | 3.46 | 77.80 | 3.54 | 66.65 | 5.44 |
| G018031 | 81.40 | 3.96 | 84.00 | 1.13 | 82.40 | 0.42 | 75.55 | 0.49 |
| G018032 | 77.00 | 2.97 | 79.70 | 0.14 | 79.30 | 0.42 | 70.35 | 0.92 |
| G018033 | 81.15 | 3.04 | 82.05 | 0.35 | 81.60 | 0.57 | 73.20 | 1.27 |
| G018034 | 96.87 | 0.43 | 93.49 | 0.88 | 92.67 | 0.99 | 92.96 | 1.63 |
| G018035 | 94.39 | 0.64 | 88.20 | 1.13 | 88.64 | 2.03 | 83.70 | 2.97 |
| G018036 | 73.80 | 0.28 | 76.45 | 2.19 | 77.55 | 2.90 | 65.30 | 3.82 |
| G018037 | 71.80 | 0.14 | 72.25 | 5.16 | 73.85 | 5.73 | 61.40 | 7.64 |
| G018038 | 70.25 | 7.85 | 74.95 | 1.06 | 75.65 | 2.05 | 63.65 | 2.62 |
| G018039 | 94.72 | 0.49 | 91.67 | 0.05 | 91.17 | 0.51 | 89.20 | 0.99 |
| G018040 | 77.30 | 1.13 | 81.70 | 0.42 | 81.60 | 0.57 | 72.35 | 0.64 |
| G018041 | 75.10 | 1.70 | 80.15 | 0.35 | 80.15 | 0.21 | 70.50 | 0.85 |
| G018042 | 89.35 | 0.49 | 86.60 | 0.14 | 87.90 | 0.14 | 80.50 | 0.00 |
| G018043 | 85.50 | 1.13 | 84.00 | 1.56 | 84.95 | 0.35 | 77.55 | 0.64 |
| G018044 | 88.20 | 0.99 | 89.01 | 1.57 | 89.25 | 0.78 | 83.95 | 2.19 |
| G018045 | 76.15 | 1.34 | 82.35 | 2.33 | 81.60 | 2.26 | 72.50 | 3.25 |
| G018046 | 87.20 | 0.42 | 88.65 | 0.21 | 89.00 | 0.57 | 82.80 | 1.27 |
| G018047 | 79.95 | 1.63 | 84.60 | 2.12 | 84.25 | 2.76 | 76.25 | 3.61 |
| G018048 | 88.50 | 2.12 | 88.00 | 1.13 | 89.50 | 0.42 | 81.60 | 0.14 |
| G018049 | 81.40 | 3.25 | 81.40 | 4.24 | 82.60 | 5.23 | 74.00 | 5.66 |
| G018050 | 77.75 | 0.49 | 78.60 | 0.85 | 80.85 | 0.07 | 70.25 | 0.35 |
| G018051 | 81.20 | 1.56 | 81.95 | 1.48 | 84.20 | 1.13 | 73.55 | 3.04 |
| G018052 | 76.30 | 1.56 | 81.70 | 1.27 | 81.10 | 1.56 | 70.90 | 2.69 |
| G018053 | 77.25 | 1.06 | 81.40 | 0.85 | 82.35 | 1.06 | 71.55 | 1.63 |
| G018054 | 90.23 | 0.14 | 89.00 | 0.99 | 89.25 | 0.64 | 84.50 | 0.28 |
| G018055 | 73.80 | 3.39 | 79.20 | 0.99 | 79.85 | 1.06 | 67.80 | 1.41 |
| G018056 | 73.40 | 2.97 | 83.15 | 0.35 | 83.15 | 0.78 | 73.15 | 0.78 |
| G018057 | 73.95 | 1.63 | 81.40 | 2.97 | 81.45 | 1.91 | 71.30 | 3.11 |
| G018058 | 73.40 | 3.25 | 80.90 | 0.99 | 81.05 | 0.07 | 69.15 | 0.35 |
| G018059 | 75.15 | 4.17 | 81.70 | 0.14 | 81.55 | 0.07 | 71.10 | 1.13 |
| G018060 | 75.90 | 2.69 | 81.30 | 0.28 | 81.55 | 0.78 | 72.05 | 1.91 |
| G018061 | 71.65 | 2.05 | 81.60 | 1.41 | 80.65 | 1.06 | 72.00 | 1.41 |
| G018062 | 70.75 | 2.33 | 75.05 | 0.64 | 76.30 | 1.56 | 64.65 | 2.33 |
| G018063 | 78.45 | 0.92 | 80.95 | 0.92 | 82.20 | 0.85 | 72.50 | 0.14 |
| G018064 | 76.15 | 0.78 | 82.50 | 0.00 | 82.90 | 0.28 | 73.90 | 0.28 |
| G018065 | 77.65 | 0.21 | 82.60 | 0.42 | 83.85 | 0.35 | 74.05 | 0.07 |
| G018066 | 72.60 | 0.00 | 82.30 | 0.28 | 83.05 | 0.64 | 73.60 | 0.14 |
| G018067 | 97.57 | 0.81 | 98.24 | 0.19 | 97.73 | 0.28 | 98.83 | 0.04 |
| G018068 | 89.05 | 0.64 | 90.79 | 0.52 | 90.81 | 1.02 | 87.20 | 1.27 |
| G018069 | 75.90 | 2.55 | 81.55 | 0.92 | 82.15 | 0.35 | 71.85 | 1.20 |
| G018070 | 75.30 | 1.27 | 82.05 | 1.20 | 82.15 | 0.64 | 71.20 | 0.42 |
| G018071 | 77.40 | 3.39 | 83.35 | 1.77 | 84.75 | 1.91 | 74.65 | 0.92 |
| G018072 | 72.05 | 1.63 | 81.45 | 0.78 | 81.95 | 1.91 | 71.95 | 2.62 |
| G018073 | 72.35 | 0.07 | 79.30 | 1.13 | 79.25 | 2.19 | 69.40 | 2.26 |
| G018074 | 67.25 | 2.05 | 75.25 | 0.49 | 76.40 | 0.57 | 64.35 | 0.35 |
| G018075 | 96.86 | 1.46 | 97.33 | 0.14 | 96.08 | 0.31 | 98.35 | 0.04 |
| G018076 | 94.26 | 4.87 | 97.96 | 0.21 | 97.57 | 0.15 | 98.55 | 0.06 |
| G018077 | 74.75 | 2.76 | 81.65 | 0.35 | 82.25 | 0.21 | 72.25 | 1.34 |
| G018078 | 94.30 | 0.48 | 94.87 | 0.31 | 95.03 | 0.06 | 93.43 | 0.24 |
| G018079 | 86.00 | 1.56 | 89.55 | 0.35 | 89.80 | 0.28 | 84.25 | 0.78 |
| G018080 | 80.50 | 3.96 | 85.50 | 1.70 | 85.45 | 1.63 | 78.35 | 0.78 |
| G018081 | 79.85 | 1.06 | 86.05 | 1.20 | 86.00 | 0.85 | 78.70 | 0.57 |
| G018082 | 80.10 | 0.14 | 83.10 | 0.42 | 84.05 | 1.20 | 75.65 | 1.91 |
| G018083 | 86.75 | 1.34 | 89.50 | 0.28 | 89.35 | 0.21 | 84.55 | 0.21 |
| G018084 | 84.60 | 3.25 | 87.35 | 1.34 | 87.95 | 0.92 | 82.00 | 1.27 |
| G018085 | 86.95 | 2.76 | 90.35 | 2.05 | 90.03 | 1.03 | 86.30 | 2.26 |
| G018086 | 68.15 | 1.34 | 78.60 | 0.42 | 75.95 | 0.07 | 66.25 | 0.92 |
| G018087 | 61.40 | 2.26 | 74.35 | 2.33 | 72.65 | 2.62 | 61.85 | 1.63 |
| G018088 | 88.80 | 0.85 | 91.11 | 0.28 | 91.88 | 0.11 | 87.60 | 1.41 |
| G018089 | 81.75 | 4.17 | 89.00 | 1.83 | 89.28 | 1.53 | 83.75 | 1.77 |
| G018090 | 81.65 | 1.20 | 90.11 | 0.29 | 89.98 | 0.25 | 85.35 | 1.34 |
| G018091 | 97.02 | 0.49 | 98.63 | 0.03 | 98.15 | 0.12 | 98.98 | 0.08 |
| G018092 | 71.40 | 5.23 | 81.90 | 0.71 | 82.40 | 0.85 | 73.45 | 0.92 |
| G018093 | 94.15 | 1.20 | 96.36 | 0.21 | 96.36 | 0.02 | 95.70 | 0.18 |
| G018094 | 77.50 | 2.12 | 84.55 | 1.63 | 85.25 | 1.48 | 76.50 | 0.14 |
| G018095 | 69.50 | 0.00 | 83.05 | 0.07 | 82.70 | 0.14 | 73.90 | 0.99 |
| G018096 | 69.25 | 0.21 | 83.50 | 0.99 | 83.45 | 1.06 | 75.25 | 2.05 |
| G018097 | 68.55 | 4.74 | 81.60 | 2.83 | 82.20 | 1.84 | 73.25 | 3.75 |
| G018098 | 66.65 | 0.21 | 78.85 | 0.21 | 79.75 | 0.21 | 68.75 | 1.20 |
| G018099 | 65.05 | 3.18 | 73.70 | 4.95 | 74.55 | 4.31 | 62.40 | 4.53 |
| G018100 | 95.51 | 0.40 | 95.99 | 0.19 | 95.62 | 0.92 | 96.21 | 0.18 |
| G018101 | 93.00 | 0.33 | 93.70 | 0.01 | 94.03 | 0.25 | 93.11 | 0.28 |
| G018102 | 96.55 | 0.33 | 96.31 | 0.64 | 96.34 | 0.08 | 96.58 | 0.08 |
| G018103 | 96.92 | 1.46 | 97.58 | 0.02 | 97.53 | 0.08 | 98.36 | 0.40 |
| G018104 | 72.00 | 0.99 | 82.45 | 2.19 | 82.70 | 1.84 | 73.40 | 2.12 |
| G018105 | 71.60 | 1.98 | 82.20 | 0.71 | 81.80 | 0.28 | 73.30 | 0.00 |
| G018106 | 92.49 | 1.33 | 98.62 | 0.06 | 97.03 | 0.43 | 98.96 | 0.04 |
| G018107 | 96.55 | 1.25 | 98.14 | 0.56 | 97.53 | 0.70 | 98.98 | 0.08 |
| G018108 | 85.50 | 0.42 | 88.00 | 0.71 | 89.35 | 0.92 | 83.40 | 1.56 |
| G018109 | 84.10 | 1.56 | 84.55 | 2.19 | 86.90 | 1.27 | 79.75 | 3.32 |
| G018110 | 67.65 | 3.04 | 75.15 | 0.49 | 77.50 | 0.99 | 65.35 | 1.48 |
| G018111 | 96.49 | 0.35 | 96.85 | 1.05 | 95.24 | 1.32 | 97.03 | 0.52 |
| G018112 | 68.60 | 5.09 | 76.65 | 4.88 | 77.30 | 4.53 | 66.60 | 4.38 |
| G018113 | 72.75 | 3.89 | 79.60 | 3.54 | 80.15 | 2.76 | 70.30 | 3.96 |
| G018114 | 70.80 | 2.26 | 80.05 | 2.47 | 81.25 | 1.77 | 71.65 | 2.62 |
| G018115 | 62.40 | 1.41 | 77.40 | 0.57 | 76.90 | 1.27 | 67.00 | 1.56 |
| G018116 | 96.05 | 0.89 | 98.01 | 0.39 | 96.99 | 0.42 | 98.32 | 0.21 |
| G018117 | 96.93 | 1.27 | 97.63 | 0.52 | 97.19 | 0.87 | 98.96 | 0.25 |
| G018118 | 97.25 | 0.51 | 97.71 | 0.25 | 96.94 | 0.44 | 98.91 | 0.26 |
| G018119 | 94.78 | 1.02 | 96.29 | 1.11 | 95.57 | 1.05 | 97.00 | 1.20 |
| G018120 | 96.04 | 1.87 | 97.61 | 0.06 | 97.42 | 0.56 | 98.94 | 0.07 |
| G018121 | 95.26 | 0.62 | 97.12 | 0.41 | 96.44 | 0.23 | 97.85 | 0.45 |

On day 4 post-editing, DNA samples were subjected to PCR and subsequent NGS analysis, as described in Example 1. Table 20 shows CIITA editing outcomes in T cells edited with BC22n.

TABLE 20
Mean percent editing at CIITA locus with BC22n. (n = 2)

| Guide | % C to T | | % C to A/G | | % Indels | |
|---|---|---|---|---|---|---|
| ID | Mean | SD | Mean | SD | Mean | SD |
| G000502 | 84.63 | 2.39 | 0.82 | 0.19 | 1.26 | 0.37 |
| G018021 | 91.14 | 2.38 | 2.45 | 0.52 | 3.22 | 2.60 |
| G018022 | 93.68 | 3.27 | 1.83 | 0.33 | 1.46 | 0.10 |
| G018023 | 92.37 | 1.73 | 2.25 | 0.07 | 2.31 | 0.52 |
| G018024 | 96.06 | 1.54 | 0.41 | 0.37 | 0.97 | 0.07 |
| G018025 | 96.88 | 0.78 | 0.63 | 0.12 | 0.43 | 0.06 |
| G018026 | 89.15 | 1.37 | 1.66 | 0.04 | 7.20 | 1.31 |
| G018027 | 88.89 | 3.13 | 1.02 | 0.04 | 5.37 | 2.62 |
| G018028 | 91.28 | 2.09 | 0.59 | 0.24 | 2.96 | 2.02 |
| G018029 | 22.86 | 0.72 | 0.43 | 0.07 | 0.17 | 0.02 |
| G018030 | 79.89 | 2.66 | 0.36 | 0.51 | 0.18 | 0.25 |
| G018031 | 90.43 | 1.35 | 0.47 | 0.38 | 1.52 | 0.07 |
| G018032 | 64.78 | 2.64 | 1.39 | 0.69 | 1.06 | 0.50 |
| G018033 | 88.94 | 0.84 | 1.98 | 0.29 | 1.97 | 0.44 |
| G018034 | 89.00 | 1.56 | 1.09 | 0.11 | 6.23 | 0.88 |
| G018035 | 95.10 | 1.59 | 0.50 | 0.25 | 0.66 | 0.17 |
| G018036 | 83.69 | 0.01 | 1.91 | 0.30 | 0.53 | 0.20 |
| G018037 | 94.50 | 0.54 | 1.64 | 0.03 | 1.18 | 0.23 |
| G018038 | 90.91 | 0.00 | 1.11 | 0.00 | 3.74 | 0.00 |
| G018039 | 94.88 | 0.00 | 0.60 | 0.00 | 0.41 | 0.00 |
| G018040 | 44.77 | 1.52 | 0.33 | 0.14 | 0.50 | 0.03 |
| G018041 | 66.56 | 2.05 | 0.60 | 0.30 | 0.27 | 0.20 |
| G018042 | 90.79 | 1.10 | 1.28 | 0.47 | 1.64 | 0.35 |
| G018043 | 94.94 | 0.55 | 0.57 | 0.03 | 0.40 | 0.06 |
| G018044 | 91.71 | 1.61 | 0.89 | 0.40 | 1.25 | 0.20 |
| G018045 | 90.26 | 1.59 | 1.46 | 0.54 | 3.91 | 1.89 |
| G018046 | 94.59 | 1.33 | 0.76 | 0.05 | 2.03 | 0.64 |
| G018047 | 93.84 | 1.32 | 0.46 | 0.08 | 2.03 | 0.64 |
| G018048 | 95.20 | 2.58 | 0.54 | 0.52 | 0.62 | 0.30 |
| G018049 | 89.68 | 2.09 | 0.17 | 0.23 | 0.45 | 0.30 |
| G018050 | 95.74 | 0.65 | 0.31 | 0.22 | 0.84 | 0.39 |
| G018051 | 92.47 | 0.90 | 0.54 | 0.76 | 0.73 | 0.31 |
| G018052 | 93.14 | 0.85 | 1.39 | 0.91 | 0.85 | 0.78 |
| G018053 | 78.88 | 3.77 | 0.74 | 0.13 | 0.52 | 0.34 |
| G018054 | 92.69 | 1.11 | 0.79 | 0.10 | 2.55 | 0.46 |
| G018055 | 89.68 | 1.52 | 0.48 | 0.57 | 1.05 | 0.36 |
| G018056 | 94.00 | 1.10 | 0.45 | 0.14 | 1.85 | 0.99 |
| G018057 | 89.87 | 0.49 | 0.72 | 0.04 | 1.31 | 0.31 |
| G018058 | 91.04 | 0.77 | 0.59 | 0.12 | 1.46 | 0.28 |
| G018059 | 49.64 | 3.56 | 0.46 | 0.19 | 1.56 | 0.43 |
| G018060 | 97.65 | 0.65 | 1.22 | 0.17 | 1.13 | 0.47 |
| G018061 | 92.52 | 0.29 | 0.89 | 0.33 | 2.76 | 0.52 |
| G018062 | 88.19 | 0.72 | 0.84 | 0.18 | 6.65 | 0.41 |
| G018063 | 92.94 | 0.63 | 1.54 | 0.20 | 0.94 | 0.38 |
| G018064 | 94.14 | 1.12 | 1.38 | 0.31 | 0.97 | 0.09 |
| G018065 | 93.20 | 1.50 | 1.32 | 0.57 | 0.91 | 0.23 |
| G018066 | 91.62 | 0.45 | 1.69 | 0.06 | 0.45 | 0.17 |
| G018067 | 92.72 | 1.43 | 3.06 | 0.48 | 0.42 | 0.15 |
| G018068 | 93.33 | 0.49 | 0.62 | 0.08 | 0.27 | 0.06 |
| G018069 | 93.83 | 0.77 | 0.94 | 0.33 | 0.99 | 0.07 |
| G018070 | 94.61 | 0.57 | 0.59 | 0.08 | 1.33 | 0.35 |
| G018071 | 87.61 | 0.46 | 0.26 | 0.22 | 1.10 | 0.29 |
| G018072 | 92.86 | 0.84 | 1.76 | 0.04 | 0.34 | 0.06 |
| G018073 | 87.03 | 0.52 | 1.48 | 0.33 | 0.40 | 0.15 |
| G018074 | 80.06 | 11.41 | 0.42 | 0.13 | 0.46 | 0.24 |
| G018075 | 92.85 | 0.27 | 0.59 | 0.37 | 0.61 | 0.08 |
| G018076 | 95.88 | 1.59 | 0.56 | 0.38 | 0.61 | 0.30 |
| G018077 | 0.23 | 0.12 | 0.55 | 0.26 | 0.13 | 0.08 |
| G018078 | 94.97 | 1.42 | 0.48 | 0.16 | 0.44 | 0.22 |
| G018079 | 89.45 | 1.79 | 0.38 | 0.26 | 0.81 | 0.47 |
| G018080 | 80.86 | 3.14 | 0.22 | 0.15 | 0.58 | 0.35 |
| G018081 | 93.61 | 0.63 | 0.17 | 0.14 | 0.91 | 0.44 |
| G018082 | 93.15 | 1.21 | 0.16 | 0.07 | 1.80 | 0.50 |
| G018083 | 78.69 | 0.88 | 0.29 | 0.13 | 2.73 | 0.50 |
| G018084 | 81.88 | 0.70 | 0.43 | 0.19 | 1.39 | 0.60 |
| G018085 | 93.17 | 1.26 | 0.44 | 0.16 | 2.62 | 0.43 |
| G018086 | 95.03 | 1.07 | 0.61 | 0.20 | 1.39 | 0.44 |
| G018087 | 94.37 | 1.49 | 1.07 | 0.79 | 1.41 | 0.21 |
| G018088 | 91.62 | 1.31 | 1.01 | 0.43 | 1.91 | 0.99 |
| G018089 | 94.41 | 1.17 | 0.75 | 0.41 | 3.04 | 1.52 |
| G018090 | 93.66 | 0.82 | 0.54 | 0.28 | 3.12 | 0.96 |
| G018091 | 96.82 | 0.00 | 0.60 | 0.00 | 0.77 | 0.00 |
| G018092 | 90.55 | 1.58 | 0.74 | 0.27 | 2.02 | 0.34 |
| G018093 | 94.46 | 0.49 | 1.13 | 0.13 | 0.96 | 0.28 |
| G018094 | 82.55 | 5.17 | 0.55 | 0.11 | 12.35 | 6.02 |
| G018095 | 94.60 | 0.85 | 1.94 | 0.68 | 0.75 | 0.21 |
| G018096 | 54.68 | 0.62 | 1.99 | 0.16 | 0.38 | 0.12 |
| G018097 | 91.89 | 2.73 | 0.96 | 0.89 | 1.01 | 0.80 |
| G018098 | 70.42 | 0.00 | 0.35 | 0.00 | 0.00 | 0.00 |
| G018099 | 96.84 | 0.16 | 0.43 | 0.10 | 0.64 | 0.16 |
| G018100 | 93.24 | 1.08 | 0.77 | 0.20 | 3.07 | 0.06 |
| G018101 | 92.34 | 0.51 | 2.16 | 0.73 | 3.06 | 0.06 |
| G018102 | 91.87 | 0.99 | 0.42 | 0.34 | 0.61 | 0.16 |
| G018103 | 94.50 | 1.22 | 0.59 | 0.22 | 1.25 | 0.53 |
| G018104 | 94.14 | 0.76 | 0.31 | 0.14 | 1.09 | 0.65 |
| G018105 | 95.51 | 0.98 | 0.42 | 0.14 | 0.82 | 0.21 |
| G018106 | 94.77 | 0.90 | 1.12 | 0.18 | 2.15 | 0.28 |
| G018107 | 94.66 | 0.57 | 0.80 | 0.03 | 2.09 | 0.04 |
| G018108 | 94.77 | 1.14 | 1.23 | 0.70 | 1.55 | 0.96 |
| G018109 | 94.40 | 1.37 | 1.06 | 0.91 | 1.80 | 0.67 |
| G018110 | 92.19 | 1.30 | 1.86 | 0.34 | 4.04 | 0.77 |
| G018111 | 91.96 | 3.12 | 0.72 | 0.17 | 5.54 | 3.11 |
| G018112 | 91.54 | 1.34 | 0.80 | 0.15 | 5.69 | 0.44 |
| G018113 | 96.42 | 0.45 | 0.43 | 0.22 | 1.40 | 0.35 |
| G018114 | 95.95 | 0.89 | 0.79 | 0.32 | 0.27 | 0.20 |
| G018115 | 92.89 | 1.00 | 0.59 | 0.16 | 1.10 | 0.40 |
| G018116 | 91.94 | 0.57 | 0.99 | 0.54 | 0.61 | 0.24 |
| G018117 | 95.48 | 1.10 | 0.62 | 0.31 | 0.55 | 0.31 |
| G018118 | 96.20 | 0.38 | 0.43 | 0.31 | 0.46 | 0.17 |
| G018119 | 83.25 | 1.31 | 0.43 | 0.08 | 0.44 | 0.25 |
| G018120 | 97.00 | 0.72 | 0.42 | 0.26 | 0.38 | 0.20 |
| G018121 | 95.42 | 0.58 | 0.50 | 0.13 | 0.42 | 0.15 |

### Example 12: Screening CIITA sgRNAs in Dose-Response with BC22n in T Cells

Select CIITA sgRNAs identified in Example 11 were further assayed for base editing efficacy at multiple guide concentrations in T cells. The potency of each was assayed for genome editing efficacy by NGS or by disruption of surface protein expression of HLA-DR, DP, DQ by flow cytometry.

### 12.1 T Cell Preparation

Healthy human donor apheresis was obtained commercially (Hemacare), and cells were washed and resuspended in in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512), 1× Penicillin-Streptomycin, 1× Glutamax, 10 mM HEPES, 200 U/mL recombinant human interleukin-2 (Peprotech, Cat. 200-02), 5 ng/mL recombinant human interleukin 7 (Peprotech, Cat. 200-07), and 5 ng/mL recombinant human interleukin 15 (Peprotech, Cat. 200-15). T cells were rested in this media for 24 hours, at which time they were activated with T Cell TransAct™ human reagent (Miltenyi, Cat. 130-111-160) added at a 1:100 ratio by volume. T cells were activated for 48 hours prior to electroporation.

12.2 T Cell Editing with RNA Electroporation

Solutions containing mRNAs encoding BC22n (SEQ ID NO: 804 or 805) and UGI (SEQ ID NO: 807 or 808) were prepared in P3 buffer. 100 μM CIITA targeting sgRNAs were removed from their storage plates and denatured for 2 minutes at 95° C. and incubated at room temperature for 5 minutes. Forty-eight hours post activation, T cells were harvested, centrifuged, and resuspended at a concentration of 12.5×10\hat{ }⁶ T cells/mL in P3 electroporation buffer (Lonza). Each sgRNA was serially diluted in ratio of 1:2 in P3 electroporation buffer starting from 60 pmols in a 96-well PCR plate in duplicate. Following dilution, 1×10\hat{ }⁵ T cells, 20 ng/μL of BC22n mRNAs, and 20 ng/μL of UGI mRNA were mixed with sgRNA plate to make the final volume of 20 μL of P3 electroporation buffer. This mix was transferred to 4 corresponding 96-well Nucleofector™ plates and electroporated using the manufacturer's pulse code. Electroporated T cells were immediately rested in 80 μL of CTS Optimizer T cell growth media without cytokines for 15 minutes before being transferred to new flat-bottom 96-well plates containing an additional 80 μL of CTS OpTmizer T cell growth media supplemented with 2× cytokines. The resulting plates were incubated at 37° C. for 7 days. On day 4 post-electroporation, cells were split 1:2 in two U-bottom plates, and one plate was collected for NGS sequencing, while the other plate was replenished with CTS Optimizer fresh media with 1× cytokines. This plate was used for flow cytometry on Day 7.

### 12.3 Flow Cytometry and NGS Sequencing

On day 7 post-editing, T cells were assayed by flow cytometry to determine surface expression of HLA-DR, DP, DQ. Briefly, T cells were incubated for 30 minutes at 4° C. with a mixture of antibodies diluted in cell staining buffer (BioLegend, Cat. No. 420201). Antibodies against CD3 (BioLegend, Cat. No. 317336), CD4 (BioLegend, Cat. No. 317434), CD8 (BioLegend, Cat. No. 301046), and Viakrome (Beckman Coulter, Cat. No. C36628) were diluted at 1:100, and antibodies against HLA II-DR, DP, DQ (BioLegend, Cat. No. 361714) were diluted at 1:50. Cells were subsequently washed, resuspended in 100 μL of cell staining buffer and processed on a Cytoflex flow cytometer (Beckman Coulter). Flow cytometry data was analyzed using the FlowJo software package. T cells were gated based on size, shape, viability, CD8, and HLA-DR, DP, DQ.

Table 21 shows CIITA editing outcomes and the percentage of T cells negative for HLA-DR, DP, DQ in T cells following base editing with BC22n.

TABLE 21
Percent editing and percent of HLA II-DP, DQ, DR negative cells following CIITA
editing with BC22n base editor

| | sgRNA | C > T | | | % HLA II-DR, DP, DQ | | | % CD74 | | |
|---|---|---|---|---|---|---|---|---|---|---|
| | (pmols) | Ave | SD | N | Ave | SD | N | Ave | SD | N |
| G018067 | 60 | 95.58% | 0.35% | 2 | 99.40 | 0.00 | 2 | 91.45 | 1.34 | 2 |
| | 30 | 94.13% | 0.08% | 2 | 98.90 | 0.42 | 2 | 91.75 | 1.63 | 2 |
| | 15 | 91.49% | 0.05% | 2 | 95.95 | 0.21 | 2 | 89.45 | 0.07 | 2 |
| | 7.5 | 77.17% | 1.50% | 2 | 83.95 | 0.92 | 2 | 76.6 | 0.57 | 2 |
| | 3.75 | 56.15% | 1.07% | 2 | 71.00 | 0.71 | 2 | 63.65 | 4.03 | 2 |
| | 1.88 | 32.76% | 0.32% | 2 | 57.65 | 3.46 | 2 | 50.6 | 3.39 | 2 |
| | 0.94 | 17.47% | 0.95% | 2 | 50.85 | 3.04 | 2 | 46.1 | 3.82 | 2 |
| | 0 | 0.41% | 0.01% | 2 | 49.45 | 5.16 | 2 | 47.85 | 1.91 | 2 |
| G018075 | 60 | 96.12% | 1.35% | 2 | 98.25 | 0.07 | 2 | 92 | 1.27 | 2 |
| | 30 | 94.07% | 3.53% | 2 | 97.45 | 1.06 | 2 | 88.65 | 2.76 | 2 |
| | 15 | 92.08% | 0.90% | 2 | 97.10 | 0.14 | 2 | 87.25 | 2.05 | 2 |
| | 7.5 | 83.76% | 3.28% | 2 | 90.65 | 0.21 | 2 | 80.1 | 2.69 | 2 |
| | 3.75 | 67.35% | 2.13% | 2 | 78.85 | 0.21 | 2 | 67.1 | 2.97 | 2 |
| | 1.88 | 42.66% | 2.51% | 2 | 63.65 | 0.07 | 2 | 52.55 | 2.33 | 2 |
| | 0.94 | 25.54% | 0.32% | 2 | 56.55 | 3.75 | 2 | 43.1 | 3.25 | 2 |
| | 0 | 0.23% | 0.02% | 2 | 48.70 | 1.84 | 2 | 42.1 | 0.42 | 2 |
| G018091 | 60 | 97.80% | ND | 2 | 99.65 | 0.21 | 2 | 92.2 | 0.57 | 2 |
| | 30 | 96.32% | 0.91% | 2 | 97.50 | 0.28 | 2 | 88.7 | 1.7 | 2 |
| | 15 | 84.48% | 2.73% | 2 | 91.90 | 0.71 | 2 | 82.1 | 0.71 | 2 |
| | 7.5 | 76.56% | 0.49% | 2 | 78.05 | 1.63 | 2 | 64.15 | 0.35 | 2 |
| | 3.75 | 52.08% | 1.50% | 2 | 67.00 | 1.56 | 2 | 46.8 | 1.84 | 2 |
| | 1.88 | 30.00% | 2.36% | 2 | 58.80 | 2.55 | 2 | 40.7 | 2.4 | 2 |
| | 0.94 | 15.01% | 1.12% | 2 | 50.70 | 2.26 | 2 | 33 | 2.83 | 2 |
| | 0 | 0.00% | 0.00% | 2 | 47.90 | 1.70 | 2 | 39 | 0 | 2 |
| G018100 | 60 | 95.55% | 0.47% | 2 | 94.30 | 0.57 | 2 | 85.3 | 2.69 | 2 |
| | 30 | 95.22% | 1.02% | 2 | 91.75 | 0.78 | 2 | 79.1 | 0.85 | 2 |
| | 15 | 93.33% | 0.49% | 2 | 90.80 | 0.99 | 2 | 77.1 | 0.28 | 2 |
| | 7.5 | 88.77% | 0.15% | 2 | 84.40 | 0.85 | 2 | 69.9 | 1.56 | 2 |
| | 3.75 | 71.03% | 4.26% | 2 | 75.20 | 2.40 | 2 | 54.4 | 0.57 | 2 |
| | 1.88 | 46.87% | 1.95% | 2 | 63.80 | 0.28 | 2 | 45.7 | 0.28 | 2 |
| | 0.94 | 27.20% | 0.21% | 2 | 54.80 | 1.56 | 2 | 36.5 | 2.4 | 2 |
| | 0 | 0.17% | 0.04% | 2 | 52.25 | 1.63 | 2 | 42.8 | 0 | 2 |
| G018102 | 60 | 93.32% | 0.10% | 2 | 93.00 | 0.14 | 2 | 84.65 | 0.64 | 2 |
| | 30 | 77.08% | 2.22% | 2 | 80.40 | 1.84 | 2 | 68.7 | 2.55 | 2 |
| | 15 | 52.42% | 1.28% | 2 | 70.50 | 0.28 | 2 | 55.4 | 0.85 | 2 |
| | 7.5 | 31.87% | 0.20% | 2 | 58.80 | 4.53 | 2 | 39.4 | 4.38 | 2 |
| | 3.75 | 16.76% | 0.26% | 2 | 56.85 | 1.63 | 2 | 33.65 | 0.07 | 2 |
| | 1.88 | 8.18% | 0.06% | 2 | 54.15 | 0.21 | 2 | 30.35 | 0.92 | 2 |
| | 0.94 | 4.12% | 0.66% | 2 | 52.25 | 3.32 | 2 | 32.2 | 0.57 | 2 |
| | 0 | 0.28% | 0.12% | 2 | 50.00 | 3.68 | 2 | 42.45 | 4.17 | 2 |
| G018103 | 60 | 96.34% | 0.21% | 2 | 98.85 | 0.07 | 2 | 89.2 | 2.12 | 2 |
| | 30 | 90.93% | 0.17% | 2 | 95.15 | 0.92 | 2 | 84.95 | 0.07 | 2 |
| | 15 | 80.01% | 0.34% | 2 | 87.95 | 1.63 | 2 | 76.7 | 0.14 | 2 |
| | 7.5 | 59.92% | 0.48% | 2 | 70.90 | 2.97 | 2 | 56.45 | 3.18 | 2 |
| | 3.75 | 36.07% | 1.23% | 2 | 62.05 | 2.33 | 2 | 44.65 | 0.49 | 2 |
| | 1.88 | 19.59% | 2.15% | 2 | 58.65 | 2.76 | 2 | 38.2 | 1.41 | 2 |
| | 0.94 | 7.98% | 0.08% | 2 | 52.75 | 0.07 | 2 | 33.25 | 2.62 | 2 |
| | 0 | 0.22% | 0.03% | 2 | 50.95 | 0.64 | 2 | 43.1 | 2.69 | 2 |
| G018107 | 60 | ND | ND | 2 | 99.50 | 0.14 | 2 | 89.05 | 0.07 | 2 |
| | 30 | ND | ND | 2 | 99.05 | 0.07 | 2 | 91.1 | 0.85 | 2 |
| | 15 | ND | ND | 2 | 97.05 | 0.64 | 2 | 90 | 3.25 | 2 |
| | 7.5 | ND | ND | 2 | 90.45 | 0.07 | 2 | 83.25 | 2.76 | 2 |
| | 3.75 | ND | ND | 2 | 79.60 | 0.99 | 2 | 72.3 | 2.4 | 2 |
| | 1.88 | ND | ND | 2 | 66.20 | 2.26 | 2 | 58.7 | 4.81 | 2 |
| | 0.94 | ND | ND | 2 | 53.40 | 1.41 | 2 | 45.55 | 3.89 | 2 |
| | 0 | ND | ND | 2 | 51.65 | 2.05 | 2 | 47.35 | 4.03 | 2 |
| G018111 | 60 | 96.87% | 0.26% | 2 | 95.65 | 0.49 | 2 | 92.45 | 0.35 | 2 |
| | 30 | 95.44% | 0.07% | 2 | 89.40 | 0.71 | 2 | 86.5 | 0.42 | 2 |
| | 15 | 89.11% | 0.91% | 2 | 81.35 | 0.07 | 2 | 75.75 | 2.62 | 2 |
| | 7.5 | 71.93% | 0.55% | 2 | 70.05 | 3.32 | 2 | 61.15 | 0.21 | 2 |
| | 3.75 | 47.48% | 0.33% | 2 | 56.45 | 5.02 | 2 | 48.5 | 0.14 | 2 |
| | 1.88 | 25.72% | 1.46% | 2 | 59.00 | 3.11 | 2 | 44.95 | 0.07 | 2 |
| | 0.94 | 12.27% | 0.79% | 2 | 51.30 | 3.39 | 2 | 42.3 | 0.99 | 2 |
| | 0 | 0.15% | 0.01% | 2 | 51.00 | 3.25 | 2 | 45.1 | 1.41 | 2 |
| G018116 | 60 | 86.65% | 0.01% | 2 | 92.95 | 0.35 | 2 | 87.7 | 0.42 | 2 |
| | 30 | 62.59% | 0.13% | 2 | 79.90 | 1.84 | 2 | 69.75 | 2.9 | 2 |
| | 15 | 40.44% | 1.09% | 2 | 69.90 | 0.71 | 2 | 60.15 | 1.34 | 2 |
| | 7.5 | 23.19% | 2.46% | 2 | 60.35 | 4.74 | 2 | 45.45 | 0.92 | 2 |
| | 3.75 | 10.65% | 0.83% | 2 | 55.45 | 1.91 | 2 | 40.2 | 1.41 | 2 |
| | 1.88 | 5.63% | 0.47% | 2 | 56.85 | 1.06 | 2 | 40.75 | 0.64 | 2 |
| | 0.94 | 1.93% | 0.33% | 2 | 57.80 | 3.96 | 2 | 40 | 0.14 | 2 |
| | 0 | 0.19% | 0.10% | 2 | 50.55 | 0.21 | 2 | 47 | 1.41 | 2 |
| G018117 | 60 | 98.01% | 0.17% | 2 | 99.65 | 0.07 | 2 | 93.65 | 0.49 | 2 |
| | 30 | 97.14% | 0.69% | 2 | 99.20 | 0.14 | 2 | 92.35 | 0.07 | 2 |
| | 15 | 93.65% | 0.39% | 2 | 97.75 | 0.64 | 2 | 91.55 | 0.21 | 2 |
| | 7.5 | 81.77% | 0.50% | 2 | 91.60 | 0.42 | 2 | 81.85 | 0.35 | 2 |
| | 3.75 | 56.56% | 2.70% | 2 | 80.55 | 3.46 | 2 | 69.25 | 3.32 | 2 |
| | 1.88 | 33.70% | 3.00% | 2 | 69.30 | 1.13 | 2 | 56.8 | 2.69 | 2 |
| | 0.94 | 17.14% | 0.83% | 2 | 60.40 | 1.84 | 2 | 48.25 | 1.34 | 2 |
| | 0 | 0.18% | 0.01% | 2 | 51.95 | 2.76 | 2 | 46.5 | 1.13 | 2 |
| G018118 | 60 | 98.36% | 0.27% | 2 | 99.65 | 0.07 | 2 | 94.35 | 0.49 | 2 |
| | 30 | 97.70% | 0.19% | 2 | 99.45 | 0.21 | 2 | 88.35 | 8.13 | 2 |
| | 15 | 94.59% | 1.64% | 2 | 98.30 | 0.14 | 2 | 90.45 | 0.21 | 2 |
| | 7.5 | 83.77% | 2.47% | 2 | 92.80 | 1.13 | 2 | 81.2 | 0.57 | 2 |
| | 3.75 | 64.07% | 0.54% | 2 | 81.25 | 1.06 | 2 | 71.5 | 1.56 | 2 |
| | 1.88 | 40.72% | 2.16% | 2 | 70.65 | 2.90 | 2 | 61.15 | 3.18 | 2 |
| | 0.94 | 22.33% | 3.64% | 2 | 63.90 | 0.85 | 2 | 51.05 | 2.76 | 2 |
| | 0 | 0.14% | 0.02% | 2 | 52.95 | 1.48 | 2 | 47.85 | 1.77 | 2 |
| G018120 | 60 | 98.22% | 0.32% | 2 | 99.10 | 0.14 | 2 | 91.65 | 0.78 | 2 |
| | 30 | 96.96% | 0.43% | 2 | 99.15 | 0.07 | 2 | 89.1 | 0.57 | 2 |
| | 15 | 93.71% | 0.19% | 2 | 97.05 | 0.21 | 2 | 88.15 | 0.64 | 2 |
| | 7.5 | 82.19% | 1.50% | 2 | 90.25 | 0.78 | 2 | 80 | 0.99 | 2 |
| | 3.75 | 61.25% | 1.50% | 2 | 80.30 | 1.56 | 2 | 66.45 | 1.77 | 2 |
| | 1.88 | 37.98% | 1.29% | 2 | 68.85 | 0.78 | 2 | 52.75 | 3.04 | 2 |
| | 0.94 | 18.96% | 0.67% | 2 | 59.85 | 0.64 | 2 | 47.25 | 2.19 | 2 |
| | 0 | 0.17% | 0.02% | 2 | 49.60 | 0.57 | 2 | 46.1 | 0.99 | 2 |
| G018121 | 60 | 97.55% | 0.01% | 2 | 97.25 | 0.35 | 2 | 87.45 | 0.35 | 2 |
| | 30 | 92.24% | 0.98% | 2 | 92.50 | 0.71 | 2 | 83.6 | 3.96 | 2 |
| | 15 | 78.58% | 0.52% | 2 | 84.90 | 0.28 | 2 | 76.85 | 2.9 | 2 |
| | 7.5 | 57.37% | 0.50% | 2 | 73.75 | 3.04 | 2 | 64.5 | 3.25 | 2 |
| | 3.75 | 35.06% | 0.53% | 2 | 60.70 | 0.00 | 2 | 50.5 | 3.25 | 2 |
| | 1.88 | 18.80% | 2.81% | 2 | 61.00 | 1.41 | 2 | 45.95 | 4.31 | 2 |
| | 0.94 | 9.25% | 0.17% | 2 | 56.95 | 4.31 | 2 | 42.8 | 1.7 | 2 |
| | 0 | 0.21% | 0.00% | 2 | 47.45 | 3.04 | 2 | 44.55 | 1.34 | 2 |

### Example 13. Off-Target Analysis of CIITA Splice Guides

T Cells from Example 10 were screened for validation of off-target genomic sites targeting CIITA and was performed according to the Integrated DNA Technologies, IDT rhAmpSeq rhPCR Protocol. In this experiment, 3 sgRNA targeting CIITA were screened for validation of off-target profiles. The number of validated off-target sites for sgRNAs targeting CIITA guides (G018082, G018081, and G018034) were shown in Table 22. Off-target sites were validated if the p value was less than 0.05 percent indel. Of the 108 off-target sites identified for the sgRNA targeting G018082, 0 sites were validated. Of the 111 off-target sites identified for the sgRNA targeting G018081, 3 sites were validated. Of the 120 off-target sites identified for the sgRNA targeting G018034, 0 sites were validated.

TABLE 22
Off-Target Site Validation of CIITA Splice Guides

| | | | (SEQ | Off- | Sites |
|---|---|---|---|---|---|
| gRNA | | Guide | ID | Target | Vali- |
| ID | Target | Sequence | NO) | Sites | dated |
| G018082 | CIITA | UGUGUCACCC | 62 | 108 | 0 |
| | | GUUUCAGGUG | | | |
| G018081 | CIITA | CUGUGUCACC | 61 | 111 | 3 |
| | | CGUUUCAGGU | | | |
| G018034 | CIITA | AGGGAGGCUU | 14 | 120 | 0 |
| | | AUGCCAAUAU | | | |

### Additional Embodiments

The disclosure further includes the following embodiments.

Embodiment 1 is an engineered cell, which has reduced or eliminated surface expression of MHC class II relative to an unmodified cell, comprising a genetic modification in the CIITA gene, wherein the genetic modification comprises at least one nucleotide of a splice site within the genomic coordinates chr16:10902171-10923242.

Embodiment 2 is the engineered cell of embodiment 1, wherein the genetic modification comprises a modification of at least one nucleotide of a splice acceptor site.

Embodiment 3 is the engineered cell of embodiment 2, wherein the one nucleotide is A.

Embodiment 4 is the engineered cell of embodiment 2, wherein the one nucleotide is G.

Embodiment 5 is the engineered cell of embodiment 1, wherein the genetic modification comprises a modification of at least one nucleotide of a splice donor site.

Embodiment 6 is the engineered cell of embodiment 5, wherein the one nucleotide is G.

Embodiment 7 is the engineered cell of embodiment 5, wherein the one nucleotide is T.

Embodiment 8 is the engineered cell of any one of the preceding embodiments, wherein the genetic modification comprises a modification of a splice site boundary nucleotide.

Embodiment 9 is the engineered cell of any one of the preceding embodiments, wherein the genetic modification comprises at least 5 contiguous nucleotides within the genomic coordinates chr16:10902171-10923242.

Embodiment 10 is the engineered cell of any one of the preceding embodiments, wherein the genetic modification comprises at least 6, 7, 8, 9, or 10 contiguous nucleotides within the genomic coordinates chr16:10902171-10923242.

Embodiment 11 is the engineered cell of any one of the preceding embodiments, wherein the genetic modification comprises at least one C to T substitution or at least one A to G substitution within the genomic coordinates chr16:10902171-10923242.

Embodiment 12 is the engineered cell of embodiment 1, wherein the genetic modification comprises at least one nucleotide of a splice site within the genomic coordinates chr16:10903873-10923242.

Embodiment 13 is the engineered cell of any one of the preceding embodiments, wherein the genetic modification comprises at least one nucleotide of a splice site within the genomic coordinates chr:16:10906485-10923242.

Embodiment 14 is the engineered cell of any one of the preceding embodiments, wherein the genetic modification comprises at least one nucleotide of a splice site within the genomic coordinates chr16:10908130-10923242.

Embodiment 15 is the engineered cell of any one of the preceding embodiments, wherein the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150.

Embodiment 16 is the engineered cell of any one of the preceding embodiments, wherein the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, and chr16:10922478-10922498.

Embodiment 17 is the engineered cell of any one of the preceding embodiments, wherein the genetic modification comprises at least one nucleotide of a splice site within the genomic coordinates chosen from: chr16:10908132-10908152, chr16:10908131-10908151, chr16:10916456-10916476, and chr16:10918504-10918524.

Embodiment 18 is the engineered cell of any one of the preceding embodiments, wherein the genetic modification comprises at least one nucleotide of a splice site within the genomic coordinates chr16:10908132-10908152.

Embodiment 19 is the engineered cell of any one of the preceding embodiments, wherein the genetic modification comprises at least one nucleotide of a splice site within the genomic coordinates chr16:10908131-10908151.

Embodiment 20 is the engineered cell of any one of the preceding embodiments, wherein the genetic modification comprises at least one nucleotide of a splice site within the genomic coordinates chr16:10916456-10916476.

Embodiment 21 is the engineered cell of any one of the preceding embodiments, wherein the genetic modification comprises at least one nucleotide of a splice site within the genomic coordinates chr16:10918504-10918524.

Embodiment 22 is the engineered cell of any one of the preceding embodiments, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-10906507.

Embodiment 23 is the engineered cell of any one of the preceding embodiments, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, and chr16:10922153-10922173.

Embodiment 24 is the engineered cell of any one of the preceding embodiments, wherein the genetic modification comprises at least one nucleotide of a splice site within the genomic coordinates chosen from: chr16:10918504-10918524, chr16:10923218-10923238, and chr16:10923219-10923239.

Embodiment 25 is the engineered cell of any one of the preceding embodiments, wherein the genetic modification comprises at least one nucleotide of a splice site within the genomic coordinates chr16:10918504-10918524.

Embodiment 26 is the engineered cell of any one of the preceding embodiments, wherein the genetic modification comprises at least one nucleotide of a splice site within the genomic coordinates chr16:10923218-10923238.

Embodiment 27 is the engineered cell of any one of the preceding embodiments, wherein the genetic modification comprises at least one nucleotide of a splice site within the genomic coordinates chr16:10923219-10923239.

Embodiment 28 is an engineered cell, which has reduced or eliminated surface expression of MHC class II relative to an unmodified cell, comprising a genetic modification in the CIITA gene, wherein the genetic modification comprises an indel, a C to T substitution, or an A to G substitution within the genomic coordinates chosen from: chr16:10895410-10895430, chr16:10898649-10898669, chr16:10898658-10898678, chr16:10902171-10902191, chr16:10902173-10902193, chr16:10902174-10902194, chr16:10902179-10902199, chr16:10902183-10902203, chr16:10902184-10902204, chr16:10902644-10902664, chr16:10902779-10902799, chr16:10902788-10902808, chr16:10902789-10902809, chr16:10902790-10902810, chr16:10902795-10902815, chr16:10902799-10902819, chr16:10903708-10903728, chr16:10903713-10903733, chr16:10903718-10903738, chr16:10903721-10903741, chr16:10903723-10903743, chr16:10903724-10903744, chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242.

Embodiment 29 is the engineered cell of embodiment 28, wherein the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150.

Embodiment 30 is the engineered cell of embodiment 28, wherein the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, and chr16:10922478-10922498.

Embodiment 31 is the engineered cell of embodiment 28, wherein the genetic modification comprises at least one nucleotide of a splice site within the genomic coordinates chosen from: chr16:10908132-10908152, chr16:10908131-10908151, chr16:10916456-10916476, and chr16:10918504-10918524.

Embodiment 32 is the engineered cell of embodiment 28, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-10906507.

Embodiment 33 is the engineered cell of embodiment 28, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, and chr16:10922153-10922173.

Embodiment 34 is the engineered cell of embodiment 28, wherein the genetic modification comprises at least one nucleotide of a splice site within the genomic coordinates chosen from: chr16:10918504-10918524, chr16:10923218-10923238, and chr16:10923219-10923239.

Embodiment 35 is the engineered cell of any one of embodiments 28-34, wherein the genetic modification comprises at least 5 contiguous nucleotides within the genomic coordinates.

Embodiment 36 is the engineered cell of any one of embodiments 28-35, wherein the genetic modification comprises at least 6, 7, 8, 9, or 10 contiguous nucleotides within the genomic coordinates.

Embodiment 37 is the engineered cell of any one of embodiments 28-36, wherein the genetic modification comprises at least one C to T substitution or at least one A to G substitution within the genomic coordinates.

Embodiment 38 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10895410-10895430, chr16:10898649-10898669, chr16:10898658-10898678, chr16:10902171-10902191, chr16:10902173-10902193, chr16:10902174-10902194, chr16:10902179-10902199, chr16:10902183-10902203, chr16:10902184-10902204, chr16:10902644-10902664, chr16:10902779-10902799, chr16:10902788-10902808, chr16:10902789-10902809, chr16:10902790-10902810, chr16:10902795-10902815, chr16:10902799-10902819, chr16:10903708-10903728, chr16:10903713-10903733, chr16:10903718-10903738, chr16:10903721-10903741, chr16:10903723-10903743, chr16:10903724-10903744, chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242.

Embodiment 39 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242.

Embodiment 40 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242.

Embodiment 41 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242.

Embodiment 42 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150.

Embodiment 43 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, and chr16:10922478-10922498.

Embodiment 44 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10908132-10908152, chr16:10908131-10908151, chr16:10916456-10916476, and chr16:10918504-10918524.

Embodiment 45 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10908132-10908152.

Embodiment 46 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10908131-10908151.

Embodiment 47 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10916456-10916476.

Embodiment 48 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10918504-10918524.

Embodiment 49 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-10906507.

Embodiment 50 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, and chr16:10922153-10922173.

Embodiment 51 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chosen from: chr16:10918504-10918524, chr16:10923218-10923238, and chr16:10923219-10923239.

Embodiment 52 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10918504-10918524.

Embodiment 53 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10923218-10923238.

Embodiment 54 is the engineered cell of any one of the preceding embodiments, wherein the MHC class II expression is reduced or eliminated by a gene editing system that binds to a CIITA genomic target sequence comprising at least 5 contiguous nucleotides within the genomic coordinates chr16:10923219-10923239.

Embodiment 55 is the engineered cell of any one of embodiments 38-54, wherein the CIITA genomic target sequence comprises at least 10 contiguous nucleotides within the genomic coordinates.

Embodiment 56 is the engineered cell of any one of embodiments 38-55, wherein the CIITA genomic target sequence comprises at least 15 contiguous nucleotides within the genomic coordinates.

Embodiment 57 is the engineered cell of any one of embodiments 38-56, wherein the gene editing system comprises an RNA-guided DNA-binding agent.

Embodiment 58 is the engineered cell of embodiment 57, wherein the RNA-guided DNA-binding agent comprises a Cas9 protein, such as an S. pyogenes Cas9.

Embodiment 59 is the engineered cell of any one of the preceding embodiments, wherein the genetic modification inactivates a splice site.

Embodiment 60 is the engineered cell of any one of the preceding embodiments, wherein the genetic modification comprises a deletion at a splice site nucleotide.

Embodiment 61 is the engineered cell of any one of the preceding embodiments, wherein the genetic modification comprises a substitution at a splice site nucleotide.

Embodiment 62 is the engineered cell of any one of the preceding embodiments, wherein the engineered cell further has reduced or eliminated surface expression of MHC class I.

Embodiment 63 is the engineered cell of any one of the preceding embodiments, wherein the engineered cell comprises a genetic modification in the beta-2-microglobulin (B2M) gene.

Embodiment 64 is the engineered cell of any one of the preceding embodiments, wherein the engineered cell comprises a genetic modification in an HLA-A gene.

Embodiment 65 is the engineered cell of any one of the preceding embodiments, wherein the engineered cell further comprises an exogenous nucleic acid.

Embodiment 66 is the engineered cell of any one of the preceding embodiments, wherein the engineered cell comprises an exogenous nucleic acid encoding a targeting receptor that is expressed on the surface of the engineered cell.

Embodiment 67 is the engineered cell of embodiment 66, wherein the targeting receptor is a CAR.

Embodiment 68 is the engineered cell of embodiment 66, wherein the targeting receptor is a TCR.

Embodiment 69 is the engineered cell of embodiment 66, wherein the targeting receptor is a WT1 TCR.

Embodiment 70 is the engineered cell of any one of the preceding embodiments, wherein the engineered cell further comprises an exogenous nucleic acid encoding a polypeptide that is secreted by the engineered cell.

Embodiment 71 is the engineered cell of any one of the preceding embodiments, wherein the engineered cell is a human cell.

Embodiment 72 is the engineered cell of any one of the preceding embodiments, wherein the engineered cell is an immune cell.

Embodiment 73 is the engineered cell of any one of the preceding embodiments, wherein the engineered cell is a monocyte, macrophage, mast cell, dendritic cell, or granulocyte.

Embodiment 74 is the engineered cell of any one of the preceding embodiments, wherein the engineered cell is a lymphocyte.

Embodiment 75 is the engineered cell of embodiment 74, wherein the engineered cell is a T cell.

Embodiment 76 is the engineered cell of embodiment 75, wherein the engineered cell further has reduced or eliminated expression of an endogenous T-cell receptor (TCR) protein relative to an unmodified cell.

Embodiment 77 is the engineered cell of embodiment 76, wherein the cell has reduced or eliminated expression of a TRAC protein relative to an unmodified cell.

Embodiment 78 is the engineered cell of any one of embodiments 76-77, wherein the cell has reduced expression of a TRBC protein relative to an unmodified cell.

Embodiment 79 is a pharmaceutical composition comprising the engineered cell of any one of the preceding embodiments.

Embodiment 80 is a population of cells comprising the engineered cell of any one of the preceding embodiments.

Embodiment 81 is a pharmaceutical composition comprising a population of cells, wherein the population of cells comprises an engineered cell of any one of the preceding embodiments.

Embodiment 82 is the population of cells of embodiment 80 or pharmaceutical composition of embodiment 81, wherein the population of cells is at least 65% MHC class II negative as measured by flow cytometry.

Embodiment 83 is the population of cells of embodiment 80 or pharmaceutical composition of embodiment 81, wherein the population of cells is at least 70% MHC class II negative as measured by flow cytometry.

Embodiment 84 is the population of cells of embodiment 80 or pharmaceutical composition of embodiment 81, wherein the population of cells is at least 80% MHC class II negative as measured by flow cytometry.

Embodiment 85 is the population of cells of embodiment 80 or pharmaceutical composition of embodiment 81, wherein the population of cells is at least 90% MHC class II negative as measured by flow cytometry.

Embodiment 86 is the population of cells of embodiment 80 or pharmaceutical composition of embodiment 81, wherein the population of cells is at least 92% MHC class II negative as measured by flow cytometry.

Embodiment 87 is the population of cells of embodiment 80 or pharmaceutical composition of embodiment 81, wherein the population of cells is at least 93% MHC class II negative as measured by flow cytometry.

Embodiment 88 is the population of cells of embodiment 80 or pharmaceutical composition of embodiment 81, wherein the population of cells is at least 94% MHC class II negative as measured by flow cytometry.

Embodiment 89 is the population of cells or pharmaceutical composition of any of embodiment 80-88, wherein the population of cells is at least 95% endogenous TCR protein negative as measured by flow cytometry.

Embodiment 90 is the population of cells or pharmaceutical composition of any of embodiment 80-89, wherein the population of cells is at least 97% endogenous TCR protein negative as measured by flow cytometry.

Embodiment 91 is the population of cells or pharmaceutical composition of any of embodiment 80-90, wherein the population of cells is at least 98% endogenous TCR protein negative as measured by flow cytometry.

Embodiment 92 is the population of cells or pharmaceutical composition of any of embodiment 80-91, wherein the population of cells is at least 99% endogenous TCR protein negative as measured by flow cytometry.

Embodiment 93 is a method of administering the engineered cell, population of cells, or pharmaceutical composition of any one of the preceding embodiments to a subject in need thereof.

Embodiment 94 is a method of administering the engineered cell, population of cells, or pharmaceutical composition of any one of the preceding embodiments to a subject as an adoptive cell transfer (ACT) therapy.

Embodiment 95 is a composition comprising: a) a CIITA guide RNA comprising a guide sequence that i) targets a CIITA genomic target sequence that comprises at least one nucleotide of a splice site, or ii) directs an RNA-guided DNA binding agent to make a cut in a CIITA genomic target sequence that is 5 nucleotides or less from a splice site boundary nucleotide; wherein the CIITA guide RNA targets a CIITA genomic target sequence comprising at least 10 contiguous nucleotides within the genomic coordinates chr16:10902171-10923242.

Embodiment 96 is a composition comprising: a) a CIITA guide RNA (gRNA) comprising i) a guide sequence selected from SEQ ID NOs: 1-101; or ii) at least 17, 18, 19, or 20 contiguous nucleotides of a sequence selected from SEQ ID NOs: 1-101; or iii) a guide sequence at least 95%, 90%, or 85% identical to a sequence selected from SEQ ID NOs: 1-101; or iv) a sequence that comprises 10 contiguous nucleotides ±10 nucleotides of a genomic coordinate listed in Table 1; or v) at least 17, 18, 19, or 20 contiguous nucleotides of a sequence from (iv); or vi) a guide sequence that is at least 95%, 90%, or 85% identical to a sequence selected from (v).

Embodiment 97 is a composition comprising: a) a CIITA guide RNA that is a single-guide RNA (sgRNA) comprising i) a guide sequence selected from SEQ ID NOs: 1-101; or ii) at least 17, 18, 19, or 20 contiguous nucleotides of a sequence selected from SEQ ID NOs: 1-101; or iii) a guide sequence at least 95%, 90%, or 85% identical to a sequence selected from SEQ ID NOs: 1-101; or iv) a sequence that comprises 10 contiguous nucleotides ±10 nucleotides of a genomic coordinate listed in Table 1; or v) at least 17, 18, 19, or 20 contiguous nucleotides of a sequence from (iv); or vi) a guide sequence that is at least 95%, 90%, or 85% identical to a sequence selected from (v).

Embodiment 98 is the composition of any one of embodiments 95-97, wherein the CIITA guide RNA is an S. pyogenes Cas9 guide RNA.

Embodiment 99 is the composition of any one of embodiments 95-98, further comprising an RNA-guided DNA binding agent or nucleic acid encoding an RNA-guided DNA binding agent.

Embodiment 100 is the composition of embodiment 99, wherein the nucleic acid encoding an RNA-guided DNA binding agent is an mRNA that encoding the RNA-guided DNA binding agent.

Embodiment 101 is the composition of any one of embodiments 99-100, wherein the RNA-guided DNA binding agent comprises an S. pyogenes Cas9.

Embodiment 102 is the composition of any one of embodiments 99-101, wherein the RNA-guided DNA binding agent comprises a deaminase region.

Embodiment 103 is the composition of any one of embodiments 99-101, wherein the RNA-guided DNA binding agent comprises an APOBEC3A deaminase (A3A) and an RNA-guided nickase.

Embodiment 104 is the composition of embodiment 103, wherein the RNA-guided nickase is an S. pyogenes Cas9 nickase.

Embodiment 105 is the composition of any one of embodiments 102-104, further comprising a uracil glycosylase inhibitor (UGI).

Embodiment 106 is the composition of any one of embodiments 102-105, wherein the RNA-guided DNA binding agent generates a cytosine (C) to thymine (T) conversion with the CIITA genomic target sequence.

Embodiment 107 is the composition of any one of embodiments 102-105, wherein the RNA-guided DNA binding agent generates an adenine (A) to guanine (G) conversion with the CIITA genomic target sequence.

Embodiment 108 is the composition of any one of embodiments 99-107, wherein the CIITA guide RNA targets a CIITA genomic target sequence that comprises at least one nucleotide of a splice acceptor site.

Embodiment 109 is the composition of embodiment 108, wherein the one nucleotide is A.

Embodiment 110 is the composition of embodiment 108, wherein the one nucleotide is G.

Embodiment 111 is the composition of embodiment 108, wherein the one nucleotide is the splice site boundary nucleotide at the splice acceptor site.

Embodiment 112 is the composition of any one of embodiments 99-107, wherein the CIITA guide RNA targets a CIITA genomic target sequence that comprises at least one nucleotide of a splice donor site.

Embodiment 113 is the composition of embodiment 112, wherein the one nucleotide is G.

Embodiment 114 is the composition of embodiment 112, wherein the one nucleotide is T.

Embodiment 115 is the composition of embodiment 112, wherein the one nucleotide is the splice site boundary nucleotide at the splice donor site.

Embodiment 116 is the composition of any one of embodiments 99-115, wherein the CIITA guide RNA comprises a guide sequence that directs an RNA-guided DNA binding agent to make a cut in a CIITA genomic target sequence that is 4 nucleotides or less from a splice site boundary nucleotide.

Embodiment 117 is the composition of any one of embodiments 99-115, wherein the CIITA guide RNA comprises a guide sequence that directs an RNA-guided DNA binding agent to make a cut in a CIITA genomic target sequence that is 3 nucleotides or less from a splice site boundary nucleotide.

Embodiment 118 is the composition of any one of embodiments 99-117, wherein the CIITA guide RNA comprises a guide sequence that directs an RNA-guided DNA binding agent to make a cut in a CIITA genomic target sequence that is 2 nucleotides or less from a splice site boundary nucleotide.

Embodiment 119 is the composition of any one of embodiments 99-118, wherein the CIITA guide RNA comprises a guide sequence that directs an RNA-guided DNA binding agent to make a cut in a CIITA genomic target sequence that is 1 nucleotide or less from a splice site boundary nucleotide.

Embodiment 120 is the composition of any one of embodiments 99-119, wherein the CIITA guide RNA comprises a guide sequence that directs an RNA-guided DNA binding agent to make a cut in a CIITA genomic target sequence at a splice site boundary nucleotide.

Embodiment 121 is a method of making an engineered cell, which has reduced or eliminated surface expression of MHC class II protein relative to an unmodified cell, comprising contacting a cell with a composition of any of embodiments 99-120.

Embodiment 122 is a method of reducing surface expression of MHC class II protein in an engineered cell relative to an unmodified cell, comprising contacting a cell with a composition of any of embodiments 99-120.

Embodiment 123 is the method of any one of embodiments 121-122, further comprising reducing or eliminating the surface expression of MHC class I protein in the cell relative to an unmodified cell.

Embodiment 124 is the method of any one of embodiments 121-122, further comprising reducing or eliminating the surface expression of B2M protein in the cell relative to an unmodified cell.

Embodiment 125 is the method of any one of embodiments 121-122, further comprising reducing or eliminating the surface expression of HLA-A protein in the cell relative to an unmodified cell.

Embodiment 126 is the method of any one of embodiments 122-125, further comprising reducing or eliminating the surface expression of a TCR protein in the cell relative to an unmodified cell.

Embodiment 127 is the method of any one of embodiments 122-126, further comprising contacting the cell with an exogenous nucleic acid.

Embodiment 128 is the method of embodiment 127, further comprising contacting the cell with an exogenous nucleic acid encoding a targeting receptor.

Embodiment 129 is the method of embodiment 127, further comprising contacting the cell with an exogenous nucleic acid encoding a polypeptide that is secreted by the cell.

Embodiment 130 is the engineered cell, population of cells, pharmaceutical composition, or method of any one of the preceding embodiments, wherein the cell is an allogeneic cell.

Embodiment 131 is the engineered cell, population of cells, pharmaceutical composition, or method of any one of the preceding embodiments, wherein the cell is a primary cell.

Embodiment 132 is the engineered cell, population of cells, pharmaceutical composition, or method of any one of the preceding embodiments, wherein the cell is a CD4+ T cell.

Embodiment 133 is the engineered cell, population of cells, pharmaceutical composition, or method of any one of the preceding embodiments, wherein the cell is a CD8+ T cell.

Embodiment 134 is the engineered cell, population of cells, pharmaceutical composition, or method of any one of the preceding embodiments, wherein the cell is a memory T cell.

Embodiment 135 is the engineered cell, population of cells, pharmaceutical composition, or method of any one of the preceding embodiments, wherein the cell is a B cell.

Embodiment 136 is the engineered cell, population of cells, pharmaceutical composition, or method of any one of the preceding embodiments, wherein the cell is a plasma B cell.

Embodiment 137 is the engineered cell, population of cells, pharmaceutical composition, or method of any one of the preceding embodiments, wherein the cell is memory B cell.

Embodiment 138 is the engineered cell, population of cells, pharmaceutical composition, or method of any one of the preceding embodiments, wherein the cell is a hematopoietic stem cell (HSC).

Embodiment 139 is the engineered cell, population of cells, pharmaceutical composition, or method of any one of the preceding embodiments, wherein the cell is an activated cell.

Embodiment 140 is the engineered cell, population of cells, pharmaceutical composition, or method of any one of the preceding embodiments, wherein the cell is a non-activated cell.

Embodiment 141 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule.

Embodiment 142 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule binds to an inhibitory receptor on an NK cell.

Embodiment 143 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule binds to NKG2A on an NK cell.

Embodiment 144 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule is a non-classical MHC class I molecule.

Embodiment 145 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule is HLA-E.

Embodiment 146 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule is a fusion protein.

Embodiment 147 is the engineered cell, population of cells, pharmaceutical composition, or method of any one of the preceding embodiments, comprising an exogenous nucleic acid or contacting the cell with an exogenous nucleic acid, wherein the exogenous nucleic acid encodes an NK cell inhibitor molecule, wherein the NK cell inhibitor molecule is a fusion protein comprising HLA-E and B2M.

Embodiment 148 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is an antibody or antibody fragment.

Embodiment 149 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a full-length IgG antibody.

Embodiment 150 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a single chain antibody.

Embodiment 151 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a neutralizing antibody.

Embodiment 152 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a therapeutic polypeptide.

Embodiment 153 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is an enzyme.

Embodiment 154 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a cytokine.

Embodiment 155 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a chemokine.

Embodiment 156 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a fusion protein.

Embodiment 157 is the engineered cell, population of cells, composition, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a T cell receptor (TCR).

Embodiment 158 is the engineered cell, population of cells, composition, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a genetically modified TCR.

Embodiment 159 is the engineered cell, population of cells, composition, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is the WT1 TCR.

Embodiment 160 is the engineered cell, population of cells, composition, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a CAR.

Embodiment 161 is the engineered cell, population of cells, pharmaceutical composition, or method of any one of the preceding embodiments, wherein the CIITA guide RNA is provided to the cell in a vector.

Embodiment 162 is the engineered cell, population of cells, pharmaceutical composition, or method of any one of the preceding embodiments, wherein the CIITA RNA-guided DNA binding agent is provided to the cell in a vector, optionally in the same vector as the CIITA guide RNA.

Embodiment 163 is the engineered cell, population of cells, pharmaceutical composition, or method of any one of the preceding embodiments, wherein the exogenous nucleic acid is provided to the cell in a vector.

Embodiment 164 is the engineered cell, population of cells, pharmaceutical composition, or method of embodiment 163, wherein the vector is a viral vector.

Embodiment 165 is the engineered cell, population of cells, pharmaceutical composition, or method of embodiment 163, wherein the vector is a non-viral vector.

Embodiment 166 is the engineered cell, population of cells, pharmaceutical composition, or method of embodiment 164, wherein the vector is a lentiviral vector.

Embodiment 167 is the engineered cell, population of cells, pharmaceutical composition, or method of any one of embodiment 164, wherein the vector is an AAV.

Embodiment 168 is the engineered cell, population of cells, pharmaceutical composition, or method of any one of the preceding embodiments, wherein the guide RNA is provided to the cell in a lipid nucleic acid assembly composition, optionally in the same lipid nucleic acid assembly composition as an RNA-guided DNA binding agent.

Embodiment 169 is the engineered cell, population of cells, pharmaceutical composition, or method of any one of the preceding embodiments, wherein the exogenous nucleic acid is provided to the cell in a lipid nucleic acid assembly composition.

Embodiment 170 is the engineered cell, population of cells, pharmaceutical composition, or method of embodiment 168 or 169, wherein the lipid nucleic acid assembly composition is a lipid nanoparticle (LNP).

Embodiment 171 is the engineered cell, population of cells, pharmaceutical composition, or method of any one of the preceding embodiments, wherein the exogenous nucleic acid is integrated into the genome of the cell.

Embodiment 172 is the engineered cell, population of cells, pharmaceutical composition, or method of any one of the preceding embodiments, wherein the exogenous nucleic acid is integrated into the genome of the cell by homologous recombination (HR).

Embodiment 173 is the engineered cell, population of cells, pharmaceutical composition, or method of any one of the preceding embodiments, wherein the exogenous nucleic acid is integrated into a safe harbor locus in the genome of the cell.

Embodiment 174 is the engineered cell, population of cells, composition, pharmaceutical composition, or method of any one of the preceding embodiments, wherein the CIITA guide RNA comprises SEQ ID NO: 1.

Embodiment 175 is the engineered cell, population of cells, composition, pharmaceutical composition, or method of any one of the preceding embodiments, wherein the CIITA guide RNA comprises SEQ ID NO: 2.

Embodiment 176 is the engineered cell, population of cells, composition, pharmaceutical composition, or method of any one of the preceding embodiments, wherein the CIITA guide RNA comprises SEQ ID NO: 3.

Embodiment 177 is the engineered cell, population of cells, composition, pharmaceutical composition, or method of any one of the preceding embodiments, wherein the CIITA guide RNA comprises SEQ ID NO: 4.

Embodiment 178 is the engineered cell, population of cells, composition, pharmaceutical composition, or method of any one of the preceding embodiments, wherein the CIITA guide RNA comprises SEQ ID NO: 5.

Embodiment 179 is the engineered cell, population of cells, composition, pharmaceutical composition, or method of any one of the preceding embodiments, wherein the CIITA guide RNA comprises SEQ ID NO: 6.

Embodiment 180 is the engineered cell, population of cells, composition, pharmaceutical composition, or method of any one of the preceding embodiments, wherein the CIITA guide RNA comprises SEQ ID NO: 7.

Embodiment 181 is the engineered cell, population of cells, composition, pharmaceutical composition, or method of any one of the preceding embodiments, wherein the CIITA guide RNA comprises SEQ ID NO: 8.

Embodiment 182 is the engineered cell, population of cells, composition, pharmaceutical composition, or method of any one of the preceding embodiments, wherein the CIITA guide RNA comprises SEQ ID NO: 9.

Embodiment 183 is the engineered cell, population of cells, composition, pharmaceutical composition, or method of any one of the preceding embodiments, wherein the CIITA guide RNA comprises SEQ ID NO: 10.

Embodiment 184 is the engineered cell, population of cells, composition, pharmaceutical composition, or method of any one of the preceding embodiments, wherein the CIITA guide RNA comprises SEQ ID NO: 11.

Embodiment 185 is the engineered cell, population of cells, composition, pharmaceutical composition, or method of any one of the preceding embodiments, wherein the CIITA guide RNA comprises SEQ ID NO: 12.

Embodiment 186 is the engineered cell, population of cells, composition, pharmaceutical composition, or method of any one of the preceding embodiments, wherein the CIITA guide RNA comprises SEQ ID NO: 13.

Embodiment 187 is the engineered cell, population of cells, composition, pharmaceutical composition, or method of any one of the preceding embodiments, wherein the CIITA guide RNA comprises SEQ ID NO: 14.

Embodiment 188 is the engineered cell, population of cells, composition, pharmaceutical composition, or method of any one of the preceding embodiments, wherein the CIITA guide RNA comprises SEQ ID NO: 15.

Embodiment 189 is the engineered cell, population of cells, composition, pharmaceutical composition, or method of any one of the preceding embodiments, wherein the CIITA guide RNA comprises SEQ ID NO: 16.

Embodiment 190 is the engineered cell, population of cells, composition, pharmaceutical composition, or method of any one of the preceding embodiments, wherein the CIITA guide RNA comprises SEQ ID NO: 17.

Embodiment 191 is the engineered cell, population of cells, composition, pharmaceutical composition, or method of any one of the preceding embodiments, wherein the CIITA guide RNA comprises SEQ ID NO: 18.

Embodiment 192 is the engineered cell, population of cells, composition, pharmaceutical composition, or method of any one of the preceding embodiments, wherein the CIITA guide RNA comprises SEQ ID NO: 19.

Embodiment 193 is the engineered cell, population of cells, composition, pharmaceutical composition, or method of any one of the preceding embodiments, wherein the CIITA guide RNA comprises SEQ ID NO: 20.

Embodiment 194 is the engineered cell, population of cells, composition, pharmaceutical composition, or method of any one of the preceding embodiments, wherein the CIITA guide RNA comprises SEQ ID NO: 21.

Embodiment 195 is the engineered cell, population of cells, composition, pharmaceutical composition, or method of any one of the preceding embodiments, wherein the CIITA guide RNA comprises SEQ ID NO: 22.

Embodiment 196 is the engineered cell, population of cells, composition, pharmaceutical composition, or method of any one of the preceding embodiments, wherein the CIITA guide RNA comprises SEQ ID NO: 23.

Embodiment 197 is the engineered cell, population of cells, composition, pharmaceutical composition, or method of any one of the preceding embodiments, wherein the CIITA guide RNA comprises SEQ ID NO: 24.

Embodiment 198 is the engineered cell, population of cells, composition, pharmaceutical composition, or method of any one of the preceding embodiments, wherein the CIITA guide RNA comprises SEQ ID NO: 25.

Embodiment 199 is the engineered cell, population of cells, composition, pharmaceutical composition, or method of any one of the preceding embodiments, wherein the CIITA guide RNA comprises SEQ ID NO: 26.

Embodiment 200 is the engineered cell, population of cells, composition, pharmaceutical composition, or method of any one of the preceding embodiments, wherein the CIITA guide RNA comprises SEQ ID NO: 27.

Embodiment 201 is the engineered cell, population of cells, composition, pharmaceutical composition, or method of any one of the preceding embodiments, wherein the CIITA guide RNA comprises SEQ ID NO: 28.

Embodiment 202 is the engineered cell, population of cells, composition, pharmaceutical composition, or method of any one of the preceding embodiments, wherein the CIITA guide RNA comprises SEQ ID NO: 29.

Embodiment 203 is the engineered cell, population of cells, composition, pharmaceutical composition, or method of any one of the preceding embodiments, wherein the CIITA guide RNA comprises SEQ ID NO: 30.

Embodiment 204 is the engineered cell, population of cells, composition, pharmaceutical composition, or method of any one of the preceding embodiments, wherein the CIITA guide RNA comprises SEQ ID NO: 31.

Embodiment 205 is the engineered cell, population of cells, composition, pharmaceutical composition, or method of any one of the preceding embodiments, wherein the CIITA guide RNA comprises SEQ ID NO: 32.

Embodiment 206 is the engineered cell, population of cells, composition, pharmaceutical composition, or method of any one of the preceding embodiments, wherein the CIITA guide RNA comprises SEQ ID NO: 33.

Embodiment 207 is the engineered cell, population of cells, composition, pharmaceutical composition, or method of any one of the preceding embodiments, wherein the CIITA guide RNA comprises SEQ ID NO: 34.

Embodiment 208 is the engineered cell, population of cells, composition, pharmaceutical composition, or method of any one of the preceding embodiments, wherein the CIITA guide RNA comprises SEQ ID NO: 35.

Embodiment 209 is the engineered cell, population of cells, composition, pharmaceutical composition, or method of any one of the preceding embodiments, wherein the CIITA guide RNA comprises SEQ ID NO: 36.

Embodiment 210 is the engineered cell, population of cells, composition, pharmaceutical composition, or method of any one of the preceding embodiments, wherein the CIITA guide RNA comprises SEQ ID NO: 37.

Embodiment 211 is the engineered cell, population of cells, composition, pharmaceutical composition, or method of any one of the preceding embodiments, wherein the CIITA guide RNA comprises SEQ ID NO: 38.

Embodiment 212 is the engineered cell, population of cells, composition, pharmaceutical composition, or method of any one of the preceding embodiments, wherein the CIITA guide RNA comprises SEQ ID NO: 39.

Embodiment 213 is the engineered cell, population of cells, composition, pharmaceutical composition, or method of any one of the preceding embodiments, wherein the CIITA guide RNA comprises SEQ ID NO: 40.

Embodiment 214 is the engineered cell, population of cells, composition, pharmaceutical composition, or method of any one of the preceding embodiments, wherein the CIITA guide RNA comprises SEQ ID NO: 41.

Embodiment 215 is the engineered cell, population of cells, composition, pharmaceutical composition, or method of any one of the preceding embodiments, wherein the CIITA guide RNA comprises SEQ ID NO: 42.

Embodiment 216 is the engineered cell, population of cells, composition, pharmaceutical composition, or method of any one of the preceding embodiments, wherein the CIITA guide RNA comprises SEQ ID NO: 43.

Embodiment 217 is the engineered cell, population of cells, composition, pharmaceutical composition, or method of any one of the preceding embodiments, wherein the CIITA guide RNA comprises SEQ ID NO: 44.

Embodiment 218 is the engineered cell, population of cells, composition, pharmaceutical composition, or method of any one of the preceding embodiments, wherein the CIITA guide RNA comprises SEQ ID NO: 45.

Embodiment 219 is the engineered cell, population of cells, composition, pharmaceutical composition, or method of any one of the preceding embodiments, wherein the CIITA guide RNA comprises SEQ ID NO: 46.

Embodiment 220 is the engineered cell, population of cells, composition, pharmaceutical composition, or method of any one of the preceding embodiments, wherein the CIITA guide RNA comprises SEQ ID NO: 47.

Embodiment 221 is the engineered cell, population of cells, composition, pharmaceutical composition, or method of any one of the preceding embodiments, wherein the CIITA guide RNA comprises SEQ ID NO: 48.

Embodiment 222 is the engineered cell, population of cells, composition, pharmaceutical composition, or method of any one of the preceding embodiments, wherein the CIITA guide RNA comprises SEQ ID NO: 49.

Embodiment 223 is the engineered cell, population of cells, composition, pharmaceutical composition, or method of any one of the preceding embodiments, wherein the CIITA guide RNA comprises SEQ ID NO: 50.

Embodiment 224 is the engineered cell, population of cells, composition, pharmaceutical composition, or method of any one of the preceding embodiments, wherein the CIITA guide RNA comprises SEQ ID NO: 51.

Embodiment 225 is the engineered cell, population of cells, composition, pharmaceutical composition, or method of any one of the preceding embodiments, wherein the CIITA guide RNA comprises SEQ ID NO: 52.

Embodiment 226 is the engineered cell, population of cells, composition, pharmaceutical composition, or method of any one of the preceding embodiments, wherein the CIITA guide RNA comprises SEQ ID NO: 53.

Embodiment 227 is the engineered cell, population of cells, composition, pharmaceutical composition, or method of any one of the preceding embodiments, wherein the CIITA guide RNA comprises SEQ ID NO: 54.

Embodiment 228 is the engineered cell, population of cells, composition, pharmaceutical composition, or method of any one of the preceding embodiments, wherein the CIITA guide RNA comprises SEQ ID NO: 55.

Embodiment 229 is the engineered cell, population of cells, composition, pharmaceutical composition, or method of any one of the preceding embodiments, wherein the CIITA guide RNA comprises SEQ ID NO: 56.

Embodiment 230 is the engineered cell, population of cells, composition, pharmaceutical composition, or method of any one of the preceding embodiments, wherein the CIITA guide RNA comprises SEQ ID NO: 57.

Embodiment 231 is the engineered cell, population of cells, composition, pharmaceutical composition, or method of any one of the preceding embodiments, wherein the CIITA guide RNA comprises SEQ ID NO: 58.

Embodiment 232 is the engineered cell, population of cells, composition, pharmaceutical composition, or method of any one of the preceding embodiments, wherein the CIITA guide RNA comprises SEQ ID NO: 59.

Embodiment 233 is the engineered cell, population of cells, composition, pharmaceutical composition, or method of any one of the preceding embodiments, wherein the CIITA guide RNA comprises SEQ ID NO: 60.

Embodiment 234 is the engineered cell, population of cells, composition, pharmaceutical composition, or method of any one of the preceding embodiments, wherein the CIITA guide RNA comprises SEQ ID NO: 61.

Embodiment 235 is the engineered cell, population of cells, composition, pharmaceutical composition, or method of any one of the preceding embodiments, wherein the CIITA guide RNA comprises SEQ ID NO: 62.

Embodiment 236 is the engineered cell, population of cells, composition, pharmaceutical composition, or method of any one of the preceding embodiments, wherein the CIITA guide RNA comprises SEQ ID NO: 63.

Embodiment 237 is the engineered cell, population of cells, composition, pharmaceutical composition, or method of any one of the preceding embodiments, wherein the CIITA guide RNA comprises SEQ ID NO: 64.

Embodiment 238 is the engineered cell, population of cells, composition, pharmaceutical composition, or method of any one of the preceding embodiments, wherein the CIITA guide RNA comprises SEQ ID NO: 65.

Embodiment 239 is the engineered cell, population of cells, composition, pharmaceutical composition, or method of any one of the preceding embodiments, wherein the CIITA guide RNA comprises SEQ ID NO: 66.

Embodiment 240 is the engineered cell, population of cells, composition, pharmaceutical composition, or method of any one of the preceding embodiments, wherein the CIITA guide RNA comprises SEQ ID NO: 67.

Embodiment 241 is the engineered cell, population of cells, composition, pharmaceutical composition, or method of any one of the preceding embodiments, wherein the CIITA guide RNA comprises SEQ ID NO: 68.

Embodiment 242 is the engineered cell, population of cells, composition, pharmaceutical composition, or method of any one of the preceding embodiments, wherein the CIITA guide RNA comprises SEQ ID NO: 69.

Embodiment 243 is the engineered cell, population of cells, composition, pharmaceutical composition, or method of any one of the preceding embodiments, wherein the CIITA guide RNA comprises SEQ ID NO: 70.

Embodiment 244 is the engineered cell, population of cells, composition, pharmaceutical composition, or method of any one of the preceding embodiments, wherein the CIITA guide RNA comprises SEQ ID NO: 71.

Embodiment 245 is the engineered cell, population of cells, composition, pharmaceutical composition, or method of any one of the preceding embodiments, wherein the CIITA guide RNA comprises SEQ ID NO: 72.

Embodiment 246 is the engineered cell, population of cells, composition, pharmaceutical composition, or method of any one of the preceding embodiments, wherein the CIITA guide RNA comprises SEQ ID NO: 73.

Embodiment 247 is the engineered cell, population of cells, composition, pharmaceutical composition, or method of any one of the preceding embodiments, wherein the CIITA guide RNA comprises SEQ ID NO: 74.

Embodiment 248 is the engineered cell, population of cells, composition, pharmaceutical composition, or method of any one of the preceding embodiments, wherein the CIITA guide RNA comprises SEQ ID NO: 75.

Embodiment 249 is the engineered cell, population of cells, composition, pharmaceutical composition, or method of any one of the preceding embodiments, wherein the CIITA guide RNA comprises SEQ ID NO: 76.

Embodiment 250 is the engineered cell, population of cells, composition, pharmaceutical composition, or method of any one of the preceding embodiments, wherein the CIITA guide RNA comprises SEQ ID NO: 77.

Embodiment 251 is the engineered cell, population of cells, composition, pharmaceutical composition, or method of any one of the preceding embodiments, wherein the CIITA guide RNA comprises SEQ ID NO: 78.

Embodiment 252 is the engineered cell, population of cells, composition, pharmaceutical composition, or method of any one of the preceding embodiments, wherein the CIITA guide RNA comprises SEQ ID NO: 79.

Embodiment 253 is the engineered cell, population of cells, composition, pharmaceutical composition, or method of any one of the preceding embodiments, wherein the CIITA guide RNA comprises SEQ ID NO: 80.

Embodiment 254 is the engineered cell, population of cells, composition, pharmaceutical composition, or method of any one of the preceding embodiments, wherein the CIITA guide RNA comprises SEQ ID NO: 81.

Embodiment 255 is the engineered cell, population of cells, composition, pharmaceutical composition, or method of any one of the preceding embodiments, wherein the CIITA guide RNA comprises SEQ ID NO: 82.

Embodiment 256 is the engineered cell, population of cells, composition, pharmaceutical composition, or method of any one of the preceding embodiments, wherein the CIITA guide RNA comprises SEQ ID NO: 83.

Embodiment 257 is the engineered cell, population of cells, composition, pharmaceutical composition, or method of any one of the preceding embodiments, wherein the CIITA guide RNA comprises SEQ ID NO: 84.

Embodiment 258 is the engineered cell, population of cells, composition, pharmaceutical composition, or method of any one of the preceding embodiments, wherein the CIITA guide RNA comprises SEQ ID NO: 85.

Embodiment 259 is the engineered cell, population of cells, composition, pharmaceutical composition, or method of any one of the preceding embodiments, wherein the CIITA guide RNA comprises SEQ ID NO: 86.

Embodiment 260 is the engineered cell, population of cells, composition, pharmaceutical composition, or method of any one of the preceding embodiments, wherein the CIITA guide RNA comprises SEQ ID NO: 87.

Embodiment 261 is the engineered cell, population of cells, composition, pharmaceutical composition, or method of any one of the preceding embodiments, wherein the CIITA guide RNA comprises SEQ ID NO: 88.

Embodiment 262 is the engineered cell, population of cells, composition, pharmaceutical composition, or method of any one of the preceding embodiments, wherein the CIITA guide RNA comprises SEQ ID NO: 89.

Embodiment 263 is the engineered cell, population of cells, composition, pharmaceutical composition, or method of any one of the preceding embodiments, wherein the CIITA guide RNA comprises SEQ ID NO: 90.

Embodiment 264 is the engineered cell, population of cells, composition, pharmaceutical composition, or method of any one of the preceding embodiments, wherein the CIITA guide RNA comprises SEQ ID NO: 91.

Embodiment 265 is the engineered cell, population of cells, composition, pharmaceutical composition, or method of any one of the preceding embodiments, wherein the CIITA guide RNA comprises SEQ ID NO: 92.

Embodiment 266 is the engineered cell, population of cells, composition, pharmaceutical composition, or method of any one of the preceding embodiments, wherein the CIITA guide RNA comprises SEQ ID NO: 93.

Embodiment 267 is the engineered cell, population of cells, composition, pharmaceutical composition, or method of any one of the preceding embodiments, wherein the CIITA guide RNA comprises SEQ ID NO: 94.

Embodiment 268 is the engineered cell, population of cells, composition, pharmaceutical composition, or method of any one of the preceding embodiments, wherein the CIITA guide RNA comprises SEQ ID NO: 95.

Embodiment 269 is the engineered cell, population of cells, composition, pharmaceutical composition, or method of any one of the preceding embodiments, wherein the CIITA guide RNA comprises SEQ ID NO: 96.

Embodiment 270 is the engineered cell, population of cells, composition, pharmaceutical composition, or method of any one of the preceding embodiments, wherein the CIITA guide RNA comprises SEQ ID NO: 97.

Embodiment 271 is the engineered cell, population of cells, composition, pharmaceutical composition, or method of any one of the preceding embodiments, wherein the CIITA guide RNA comprises SEQ ID NO: 98.

Embodiment 272 is the engineered cell, population of cells, composition, pharmaceutical composition, or method of any one of the preceding embodiments, wherein the CIITA guide RNA comprises SEQ ID NO: 99.

Embodiment 273 is the engineered cell, population of cells, composition, pharmaceutical composition, or method of any one of the preceding embodiments, wherein the CIITA guide RNA comprises SEQ ID NO: 100.

Embodiment 274 is the engineered cell, population of cells, composition, pharmaceutical composition, or method of any one of the preceding embodiments, wherein the CIITA guide RNA comprises SEQ ID NO: 101.

Embodiment 275 is the engineered cell, population of cells, composition, pharmaceutical composition, or method of any one of the preceding embodiments, wherein the CIITA guide RNA comprises at least one modification.

Embodiment 276 is the engineered cell, population of cells, composition, pharmaceutical composition, or method of any one of the preceding embodiments, wherein the CIITA guide RNA comprises at least one modification, wherein the at least one modification includes a 2′-O-methyl (2′-O-Me) modified nucleotide.

Embodiment 277 is the engineered cell, population of cells, composition, pharmaceutical composition, or method of any one of the preceding embodiments, wherein the CIITA guide RNA comprises at least one modification, comprising a phosphorothioate (PS) bond between nucleotides.

Embodiment 278 is the engineered cell, population of cells, composition, pharmaceutical composition, or method of any one of the preceding embodiments, wherein the CIITA guide RNA comprises at least one modification, comprising a 2′-fluoro (2′-F) modified nucleotide.

Embodiment 279 is the engineered cell, population of cells, composition, pharmaceutical composition, or method of any one of the preceding embodiments, wherein the CIITA guide RNA comprises at least one modification, comprising a modification at one or more of the first five nucleotides at the 5′ end of the guide RNA.

Embodiment 280 is the engineered cell, population of cells, composition, pharmaceutical composition, or method of any one of the preceding embodiments, wherein the CIITA guide RNA comprises at least one modification, comprising a modification at one or more of the last five nucleotides at the 3′ end of the guide RNA.

Embodiment 281 is the engineered cell, population of cells, composition, pharmaceutical composition, or method of any one of the preceding embodiments, wherein the CIITA guide RNA comprises at least one modification, comprising a PS bond between the first four nucleotides of the guide RNA.

Embodiment 282 is the engineered cell, population of cells, composition, pharmaceutical composition, or method of any one of the preceding embodiments, wherein the CIITA guide RNA comprises at least one modification, comprising a PS bond between the last four nucleotides of the guide RNA.

Embodiment 283 is the engineered cell, population of cells, composition, pharmaceutical composition, or method of any one of the preceding embodiments, wherein the CIITA guide RNA comprises at least one modification, comprising a 2′-O-Me modified nucleotide at the first three nucleotides at the 5′ end of the guide RNA.

Embodiment 284 is the engineered cell, population of cells, composition, pharmaceutical composition, or method of any one of the preceding embodiments, wherein the CIITA guide RNA comprises at least one modification, comprising a 2′-O-Me modified nucleotide at the last three nucleotides at the 3′ end of the guide RNA.

Embodiment 285 is an engineered cell or population of cells comprising a genetic modification that includes an indel within the genomic region targeted by the CIITA guide RNA of any of the preceding embodiments.

Embodiment 286 is an engineered cell or population of cells comprising a genetic modification that includes a C to T substitution or an A to G substitution within the genomic region targeted by the CIITA guide RNA of any of the preceding embodiments.

Embodiment 287 is the engineered cell, population of cells, composition, pharmaceutical composition, or method of any one of the preceding embodiments, for use to express a TCR with specificity for a polypeptide expressed by cancer cells.

Embodiment 288 is the engineered cell, population of cells, composition, pharmaceutical composition, or method of any one of the preceding embodiments, for use in administering to a subject as an adoptive cell transfer (ACT) therapy.

Embodiment 289 is the engineered cell, population of cells, composition, pharmaceutical composition, or method of any one of the preceding embodiments, for use in treating a subject with cancer.

Embodiment 290 is the engineered cell, population of cells, composition, pharmaceutical composition, or method of any one of the preceding embodiments, for use in treating a subject with an infectious disease.

Embodiment 291 is the engineered cell, population of cells, composition, pharmaceutical composition, or method of any one of the preceding embodiments, for use in treating a subject with an autoimmune disease.

Embodiment 292 is the engineered cell, population of cells, pharmaceutical composition, or method of any one of the preceding embodiments, wherein the genetic modification comprises an indel.

Embodiment 293 is the engineered cell, population of cells, pharmaceutical composition, or method of any one of the preceding embodiments, wherein the genetic modification comprises a C to T substitution.

Embodiment 294 is the engineered cell, population of cells, pharmaceutical composition, or method of any one of the preceding embodiments, wherein the genetic modification comprises an A to G substitution.

Embodiment 295 is the engineered cell, population of cells, pharmaceutical composition, or method of any one of the preceding embodiments, wherein the cell is homozygous for HLA-B and homozygous for HLA-C.

Embodiment 296 is the engineered cell, population of cells, pharmaceutical composition, or method of any one of the preceding embodiments, wherein the cell further comprises a genetic modification in an HLA-A gene, wherein the cell is homozygous for HLA-B and homozygous for HLA-C, and wherein the genetic modification in the HLA-A gene comprises at least one nucleotide within the genomic coordinates chosen from: a) chr6:29942854 to chr6:29942913 and b) chr6:29943518 to chr6: 29943619.

Embodiment 297 is the engineered cell, population of cells, pharmaceutical composition, or method of any one of the preceding embodiments, wherein the cell further comprises a genetic modification in an HLA-A gene, and wherein the genetic modification in the HLA-A gene comprises at least one nucleotide within the genomic coordinates chosen from: chr6:29942864 to chr6: 29942903.

Embodiment 298 is the engineered cell, population of cells, pharmaceutical composition, or method of any one of the preceding embodiments, wherein the cell further comprises a genetic modification in an HLA-A gene, and wherein the genetic modification in the HLA-A gene comprises at least one nucleotide within the genomic coordinates chosen from: chr6:29943528 to chr6:29943609.

Embodiment 299 is the engineered cell, population of cells, pharmaceutical composition, or method of any one of the preceding embodiments, wherein the cell further comprises a genetic modification in an HLA-A gene, and wherein the genetic modification in the HLA-A gene comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Embodiment 300 is the engineered cell, population of cells, pharmaceutical composition, or method of any one of the preceding embodiments, wherein the cell further comprises a genetic modification in an HLA-A gene, and wherein the genetic modification in the HLA-A gene comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Embodiment 301 is the engineered cell, population of cells, pharmaceutical composition, or method of any one of the preceding embodiments, wherein the HLA-A expression of the cell is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Embodiment 302 is a method of making an engineered cell, which has reduced or eliminated surface expression of MHC class II protein and HLA-A protein relative to an unmodified cell, comprising: a) contacting the cell with a CIITA guide RNA, wherein the guide RNA comprises a guide sequence selected from SEQ ID NOs: 1-101; b) contacting the cell with an HLA-A guide RNA, wherein the HLA-A guide RNA comprises a guide sequence selected from any one of SEQ ID NOs: 2001-2095; and c) optionally contacting the cell with an RNA-guided DNA binding agent or nucleic acid encoding an RNA-guided DNA binding agent; thereby reducing or eliminating the surface expression of MHC class II protein and HLA-A protein in the cell relative to an unmodified cell.

Embodiment 303 is the method of the immediately preceding embodiment, comprising contacting the cell with an RNA-guided DNA binding agent or nucleic acid encoding an RNA-guided DNA binding agent, optionally wherein the RNA-guided DNA binding agent comprises an S. pyogenes Cas9.

Embodiment 304 is the engineered cell, population of cells, pharmaceutical composition, or method of any one of the preceding embodiments, wherein the HLA-B is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02.

Embodiment 305 is the engineered cell, population of cells, pharmaceutical composition, or method of any one of the preceding embodiments, wherein the HLA-C is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

Embodiment 306 is the engineered cell, population of cells, pharmaceutical composition, or method of any one of the preceding embodiments, wherein the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

Embodiment 307 is the engineered cell, population of cells, pharmaceutical composition, or method of any one of the preceding embodiments, wherein the HLA-B and HLA-C alleles are selected from any one of the following HLA-B and HLA-C alleles: HLA-B*07:02 and HLA-C*07:02; HLA-B*08:01 and HLA-C*07:01; HLA-B*44:02 and HLA-C*05:01; HLA-B*35:01 and HLA-C*04:01; HLA-B*40:01 and HLA-C*03:04; HLA-B*57:01 and HLA-C*06:02; HLA-B*14:02 and HLA-C*08:02; HLA-B*15:01 and HLA-C*03:03; HLA-B*13:02 and HLA-C*06:02; HLA-B*44:03 and HLA-C*16:01; HLA-B*38:01 and HLA-C*12:03; HLA-B*18:01 and HLA-C*07:01; HLA-B*44:03 and HLA-C*04:01; HLA-B*51:01 and HLA-C*15:02; HLA-B*49:01 and HLA-C*07:01; HLA-B*15:01 and HLA-C*03:04; HLA-B*18:01 and HLA-C*12:03; HLA-B*27:05 and HLA-C*02:02; HLA-B*35:03 and HLA-C*04:01; HLA-B*18:01 and HLA-C*05:01; HLA-B*52:01 and HLA-C*12:02; HLA-B*51:01 and HLA-C*14:02; HLA-B*37:01 and HLA-C*06:02; HLA-B*53:01 and HLA-C*04:01; HLA-B*55:01 and HLA-C*03:03; HLA-B*44:02 and HLA-C*07:04; HLA-B*44:03 and HLA-C*07:01; HLA-B*35:02 and HLA-C*04:01; HLA-B*15:01 and HLA-C*04:01; and HLA-B*40:02 and HLA-C*02:02.

Embodiment 308 is the engineered cell, population of cells, pharmaceutical composition, or method of any one of the preceding embodiments, wherein the HLA-B and HLA-C alleles are HLA-B*07:02 and HLA-C*07:02.

Embodiment 309 is the engineered cell, population of cells, pharmaceutical composition, or method of any one of the preceding embodiments, wherein the HLA-B and HLA-C alleles are HLA-B*08:01 and HLA-C*07:01.

Embodiment 310 is the engineered cell, population of cells, pharmaceutical composition, or method of any one of the preceding embodiments, wherein the HLA-B and HLA-C alleles are HLA-B*44:02 and HLA-C*05:01.

Embodiment 311 is the engineered cell, population of cells, pharmaceutical composition, or method of any one of the preceding embodiments, wherein the HLA-B and HLA-C alleles are HLA-B*35:01 and HLA-C*04:01.

TABLE 23
ADDITIONAL SEQUENCES

| | SEQ | |
|---|---|---|
| | ID | |
| | NO | Sequence |
| G000529 | 701 | mG*mG*mC*CACGGAGCGAGACAUCUGUUUUAGAmGmCmUmA |
| gRNA | | mGmAmAmAmUmAmGmCAAGUUAAAAUAAGGCUAGUCCGUUAU |
| | | CAmAmCmUmUmGmAmAmAmAmAmGmUmGmGmCmAmCmCmGmA |
| | | mGmUmCmGmGmUmGmCmU*mU*mU*mU |
| G013674 | 702 | mU*mU*mC*UAGGGGCCCCAACUCCAGUUUUAGAmGmCmUmA |
| gRNA | | mGmAmAmAmUmAmGmCAAGUUAAAAUAAGGCUAGUCCGUUAU |
| | | CAmAmCmUmUmGmAmAmAmAmAmGmUmGmGmCmAmCmCmGmA |
| | | mGmUmCmGmGmUmGmCmU*mU*mU*mU |
| G012086 | 703 | mA*mG*mA*GUCUCUCAGCUGGUACAGUUUUAGAmGmCmUmA |
| gRNA | | mGmAmAmAmUmAmGmCAAGUUAAAAUAAGGCUAGUCCGUUAU |
| | | CAmAmCmUmUmGmAmAmAmAmAmGmUmGmGmCmAmCmCmGmA |
| | | mGmUmCmGmGmUmGmCmU*mU*mU*mU |
| G015995 | 704 | mU*mU*mA*CCCCACUUAACUAUCUUGUUUUAGAmGmCmUmA |
| gRNA | | mGmAmAmAmUmAmGmCAAGUUAAAAUAAGGCUAGUCCGUUAU |
| | | CAmAmCmUmUmGmAmAmAmAmAmGmUmGmGmCmAmCmCmGmA |
| | | mGmUmCmGmGmUmGmCmU*mU*mU*mU |
| G016017 | 705 | mC*mC*mA*CUCUGCCCCAUGGGCUCGUUUUAGAmGmCmUmA |
| gRNA | | mGmAmAmAmUmAmGmCAAGUUAAAAUAAGGCUAGUCCGUUAU |
| | | CAmAmCmUmUmGmAmAmAmAmAmGmUmGmGmCmAmCmCmGmA |
| | | mGmUmCmGmGmUmGmCmU*mU*mU*mU |
| G016206 | 706 | mC*mG*mC*UGUCAAGUCCAGUUCUAGUUUUAGAmGmCmUmA |
| gRNA | | mGmAmAmAmUmAmGmCAAGUUAAAAUAAGGCUAGUCCGUUAU |
| | | CAmAmCmUmUmGmAmAmAmAmAmGmUmGmGmCmAmCmCmGmA |
| | | mGmUmCmGmGmUmGmCmU*mU*mU*mU |
| G000644 | 707 | mG*mA*mG*UCCGAGCAGAAGAAGAAGUUUUAGAmGmCmUmA |
| gRNA | | mGmAmAmAmUmAmGmCAAGUUAAAAUAAGGCUAGUCCGUUAU |
| | | CAmAmCmUmUmGmAmAmAmAmAmGmUmGmGmCmAmCmCmGmA |
| | | mGmUmCmGmGmUmGmCmU*mU*mU*mU |
| G000645 | 708 | mG*mA*mC*CCCCUCCACCCCGCCUCGUUUUAGAmGmCmUmA |
| gRNA | | mGmAmAmAmUmAmGmCAAGUUAAAAUAAGGCUAGUCCGUUAU |
| | | CAmAmCmUmUmGmAmAmAmAmAmGmUmGmGmCmAmCmCmGmA |
| | | mGmUmCmGmGmUmGmCmU*mU*mU*mU |
| G000646 | 709 | mG*mA*mC*UUGUUUUCAUUGUUCUCGUUUUAGAmGmCmUmA |
| gRNA | | mGmAmAmAmUmAmGmCAAGUUAAAAUAAGGCUAGUCCGUUAU |
| | | CAmAmCmUmUmGmAmAmAmAmAmGmUmGmGmCmAmCmCmGmA |
| | | mGmUmCmGmGmUmGmCmU*mU*mU*mU |
| Recombinant | 800 | MDKKYSIGLDIGTNSVGWAVITDEYKVPSKKFKVLGNTDRHSIKKNL |
| Cas9- | | IGALLFDSGETAEATRLKRTARRRYTRRKNRICYLQEIFSNEMAKVD |
| NLS amino | | DSFFHRLEESFLVEEDKKHERHPIFGNIVDEVAYHEKYPTIYHLRKKL |
| acid | | VDSTDKADLRLIYLALAHMIKFRGHFLIEGDLNPDNSDVDKLFIQLV |
| sequence | | QTYNQLFEENPINASGVDAKAILSARLSKSRRLENLIAQLPGEKKNGL |
| | | FGNLIALSLGLTPNFKSNFDLAEDAKLQLSKDTYDDDLDNLLAQIGD |
| | | QYADLFLAAKNLSDAILLSDILRVNTEITKAPLSASMIKRYDEHHQDL |
| | | TLLKALVRQQLPEKYKEIFFDQSKNGYAGYIDGGASQEEFYKFIKPIL |
| | | EKMDGTEELLVKLNREDLLRKQRTFDNGSIPHQIHLGELHAILRRQE |
| | | DFYPFLKDNREKIEKILTFRIPYYVGPLARGNSRFAWMTRKSEETITP |
| | | WNFEEVVDKGASAQSFIERMTNFDKNLPNEKVLPKHSLLYEYFTVY |
| | | NELTKVKYVTEGMRKPAFLSGEQKKAIVDLLFKTNRKVTVKQLKED |
| | | YFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRL |
| | | SRKLINGIRDKQSGKTILDFLKSDGFANRNFMQLIHDDSLTFKEDIQK |
| | | AQVSGQGDSLHEHIANLAGSPAIKKGILQTVKVVDELVKVMGRHKP |
| | | ENIVIEMARENQTTQKGQKNSRERMKRIEEGIKELGSQILKEHPVENT |
| | | QLQNEKLYLYYLQNGRDMYVDQELDINRLSDYDVDHIVPQSFLKDD |
| | | SIDNKVLTRSDKNRGKSDNVPSEEVVKKMKNYWRQLLNAKLITQRK |
| | | FDNLTKAERGGLSELDKAGFIKRQLVETRQITKHVAQILDSRMNTKY |
| | | DENDKLIREVKVITLKSKLVSDFRKDFQFYKVREINNYHHAHDAYLN |
| | | AVVGTALIKKYPKLESEFVYGDYKVYDVRKMIAKSEQEIGKATAKY |
| | | FFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKY |
| | | GGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNEL |
| | | ALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPA |
| | | AFKYFDTTIDRKRYTSTKEVLDATLIHQSITGLYETRIDLSQLGGDGG |
| | | GSPKKKRKV |
| ORF | 801 | ATGGACAAGAAGTACAGCATCGGACTGGACATCGGAACAAACAG |
| encoding | | CGTCGGATGGGCAGTCATCACAGACGAATACAAGGTCCCGAGCA |
| Sp. Cas9 | | AGAAGTTCAAGGTCCTGGGAAACACAGACAGACACAGCATCAAG |
| | | AAGAACCTGATCGGAGCACTGCTGTTCGACAGCGGAGAAACAGC |
| | | AGAAGCAACAAGACTGAAGAGAACAGCAAGAAGAAGATACACA |
| | | AGAAGAAAGAACAGAATCTGCTACCTGCAGGAAATCTTCAGCAA |
| | | CGAAATGGCAAAGGTCGACGACAGCTTCTTCCACAGACTGGAAG |
| | | AAAGCTTCCTGGTCGAAGAAGACAAGAAGCACGAAAGACACCCG |
| | | ATCTTCGGAAACATCGTCGACGAAGTCGCATACCACGAAAAGTAC |
| | | CCGACAATCTACCACCTGAGAAAGAAGCTGGTCGACAGCACAGA |
| | | CAAGGCAGACCTGAGACTGATCTACCTGGCACTGGCACACATGAT |
| | | CAAGTTCAGAGGACACTTCCTGATCGAAGGAGACCTGAACCCGG |
| | | ACAACAGCGACGTCGACAAGCTGTTCATCCAGCTGGTCCAGACAT |
| | | ACAACCAGCTGTTCGAAGAAAACCCGATCAACGCAAGCGGAGTC |
| | | GACGCAAAGGCAATCCTGAGCGCAAGACTGAGCAAGAGCAGAAG |
| | | ACTGGAAAACCTGATCGCACAGCTGCCGGGAGAAAAGAAGAACG |
| | | GACTGTTCGGAAACCTGATCGCACTGAGCCTGGGACTGACACCGA |
| | | ACTTCAAGAGCAACTTCGACCTGGCAGAAGACGCAAAGCTGCAG |
| | | CTGAGCAAGGACACATACGACGACGACCTGGACAACCTGCTGGC |
| | | ACAGATCGGAGACCAGTACGCAGACCTGTTCCTGGCAGCAAAGA |
| | | ACCTGAGCGACGCAATCCTGCTGAGCGACATCCTGAGAGTCAACA |
| | | CAGAAATCACAAAGGCACCGCTGAGCGCAAGCATGATCAAGAGA |
| | | TACGACGAACACCACCAGGACCTGACACTGCTGAAGGCACTGGTC |
| | | AGACAGCAGCTGCCGGAAAAGTACAAGGAAATCTTCTTCGACCA |
| | | GAGCAAGAACGGATACGCAGGATACATCGACGGAGGAGCAAGCC |
| | | AGGAAGAATTCTACAAGTTCATCAAGCCGATCCTGGAAAAGATG |
| | | GACGGAACAGAAGAACTGCTGGTCAAGCTGAACAGAGAAGACCT |
| | | GCTGAGAAAGCAGAGAACATTCGACAACGGAAGCATCCCGCACC |
| | | AGATCCACCTGGGAGAACTGCACGCAATCCTGAGAAGACAGGAA |
| | | GACTTCTACCCGTTCCTGAAGGACAACAGAGAAAAGATCGAAAA |
| | | GATCCTGACATTCAGAATCCCGTACTACGTCGGACCGCTGGCAAG |
| | | AGGAAACAGCAGATTCGCATGGATGACAAGAAAGAGCGAAGAAA |
| | | CAATCACACCGTGGAACTTCGAAGAAGTCGTCGACAAGGGAGCA |
| | | AGCGCACAGAGCTTCATCGAAAGAATGACAAACTTCGACAAGAA |
| | | CCTGCCGAACGAAAAGGTCCTGCCGAAGCACAGCCTGCTGTACGA |
| | | ATACTTCACAGTCTACAACGAACTGACAAAGGTCAAGTACGTCAC |
| | | AGAAGGAATGAGAAAGCCGGCATTCCTGAGCGGAGAACAGAAGA |
| | | AGGCAATCGTCGACCTGCTGTTCAAGACAAACAGAAAGGTCACA |
| | | GTCAAGCAGCTGAAGGAAGACTACTTCAAGAAGATCGAATGCTTC |
| | | GACAGCGTCGAAATCAGCGGAGTCGAAGACAGATTCAACGCAAG |
| | | CCTGGGAACATACCACGACCTGCTGAAGATCATCAAGGACAAGG |
| | | ACTTCCTGGACAACGAAGAAAACGAAGACATCCTGGAAGACATC |
| | | GTCCTGACACTGACACTGTTCGAAGACAGAGAAATGATCGAAGA |
| | | AAGACTGAAGACATACGCACACCTGTTCGACGACAAGGTCATGA |
| | | AGCAGCTGAAGAGAAGAAGATACACAGGATGGGGAAGACTGAGC |
| | | AGAAAGCTGATCAACGGAATCAGAGACAAGCAGAGCGGAAAGAC |
| | | AATCCTGGACTTCCTGAAGAGCGACGGATTCGCAAACAGAAACTT |
| | | CATGCAGCTGATCCACGACGACAGCCTGACATTCAAGGAAGACAT |
| | | CCAGAAGGCACAGGTCAGCGGACAGGGAGACAGCCTGCACGAAC |
| | | ACATCGCAAACCTGGCAGGAAGCCCGGCAATCAAGAAGGGAATC |
| | | CTGCAGACAGTCAAGGTCGTCGACGAACTGGTCAAGGTCATGGG |
| | | AAGACACAAGCCGGAAAACATCGTCATCGAAATGGCAAGAGAAA |
| | | ACCAGACAACACAGAAGGGACAGAAGAACAGCAGAGAAAGAAT |
| | | GAAGAGAATCGAAGAAGGAATCAAGGAACTGGGAAGCCAGATCC |
| | | TGAAGGAACACCCGGTCGAAAACACACAGCTGCAGAACGAAAAG |
| | | CTGTACCTGTACTACCTGCAGAACGGAAGAGACATGTACGTCGAC |
| | | CAGGAACTGGACATCAACAGACTGAGCGACTACGACGTCGACCA |
| | | CATCGTCCCGCAGAGCTTCCTGAAGGACGACAGCATCGACAACAA |
| | | GGTCCTGACAAGAAGCGACAAGAACAGAGGAAAGAGCGACAAC |
| | | GTCCCGAGCGAAGAAGTCGTCAAGAAGATGAAGAACTACTGGAG |
| | | ACAGCTGCTGAACGCAAAGCTGATCACACAGAGAAAGTTCGACA |
| | | ACCTGACAAAGGCAGAGAGAGGAGGACTGAGCGAACTGGACAAG |
| | | GCAGGATTCATCAAGAGACAGCTGGTCGAAACAAGACAGATCAC |
| | | AAAGCACGTCGCACAGATCCTGGACAGCAGAATGAACACAAAGT |
| | | ACGACGAAAACGACAAGCTGATCAGAGAAGTCAAGGTCATCACA |
| | | CTGAAGAGCAAGCTGGTCAGCGACTTCAGAAAGGACTTCCAGTTC |
| | | TACAAGGTCAGAGAAATCAACAACTACCACCACGCACACGACGC |
| | | ATACCTGAACGCAGTCGTCGGAACAGCACTGATCAAGAAGTACCC |
| | | GAAGCTGGAAAGCGAATTCGTCTACGGAGACTACAAGGTCTACG |
| | | ACGTCAGAAAGATGATCGCAAAGAGCGAACAGGAAATCGGAAAG |
| | | GCAACAGCAAAGTACTTCTTCTACAGCAACATCATGAACTTCTTC |
| | | AAGACAGAAATCACACTGGCAAACGGAGAAATCAGAAAGAGACC |
| | | GCTGATCGAAACAAACGGAGAAACAGGAGAAATCGTCTGGGACA |
| | | AGGGAAGAGACTTCGCAACAGTCAGAAAGGTCCTGAGCATGCCG |
| | | CAGGTCAACATCGTCAAGAAGACAGAAGTCCAGACAGGAGGATT |
| | | CAGCAAGGAAAGCATCCTGCCGAAGAGAAACAGCGACAAGCTGA |
| | | TCGCAAGAAAGAAGGACTGGGACCCGAAGAAGTACGGAGGATTC |
| | | GACAGCCCGACAGTCGCATACAGCGTCCTGGTCGTCGCAAAGGTC |
| | | GAAAAGGGAAAGAGCAAGAAGCTGAAGAGCGTCAAGGAACTGCT |
| | | GGGAATCACAATCATGGAAAGAAGCAGCTTCGAAAAGAACCCGA |
| | | TCGACTTCCTGGAAGCAAAGGGATACAAGGAAGTCAAGAAGGAC |
| | | CTGATCATCAAGCTGCCGAAGTACAGCCTGTTCGAACTGGAAAAC |
| | | GGAAGAAAGAGAATGCTGGCAAGCGCAGGAGAACTGCAGAAGG |
| | | GAAACGAACTGGCACTGCCGAGCAAGTACGTCAACTTCCTGTACC |
| | | TGGCAAGCCACTACGAAAAGCTGAAGGGAAGCCCGGAAGACAAC |
| | | GAACAGAAGCAGCTGTTCGTCGAACAGCACAAGCACTACCTGGA |
| | | CGAAATCATCGAACAGATCAGCGAATTCAGCAAGAGAGTCATCCT |
| | | GGCAGACGCAAACCTGGACAAGGTCCTGAGCGCATACAACAAGC |
| | | ACAGAGACAAGCCGATCAGAGAACAGGCAGAAAACATCATCCAC |
| | | CTGTTCACACTGACAAACCTGGGAGCACCGGCAGCATTCAAGTAC |
| | | TTCGACACAACAATCGACAGAAAGAGATACACAAGCACAAAGGA |
| | | AGTCCTGGACGCAACACTGATCCACCAGAGCATCACAGGACTGTA |
| | | CGAAACAAGAATCGACCTGAGCCAGCTGGGAGGAGACGGAGGAG |
| | | GAAGCCCGAAGAAGAAGAGAAAGGTCTAG |
| ORF | 802 | ATGGACAAGAAGTACTCCATCGGCCTGGACATCGGCACCAACTCC |
| encoding | | GTGGGCTGGGCCGTGATCACCGACGAGTACAAGGTGCCCTCCAAG |
| Sp. Cas9 | | AAGTTCAAGGTGCTGGGCAACACCGACCGGCACTCCATCAAGAA |
| | | GAACCTGATCGGCGCCCTGCTGTTCGACTCCGGCGAGACCGCCGA |
| | | GGCCACCCGGCTGAAGCGGACCGCCCGGCGGCGGTACACCCGGC |
| | | GGAAGAACCGGATCTGCTACCTGCAGGAGATCTTCTCCAACGAGA |
| | | TGGCCAAGGTGGACGACTCCTTCTTCCACCGGCTGGAGGAGTCCT |
| | | TCCTGGTGGAGGAGGACAAGAAGCACGAGCGGCACCCCATCTTC |
| | | GGCAACATCGTGGACGAGGTGGCCTACCACGAGAAGTACCCCAC |
| | | CATCTACCACCTGCGGAAGAAGCTGGTGGACTCCACCGACAAGGC |
| | | CGACCTGCGGCTGATCTACCTGGCCCTGGCCCACATGATCAAGTT |
| | | CCGGGGCCACTTCCTGATCGAGGGCGACCTGAACCCCGACAACTC |
| | | CGACGTGGACAAGCTGTTCATCCAGCTGGTGCAGACCTACAACCA |
| | | GCTGTTCGAGGAGAACCCCATCAACGCCTCCGGCGTGGACGCCAA |
| | | GGCCATCCTGTCCGCCCGGCTGTCCAAGTCCCGGCGGCTGGAGAA |
| | | CCTGATCGCCCAGCTGCCCGGCGAGAAGAAGAACGGCCTGTTCGG |
| | | CAACCTGATCGCCCTGTCCCTGGGCCTGACCCCCAACTTCAAGTC |
| | | CAACTTCGACCTGGCCGAGGACGCCAAGCTGCAGCTGTCCAAGGA |
| | | CACCTACGACGACGACCTGGACAACCTGCTGGCCCAGATCGGCGA |
| | | CCAGTACGCCGACCTGTTCCTGGCCGCCAAGAACCTGTCCGACGC |
| | | CATCCTGCTGTCCGACATCCTGCGGGTGAACACCGAGATCACCAA |
| | | GGCCCCCCTGTCCGCCTCCATGATCAAGCGGTACGACGAGCACCA |
| | | CCAGGACCTGACCCTGCTGAAGGCCCTGGTGCGGCAGCAGCTGCC |
| | | CGAGAAGTACAAGGAGATCTTCTTCGACCAGTCCAAGAACGGCTA |
| | | CGCCGGCTACATCGACGGCGGCGCCTCCCAGGAGGAGTTCTACAA |
| | | GTTCATCAAGCCCATCCTGGAGAAGATGGACGGCACCGAGGAGC |
| | | TGCTGGTGAAGCTGAACCGGGAGGACCTGCTGCGGAAGCAGCGG |
| | | ACCTTCGACAACGGCTCCATCCCCCACCAGATCCACCTGGGCGAG |
| | | CTGCACGCCATCCTGCGGCGGCAGGAGGACTTCTACCCCTTCCTG |
| | | AAGGACAACCGGGAGAAGATCGAGAAGATCCTGACCTTCCGGAT |
| | | CCCCTACTACGTGGGCCCCCTGGCCCGGGGCAACTCCCGGTTCGC |
| | | CTGGATGACCCGGAAGTCCGAGGAGACCATCACCCCCTGGAACTT |
| | | CGAGGAGGTGGTGGACAAGGGCGCCTCCGCCCAGTCCTTCATCGA |
| | | GCGGATGACCAACTTCGACAAGAACCTGCCCAACGAGAAGGTGC |
| | | TGCCCAAGCACTCCCTGCTGTACGAGTACTTCACCGTGTACAACG |
| | | AGCTGACCAAGGTGAAGTACGTGACCGAGGGCATGCGGAAGCCC |
| | | GCCTTCCTGTCCGGCGAGCAGAAGAAGGCCATCGTGGACCTGCTG |
| | | TTCAAGACCAACCGGAAGGTGACCGTGAAGCAGCTGAAGGAGGA |
| | | CTACTTCAAGAAGATCGAGTGCTTCGACTCCGTGGAGATCTCCGG |
| | | CGTGGAGGACCGGTTCAACGCCTCCCTGGGCACCTACCACGACCT |
| | | GCTGAAGATCATCAAGGACAAGGACTTCCTGGACAACGAGGAGA |
| | | ACGAGGACATCCTGGAGGACATCGTGCTGACCCTGACCCTGTTCG |
| | | AGGACCGGGAGATGATCGAGGAGCGGCTGAAGACCTACGCCCAC |
| | | CTGTTCGACGACAAGGTGATGAAGCAGCTGAAGCGGCGGCGGTA |
| | | CACCGGCTGGGGCCGGCTGTCCCGGAAGCTGATCAACGGCATCCG |
| | | GGACAAGCAGTCCGGCAAGACCATCCTGGACTTCCTGAAGTCCGA |
| | | CGGCTTCGCCAACCGGAACTTCATGCAGCTGATCCACGACGACTC |
| | | CCTGACCTTCAAGGAGGACATCCAGAAGGCCCAGGTGTCCGGCCA |
| | | GGGCGACTCCCTGCACGAGCACATCGCCAACCTGGCCGGCTCCCC |
| | | CGCCATCAAGAAGGGCATCCTGCAGACCGTGAAGGTGGTGGACG |
| | | AGCTGGTGAAGGTGATGGGCCGGCACAAGCCCGAGAACATCGTG |
| | | ATCGAGATGGCCCGGGAGAACCAGACCACCCAGAAGGGCCAGAA |
| | | GAACTCCCGGGAGCGGATGAAGCGGATCGAGGAGGGCATCAAGG |
| | | AGCTGGGCTCCCAGATCCTGAAGGAGCACCCCGTGGAGAACACC |
| | | CAGCTGCAGAACGAGAAGCTGTACCTGTACTACCTGCAGAACGGC |
| | | CGGGACATGTACGTGGACCAGGAGCTGGACATCAACCGGCTGTCC |
| | | GACTACGACGTGGACCACATCGTGCCCCAGTCCTTCCTGAAGGAC |
| | | GACTCCATCGACAACAAGGTGCTGACCCGGTCCGACAAGAACCG |
| | | GGGCAAGTCCGACAACGTGCCCTCCGAGGAGGTGGTGAAGAAGA |
| | | TGAAGAACTACTGGCGGCAGCTGCTGAACGCCAAGCTGATCACCC |
| | | AGCGGAAGTTCGACAACCTGACCAAGGCCGAGCGGGGCGGCCTG |
| | | TCCGAGCTGGACAAGGCCGGCTTCATCAAGCGGCAGCTGGTGGA |
| | | GACCCGGCAGATCACCAAGCACGTGGCCCAGATCCTGGACTCCCG |
| | | GATGAACACCAAGTACGACGAGAACGACAAGCTGATCCGGGAGG |
| | | TGAAGGTGATCACCCTGAAGTCCAAGCTGGTGTCCGACTTCCGGA |
| | | AGGACTTCCAGTTCTACAAGGTGCGGGAGATCAACAACTACCACC |
| | | ACGCCCACGACGCCTACCTGAACGCCGTGGTGGGCACCGCCCTGA |
| | | TCAAGAAGTACCCCAAGCTGGAGTCCGAGTTCGTGTACGGCGACT |
| | | ACAAGGTGTACGACGTGCGGAAGATGATCGCCAAGTCCGAGCAG |
| | | GAGATCGGCAAGGCCACCGCCAAGTACTTCTTCTACTCCAACATC |
| | | ATGAACTTCTTCAAGACCGAGATCACCCTGGCCAACGGCGAGATC |
| | | CGGAAGCGGCCCCTGATCGAGACCAACGGCGAGACCGGCGAGAT |
| | | CGTGTGGGACAAGGGCCGGGACTTCGCCACCGTGCGGAAGGTGC |
| | | TGTCCATGCCCCAGGTGAACATCGTGAAGAAGACCGAGGTGCAG |
| | | ACCGGCGGCTTCTCCAAGGAGTCCATCCTGCCCAAGCGGAACTCC |
| | | GACAAGCTGATCGCCCGGAAGAAGGACTGGGACCCCAAGAAGTA |
| | | CGGCGGCTTCGACTCCCCCACCGTGGCCTACTCCGTGCTGGTGGT |
| | | GGCCAAGGTGGAGAAGGGCAAGTCCAAGAAGCTGAAGTCCGTGA |
| | | AGGAGCTGCTGGGCATCACCATCATGGAGCGGTCCTCCTTCGAGA |
| | | AGAACCCCATCGACTTCCTGGAGGCCAAGGGCTACAAGGAGGTG |
| | | AAGAAGGACCTGATCATCAAGCTGCCCAAGTACTCCCTGTTCGAG |
| | | CTGGAGAACGGCCGGAAGCGGATGCTGGCCTCCGCCGGCGAGCT |
| | | GCAGAAGGGCAACGAGCTGGCCCTGCCCTCCAAGTACGTGAACTT |
| | | CCTGTACCTGGCCTCCCACTACGAGAAGCTGAAGGGCTCCCCCGA |
| | | GGACAACGAGCAGAAGCAGCTGTTCGTGGAGCAGCACAAGCACT |
| | | ACCTGGACGAGATCATCGAGCAGATCTCCGAGTTCTCCAAGCGGG |
| | | TGATCCTGGCCGACGCCAACCTGGACAAGGTGCTGTCCGCCTACA |
| | | ACAAGCACCGGGACAAGCCCATCCGGGAGCAGGCCGAGAACATC |
| | | ATCCACCTGTTCACCCTGACCAACCTGGGCGCCCCCGCCGCCTTC |
| | | AAGTACTTCGACACCACCATCGACCGGAAGCGGTACACCTCCACC |
| | | AAGGAGGTGCTGGACGCCACCCTGATCCACCAGTCCATCACCGGC |
| | | CTGTACGAGACCCGGATCGACCTGTCCCAGCTGGGCGGCGACGGC |
| | | GGCGGCTCCCCCAAGAAGAAGCGGAAGGTGTGA |
| Open | 803 | AUGGACAAGAAGUACUCCAUCGGCCUGGACAUCGGCACCAACUC |
| reading | | CGUGGGCUGGGCCGUGAUCACCGACGAGUACAAGGUGCCCUCCA |
| frame for | | AGAAGUUCAAGGUGCUGGGCAACACCGACCGGCACUCCAUCAA |
| Cas9 with | | GAAGAACCUGAUCGGCGCCCUGCUGUUCGACUCCGGCGAGACCG |
| Hibit tag | | CCGAGGCCACCCGGCUGAAGCGGACCGCCCGGCGGCGGUACACC |
| | | CGGCGGAAGAACCGGAUCUGCUACCUGCAGGAGAUCUUCUCCA |
| | | ACGAGAUGGCCAAGGUGGACGACUCCUUCUUCCACCGGCUGGA |
| | | GGAGUCCUUCCUGGUGGAGGAGGACAAGAAGCACGAGCGGCAC |
| | | CCCAUCUUCGGCAACAUCGUGGACGAGGUGGCCUACCACGAGAA |
| | | GUACCCCACCAUCUACCACCUGCGGAAGAAGCUGGUGGACUCCA |
| | | CCGACAAGGCCGACCUGCGGCUGAUCUACCUGGCCCUGGCCCAC |
| | | AUGAUCAAGUUCCGGGGCCACUUCCUGAUCGAGGGCGACCUGA |
| | | ACCCCGACAACUCCGACGUGGACAAGCUGUUCAUCCAGCUGGUG |
| | | CAGACCUACAACCAGCUGUUCGAGGAGAACCCCAUCAACGCCUC |
| | | CGGCGUGGACGCCAAGGCCAUCCUGUCCGCCCGGCUGUCCAAGU |
| | | CCCGGCGGCUGGAGAACCUGAUCGCCCAGCUGCCCGGCGAGAAG |
| | | AAGAACGGCCUGUUCGGCAACCUGAUCGCCCUGUCCCUGGGCCU |
| | | GACCCCCAACUUCAAGUCCAACUUCGACCUGGCCGAGGACGCCA |
| | | AGCUGCAGCUGUCCAAGGACACCUACGACGACGACCUGGACAAC |
| | | CUGCUGGCCCAGAUCGGCGACCAGUACGCCGACCUGUUCCUGGC |
| | | CGCCAAGAACCUGUCCGACGCCAUCCUGCUGUCCGACAUCCUGC |
| | | GGGUGAACACCGAGAUCACCAAGGCCCCCCUGUCCGCCUCCAUG |
| | | AUCAAGCGGUACGACGAGCACCACCAGGACCUGACCCUGCUGAA |
| | | GGCCCUGGUGCGGCAGCAGCUGCCCGAGAAGUACAAGGAGAUC |
| | | UUCUUCGACCAGUCCAAGAACGGCUACGCCGGCUACAUCGACGG |
| | | CGGCGCCUCCCAGGAGGAGUUCUACAAGUUCAUCAAGCCCAUCC |
| | | UGGAGAAGAUGGACGGCACCGAGGAGCUGCUGGUGAAGCUGAA |
| | | CCGGGAGGACCUGCUGCGGAAGCAGCGGACCUUCGACAACGGCU |
| | | CCAUCCCCCACCAGAUCCACCUGGGCGAGCUGCACGCCAUCCUG |
| | | CGGCGGCAGGAGGACUUCUACCCCUUCCUGAAGGACAACCGGGA |
| | | GAAGAUCGAGAAGAUCCUGACCUUCCGGAUCCCCUACUACGUG |
| | | GGCCCCCUGGCCCGGGGCAACUCCCGGUUCGCCUGGAUGACCCG |
| | | GAAGUCCGAGGAGACCAUCACCCCCUGGAACUUCGAGGAGGUG |
| | | GUGGACAAGGGCGCCUCCGCCCAGUCCUUCAUCGAGCGGAUGAC |
| | | CAACUUCGACAAGAACCUGCCCAACGAGAAGGUGCUGCCCAAGC |
| | | ACUCCCUGCUGUACGAGUACUUCACCGUGUACAACGAGCUGACC |
| | | AAGGUGAAGUACGUGACCGAGGGCAUGCGGAAGCCCGCCUUCC |
| | | UGUCCGGCGAGCAGAAGAAGGCCAUCGUGGACCUGCUGUUCAA |
| | | GACCAACCGGAAGGUGACCGUGAAGCAGCUGAAGGAGGACUAC |
| | | UUCAAGAAGAUCGAGUGCUUCGACUCCGUGGAGAUCUCCGGCG |
| | | UGGAGGACCGGUUCAACGCCUCCCUGGGCACCUACCACGACCUG |
| | | CUGAAGAUCAUCAAGGACAAGGACUUCCUGGACAACGAGGAGA |
| | | ACGAGGACAUCCUGGAGGACAUCGUGCUGACCCUGACCCUGUUC |
| | | GAGGACCGGGAGAUGAUCGAGGAGCGGCUGAAGACCUACGCCC |
| | | ACCUGUUCGACGACAAGGUGAUGAAGCAGCUGAAGCGGCGGCG |
| | | GUACACCGGCUGGGGCCGGCUGUCCCGGAAGCUGAUCAACGGCA |
| | | UCCGGGACAAGCAGUCCGGCAAGACCAUCCUGGACUUCCUGAAG |
| | | UCCGACGGCUUCGCCAACCGGAACUUCAUGCAGCUGAUCCACGA |
| | | CGACUCCCUGACCUUCAAGGAGGACAUCCAGAAGGCCCAGGUGU |
| | | CCGGCCAGGGCGACUCCCUGCACGAGCACAUCGCCAACCUGGCC |
| | | GGCUCCCCCGCCAUCAAGAAGGGCAUCCUGCAGACCGUGAAGGU |
| | | GGUGGACGAGCUGGUGAAGGUGAUGGGCCGGCACAAGCCCGAG |
| | | AACAUCGUGAUCGAGAUGGCCCGGGAGAACCAGACCACCCAGA |
| | | AGGGCCAGAAGAACUCCCGGGAGCGGAUGAAGCGGAUCGAGGA |
| | | GGGCAUCAAGGAGCUGGGCUCCCAGAUCCUGAAGGAGCACCCCG |
| | | UGGAGAACACCCAGCUGCAGAACGAGAAGCUGUACCUGUACUA |
| | | CCUGCAGAACGGCCGGGACAUGUACGUGGACCAGGAGCUGGAC |
| | | AUCAACCGGCUGUCCGACUACGACGUGGACCACAUCGUGCCCCA |
| | | GUCCUUCCUGAAGGACGACUCCAUCGACAACAAGGUGCUGACCC |
| | | GGUCCGACAAGAACCGGGGCAAGUCCGACAACGUGCCCUCCGAG |
| | | GAGGUGGUGAAGAAGAUGAAGAACUACUGGCGGCAGCUGCUGA |
| | | ACGCCAAGCUGAUCACCCAGCGGAAGUUCGACAACCUGACCAAG |
| | | GCCGAGCGGGGCGGCCUGUCCGAGCUGGACAAGGCCGGCUUCAU |
| | | CAAGCGGCAGCUGGUGGAGACCCGGCAGAUCACCAAGCACGUG |
| | | GCCCAGAUCCUGGACUCCCGGAUGAACACCAAGUACGACGAGAA |
| | | CGACAAGCUGAUCCGGGAGGUGAAGGUGAUCACCCUGAAGUCC |
| | | AAGCUGGUGUCCGACUUCCGGAAGGACUUCCAGUUCUACAAGG |
| | | UGCGGGAGAUCAACAACUACCACCACGCCCACGACGCCUACCUG |
| | | AACGCCGUGGUGGGCACCGCCCUGAUCAAGAAGUACCCCAAGCU |
| | | GGAGUCCGAGUUCGUGUACGGCGACUACAAGGUGUACGACGUG |
| | | CGGAAGAUGAUCGCCAAGUCCGAGCAGGAGAUCGGCAAGGCCA |
| | | CCGCCAAGUACUUCUUCUACUCCAACAUCAUGAACUUCUUCAAG |
| | | ACCGAGAUCACCCUGGCCAACGGCGAGAUCCGGAAGCGGCCCCU |
| | | GAUCGAGACCAACGGCGAGACCGGCGAGAUCGUGUGGGACAAG |
| | | GGCCGGGACUUCGCCACCGUGCGGAAGGUGCUGUCCAUGCCCCA |
| | | GGUGAACAUCGUGAAGAAGACCGAGGUGCAGACCGGCGGCUUC |
| | | UCCAAGGAGUCCAUCCUGCCCAAGCGGAACUCCGACAAGCUGAU |
| | | CGCCCGGAAGAAGGACUGGGACCCCAAGAAGUACGGCGGCUUC |
| | | GACUCCCCCACCGUGGCCUACUCCGUGCUGGUGGUGGCCAAGGU |
| | | GGAGAAGGGCAAGUCCAAGAAGCUGAAGUCCGUGAAGGAGCUG |
| | | CUGGGCAUCACCAUCAUGGAGCGGUCCUCCUUCGAGAAGAACCC |
| | | CAUCGACUUCCUGGAGGCCAAGGGCUACAAGGAGGUGAAGAAG |
| | | GACCUGAUCAUCAAGCUGCCCAAGUACUCCCUGUUCGAGCUGGA |
| | | GAACGGCCGGAAGCGGAUGCUGGCCUCCGCCGGCGAGCUGCAGA |
| | | AGGGCAACGAGCUGGCCCUGCCCUCCAAGUACGUGAACUUCCUG |
| | | UACCUGGCCUCCCACUACGAGAAGCUGAAGGGCUCCCCCGAGGA |
| | | CAACGAGCAGAAGCAGCUGUUCGUGGAGCAGCACAAGCACUAC |
| | | CUGGACGAGAUCAUCGAGCAGAUCUCCGAGUUCUCCAAGCGGG |
| | | UGAUCCUGGCCGACGCCAACCUGGACAAGGUGCUGUCCGCCUAC |
| | | AACAAGCACCGGGACAAGCCCAUCCGGGAGCAGGCCGAGAACAU |
| | | CAUCCACCUGUUCACCCUGACCAACCUGGGCGCCCCCGCCGCCU |
| | | UCAAGUACUUCGACACCACCAUCGACCGGAAGCGGUACACCUCC |
| | | ACCAAGGAGGUGCUGGACGCCACCCUGAUCCACCAGUCCAUCAC |
| | | CGGCCUGUACGAGACCCGGAUCGACCUGUCCCAGCUGGGCGGCG |
| | | ACGGCGGCGGCUCCCCCAAGAAGAAGCGGAAGGUGUCCGAGUCC |
| | | GCCACCCCCGAGUCCGUGUCCGGCUGGCGGCUGUUCAAGAAGAU |
| | | CUCCUGA |
| Open | 804 | AUGGAGGCCUCCCCCGCCUCCGGCCCCCGGCACCUGAUGGACCC |
| Reading | | CCACAUCUUCACCUCCAACUUCAACAACGGCAUCGGCCGGCACA |
| frame for | | AGACCUACCUGUGCUACGAGGUGGAGCGGCUGGACAACGGCAC |
| BC22n | | CUCCGUGAAGAUGGACCAGCACCGGGGCUUCCUGCACAACCAGG |
| | | CCAAGAACCUGCUGUGCGGCUUCUACGGCCGGCACGCCGAGCUG |
| | | CGGUUCCUGGACCUGGUGCCCUCCCUGCAGCUGGACCCCGCCCA |
| | | GAUCUACCGGGUGACCUGGUUCAUCUCCUGGUCCCCCUGCUUCU |
| | | CCUGGGGCUGCGCCGGCGAGGUGCGGGCCUUCCUGCAGGAGAAC |
| | | ACCCACGUGCGGCUGCGGAUCUUCGCCGCCCGGAUCUACGACUA |
| | | CGACCCCCUGUACAAGGAGGCCCUGCAGAUGCUGCGGGACGCCG |
| | | GCGCCCAGGUGUCCAUCAUGACCUACGACGAGUUCAAGCACUGC |
| | | UGGGACACCUUCGUGGACCACCAGGGCUGCCCCUUCCAGCCCUG |
| | | GGACGGCCUGGACGAGCACUCCCAGGCCCUGUCCGGCCGGCUGC |
| | | GGGCCAUCCUGCAGAACCAGGGCAACUCCGGCUCCGAGACCCCC |
| | | GGCACCUCCGAGUCCGCCACCCCCGAGUCCGACAAGAAGUACUC |
| | | CAUCGGCCUGGCCAUCGGCACCAACUCCGUGGGCUGGGCCGUGA |
| | | UCACCGACGAGUACAAGGUGCCCUCCAAGAAGUUCAAGGUGCU |
| | | GGGCAACACCGACCGGCACUCCAUCAAGAAGAACCUGAUCGGCG |
| | | CCCUGCUGUUCGACUCCGGCGAGACCGCCGAGGCCACCCGGCUG |
| | | AAGCGGACCGCCCGGCGGCGGUACACCCGGCGGAAGAACCGGAU |
| | | CUGCUACCUGCAGGAGAUCUUCUCCAACGAGAUGGCCAAGGUG |
| | | GACGACUCCUUCUUCCACCGGCUGGAGGAGUCCUUCCUGGUGGA |
| | | GGAGGACAAGAAGCACGAGCGGCACCCCAUCUUCGGCAACAUCG |
| | | UGGACGAGGUGGCCUACCACGAGAAGUACCCCACCAUCUACCAC |
| | | CUGCGGAAGAAGCUGGUGGACUCCACCGACAAGGCCGACCUGCG |
| | | GCUGAUCUACCUGGCCCUGGCCCACAUGAUCAAGUUCCGGGGCC |
| | | ACUUCCUGAUCGAGGGCGACCUGAACCCCGACAACUCCGACGUG |
| | | GACAAGCUGUUCAUCCAGCUGGUGCAGACCUACAACCAGCUGU |
| | | UCGAGGAGAACCCCAUCAACGCCUCCGGCGUGGACGCCAAGGCC |
| | | AUCCUGUCCGCCCGGCUGUCCAAGUCCCGGCGGCUGGAGAACCU |
| | | GAUCGCCCAGCUGCCCGGCGAGAAGAAGAACGGCCUGUUCGGCA |
| | | ACCUGAUCGCCCUGUCCCUGGGCCUGACCCCCAACUUCAAGUCC |
| | | AACUUCGACCUGGCCGAGGACGCCAAGCUGCAGCUGUCCAAGGA |
| | | CACCUACGACGACGACCUGGACAACCUGCUGGCCCAGAUCGGCG |
| | | ACCAGUACGCCGACCUGUUCCUGGCCGCCAAGAACCUGUCCGAC |
| | | GCCAUCCUGCUGUCCGACAUCCUGCGGGUGAACACCGAGAUCAC |
| | | CAAGGCCCCCCUGUCCGCCUCCAUGAUCAAGCGGUACGACGAGC |
| | | ACCACCAGGACCUGACCCUGCUGAAGGCCCUGGUGCGGCAGCAG |
| | | CUGCCCGAGAAGUACAAGGAGAUCUUCUUCGACCAGUCCAAGA |
| | | ACGGCUACGCCGGCUACAUCGACGGCGGCGCCUCCCAGGAGGAG |
| | | UUCUACAAGUUCAUCAAGCCCAUCCUGGAGAAGAUGGACGGCA |
| | | CCGAGGAGCUGCUGGUGAAGCUGAACCGGGAGGACCUGCUGCG |
| | | GAAGCAGCGGACCUUCGACAACGGCUCCAUCCCCCACCAGAUCC |
| | | ACCUGGGCGAGCUGCACGCCAUCCUGCGGCGGCAGGAGGACUUC |
| | | UACCCCUUCCUGAAGGACAACCGGGAGAAGAUCGAGAAGAUCC |
| | | UGACCUUCCGGAUCCCCUACUACGUGGGCCCCCUGGCCCGGGGC |
| | | AACUCCCGGUUCGCCUGGAUGACCCGGAAGUCCGAGGAGACCAU |
| | | CACCCCCUGGAACUUCGAGGAGGUGGUGGACAAGGGCGCCUCCG |
| | | CCCAGUCCUUCAUCGAGCGGAUGACCAACUUCGACAAGAACCUG |
| | | CCCAACGAGAAGGUGCUGCCCAAGCACUCCCUGCUGUACGAGUA |
| | | CUUCACCGUGUACAACGAGCUGACCAAGGUGAAGUACGUGACC |
| | | GAGGGCAUGCGGAAGCCCGCCUUCCUGUCCGGCGAGCAGAAGA |
| | | AGGCCAUCGUGGACCUGCUGUUCAAGACCAACCGGAAGGUGAC |
| | | CGUGAAGCAGCUGAAGGAGGACUACUUCAAGAAGAUCGAGUGC |
| | | UUCGACUCCGUGGAGAUCUCCGGCGUGGAGGACCGGUUCAACG |
| | | CCUCCCUGGGCACCUACCACGACCUGCUGAAGAUCAUCAAGGAC |
| | | AAGGACUUCCUGGACAACGAGGAGAACGAGGACAUCCUGGAGG |
| | | ACAUCGUGCUGACCCUGACCCUGUUCGAGGACCGGGAGAUGAU |
| | | CGAGGAGCGGCUGAAGACCUACGCCCACCUGUUCGACGACAAGG |
| | | UGAUGAAGCAGCUGAAGCGGCGGCGGUACACCGGCUGGGGCCG |
| | | GCUGUCCCGGAAGCUGAUCAACGGCAUCCGGGACAAGCAGUCCG |
| | | GCAAGACCAUCCUGGACUUCCUGAAGUCCGACGGCUUCGCCAAC |
| | | CGGAACUUCAUGCAGCUGAUCCACGACGACUCCCUGACCUUCAA |
| | | GGAGGACAUCCAGAAGGCCCAGGUGUCCGGCCAGGGCGACUCCC |
| | | UGCACGAGCACAUCGCCAACCUGGCCGGCUCCCCCGCCAUCAAG |
| | | AAGGGCAUCCUGCAGACCGUGAAGGUGGUGGACGAGCUGGUGA |
| | | AGGUGAUGGGCCGGCACAAGCCCGAGAACAUCGUGAUCGAGAU |
| | | GGCCCGGGAGAACCAGACCACCCAGAAGGGCCAGAAGAACUCCC |
| | | GGGAGCGGAUGAAGCGGAUCGAGGAGGGCAUCAAGGAGCUGGG |
| | | CUCCCAGAUCCUGAAGGAGCACCCCGUGGAGAACACCCAGCUGC |
| | | AGAACGAGAAGCUGUACCUGUACUACCUGCAGAACGGCCGGGA |
| | | CAUGUACGUGGACCAGGAGCUGGACAUCAACCGGCUGUCCGAC |
| | | UACGACGUGGACCACAUCGUGCCCCAGUCCUUCCUGAAGGACGA |
| | | CUCCAUCGACAACAAGGUGCUGACCCGGUCCGACAAGAACCGGG |
| | | GCAAGUCCGACAACGUGCCCUCCGAGGAGGUGGUGAAGAAGAU |
| | | GAAGAACUACUGGCGGCAGCUGCUGAACGCCAAGCUGAUCACCC |
| | | AGCGGAAGUUCGACAACCUGACCAAGGCCGAGCGGGGCGGCCU |
| | | GUCCGAGCUGGACAAGGCCGGCUUCAUCAAGCGGCAGCUGGUG |
| | | GAGACCCGGCAGAUCACCAAGCACGUGGCCCAGAUCCUGGACUC |
| | | CCGGAUGAACACCAAGUACGACGAGAACGACAAGCUGAUCCGG |
| | | GAGGUGAAGGUGAUCACCCUGAAGUCCAAGCUGGUGUCCGACU |
| | | UCCGGAAGGACUUCCAGUUCUACAAGGUGCGGGAGAUCAACAA |
| | | CUACCACCACGCCCACGACGCCUACCUGAACGCCGUGGUGGGCA |
| | | CCGCCCUGAUCAAGAAGUACCCCAAGCUGGAGUCCGAGUUCGUG |
| | | UACGGCGACUACAAGGUGUACGACGUGCGGAAGAUGAUCGCCA |
| | | AGUCCGAGCAGGAGAUCGGCAAGGCCACCGCCAAGUACUUCUUC |
| | | UACUCCAACAUCAUGAACUUCUUCAAGACCGAGAUCACCCUGGC |
| | | CAACGGCGAGAUCCGGAAGCGGCCCCUGAUCGAGACCAACGGCG |
| | | AGACCGGCGAGAUCGUGUGGGACAAGGGCCGGGACUUCGCCAC |
| | | CGUGCGGAAGGUGCUGUCCAUGCCCCAGGUGAACAUCGUGAAG |
| | | AAGACCGAGGUGCAGACCGGCGGCUUCUCCAAGGAGUCCAUCCU |
| | | GCCCAAGCGGAACUCCGACAAGCUGAUCGCCCGGAAGAAGGACU |
| | | GGGACCCCAAGAAGUACGGCGGCUUCGACUCCCCCACCGUGGCC |
| | | UACUCCGUGCUGGUGGUGGCCAAGGUGGAGAAGGGCAAGUCCA |
| | | AGAAGCUGAAGUCCGUGAAGGAGCUGCUGGGCAUCACCAUCAU |
| | | GGAGCGGUCCUCCUUCGAGAAGAACCCCAUCGACUUCCUGGAGG |
| | | CCAAGGGCUACAAGGAGGUGAAGAAGGACCUGAUCAUCAAGCU |
| | | GCCCAAGUACUCCCUGUUCGAGCUGGAGAACGGCCGGAAGCGG |
| | | AUGCUGGCCUCCGCCGGCGAGCUGCAGAAGGGCAACGAGCUGGC |
| | | CCUGCCCUCCAAGUACGUGAACUUCCUGUACCUGGCCUCCCACU |
| | | ACGAGAAGCUGAAGGGCUCCCCCGAGGACAACGAGCAGAAGCA |
| | | GCUGUUCGUGGAGCAGCACAAGCACUACCUGGACGAGAUCAUC |
| | | GAGCAGAUCUCCGAGUUCUCCAAGCGGGUGAUCCUGGCCGACGC |
| | | CAACCUGGACAAGGUGCUGUCCGCCUACAACAAGCACCGGGACA |
| | | AGCCCAUCCGGGAGCAGGCCGAGAACAUCAUCCACCUGUUCACC |
| | | CUGACCAACCUGGGCGCCCCCGCCGCCUUCAAGUACUUCGACAC |
| | | CACCAUCGACCGGAAGCGGUACACCUCCACCAAGGAGGUGCUGG |
| | | ACGCCACCCUGAUCCACCAGUCCAUCACCGGCCUGUACGAGACC |
| | | CGGAUCGACCUGUCCCAGCUGGGCGGCGACGGCGGCGGCUCCCC |
| | | CAAGAAGAAGCGGAAGGUGUGA |
| Open | 805 | AUGGAGGCCUCCCCCGCCUCCGGCCCCCGGCACCUGAUGGACCC |
| reading | | CCACAUCUUCACCUCCAACUUCAACAACGGCAUCGGCCGGCACA |
| frame for | | AGACCUACCUGUGCUACGAGGUGGAGCGGCUGGACAACGGCAC |
| BC22n | | CUCCGUGAAGAUGGACCAGCACCGGGGCUUCCUGCACAACCAGG |
| with Hibit | | CCAAGAACCUGCUGUGCGGCUUCUACGGCCGGCACGCCGAGCUG |
| tag | | CGGUUCCUGGACCUGGUGCCCUCCCUGCAGCUGGACCCCGCCCA |
| | | GAUCUACCGGGUGACCUGGUUCAUCUCCUGGUCCCCCUGCUUCU |
| | | CCUGGGGCUGCGCCGGCGAGGUGCGGGCCUUCCUGCAGGAGAAC |
| | | ACCCACGUGCGGCUGCGGAUCUUCGCCGCCCGGAUCUACGACUA |
| | | CGACCCCCUGUACAAGGAGGCCCUGCAGAUGCUGCGGGACGCCG |
| | | GCGCCCAGGUGUCCAUCAUGACCUACGACGAGUUCAAGCACUGC |
| | | UGGGACACCUUCGUGGACCACCAGGGCUGCCCCUUCCAGCCCUG |
| | | GGACGGCCUGGACGAGCACUCCCAGGCCCUGUCCGGCCGGCUGC |
| | | GGGCCAUCCUGCAGAACCAGGGCAACUCCGGCUCCGAGACCCCC |
| | | GGCACCUCCGAGUCCGCCACCCCCGAGUCCGACAAGAAGUACUC |
| | | CAUCGGCCUGGCCAUCGGCACCAACUCCGUGGGCUGGGCCGUGA |
| | | UCACCGACGAGUACAAGGUGCCCUCCAAGAAGUUCAAGGUGCU |
| | | GGGCAACACCGACCGGCACUCCAUCAAGAAGAACCUGAUCGGCG |
| | | CCCUGCUGUUCGACUCCGGCGAGACCGCCGAGGCCACCCGGCUG |
| | | AAGCGGACCGCCCGGCGGCGGUACACCCGGCGGAAGAACCGGAU |
| | | CUGCUACCUGCAGGAGAUCUUCUCCAACGAGAUGGCCAAGGUG |
| | | GACGACUCCUUCUUCCACCGGCUGGAGGAGUCCUUCCUGGUGGA |
| | | GGAGGACAAGAAGCACGAGCGGCACCCCAUCUUCGGCAACAUCG |
| | | UGGACGAGGUGGCCUACCACGAGAAGUACCCCACCAUCUACCAC |
| | | CUGCGGAAGAAGCUGGUGGACUCCACCGACAAGGCCGACCUGCG |
| | | GCUGAUCUACCUGGCCCUGGCCCACAUGAUCAAGUUCCGGGGCC |
| | | ACUUCCUGAUCGAGGGCGACCUGAACCCCGACAACUCCGACGUG |
| | | GACAAGCUGUUCAUCCAGCUGGUGCAGACCUACAACCAGCUGU |
| | | UCGAGGAGAACCCCAUCAACGCCUCCGGCGUGGACGCCAAGGCC |
| | | AUCCUGUCCGCCCGGCUGUCCAAGUCCCGGCGGCUGGAGAACCU |
| | | GAUCGCCCAGCUGCCCGGCGAGAAGAAGAACGGCCUGUUCGGCA |
| | | ACCUGAUCGCCCUGUCCCUGGGCCUGACCCCCAACUUCAAGUCC |
| | | AACUUCGACCUGGCCGAGGACGCCAAGCUGCAGCUGUCCAAGGA |
| | | CACCUACGACGACGACCUGGACAACCUGCUGGCCCAGAUCGGCG |
| | | ACCAGUACGCCGACCUGUUCCUGGCCGCCAAGAACCUGUCCGAC |
| | | GCCAUCCUGCUGUCCGACAUCCUGCGGGUGAACACCGAGAUCAC |
| | | CAAGGCCCCCCUGUCCGCCUCCAUGAUCAAGCGGUACGACGAGC |
| | | ACCACCAGGACCUGACCCUGCUGAAGGCCCUGGUGCGGCAGCAG |
| | | CUGCCCGAGAAGUACAAGGAGAUCUUCUUCGACCAGUCCAAGA |
| | | ACGGCUACGCCGGCUACAUCGACGGCGGCGCCUCCCAGGAGGAG |
| | | UUCUACAAGUUCAUCAAGCCCAUCCUGGAGAAGAUGGACGGCA |
| | | CCGAGGAGCUGCUGGUGAAGCUGAACCGGGAGGACCUGCUGCG |
| | | GAAGCAGCGGACCUUCGACAACGGCUCCAUCCCCCACCAGAUCC |
| | | ACCUGGGCGAGCUGCACGCCAUCCUGCGGCGGCAGGAGGACUUC |
| | | UACCCCUUCCUGAAGGACAACCGGGAGAAGAUCGAGAAGAUCC |
| | | UGACCUUCCGGAUCCCCUACUACGUGGGCCCCCUGGCCCGGGGC |
| | | AACUCCCGGUUCGCCUGGAUGACCCGGAAGUCCGAGGAGACCAU |
| | | CACCCCCUGGAACUUCGAGGAGGUGGUGGACAAGGGCGCCUCCG |
| | | CCCAGUCCUUCAUCGAGCGGAUGACCAACUUCGACAAGAACCUG |
| | | CCCAACGAGAAGGUGCUGCCCAAGCACUCCCUGCUGUACGAGUA |
| | | CUUCACCGUGUACAACGAGCUGACCAAGGUGAAGUACGUGACC |
| | | GAGGGCAUGCGGAAGCCCGCCUUCCUGUCCGGCGAGCAGAAGA |
| | | AGGCCAUCGUGGACCUGCUGUUCAAGACCAACCGGAAGGUGAC |
| | | CGUGAAGCAGCUGAAGGAGGACUACUUCAAGAAGAUCGAGUGC |
| | | UUCGACUCCGUGGAGAUCUCCGGCGUGGAGGACCGGUUCAACG |
| | | CCUCCCUGGGCACCUACCACGACCUGCUGAAGAUCAUCAAGGAC |
| | | AAGGACUUCCUGGACAACGAGGAGAACGAGGACAUCCUGGAGG |
| | | ACAUCGUGCUGACCCUGACCCUGUUCGAGGACCGGGAGAUGAU |
| | | CGAGGAGCGGCUGAAGACCUACGCCCACCUGUUCGACGACAAGG |
| | | UGAUGAAGCAGCUGAAGCGGCGGCGGUACACCGGCUGGGGCCG |
| | | GCUGUCCCGGAAGCUGAUCAACGGCAUCCGGGACAAGCAGUCCG |
| | | GCAAGACCAUCCUGGACUUCCUGAAGUCCGACGGCUUCGCCAAC |
| | | CGGAACUUCAUGCAGCUGAUCCACGACGACUCCCUGACCUUCAA |
| | | GGAGGACAUCCAGAAGGCCCAGGUGUCCGGCCAGGGCGACUCCC |
| | | UGCACGAGCACAUCGCCAACCUGGCCGGCUCCCCCGCCAUCAAG |
| | | AAGGGCAUCCUGCAGACCGUGAAGGUGGUGGACGAGCUGGUGA |
| | | AGGUGAUGGGCCGGCACAAGCCCGAGAACAUCGUGAUCGAGAU |
| | | GGCCCGGGAGAACCAGACCACCCAGAAGGGCCAGAAGAACUCCC |
| | | GGGAGCGGAUGAAGCGGAUCGAGGAGGGCAUCAAGGAGCUGGG |
| | | CUCCCAGAUCCUGAAGGAGCACCCCGUGGAGAACACCCAGCUGC |
| | | AGAACGAGAAGCUGUACCUGUACUACCUGCAGAACGGCCGGGA |
| | | CAUGUACGUGGACCAGGAGCUGGACAUCAACCGGCUGUCCGAC |
| | | UACGACGUGGACCACAUCGUGCCCCAGUCCUUCCUGAAGGACGA |
| | | CUCCAUCGACAACAAGGUGCUGACCCGGUCCGACAAGAACCGGG |
| | | GCAAGUCCGACAACGUGCCCUCCGAGGAGGUGGUGAAGAAGAU |
| | | GAAGAACUACUGGCGGCAGCUGCUGAACGCCAAGCUGAUCACCC |
| | | AGCGGAAGUUCGACAACCUGACCAAGGCCGAGCGGGGCGGCCU |
| | | GUCCGAGCUGGACAAGGCCGGCUUCAUCAAGCGGCAGCUGGUG |
| | | GAGACCCGGCAGAUCACCAAGCACGUGGCCCAGAUCCUGGACUC |
| | | CCGGAUGAACACCAAGUACGACGAGAACGACAAGCUGAUCCGG |
| | | GAGGUGAAGGUGAUCACCCUGAAGUCCAAGCUGGUGUCCGACU |
| | | UCCGGAAGGACUUCCAGUUCUACAAGGUGCGGGAGAUCAACAA |
| | | CUACCACCACGCCCACGACGCCUACCUGAACGCCGUGGUGGGCA |
| | | CCGCCCUGAUCAAGAAGUACCCCAAGCUGGAGUCCGAGUUCGUG |
| | | UACGGCGACUACAAGGUGUACGACGUGCGGAAGAUGAUCGCCA |
| | | AGUCCGAGCAGGAGAUCGGCAAGGCCACCGCCAAGUACUUCUUC |
| | | UACUCCAACAUCAUGAACUUCUUCAAGACCGAGAUCACCCUGGC |
| | | CAACGGCGAGAUCCGGAAGCGGCCCCUGAUCGAGACCAACGGCG |
| | | AGACCGGCGAGAUCGUGUGGGACAAGGGCCGGGACUUCGCCAC |
| | | CGUGCGGAAGGUGCUGUCCAUGCCCCAGGUGAACAUCGUGAAG |
| | | AAGACCGAGGUGCAGACCGGCGGCUUCUCCAAGGAGUCCAUCCU |
| | | GCCCAAGCGGAACUCCGACAAGCUGAUCGCCCGGAAGAAGGACU |
| | | GGGACCCCAAGAAGUACGGCGGCUUCGACUCCCCCACCGUGGCC |
| | | UACUCCGUGCUGGUGGUGGCCAAGGUGGAGAAGGGCAAGUCCA |
| | | AGAAGCUGAAGUCCGUGAAGGAGCUGCUGGGCAUCACCAUCAU |
| | | GGAGCGGUCCUCCUUCGAGAAGAACCCCAUCGACUUCCUGGAGG |
| | | CCAAGGGCUACAAGGAGGUGAAGAAGGACCUGAUCAUCAAGCU |
| | | GCCCAAGUACUCCCUGUUCGAGCUGGAGAACGGCCGGAAGCGG |
| | | AUGCUGGCCUCCGCCGGCGAGCUGCAGAAGGGCAACGAGCUGGC |
| | | CCUGCCCUCCAAGUACGUGAACUUCCUGUACCUGGCCUCCCACU |
| | | ACGAGAAGCUGAAGGGCUCCCCCGAGGACAACGAGCAGAAGCA |
| | | GCUGUUCGUGGAGCAGCACAAGCACUACCUGGACGAGAUCAUC |
| | | GAGCAGAUCUCCGAGUUCUCCAAGCGGGUGAUCCUGGCCGACGC |
| | | CAACCUGGACAAGGUGCUGUCCGCCUACAACAAGCACCGGGACA |
| | | AGCCCAUCCGGGAGCAGGCCGAGAACAUCAUCCACCUGUUCACC |
| | | CUGACCAACCUGGGCGCCCCCGCCGCCUUCAAGUACUUCGACAC |
| | | CACCAUCGACCGGAAGCGGUACACCUCCACCAAGGAGGUGCUGG |
| | | ACGCCACCCUGAUCCACCAGUCCAUCACCGGCCUGUACGAGACC |
| | | CGGAUCGACCUGUCCCAGCUGGGCGGCGACGGCGGCGGCUCCCC |
| | | CAAGAAGAAGCGGAAGGUGUCCGAGUCCGCCACCCCCGAGUCCG |
| | | UGUCCGGCUGGCGGCUGUUCAAGAAGAUCUCCUGA |
| Open | 806 | AUGGAAGCAAGCCCGGCAAGCGGACCGAGACACCUGAUGGACCC |
| reading | | GCACAUCUUCACAAGCAACUUCAACAACGGAAUCGGAAGACAC |
| frame for | | AAGACAUACCUGUGCUACGAAGUCGAAAGACUGGACAACGGAA |
| BC22 | | CAAGCGUCAAGAUGGACCAGCACAGAGGAUUCCUGCACAACCA |
| | | GGCAAAGAACCUGCUGUGCGGAUUCUACGGAAGACACGCAGAA |
| | | CUGAGAUUCCUGGACCUGGUCCCGAGCCUGCAGCUGGACCCGGC |
| | | ACAGAUCUACAGAGUCACAUGGUUCAUCAGCUGGAGCCCGUGC |
| | | UUCAGCUGGGGAUGCGCAGGAGAAGUCAGAGCAUUUCUGCAGG |
| | | AAAACACACACGUCAGACUGAGAAUCUUCGCAGCAAGAAUCUA |
| | | CGACUACGACCCGCUGUACAAGGAAGCACUGCAGAUGCUGAGA |
| | | GACGCAGGAGCACAGGUCAGCAUCAUGACAUACGACGAAUUCA |
| | | AGCACUGCUGGGACACAUUCGUCGACCACCAGGGAUGCCCGUUC |
| | | CAGCCGUGGGACGGACUGGACGAACACAGCCAGGCACUGAGCG |
| | | GAAGACUGAGAGCAAUCCUGCAGAACCAGGGAAACAGCGGAAG |
| | | CGAAACACCGGGAACAAGCGAAAGCGCAACACCGGAAAGCGAC |
| | | AAGAAGUACAGCAUCGGACUGGCCAUCGGAACAAACAGCGUCG |
| | | GAUGGGCAGUCAUCACAGACGAAUACAAGGUCCCGAGCAAGAA |
| | | GUUCAAGGUCCUGGGAAACACAGACAGACACAGCAUCAAGAAG |
| | | AACCUGAUCGGAGCACUGCUGUUCGACAGCGGAGAAACAGCAG |
| | | AAGCAACAAGACUGAAGAGAACAGCAAGAAGAAGAUACACAAG |
| | | AAGAAAGAACAGAAUCUGCUACCUGCAGGAAAUCUUCAGCAAC |
| | | GAAAUGGCAAAGGUCGACGACAGCUUCUUCCACAGACUGGAAG |
| | | AAAGCUUCCUGGUCGAAGAAGACAAGAAGCACGAAAGACACCC |
| | | GAUCUUCGGAAACAUCGUCGACGAAGUCGCAUACCACGAAAAG |
| | | UACCCGACAAUCUACCACCUGAGAAAGAAGCUGGUCGACAGCAC |
| | | AGACAAGGCAGACCUGAGACUGAUCUACCUGGCACUGGCACAC |
| | | AUGAUCAAGUUCAGAGGACACUUCCUGAUCGAAGGAGACCUGA |
| | | ACCCGGACAACAGCGACGUCGACAAGCUGUUCAUCCAGCUGGUC |
| | | CAGACAUACAACCAGCUGUUCGAAGAAAACCCGAUCAACGCAA |
| | | GCGGAGUCGACGCAAAGGCAAUCCUGAGCGCAAGACUGAGCAA |
| | | GAGCAGAAGACUGGAAAACCUGAUCGCACAGCUGCCGGGAGAA |
| | | AAGAAGAACGGACUGUUCGGAAACCUGAUCGCACUGAGCCUGG |
| | | GACUGACACCGAACUUCAAGAGCAACUUCGACCUGGCAGAAGA |
| | | CGCAAAGCUGCAGCUGAGCAAGGACACAUACGACGACGACCUG |
| | | GACAACCUGCUGGCACAGAUCGGAGACCAGUACGCAGACCUGU |
| | | UCCUGGCAGCAAAGAACCUGAGCGACGCAAUCCUGCUGAGCGAC |
| | | AUCCUGAGAGUCAACACAGAAAUCACAAAGGCACCGCUGAGCG |
| | | CAAGCAUGAUCAAGAGAUACGACGAACACCACCAGGACCUGAC |
| | | ACUGCUGAAGGCACUGGUCAGACAGCAGCUGCCGGAAAAGUAC |
| | | AAGGAAAUCUUCUUCGACCAGAGCAAGAACGGAUACGCAGGAU |
| | | ACAUCGACGGAGGAGCAAGCCAGGAAGAAUUCUACAAGUUCAU |
| | | CAAGCCGAUCCUGGAAAAGAUGGACGGAACAGAAGAACUGCUG |
| | | GUCAAGCUGAACAGAGAAGACCUGCUGAGAAAGCAGAGAACAU |
| | | UCGACAACGGAAGCAUCCCGCACCAGAUCCACCUGGGAGAACUG |
| | | CACGCAAUCCUGAGAAGACAGGAAGACUUCUACCCGUUCCUGA |
| | | AGGACAACAGAGAAAAGAUCGAAAAGAUCCUGACAUUCAGAAU |
| | | CCCGUACUACGUCGGACCGCUGGCAAGAGGAAACAGCAGAUUC |
| | | GCAUGGAUGACAAGAAAGAGCGAAGAAACAAUCACACCGUGGA |
| | | ACUUCGAAGAAGUCGUCGACAAGGGAGCAAGCGCACAGAGCUU |
| | | CAUCGAAAGAAUGACAAACUUCGACAAGAACCUGCCGAACGAA |
| | | AAGGUCCUGCCGAAGCACAGCCUGCUGUACGAAUACUUCACAG |
| | | UCUACAACGAACUGACAAAGGUCAAGUACGUCACAGAAGGAAU |
| | | GAGAAAGCCGGCAUUCCUGAGCGGAGAACAGAAGAAGGCAAUC |
| | | GUCGACCUGCUGUUCAAGACAAACAGAAAGGUCACAGUCAAGC |
| | | AGCUGAAGGAAGACUACUUCAAGAAGAUCGAAUGCUUCGACAG |
| | | CGUCGAAAUCAGCGGAGUCGAAGACAGAUUCAACGCAAGCCUG |
| | | GGAACAUACCACGACCUGCUGAAGAUCAUCAAGGACAAGGACU |
| | | UCCUGGACAACGAAGAAAACGAAGACAUCCUGGAAGACAUCGU |
| | | CCUGACACUGACACUGUUCGAAGACAGAGAAAUGAUCGAAGAA |
| | | AGACUGAAGACAUACGCACACCUGUUCGACGACAAGGUCAUGA |
| | | AGCAGCUGAAGAGAAGAAGAUACACAGGAUGGGGAAGACUGAG |
| | | CAGAAAGCUGAUCAACGGAAUCAGAGACAAGCAGAGCGGAAAG |
| | | ACAAUCCUGGACUUCCUGAAGAGCGACGGAUUCGCAAACAGAA |
| | | ACUUCAUGCAGCUGAUCCACGACGACAGCCUGACAUUCAAGGA |
| | | AGACAUCCAGAAGGCACAGGUCAGCGGACAGGGAGACAGCCUG |
| | | CACGAACACAUCGCAAACCUGGCAGGAAGCCCGGCAAUCAAGAA |
| | | GGGAAUCCUGCAGACAGUCAAGGUCGUCGACGAACUGGUCAAG |
| | | GUCAUGGGAAGACACAAGCCGGAAAACAUCGUCAUCGAAAUGG |
| | | CAAGAGAAAACCAGACAACACAGAAGGGACAGAAGAACAGCAG |
| | | AGAAAGAAUGAAGAGAAUCGAAGAAGGAAUCAAGGAACUGGGA |
| | | AGCCAGAUCCUGAAGGAACACCCGGUCGAAAACACACAGCUGCA |
| | | GAACGAAAAGCUGUACCUGUACUACCUGCAGAACGGAAGAGAC |
| | | AUGUACGUCGACCAGGAACUGGACAUCAACAGACUGAGCGACU |
| | | ACGACGUCGACCACAUCGUCCCGCAGAGCUUCCUGAAGGACGAC |
| | | AGCAUCGACAACAAGGUCCUGACAAGAAGCGACAAGAACAGAG |
| | | GAAAGAGCGACAACGUCCCGAGCGAAGAAGUCGUCAAGAAGAU |
| | | GAAGAACUACUGGAGACAGCUGCUGAACGCAAAGCUGAUCACA |
| | | CAGAGAAAGUUCGACAACCUGACAAAGGCAGAGAGAGGAGGAC |
| | | UGAGCGAACUGGACAAGGCAGGAUUCAUCAAGAGACAGCUGGU |
| | | CGAAACAAGACAGAUCACAAAGCACGUCGCACAGAUCCUGGAC |
| | | AGCAGAAUGAACACAAAGUACGACGAAAACGACAAGCUGAUCA |
| | | GAGAAGUCAAGGUCAUCACACUGAAGAGCAAGCUGGUCAGCGA |
| | | CUUCAGAAAGGACUUCCAGUUCUACAAGGUCAGAGAAAUCAAC |
| | | AACUACCACCACGCACACGACGCAUACCUGAACGCAGUCGUCGG |
| | | AACAGCACUGAUCAAGAAGUACCCGAAGCUGGAAAGCGAAUUC |
| | | GUCUACGGAGACUACAAGGUCUACGACGUCAGAAAGAUGAUCG |
| | | CAAAGAGCGAACAGGAAAUCGGAAAGGCAACAGCAAAGUACUU |
| | | CUUCUACAGCAACAUCAUGAACUUCUUCAAGACAGAAAUCACA |
| | | CUGGCAAACGGAGAAAUCAGAAAGAGACCGCUGAUCGAAACAA |
| | | ACGGAGAAACAGGAGAAAUCGUCUGGGACAAGGGAAGAGACUU |
| | | CGCAACAGUCAGAAAGGUCCUGAGCAUGCCGCAGGUCAACAUC |
| | | GUCAAGAAGACAGAAGUCCAGACAGGAGGAUUCAGCAAGGAAA |
| | | GCAUCCUGCCGAAGAGAAACAGCGACAAGCUGAUCGCAAGAAA |
| | | GAAGGACUGGGACCCGAAGAAGUACGGAGGAUUCGACAGCCCG |
| | | ACAGUCGCAUACAGCGUCCUGGUCGUCGCAAAGGUCGAAAAGG |
| | | GAAAGAGCAAGAAGCUGAAGAGCGUCAAGGAACUGCUGGGAAU |
| | | CACAAUCAUGGAAAGAAGCAGCUUCGAAAAGAACCCGAUCGAC |
| | | UUCCUGGAAGCAAAGGGAUACAAGGAAGUCAAGAAGGACCUGA |
| | | UCAUCAAGCUGCCGAAGUACAGCCUGUUCGAACUGGAAAACGG |
| | | AAGAAAGAGAAUGCUGGCAAGCGCAGGAGAACUGCAGAAGGGA |
| | | AACGAACUGGCACUGCCGAGCAAGUACGUCAACUUCCUGUACCU |
| | | GGCAAGCCACUACGAAAAGCUGAAGGGAAGCCCGGAAGACAAC |
| | | GAACAGAAGCAGCUGUUCGUCGAACAGCACAAGCACUACCUGG |
| | | ACGAAAUCAUCGAACAGAUCAGCGAAUUCAGCAAGAGAGUCAU |
| | | CCUGGCAGACGCAAACCUGGACAAGGUCCUGAGCGCAUACAACA |
| | | AGCACAGAGACAAGCCGAUCAGAGAACAGGCAGAAAACAUCAU |
| | | CCACCUGUUCACACUGACAAACCUGGGAGCACCGGCAGCAUUCA |
| | | AGUACUUCGACACAACAAUCGACAGAAAGAGAUACACAAGCAC |
| | | AAAGGAAGUCCUGGACGCAACACUGAUCCACCAGAGCAUCACA |
| | | GGACUGUACGAAACAAGAAUCGAUCUGAGCCAGCUGGGAGGAG |
| | | ACAGCGGAGGAAGCACAAACCUGAGCGACAUCAUCGAAAAGGA |
| | | AACAGGAAAGCAGCUGGUCAUCCAGGAAAGCAUCCUGAUGCUG |
| | | CCGGAAGAAGUCGAAGAAGUCAUCGGAAACAAGCCGGAAAGCG |
| | | ACAUCCUGGUCCACACAGCAUACGACGAAAGCACAGACGAAAAC |
| | | GUCAUGCUGCUGACAAGCGACGCACCGGAAUACAAGCCGUGGG |
| | | CACUGGUCAUCCAGGACAGCAACGGAGAAAACAAGAUCAAGAU |
| | | GCUGAGCGGAGGAAGCCCGAAGAAGAAGAGAAAGGUCUAA |
| Open | 807 | AUGGGACCGAAGAAGAAGAGAAAGGUCGGAGGAGGAAGCACAA |
| reading | | ACCUGUCGGACAUCAUCGAAAAGGAAACAGGAAAGCAGCUGGU |
| frame for | | CAUCCAGGAAUCGAUCCUGAUGCUGCCGGAAGAAGUCGAAGAA |
| UGI | | GUCAUCGGAAACAAGCCGGAAUCGGACAUCCUGGUCCACACAGC |
| | | AUACGACGAAUCGACAGACGAAAACGUCAUGCUGCUGACAUCG |
| | | GACGCACCGGAAUACAAGCCGUGGGCACUGGUCAUCCAGGACUC |
| | | GAACGGAGAAAACAAGAUCAAGAUGCUGUGA |
| Open | 808 | AUGACCAACCUGUCCGACAUCAUCGAGAAGGAGACCGGCAAGC |
| reading | | AGCUGGUGAUCCAGGAGUCCAUCCUGAUGCUGCCCGAGGAGGU |
| frame for | | GGAGGAGGUGAUCGGCAACAAGCCCGAGUCCGACAUCCUGGUG |
| UGI | | CACACCGCCUACGACGAGUCCACCGACGAGAACGUGAUGCUGCU |
| | | GACCUCCGACGCCCCCGAGUACAAGCCCUGGGCCCUGGUGAUCC |
| | | AGGACUCCAACGGCGAGAACAAGAUCAAGAUGCUGUCCGGCGG |
| | | CUCCAAGCGGACCGCCGACGGCUCCGAGUUCGAGUCCCCCAAGA |
| | | AGAAGCGGAAGGUGGAGUGA |
| Amino acid | 809 | MDKKYSIGLDIGTNSVGWAVITDEYKVPSKKFKVLGNTDRHSIKKNL |
| sequence | | IGALLFDSGETAEATRLKRTARRRYTRRKNRICYLQEIFSNEMAKVD |
| for Cas9 | | DSFFHRLEESFLVEEDKKHERHPIFGNIVDEVAYHEKYPTIYHLRKKL |
| encoded by | | VDSTDKADLRLIYLALAHMIKFRGHFLIEGDLNPDNSDVDKLFIQLV |
| SEQ ID | | QTYNQLFEENPINASGVDAKAILSARLSKSRRLENLIAQLPGEKKNGL |
| Nos. 801- | | FGNLIALSLGLTPNFKSNFDLAEDAKLQLSKDTYDDDLDNLLAQIGD |
| 802 | | QYADLFLAAKNLSDAILLSDILRVNTEITKAPLSASMIKRYDEHHQDL |
| | | TLLKALVRQQLPEKYKEIFFDQSKNGYAGYIDGGASQEEFYKFIKPIL |
| | | EKMDGTEELLVKLNREDLLRKQRTFDNGSIPHQIHLGELHAILRRQE |
| | | DFYPFLKDNREKIEKILTFRIPYYVGPLARGNSRFAWMTRKSEETITP |
| | | WNFEEVVDKGASAQSFIERMTNFDKNLPNEKVLPKHSLLYEYFTVY |
| | | NELTKVKYVTEGMRKPAFLSGEQKKAIVDLLFKTNRKVTVKQLKED |
| | | YFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRL |
| | | SRKLINGIRDKQSGKTILDFLKSDGFANRNFMQLIHDDSLTFKEDIQK |
| | | AQVSGQGDSLHEHIANLAGSPAIKKGILQTVKVVDELVKVMGRHKP |
| | | ENIVIEMARENQTTQKGQKNSRERMKRIEEGIKELGSQILKEHPVENT |
| | | QLQNEKLYLYYLQNGRDMYVDQELDINRLSDYDVDHIVPQSFLKDD |
| | | SIDNKVLTRSDKNRGKSDNVPSEEVVKKMKNYWRQLLNAKLITQRK |
| | | FDNLTKAERGGLSELDKAGFIKRQLVETRQITKHVAQILDSRMNTKY |
| | | DENDKLIREVKVITLKSKLVSDFRKDFQFYKVREINNYHHAHDAYLN |
| | | AVVGTALIKKYPKLESEFVYGDYKVYDVRKMIAKSEQEIGKATAKY |
| | | FFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKY |
| | | GGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNEL |
| | | ALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPA |
| | | AFKYFDTTIDRKRYTSTKEVLDATLIHQSITGLYETRIDLSQLGGDGG |
| | | GSPKKKRKV |
| Amino acid | 810 | MDKKYSIGLDIGTNSVGWAVITDEYKVPSKKFKVLGNTDRHSIKKNL |
| sequence | | IGALLFDSGETAEATRLKRTARRRYTRRKNRICYLQEIFSNEMAKVD |
| for Cas9 | | DSFFHRLEESFLVEEDKKHERHPIFGNIVDEVAYHEKYPTIYHLRKKL |
| with Hibit | | VDSTDKADLRLIYLALAHMIKFRGHFLIEGDLNPDNSDVDKLFIQLV |
| tag | | QTYNQLFEENPINASGVDAKAILSARLSKSRRLENLIAQLPGEKKNGL |
| | | FGNLIALSLGLTPNFKSNFDLAEDAKLQLSKDTYDDDLDNLLAQIGD |
| | | QYADLFLAAKNLSDAILLSDILRVNTEITKAPLSASMIKRYDEHHQDL |
| | | TLLKALVRQQLPEKYKEIFFDQSKNGYAGYIDGGASQEEFYKFIKPIL |
| | | EKMDGTEELLVKLNREDLLRKQRTFDNGSIPHQIHLGELHAILRRQE |
| | | DFYPFLKDNREKIEKILTFRIPYYVGPLARGNSRFAWMTRKSEETITP |
| | | WNFEEVVDKGASAQSFIERMTNFDKNLPNEKVLPKHSLLYEYFTVY |
| | | NELTKVKYVTEGMRKPAFLSGEQKKAIVDLLFKTNRKVTVKQLKED |
| | | YFKKIECFDSVEISGVEDRFNASLGTYHDLLKIIKDKDFLDNEENEDIL |
| | | EDIVLTLTLFEDREMIEERLKTYAHLFDDKVMKQLKRRRYTGWGRL |
| | | SRKLINGIRDKQSGKTILDFLKSDGFANRNFMQLIHDDSLTFKEDIQK |
| | | AQVSGQGDSLHEHIANLAGSPAIKKGILQTVKVVDELVKVMGRHKP |
| | | ENIVIEMARENQTTQKGQKNSRERMKRIEEGIKELGSQILKEHPVENT |
| | | QLQNEKLYLYYLQNGRDMYVDQELDINRLSDYDVDHIVPQSFLKDD |
| | | SIDNKVLTRSDKNRGKSDNVPSEEVVKKMKNYWRQLLNAKLITQRK |
| | | FDNLTKAERGGLSELDKAGFIKRQLVETRQITKHVAQILDSRMNTKY |
| | | DENDKLIREVKVITLKSKLVSDFRKDFQFYKVREINNYHHAHDAYLN |
| | | AVVGTALIKKYPKLESEFVYGDYKVYDVRKMIAKSEQEIGKATAKY |
| | | FFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKY |
| | | GGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNEL |
| | | ALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPA |
| | | AFKYFDTTIDRKRYTSTKEVLDATLIHQSITGLYETRIDLSQLGGDGG |
| | | GSPKKKRKVSESATPESVSGWRLFKKIS |
| Amino acid | 811 | MEASPASGPRHLMDPHIFTSNFNNGIGRHKTYLCYEVERLDNGTSVK |
| sequence | | MDQHRGFLHNQAKNLLCGFYGRHAELRFLDLVPSLQLDPAQIYRVT |
| for BC22n | | WFISWSPCFSWGCAGEVRAFLQENTHVRLRIFAARIYDYDPLYKEAL |
| | | QMLRDAGAQVSIMTYDEFKHCWDTFVDHQGCPFQPWDGLDEHSQA |
| | | LSGRLRAILQNQGNSGSETPGTSESATPESDKKYSIGLAIGTNSVGWA |
| | | VITDEYKVPSKKFKVLGNTDRHSIKKNLIGALLFDSGETAEATRLKRT |
| | | ARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMI |
| | | KFRGHFLIEGDLNPDNSDVDKLFIQLVQTYNQLFEENPINASGVDAK |
| | | AILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDI |
| | | LRVNTEITKAPLSASMIKRYDEHHQDLTLLKALVRQQLPEKYKEIFFD |
| | | QSKNGYAGYIDGGASQEEFYKFIKPILEKMDGTEELLVKLNREDLLR |
| | | KQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMT |
| | | NFDKNLPNEKVLPKHSLLYEYFTVYNELTKVKYVTEGMRKPAFLSG |
| | | EQKKAIVDLLFKTNRKVTVKQLKEDYFKKIECFDSVEISGVEDRFNA |
| | | SLGTYHDLLKIIKDKDFLDNEENEDILEDIVLTLTLFEDREMIEERLKT |
| | | YAHLFDDKVMKQLKRRRYTGWGRLSRKLINGIRDKQSGKTILDFLK |
| | | SDGFANRNFMQLIHDDSLTFKEDIQKAQVSGQGDSLHEHIANLAGSP |
| | | AIKKGILQTVKVVDELVKVMGRHKPENIVIEMARENQTTQKGQKNS |
| | | RERMKRIEEGIKELGSQILKEHPVENTQLQNEKLYLYYLQNGRDMYV |
| | | DQELDINRLSDYDVDHIVPQSFLKDDSIDNKVLTRSDKNRGKSDNVP |
| | | SEEVVKKMKNYWRQLLNAKLITQRKFDNLTKAERGGLSELDKAGFI |
| | | KRQLVETRQITKHVAQILDSRMNTKYDENDKLIREVKVITLKSKLVS |
| | | DFRKDFQFYKVREINNYHHAHDAYLNAVVGTALIKKYPKLESEFVY |
| | | GDYKVYDVRKMIAKSEQEIGKATAKYFFYSNIMNFFKTEITLANGEI |
| | | RKRPLIETNGETGEIVWDKGRDFATVRKVLSMPQVNIVKKTEVQTG |
| | | GFSKESILPKRNSDKLIARKKDWDPKKYGGFDSPTVAYSVLVVAKVE |
| | | KGKSKKLKSVKELLGITIMERSSFEKNPIDFLEAKGYKEVKKDLIIKLP |
| | | KYSLFELENGRKRMLASAGELQKGNELALPSKYVNFLYLASHYEKL |
| | | KGSPEDNEQKQLFVEQHKHYLDEIIEQISEFSKRVILADANLDKVLSA |
| | | YNKHRDKPIREQAENIIHLFTLTNLGAPAAFKYFDTTIDRKRYTSTKE |
| | | VLDATLIHQSITGLYETRIDLSQLGGDGGGSPKKKRKV* |
| Amino acid | 812 | MEASPASGPRHLMDPHIFTSNFNNGIGRHKTYLCYEVERLDNGTSVK |
| sequence | | MDQHRGFLHNQAKNLLCGFYGRHAELRFLDLVPSLQLDPAQIYRVT |
| for BC22n | | WFISWSPCFSWGCAGEVRAFLQENTHVRLRIFAARIYDYDPLYKEAL |
| with Hibit | | QMLRDAGAQVSIMTYDEFKHCWDTFVDHQGCPFQPWDGLDEHSQA |
| tag | | LSGRLRAILQNQGNSGSETPGTSESATPESDKKYSIGLAIGTNSVGWA |
| | | VITDEYKVPSKKFKVLGNTDRHSIKKNLIGALLFDSGETAEATRLKRT |
| | | ARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMI |
| | | KFRGHFLIEGDLNPDNSDVDKLFIQLVQTYNQLFEENPINASGVDAK |
| | | AILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDI |
| | | LRVNTEITKAPLSASMIKRYDEHHQDLTLLKALVRQQLPEKYKEIFFD |
| | | QSKNGYAGYIDGGASQEEFYKFIKPILEKMDGTEELLVKLNREDLLR |
| | | KQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMT |
| | | NFDKNLPNEKVLPKHSLLYEYFTVYNELTKVKYVTEGMRKPAFLSG |
| | | EQKKAIVDLLFKTNRKVTVKQLKEDYFKKIECFDSVEISGVEDRFNA |
| | | SLGTYHDLLKIIKDKDFLDNEENEDILEDIVLTLTLFEDREMIEERLKT |
| | | YAHLFDDKVMKQLKRRRYTGWGRLSRKLINGIRDKQSGKTILDFLK |
| | | SDGFANRNFMQLIHDDSLTFKEDIQKAQVSGQGDSLHEHIANLAGSP |
| | | AIKKGILQTVKVVDELVKVMGRHKPENIVIEMARENQTTQKGQKNS |
| | | RERMKRIEEGIKELGSQILKEHPVENTQLQNEKLYLYYLQNGRDMYV |
| | | DQELDINRLSDYDVDHIVPQSFLKDDSIDNKVLTRSDKNRGKSDNVP |
| | | SEEVVKKMKNYWRQLLNAKLITQRKFDNLTKAERGGLSELDKAGFI |
| | | KRQLVETRQITKHVAQILDSRMNTKYDENDKLIREVKVITLKSKLVS |
| | | DFRKDFQFYKVREINNYHHAHDAYLNAVVGTALIKKYPKLESEFVY |
| | | GDYKVYDVRKMIAKSEQEIGKATAKYFFYSNIMNFFKTEITLANGEI |
| | | RKRPLIETNGETGEIVWDKGRDFATVRKVLSMPQVNIVKKTEVQTG |
| | | GFSKESILPKRNSDKLIARKKDWDPKKYGGFDSPTVAYSVLVVAKVE |
| | | KGKSKKLKSVKELLGITIMERSSFEKNPIDFLEAKGYKEVKKDLIIKLP |
| | | KYSLFELENGRKRMLASAGELQKGNELALPSKYVNFLYLASHYEKL |
| | | KGSPEDNEQKQLFVEQHKHYLDEIIEQISEFSKRVILADANLDKVLSA |
| | | YNKHRDKPIREQAENIIHLFTLTNLGAPAAFKYFDTTIDRKRYTSTKE |
| | | VLDATLIHQSITGLYETRIDLSQLGGDGGGSPKKKRKVSESATPESVS |
| | | GWRLFKKIS |
| Amino acid | 813 | MEASPASGPRHLMDPHIFTSNFNNGIGRHKTYLCYEVERLDNGTSVK |
| sequence | | MDQHRGFLHNQAKNLLCGFYGRHAELRFLDLVPSLQLDPAQIYRVT |
| for BC22 | | WFISWSPCFSWGCAGEVRAFLQENTHVRLRIFAARIYDYDPLYKEAL |
| | | QMLRDAGAQVSIMTYDEFKHCWDTFVDHQGCPFQPWDGLDEHSQA |
| | | LSGRLRAILQNQGNSGSETPGTSESATPESDKKYSIGLAIGTNSVGWA |
| | | VITDEYKVPSKKFKVLGNTDRHSIKKNLIGALLFDSGETAEATRLKRT |
| | | ARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMI |
| | | KFRGHFLIEGDLNPDNSDVDKLFIQLVQTYNQLFEENPINASGVDAK |
| | | AILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDI |
| | | LRVNTEITKAPLSASMIKRYDEHHQDLTLLKALVRQQLPEKYKEIFFD |
| | | QSKNGYAGYIDGGASQEEFYKFIKPILEKMDGTEELLVKLNREDLLR |
| | | KQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMT |
| | | NFDKNLPNEKVLPKHSLLYEYFTVYNELTKVKYVTEGMRKPAFLSG |
| | | EQKKAIVDLLFKTNRKVTVKQLKEDYFKKIECFDSVEISGVEDRFNA |
| | | SLGTYHDLLKIIKDKDFLDNEENEDILEDIVLTLTLFEDREMIEERLKT |
| | | YAHLFDDKVMKQLKRRRYTGWGRLSRKLINGIRDKQSGKTILDFLK |
| | | SDGFANRNFMQLIHDDSLTFKEDIQKAQVSGQGDSLHEHIANLAGSP |
| | | AIKKGILQTVKVVDELVKVMGRHKPENIVIEMARENQTTQKGQKNS |
| | | RERMKRIEEGIKELGSQILKEHPVENTQLQNEKLYLYYLQNGRDMYV |
| | | DQELDINRLSDYDVDHIVPQSFLKDDSIDNKVLTRSDKNRGKSDNVP |
| | | SEEVVKKMKNYWRQLLNAKLITQRKFDNLTKAERGGLSELDKAGFI |
| | | KRQLVETRQITKHVAQILDSRMNTKYDENDKLIREVKVITLKSKLVS |
| | | DFRKDFQFYKVREINNYHHAHDAYLNAVVGTALIKKYPKLESEFVY |
| | | GDYKVYDVRKMIAKSEQEIGKATAKYFFYSNIMNFFKTEITLANGEI |
| | | RKRPLIETNGETGEIVWDKGRDFATVRKVLSMPQVNIVKKTEVQTG |
| | | GFSKESILPKRNSDKLIARKKDWDPKKYGGFDSPTVAYSVLVVAKVE |
| | | KGKSKKLKSVKELLGITIMERSSFEKNPIDFLEAKGYKEVKKDLIIKLP |
| | | KYSLFELENGRKRMLASAGELQKGNELALPSKYVNFLYLASHYEKL |
| | | KGSPEDNEQKQLFVEQHKHYLDEIIEQISEFSKRVILADANLDKVLSA |
| | | YNKHRDKPIREQAENIIHLFTLTNLGAPAAFKYFDTTIDRKRYTSTKE |
| | | VLDATLIHQSITGLYETRIDLSQLGGDSGGSTNLSDIIEKETGKQLVIQ |
| | | ESILMLPEEVEEVIGNKPESDILVHTAYDESTDENVMLLTSDAPEYKP |
| | | WALVIQDSNGENKIKMLSGGSPKKKRKV |
| Amino acid | 814 | MTNLSDIIEKETGKQLVIQESILMLPEEVEEVIGNKPESDILVHTAYDE |
| sequence | | STDENVMLLTSDAPEYKPWALVIQDSNGENKIKMLSGGSKRTADGS |
| for UGI | | EFESPKKKRKVE |
| exemplary | 900 | SGSETPGTSESATPES |
| XTEN | | |
| exemplary | 901 | SGSETPGTSESA |
| XTEN | | |
| exemplary | 902 | SGSETPGTSESATPEGGSGGS |
| XTEN | | |
| amino acid | 903 | GGGGSEAAAKEAAAK |
| sequence | | |
| for | | |
| exemplary | | |
| linker | | |
| amino acid | 904 | EAAAKGGGGSGGGGS |
| sequence | | |
| for | | |
| exemplary | | |
| linker | | |
| amino acid | 905 | EAAAKEAAAKEAAAK |
| sequence | | |
| for | | |
| exemplary | | |
| linker | | |
| amino acid | 906 | GGGGSGGGGSGGGGSGGGGS |
| sequence | | |
| for | | |
| exemplary | | |
| linker | | |
| amino acid | 907 | GGGGSGGGGSEAAAKEAAAK |
| sequence | | |
| for | | |
| exemplary | | |
| linker | | |
| amino acid | 908 | GGGGSEAAAKGGGGSGGGGS |
| sequence | | |
| for | | |
| exemplary | | |
| linker | | |
| amino acid | 909 | EAAAKEAAAKEAAAKGGGGSGGGGS |
| sequence | | |
| for | | |
| exemplary | | |
| linker | | |
| amino acid | 910 | EAAAKEAAAKEAAAKEAAAK |
| sequence | | |
| for | | |
| exemplary | | |
| linker | | |
| amino acid | 911 | GGGGSEAAAKEAAAKGGGGSEAAAK |
| sequence | | |
| for | | |
| exemplary | | |
| linker | | |
| amino acid | 912 | EAAAKEAAAKGGGGSGGGGSGGGGS |
| sequence | | |
| for | | |
| exemplary | | |
| linker | | |
| amino acid | 913 | EAAAKEAAAKGGGGSGGGGSEAAAK |
| sequence | | |
| for | | |
| exemplary | | |
| linker | | |
| amino acid | 914 | GGS |
| sequence | | |
| for | | |
| exemplary | | |
| linker | | |
| amino acid | 915 | GGGGS |
| sequence | | |
| for | | |
| exemplary | | |
| linker | | |
| amino acid | 916 | EAAAK |
| sequence | | |
| for | | |
| exemplary | | |
| linker | | |
| amino acid | 917 | SEGSA |
| sequence | | |
| for | | |
| exemplary | | |
| linker | | |
| amino acid | 918 | SEGSAGTST |
| sequence | | |
| for | | |
| exemplary | | |
| linker | | |
| amino acid | 919 | GGGGSGGGGS |
| sequence | | |
| for | | |
| exemplary | | |
| linker | | |
| amino acid | 920 | GGGGSEAAAK |
| sequence | | |
| for | | |
| exemplary | | |
| linker | | |
| amino acid | 921 | EAAAKGGGGS |
| sequence | | |
| for | | |
| exemplary | | |
| linker | | |
| amino acid | 922 | EAAAKEAAAK |
| sequence | | |
| for | | |
| exemplary | | |
| linker | | |
| amino acid | 923 | SEGSAGTSTESEGSA |
| sequence | | |
| for | | |
| exemplary | | |
| linker | | |
| amino acid | 924 | GGGGSGGGGSGGGGS |
| sequence | | |
| for | | |
| exemplary | | |
| linker | | |
| amino acid | 925 | GGGGSGGGGSEAAAK |
| sequence | | |
| for | | |
| exemplary | | |
| linker | | |
| amino acid | 926 | GGGGSEAAAKGGGGS |
| sequence | | |
| for | | |
| exemplary | | |
| linker | | |
| amino acid | 927 | EAAAKGGGGSEAAAK |
| sequence | | |
| for | | |
| exemplary | | |
| linker | | |
| amino acid | 928 | EAAAKEAAAKGGGGS |
| sequence | | |
| for | | |
| exemplary | | |
| linker | | |
| amino acid | 929 | SEGSAGTSTESEGSAGTSTE |
| sequence | | |
| for | | |
| exemplary | | |
| linker | | |
| amino acid | 930 | GGGGSGGGGSGGGGSEAAAK |
| sequence | | |
| for | | |
| exemplary | | |
| linker | | |
| amino acid | 931 | GGGGSGGGGSEAAAKGGGGS |
| sequence | | |
| for | | |
| exemplary | | |
| linker | | |
| amino acid | 932 | GGGGSEAAAKGGGGSEAAAK |
| sequence | | |
| for | | |
| exemplary | | |
| linker | | |
| amino acid | 933 | GGGGSEAAAKEAAAKGGGGS |
| sequence | | |
| for | | |
| exemplary | | |
| linker | | |
| amino acid | 934 | GGGGSEAAAKEAAAKEAAAK |
| sequence | | |
| for | | |
| exemplary | | |
| linker | | |
| amino acid | 935 | EAAAKGGGGSGGGGSGGGGS |
| sequence | | |
| for | | |
| exemplary | | |
| linker | | |
| amino acid | 936 | EAAAKGGGGSGGGGSEAAAK |
| sequence | | |
| for | | |
| exemplary | | |
| linker | | |
| amino acid | 937 | EAAAKGGGGSEAAAKGGGGS |
| sequence | | |
| for | | |
| exemplary | | |
| linker | | |
| amino acid | 938 | EAAAKGGGGSEAAAKEAAAK |
| sequence | | |
| for | | |
| exemplary | | |
| linker | | |
| amino acid | 939 | EAAAKEAAAKGGGGSGGGGS |
| sequence | | |
| for | | |
| exemplary | | |
| linker | | |
| amino acid | 940 | EAAAKEAAAKGGGGSEAAAK |
| sequence | | |
| for | | |
| exemplary | | |
| linker | | |
| amino acid | 941 | EAAAKEAAAKEAAAKGGGGS |
| sequence | | |
| for | | |
| exemplary | | |
| linker | | |
| amino acid | 942 | SEGSAGTSTESEGSAGTSTESEGSA |
| sequence | | |
| for | | |
| exemplary | | |
| linker | | |
| amino acid | 943 | GGGGSGGGGSGGGGSGGGGSGGGGS |
| sequence | | |
| for | | |
| exemplary | | |
| linker | | |
| amino acid | 944 | GGGGSGGGGSGGGGSGGGGSEAAAK |
| sequence | | |
| for | | |
| exemplary | | |
| linker | | |
| amino acid | 945 | GGGGSGGGGSGGGGSEAAAKGGGGS |
| sequence | | |
| for | | |
| exemplary | | |
| linker | | |
| amino acid | 946 | GGGGSGGGGSGGGGSEAAAKEAAAK |
| sequence | | |
| for | | |
| exemplary | | |
| linker | | |
| amino acid | 947 | GGGGSGGGGSEAAAKGGGGSGGGGS |
| sequence | | |
| for | | |
| exemplary | | |
| linker | | |
| amino acid | 948 | GGGGSGGGGSEAAAKGGGGSEAAAK |
| sequence | | |
| for | | |
| exemplary | | |
| linker | | |
| amino acid | 949 | GGGGSGGGGSEAAAKEAAAKGGGGS |
| sequence | | |
| for | | |
| exemplary | | |
| linker | | |
| amino acid | 950 | GGGGSGGGGSEAAAKEAAAKEAAAK |
| sequence | | |
| for | | |
| exemplary | | |
| linker | | |
| amino acid | 951 | GGGGSEAAAKGGGGSGGGGSGGGGS |
| sequence | | |
| for | | |
| exemplary | | |
| linker | | |
| amino acid | 952 | GGGGSEAAAKGGGGSGGGGSEAAAK |
| sequence | | |
| for | | |
| exemplary | | |
| linker | | |
| amino acid | 953 | GGGGSEAAAKGGGGSEAAAKGGGGS |
| sequence | | |
| for | | |
| exemplary | | |
| linker | | |
| amino acid | 954 | GGGGSEAAAKGGGGSEAAAKEAAAK |
| sequence | | |
| for | | |
| exemplary | | |
| linker | | |
| amino acid | 955 | GGGGSEAAAKEAAAKGGGGSGGGGS |
| sequence | | |
| for | | |
| exemplary | | |
| linker | | |
| amino acid | 956 | GGGGSEAAAKEAAAKEAAAKGGGGS |
| sequence | | |
| for | | |
| exemplary | | |
| linker | | |
| amino acid | 957 | GGGGSEAAAKEAAAKEAAAKEAAAK |
| sequence | | |
| for | | |
| exemplary | | |
| linker | | |
| amino acid | 958 | EAAAKGGGGSGGGGSGGGGSGGGGS |
| sequence | | |
| for | | |
| exemplary | | |
| linker | | |
| amino acid | 959 | EAAAKGGGGSGGGGSGGGGSEAAAK |
| sequence | | |
| for | | |
| exemplary | | |
| linker | | |
| amino acid | 960 | EAAAKGGGGSGGGGSEAAAKGGGGS |
| sequence | | |
| for | | |
| exemplary | | |
| linker | | |
| amino acid | 961 | EAAAKGGGGSGGGGSEAAAKEAAAK |
| sequence | | |
| for | | |
| exemplary | | |
| linker | | |
| amino acid | 962 | EAAAKGGGGSEAAAKGGGGSGGGGS |
| sequence | | |
| for | | |
| exemplary | | |
| linker | | |
| amino acid | 963 | EAAAKGGGGSEAAAKGGGGSEAAAK |
| sequence | | |
| for | | |
| exemplary | | |
| linker | | |
| amino acid | 964 | EAAAKGGGGSEAAAKEAAAKGGGGS |
| sequence | | |
| for | | |
| exemplary | | |
| linker | | |
| amino acid | 965 | EAAAKGGGGSEAAAKEAAAKEAAAK |
| sequence | | |
| for | | |
| exemplary | | |
| linker | | |
| amino acid | 966 | EAAAKEAAAKGGGGSEAAAKGGGGS |
| sequence | | |
| for | | |
| exemplary | | |
| linker | | |
| amino acid | 967 | EAAAKEAAAKGGGGSEAAAKEAAAK |
| sequence | | |
| for | | |
| exemplary | | |
| linker | | |
| amino acid | 968 | EAAAKEAAAKEAAAKGGGGSEAAAK |
| sequence | | |
| for | | |
| exemplary | | |
| linker | | |
| amino acid | 969 | EAAAKEAAAKEAAAKEAAAKGGGGS |
| sequence | | |
| for | | |
| exemplary | | |
| linker | | |
| amino acid | 970 | EAAAKEAAAKEAAAKEAAAKEAAAK |
| sequence | | |
| for | | |
| exemplary | | |
| linker | | |
| Amino acid | 971 | SGGS |
| sequence | | |
| for | | |
| exemplary | | |
| linker | | |
| SGGS | | |
| mRNA | 972 | GGGAAGCUCAGAAUAAACGCUCAACUUUGGCCGGAUCUGCCAC |
| encoding | | CAUGGAGGCCUCCCCCGCCUCCGGCCCCCGGCACCUGAUGGACC |
| BC22n | | CCCACAUCUUCACCUCCAACUUCAACAACGGCAUCGGCCGGCAC |
| | | AAGACCUACCUGUGCUACGAGGUGGAGCGGCUGGACAACGGCA |
| | | CCUCCGUGAAGAUGGACCAGCACCGGGGCUUCCUGCACAACCAG |
| | | GCCAAGAACCUGCUGUGCGGCUUCUACGGCCGGCACGCCGAGCU |
| | | GCGGUUCCUGGACCUGGUGCCCUCCCUGCAGCUGGACCCCGCCC |
| | | AGAUCUACCGGGUGACCUGGUUCAUCUCCUGGUCCCCCUGCUUC |
| | | UCCUGGGGCUGCGCCGGCGAGGUGCGGGCCUUCCUGCAGGAGA |
| | | ACACCCACGUGCGGCUGCGGAUCUUCGCCGCCCGGAUCUACGAC |
| | | UACGACCCCCUGUACAAGGAGGCCCUGCAGAUGCUGCGGGACGC |
| | | CGGCGCCCAGGUGUCCAUCAUGACCUACGACGAGUUCAAGCACU |
| | | GCUGGGACACCUUCGUGGACCACCAGGGCUGCCCCUUCCAGCCC |
| | | UGGGACGGCCUGGACGAGCACUCCCAGGCCCUGUCCGGCCGGCU |
| | | GCGGGCCAUCCUGCAGAACCAGGGCAACUCCGGCUCCGAGACCC |
| | | CCGGCACCUCCGAGUCCGCCACCCCCGAGUCCGACAAGAAGUAC |
| | | UCCAUCGGCCUGGCCAUCGGCACCAACUCCGUGGGCUGGGCCGU |
| | | GAUCACCGACGAGUACAAGGUGCCCUCCAAGAAGUUCAAGGUG |
| | | CUGGGCAACACCGACCGGCACUCCAUCAAGAAGAACCUGAUCGG |
| | | CGCCCUGCUGUUCGACUCCGGCGAGACCGCCGAGGCCACCCGGC |
| | | UGAAGCGGACCGCCCGGCGGCGGUACACCCGGCGGAAGAACCGG |
| | | AUCUGCUACCUGCAGGAGAUCUUCUCCAACGAGAUGGCCAAGG |
| | | UGGACGACUCCUUCUUCCACCGGCUGGAGGAGUCCUUCCUGGUG |
| | | GAGGAGGACAAGAAGCACGAGCGGCACCCCAUCUUCGGCAACA |
| | | UCGUGGACGAGGUGGCCUACCACGAGAAGUACCCCACCAUCUAC |
| | | CACCUGCGGAAGAAGCUGGUGGACUCCACCGACAAGGCCGACCU |
| | | GCGGCUGAUCUACCUGGCCCUGGCCCACAUGAUCAAGUUCCGGG |
| | | GCCACUUCCUGAUCGAGGGCGACCUGAACCCCGACAACUCCGAC |
| | | GUGGACAAGCUGUUCAUCCAGCUGGUGCAGACCUACAACCAGC |
| | | UGUUCGAGGAGAACCCCAUCAACGCCUCCGGCGUGGACGCCAAG |
| | | GCCAUCCUGUCCGCCCGGCUGUCCAAGUCCCGGCGGCUGGAGAA |
| | | CCUGAUCGCCCAGCUGCCCGGCGAGAAGAAGAACGGCCUGUUCG |
| | | GCAACCUGAUCGCCCUGUCCCUGGGCCUGACCCCCAACUUCAAG |
| | | UCCAACUUCGACCUGGCCGAGGACGCCAAGCUGCAGCUGUCCAA |
| | | GGACACCUACGACGACGACCUGGACAACCUGCUGGCCCAGAUCG |
| | | GCGACCAGUACGCCGACCUGUUCCUGGCCGCCAAGAACCUGUCC |
| | | GACGCCAUCCUGCUGUCCGACAUCCUGCGGGUGAACACCGAGAU |
| | | CACCAAGGCCCCCCUGUCCGCCUCCAUGAUCAAGCGGUACGACG |
| | | AGCACCACCAGGACCUGACCCUGCUGAAGGCCCUGGUGCGGCAG |
| | | CAGCUGCCCGAGAAGUACAAGGAGAUCUUCUUCGACCAGUCCA |
| | | AGAACGGCUACGCCGGCUACAUCGACGGCGGCGCCUCCCAGGAG |
| | | GAGUUCUACAAGUUCAUCAAGCCCAUCCUGGAGAAGAUGGACG |
| | | GCACCGAGGAGCUGCUGGUGAAGCUGAACCGGGAGGACCUGCU |
| | | GCGGAAGCAGCGGACCUUCGACAACGGCUCCAUCCCCCACCAGA |
| | | UCCACCUGGGCGAGCUGCACGCCAUCCUGCGGCGGCAGGAGGAC |
| | | UUCUACCCCUUCCUGAAGGACAACCGGGAGAAGAUCGAGAAGA |
| | | UCCUGACCUUCCGGAUCCCCUACUACGUGGGCCCCCUGGCCCGG |
| | | GGCAACUCCCGGUUCGCCUGGAUGACCCGGAAGUCCGAGGAGAC |
| | | CAUCACCCCCUGGAACUUCGAGGAGGUGGUGGACAAGGGCGCC |
| | | UCCGCCCAGUCCUUCAUCGAGCGGAUGACCAACUUCGACAAGAA |
| | | CCUGCCCAACGAGAAGGUGCUGCCCAAGCACUCCCUGCUGUACG |
| | | AGUACUUCACCGUGUACAACGAGCUGACCAAGGUGAAGUACGU |
| | | GACCGAGGGCAUGCGGAAGCCCGCCUUCCUGUCCGGCGAGCAGA |
| | | AGAAGGCCAUCGUGGACCUGCUGUUCAAGACCAACCGGAAGGU |
| | | GACCGUGAAGCAGCUGAAGGAGGACUACUUCAAGAAGAUCGAG |
| | | UGCUUCGACUCCGUGGAGAUCUCCGGCGUGGAGGACCGGUUCA |
| | | ACGCCUCCCUGGGCACCUACCACGACCUGCUGAAGAUCAUCAAG |
| | | GACAAGGACUUCCUGGACAACGAGGAGAACGAGGACAUCCUGG |
| | | AGGACAUCGUGCUGACCCUGACCCUGUUCGAGGACCGGGAGAU |
| | | GAUCGAGGAGCGGCUGAAGACCUACGCCCACCUGUUCGACGACA |
| | | AGGUGAUGAAGCAGCUGAAGCGGCGGCGGUACACCGGCUGGGG |
| | | CCGGCUGUCCCGGAAGCUGAUCAACGGCAUCCGGGACAAGCAGU |
| | | CCGGCAAGACCAUCCUGGACUUCCUGAAGUCCGACGGCUUCGCC |
| | | AACCGGAACUUCAUGCAGCUGAUCCACGACGACUCCCUGACCUU |
| | | CAAGGAGGACAUCCAGAAGGCCCAGGUGUCCGGCCAGGGCGAC |
| | | UCCCUGCACGAGCACAUCGCCAACCUGGCCGGCUCCCCCGCCAU |
| | | CAAGAAGGGCAUCCUGCAGACCGUGAAGGUGGUGGACGAGCUG |
| | | GUGAAGGUGAUGGGCCGGCACAAGCCCGAGAACAUCGUGAUCG |
| | | AGAUGGCCCGGGAGAACCAGACCACCCAGAAGGGCCAGAAGAA |
| | | CUCCCGGGAGCGGAUGAAGCGGAUCGAGGAGGGCAUCAAGGAG |
| | | CUGGGCUCCCAGAUCCUGAAGGAGCACCCCGUGGAGAACACCCA |
| | | GCUGCAGAACGAGAAGCUGUACCUGUACUACCUGCAGAACGGC |
| | | CGGGACAUGUACGUGGACCAGGAGCUGGACAUCAACCGGCUGU |
| | | CCGACUACGACGUGGACCACAUCGUGCCCCAGUCCUUCCUGAAG |
| | | GACGACUCCAUCGACAACAAGGUGCUGACCCGGUCCGACAAGAA |
| | | CCGGGGCAAGUCCGACAACGUGCCCUCCGAGGAGGUGGUGAAG |
| | | AAGAUGAAGAACUACUGGCGGCAGCUGCUGAACGCCAAGCUGA |
| | | UCACCCAGCGGAAGUUCGACAACCUGACCAAGGCCGAGCGGGGC |
| | | GGCCUGUCCGAGCUGGACAAGGCCGGCUUCAUCAAGCGGCAGCU |
| | | GGUGGAGACCCGGCAGAUCACCAAGCACGUGGCCCAGAUCCUGG |
| | | ACUCCCGGAUGAACACCAAGUACGACGAGAACGACAAGCUGAU |
| | | CCGGGAGGUGAAGGUGAUCACCCUGAAGUCCAAGCUGGUGUCC |
| | | GACUUCCGGAAGGACUUCCAGUUCUACAAGGUGCGGGAGAUCA |
| | | ACAACUACCACCACGCCCACGACGCCUACCUGAACGCCGUGGUG |
| | | GGCACCGCCCUGAUCAAGAAGUACCCCAAGCUGGAGUCCGAGUU |
| | | CGUGUACGGCGACUACAAGGUGUACGACGUGCGGAAGAUGAUC |
| | | GCCAAGUCCGAGCAGGAGAUCGGCAAGGCCACCGCCAAGUACUU |
| | | CUUCUACUCCAACAUCAUGAACUUCUUCAAGACCGAGAUCACCC |
| | | UGGCCAACGGCGAGAUCCGGAAGCGGCCCCUGAUCGAGACCAAC |
| | | GGCGAGACCGGCGAGAUCGUGUGGGACAAGGGCCGGGACUUCG |
| | | CCACCGUGCGGAAGGUGCUGUCCAUGCCCCAGGUGAACAUCGUG |
| | | AAGAAGACCGAGGUGCAGACCGGCGGCUUCUCCAAGGAGUCCA |
| | | UCCUGCCCAAGCGGAACUCCGACAAGCUGAUCGCCCGGAAGAAG |
| | | GACUGGGACCCCAAGAAGUACGGCGGCUUCGACUCCCCCACCGU |
| | | GGCCUACUCCGUGCUGGUGGUGGCCAAGGUGGAGAAGGGCAAG |
| | | UCCAAGAAGCUGAAGUCCGUGAAGGAGCUGCUGGGCAUCACCA |
| | | UCAUGGAGCGGUCCUCCUUCGAGAAGAACCCCAUCGACUUCCUG |
| | | GAGGCCAAGGGCUACAAGGAGGUGAAGAAGGACCUGAUCAUCA |
| | | AGCUGCCCAAGUACUCCCUGUUCGAGCUGGAGAACGGCCGGAA |
| | | GCGGAUGCUGGCCUCCGCCGGCGAGCUGCAGAAGGGCAACGAGC |
| | | UGGCCCUGCCCUCCAAGUACGUGAACUUCCUGUACCUGGCCUCC |
| | | CACUACGAGAAGCUGAAGGGCUCCCCCGAGGACAACGAGCAGA |
| | | AGCAGCUGUUCGUGGAGCAGCACAAGCACUACCUGGACGAGAU |
| | | CAUCGAGCAGAUCUCCGAGUUCUCCAAGCGGGUGAUCCUGGCCG |
| | | ACGCCAACCUGGACAAGGUGCUGUCCGCCUACAACAAGCACCGG |
| | | GACAAGCCCAUCCGGGAGCAGGCCGAGAACAUCAUCCACCUGUU |
| | | CACCCUGACCAACCUGGGCGCCCCCGCCGCCUUCAAGUACUUCG |
| | | ACACCACCAUCGACCGGAAGCGGUACACCUCCACCAAGGAGGUG |
| | | CUGGACGCCACCCUGAUCCACCAGUCCAUCACCGGCCUGUACGA |
| | | GACCCGGAUCGACCUGUCCCAGCUGGGCGGCGACGGCGGCGGCU |
| | | CCCCCAAGAAGAAGCGGAAGGUGUGACUAGCACCAGCCUCAAG |
| | | AACACCCGAAUGGAGUCUCUAAGCUACAUAAUACCAACUUACA |
| | | CUUUACAAAAUGUUGUCCCCCAAAAUGUAGCCAUUCGUAUCUG |
| | | CUCCUAAUAAAAAGAAAGUUUCUUCACAUUCUCUCGAGAAAAA |
| | | AAAAAAAUGGAAAAAAAAAAAACGGAAAAAAAAAAAAGGUAAA |
| | | AAAAAAAAAUAUAAAAAAAAAAAACAUAAAAAAAAAAAACGAA |
| | | AAAAAAAAAACGUAAAAAAAAAAAACUCAAAAAAAAAAAAGAU |
| | | AAAAAAAAAAAACCUAAAAAAAAAAAAUGUAAAAAAAAAAAAG |
| | | GGAAAAAAAAAAAACGCAAAAAAAAAAAACACAAAAAAAAAAA |
| | | AUGCAAAAAAAAAAAAUCGAAAAAAAAAAAAUCUAAAAAAAAA |
| | | AAACGAAAAAAAAAAAACCCAAAAAAAAAAAAGACAAAAAAAA |
| | | AAAAUAGAAAAAAAAAAAAGUUAAAAAAAAAAAACUGAAAAAA |
| | | AAAAAAUUUAAAAAAAAAAAAUCUAG |
| mRNA | 973 | GGGAAGCUCAGAAUAAACGCUCAACUUUGGCCGGAUCUGCCAC |
| encoding | | CAUGGAGGCCUCCCCCGCCUCCGGCCCCCGGCACCUGAUGGACC |
| BC22n | | CCCACAUCUUCACCUCCAACUUCAACAACGGCAUCGGCCGGCAC |
| with HiBit | | AAGACCUACCUGUGCUACGAGGUGGAGCGGCUGGACAACGGCA |
| tag | | CCUCCGUGAAGAUGGACCAGCACCGGGGCUUCCUGCACAACCAG |
| | | GCCAAGAACCUGCUGUGCGGCUUCUACGGCCGGCACGCCGAGCU |
| | | GCGGUUCCUGGACCUGGUGCCCUCCCUGCAGCUGGACCCCGCCC |
| | | AGAUCUACCGGGUGACCUGGUUCAUCUCCUGGUCCCCCUGCUUC |
| | | UCCUGGGGCUGCGCCGGCGAGGUGCGGGCCUUCCUGCAGGAGA |
| | | ACACCCACGUGCGGCUGCGGAUCUUCGCCGCCCGGAUCUACGAC |
| | | UACGACCCCCUGUACAAGGAGGCCCUGCAGAUGCUGCGGGACGC |
| | | CGGCGCCCAGGUGUCCAUCAUGACCUACGACGAGUUCAAGCACU |
| | | GCUGGGACACCUUCGUGGACCACCAGGGCUGCCCCUUCCAGCCC |
| | | UGGGACGGCCUGGACGAGCACUCCCAGGCCCUGUCCGGCCGGCU |
| | | GCGGGCCAUCCUGCAGAACCAGGGCAACUCCGGCUCCGAGACCC |
| | | CCGGCACCUCCGAGUCCGCCACCCCCGAGUCCGACAAGAAGUAC |
| | | UCCAUCGGCCUGGCCAUCGGCACCAACUCCGUGGGCUGGGCCGU |
| | | GAUCACCGACGAGUACAAGGUGCCCUCCAAGAAGUUCAAGGUG |
| | | CUGGGCAACACCGACCGGCACUCCAUCAAGAAGAACCUGAUCGG |
| | | CGCCCUGCUGUUCGACUCCGGCGAGACCGCCGAGGCCACCCGGC |
| | | UGAAGCGGACCGCCCGGCGGCGGUACACCCGGCGGAAGAACCGG |
| | | AUCUGCUACCUGCAGGAGAUCUUCUCCAACGAGAUGGCCAAGG |
| | | UGGACGACUCCUUCUUCCACCGGCUGGAGGAGUCCUUCCUGGUG |
| | | GAGGAGGACAAGAAGCACGAGCGGCACCCCAUCUUCGGCAACA |
| | | UCGUGGACGAGGUGGCCUACCACGAGAAGUACCCCACCAUCUAC |
| | | CACCUGCGGAAGAAGCUGGUGGACUCCACCGACAAGGCCGACCU |
| | | GCGGCUGAUCUACCUGGCCCUGGCCCACAUGAUCAAGUUCCGGG |
| | | GCCACUUCCUGAUCGAGGGCGACCUGAACCCCGACAACUCCGAC |
| | | GUGGACAAGCUGUUCAUCCAGCUGGUGCAGACCUACAACCAGC |
| | | UGUUCGAGGAGAACCCCAUCAACGCCUCCGGCGUGGACGCCAAG |
| | | GCCAUCCUGUCCGCCCGGCUGUCCAAGUCCCGGCGGCUGGAGAA |
| | | CCUGAUCGCCCAGCUGCCCGGCGAGAAGAAGAACGGCCUGUUCG |
| | | GCAACCUGAUCGCCCUGUCCCUGGGCCUGACCCCCAACUUCAAG |
| | | UCCAACUUCGACCUGGCCGAGGACGCCAAGCUGCAGCUGUCCAA |
| | | GGACACCUACGACGACGACCUGGACAACCUGCUGGCCCAGAUCG |
| | | GCGACCAGUACGCCGACCUGUUCCUGGCCGCCAAGAACCUGUCC |
| | | GACGCCAUCCUGCUGUCCGACAUCCUGCGGGUGAACACCGAGAU |
| | | CACCAAGGCCCCCCUGUCCGCCUCCAUGAUCAAGCGGUACGACG |
| | | AGCACCACCAGGACCUGACCCUGCUGAAGGCCCUGGUGCGGCAG |
| | | CAGCUGCCCGAGAAGUACAAGGAGAUCUUCUUCGACCAGUCCA |
| | | AGAACGGCUACGCCGGCUACAUCGACGGCGGCGCCUCCCAGGAG |
| | | GAGUUCUACAAGUUCAUCAAGCCCAUCCUGGAGAAGAUGGACG |
| | | GCACCGAGGAGCUGCUGGUGAAGCUGAACCGGGAGGACCUGCU |
| | | GCGGAAGCAGCGGACCUUCGACAACGGCUCCAUCCCCCACCAGA |
| | | UCCACCUGGGCGAGCUGCACGCCAUCCUGCGGCGGCAGGAGGAC |
| | | UUCUACCCCUUCCUGAAGGACAACCGGGAGAAGAUCGAGAAGA |
| | | UCCUGACCUUCCGGAUCCCCUACUACGUGGGCCCCCUGGCCCGG |
| | | GGCAACUCCCGGUUCGCCUGGAUGACCCGGAAGUCCGAGGAGAC |
| | | CAUCACCCCCUGGAACUUCGAGGAGGUGGUGGACAAGGGCGCC |
| | | UCCGCCCAGUCCUUCAUCGAGCGGAUGACCAACUUCGACAAGAA |
| | | CCUGCCCAACGAGAAGGUGCUGCCCAAGCACUCCCUGCUGUACG |
| | | AGUACUUCACCGUGUACAACGAGCUGACCAAGGUGAAGUACGU |
| | | GACCGAGGGCAUGCGGAAGCCCGCCUUCCUGUCCGGCGAGCAGA |
| | | AGAAGGCCAUCGUGGACCUGCUGUUCAAGACCAACCGGAAGGU |
| | | GACCGUGAAGCAGCUGAAGGAGGACUACUUCAAGAAGAUCGAG |
| | | UGCUUCGACUCCGUGGAGAUCUCCGGCGUGGAGGACCGGUUCA |
| | | ACGCCUCCCUGGGCACCUACCACGACCUGCUGAAGAUCAUCAAG |
| | | GACAAGGACUUCCUGGACAACGAGGAGAACGAGGACAUCCUGG |
| | | AGGACAUCGUGCUGACCCUGACCCUGUUCGAGGACCGGGAGAU |
| | | GAUCGAGGAGCGGCUGAAGACCUACGCCCACCUGUUCGACGACA |
| | | AGGUGAUGAAGCAGCUGAAGCGGCGGCGGUACACCGGCUGGGG |
| | | CCGGCUGUCCCGGAAGCUGAUCAACGGCAUCCGGGACAAGCAGU |
| | | CCGGCAAGACCAUCCUGGACUUCCUGAAGUCCGACGGCUUCGCC |
| | | AACCGGAACUUCAUGCAGCUGAUCCACGACGACUCCCUGACCUU |
| | | CAAGGAGGACAUCCAGAAGGCCCAGGUGUCCGGCCAGGGCGAC |
| | | UCCCUGCACGAGCACAUCGCCAACCUGGCCGGCUCCCCCGCCAU |
| | | CAAGAAGGGCAUCCUGCAGACCGUGAAGGUGGUGGACGAGCUG |
| | | GUGAAGGUGAUGGGCCGGCACAAGCCCGAGAACAUCGUGAUCG |
| | | AGAUGGCCCGGGAGAACCAGACCACCCAGAAGGGCCAGAAGAA |
| | | CUCCCGGGAGCGGAUGAAGCGGAUCGAGGAGGGCAUCAAGGAG |
| | | CUGGGCUCCCAGAUCCUGAAGGAGCACCCCGUGGAGAACACCCA |
| | | GCUGCAGAACGAGAAGCUGUACCUGUACUACCUGCAGAACGGC |
| | | CGGGACAUGUACGUGGACCAGGAGCUGGACAUCAACCGGCUGU |
| | | CCGACUACGACGUGGACCACAUCGUGCCCCAGUCCUUCCUGAAG |
| | | GACGACUCCAUCGACAACAAGGUGCUGACCCGGUCCGACAAGAA |
| | | CCGGGGCAAGUCCGACAACGUGCCCUCCGAGGAGGUGGUGAAG |
| | | AAGAUGAAGAACUACUGGCGGCAGCUGCUGAACGCCAAGCUGA |
| | | UCACCCAGCGGAAGUUCGACAACCUGACCAAGGCCGAGCGGGGC |
| | | GGCCUGUCCGAGCUGGACAAGGCCGGCUUCAUCAAGCGGCAGCU |
| | | GGUGGAGACCCGGCAGAUCACCAAGCACGUGGCCCAGAUCCUGG |
| | | ACUCCCGGAUGAACACCAAGUACGACGAGAACGACAAGCUGAU |
| | | CCGGGAGGUGAAGGUGAUCACCCUGAAGUCCAAGCUGGUGUCC |
| | | GACUUCCGGAAGGACUUCCAGUUCUACAAGGUGCGGGAGAUCA |
| | | ACAACUACCACCACGCCCACGACGCCUACCUGAACGCCGUGGUG |
| | | GGCACCGCCCUGAUCAAGAAGUACCCCAAGCUGGAGUCCGAGUU |
| | | CGUGUACGGCGACUACAAGGUGUACGACGUGCGGAAGAUGAUC |
| | | GCCAAGUCCGAGCAGGAGAUCGGCAAGGCCACCGCCAAGUACUU |
| | | CUUCUACUCCAACAUCAUGAACUUCUUCAAGACCGAGAUCACCC |
| | | UGGCCAACGGCGAGAUCCGGAAGCGGCCCCUGAUCGAGACCAAC |
| | | GGCGAGACCGGCGAGAUCGUGUGGGACAAGGGCCGGGACUUCG |
| | | CCACCGUGCGGAAGGUGCUGUCCAUGCCCCAGGUGAACAUCGUG |
| | | AAGAAGACCGAGGUGCAGACCGGCGGCUUCUCCAAGGAGUCCA |
| | | UCCUGCCCAAGCGGAACUCCGACAAGCUGAUCGCCCGGAAGAAG |
| | | GACUGGGACCCCAAGAAGUACGGCGGCUUCGACUCCCCCACCGU |
| | | GGCCUACUCCGUGCUGGUGGUGGCCAAGGUGGAGAAGGGCAAG |
| | | UCCAAGAAGCUGAAGUCCGUGAAGGAGCUGCUGGGCAUCACCA |
| | | UCAUGGAGCGGUCCUCCUUCGAGAAGAACCCCAUCGACUUCCUG |
| | | GAGGCCAAGGGCUACAAGGAGGUGAAGAAGGACCUGAUCAUCA |
| | | AGCUGCCCAAGUACUCCCUGUUCGAGCUGGAGAACGGCCGGAA |
| | | GCGGAUGCUGGCCUCCGCCGGCGAGCUGCAGAAGGGCAACGAGC |
| | | UGGCCCUGCCCUCCAAGUACGUGAACUUCCUGUACCUGGCCUCC |
| | | CACUACGAGAAGCUGAAGGGCUCCCCCGAGGACAACGAGCAGA |
| | | AGCAGCUGUUCGUGGAGCAGCACAAGCACUACCUGGACGAGAU |
| | | CAUCGAGCAGAUCUCCGAGUUCUCCAAGCGGGUGAUCCUGGCCG |
| | | ACGCCAACCUGGACAAGGUGCUGUCCGCCUACAACAAGCACCGG |
| | | GACAAGCCCAUCCGGGAGCAGGCCGAGAACAUCAUCCACCUGUU |
| | | CACCCUGACCAACCUGGGCGCCCCCGCCGCCUUCAAGUACUUCG |
| | | ACACCACCAUCGACCGGAAGCGGUACACCUCCACCAAGGAGGUG |
| | | CUGGACGCCACCCUGAUCCACCAGUCCAUCACCGGCCUGUACGA |
| | | GACCCGGAUCGACCUGUCCCAGCUGGGCGGCGACGGCGGCGGCU |
| | | CCCCCAAGAAGAAGCGGAAGGUGUCCGAGUCCGCCACCCCCGAG |
| | | UCCGUGUCCGGCUGGCGGCUGUUCAAGAAGAUCUCCUGACUAG |
| | | CACCAGCCUCAAGAACACCCGAAUGGAGUCUCUAAGCUACAUAA |
| | | UACCAACUUACACUUUACAAAAUGUUGUCCCCCAAAAUGUAGC |
| | | CAUUCGUAUCUGCUCCUAAUAAAAAGAAAGUUUCUUCACAUUC |
| | | UCUCGAGAAAAAAAAAAAAUGGAAAAAAAAAAAACGGAAAAAA |
| | | AAAAAAGGUAAAAAAAAAAAAUAUAAAAAAAAAAAACAUAAAA |
| | | AAAAAAAACGAAAAAAAAAAAACGUAAAAAAAAAAAACUCAAA |
| | | AAAAAAAAAGAUAAAAAAAAAAAACCUAAAAAAAAAAAAUGUA |
| | | AAAAAAAAAAAGGGAAAAAAAAAAAACGCAAAAAAAAAAAACA |
| | | CAAAAAAAAAAAAUGCAAAAAAAAAAAAUCGAAAAAAAAAAAA |
| | | UCUAAAAAAAAAAAACGAAAAAAAAAAAACCCAAAAAAAAAAA |
| | | AGACAAAAAAAAAAAAUAGAAAAAAAAAAAAGUUAAAAAAAAA |
| | | AAACUGAAAAAAAAAAAAUUUAAAAAAAAAAAAUCUAG |
| mRNA | 974 | GGGAGACCCAAGCUGGCUAGCGUUUAAACUUAAGCUUUCCCGC |
| encoding | | AGUCGGCGUCCAGCGGCUCUGCUUGUUCGUGUGUGUGUCGUUG |
| BC22 | | CAGGCCUUAUUCGGAUCCGCCACCAUGGAAGCAAGCCCGGCAAG |
| | | CGGACCGAGACACCUGAUGGACCCGCACAUCUUCACAAGCAACU |
| | | UCAACAACGGAAUCGGAAGACACAAGACAUACCUGUGCUACGA |
| | | AGUCGAAAGACUGGACAACGGAACAAGCGUCAAGAUGGACCAG |
| | | CACAGAGGAUUCCUGCACAACCAGGCAAAGAACCUGCUGUGCG |
| | | GAUUCUACGGAAGACACGCAGAACUGAGAUUCCUGGACCUGGU |
| | | CCCGAGCCUGCAGCUGGACCCGGCACAGAUCUACAGAGUCACAU |
| | | GGUUCAUCAGCUGGAGCCCGUGCUUCAGCUGGGGAUGCGCAGG |
| | | AGAAGUCAGAGCAUUUCUGCAGGAAAACACACACGUCAGACUG |
| | | AGAAUCUUCGCAGCAAGAAUCUACGACUACGACCCGCUGUACA |
| | | AGGAAGCACUGCAGAUGCUGAGAGACGCAGGAGCACAGGUCAG |
| | | CAUCAUGACAUACGACGAAUUCAAGCACUGCUGGGACACAUUC |
| | | GUCGACCACCAGGGAUGCCCGUUCCAGCCGUGGGACGGACUGGA |
| | | CGAACACAGCCAGGCACUGAGCGGAAGACUGAGAGCAAUCCUG |
| | | CAGAACCAGGGAAACAGCGGAAGCGAAACACCGGGAACAAGCG |
| | | AAAGCGCAACACCGGAAAGCGACAAGAAGUACAGCAUCGGACU |
| | | GGCCAUCGGAACAAACAGCGUCGGAUGGGCAGUCAUCACAGAC |
| | | GAAUACAAGGUCCCGAGCAAGAAGUUCAAGGUCCUGGGAAACA |
| | | CAGACAGACACAGCAUCAAGAAGAACCUGAUCGGAGCACUGCU |
| | | GUUCGACAGCGGAGAAACAGCAGAAGCAACAAGACUGAAGAGA |
| | | ACAGCAAGAAGAAGAUACACAAGAAGAAAGAACAGAAUCUGCU |
| | | ACCUGCAGGAAAUCUUCAGCAACGAAAUGGCAAAGGUCGACGA |
| | | CAGCUUCUUCCACAGACUGGAAGAAAGCUUCCUGGUCGAAGAA |
| | | GACAAGAAGCACGAAAGACACCCGAUCUUCGGAAACAUCGUCG |
| | | ACGAAGUCGCAUACCACGAAAAGUACCCGACAAUCUACCACCUG |
| | | AGAAAGAAGCUGGUCGACAGCACAGACAAGGCAGACCUGAGAC |
| | | UGAUCUACCUGGCACUGGCACACAUGAUCAAGUUCAGAGGACA |
| | | CUUCCUGAUCGAAGGAGACCUGAACCCGGACAACAGCGACGUCG |
| | | ACAAGCUGUUCAUCCAGCUGGUCCAGACAUACAACCAGCUGUUC |
| | | GAAGAAAACCCGAUCAACGCAAGCGGAGUCGACGCAAAGGCAA |
| | | UCCUGAGCGCAAGACUGAGCAAGAGCAGAAGACUGGAAAACCU |
| | | GAUCGCACAGCUGCCGGGAGAAAAGAAGAACGGACUGUUCGGA |
| | | AACCUGAUCGCACUGAGCCUGGGACUGACACCGAACUUCAAGA |
| | | GCAACUUCGACCUGGCAGAAGACGCAAAGCUGCAGCUGAGCAA |
| | | GGACACAUACGACGACGACCUGGACAACCUGCUGGCACAGAUCG |
| | | GAGACCAGUACGCAGACCUGUUCCUGGCAGCAAAGAACCUGAG |
| | | CGACGCAAUCCUGCUGAGCGACAUCCUGAGAGUCAACACAGAA |
| | | AUCACAAAGGCACCGCUGAGCGCAAGCAUGAUCAAGAGAUACG |
| | | ACGAACACCACCAGGACCUGACACUGCUGAAGGCACUGGUCAGA |
| | | CAGCAGCUGCCGGAAAAGUACAAGGAAAUCUUCUUCGACCAGA |
| | | GCAAGAACGGAUACGCAGGAUACAUCGACGGAGGAGCAAGCCA |
| | | GGAAGAAUUCUACAAGUUCAUCAAGCCGAUCCUGGAAAAGAUG |
| | | GACGGAACAGAAGAACUGCUGGUCAAGCUGAACAGAGAAGACC |
| | | UGCUGAGAAAGCAGAGAACAUUCGACAACGGAAGCAUCCCGCA |
| | | CCAGAUCCACCUGGGAGAACUGCACGCAAUCCUGAGAAGACAG |
| | | GAAGACUUCUACCCGUUCCUGAAGGACAACAGAGAAAAGAUCG |
| | | AAAAGAUCCUGACAUUCAGAAUCCCGUACUACGUCGGACCGCU |
| | | GGCAAGAGGAAACAGCAGAUUCGCAUGGAUGACAAGAAAGAGC |
| | | GAAGAAACAAUCACACCGUGGAACUUCGAAGAAGUCGUCGACA |
| | | AGGGAGCAAGCGCACAGAGCUUCAUCGAAAGAAUGACAAACUU |
| | | CGACAAGAACCUGCCGAACGAAAAGGUCCUGCCGAAGCACAGCC |
| | | UGCUGUACGAAUACUUCACAGUCUACAACGAACUGACAAAGGU |
| | | CAAGUACGUCACAGAAGGAAUGAGAAAGCCGGCAUUCCUGAGC |
| | | GGAGAACAGAAGAAGGCAAUCGUCGACCUGCUGUUCAAGACAA |
| | | ACAGAAAGGUCACAGUCAAGCAGCUGAAGGAAGACUACUUCAA |
| | | GAAGAUCGAAUGCUUCGACAGCGUCGAAAUCAGCGGAGUCGAA |
| | | GACAGAUUCAACGCAAGCCUGGGAACAUACCACGACCUGCUGA |
| | | AGAUCAUCAAGGACAAGGACUUCCUGGACAACGAAGAAAACGA |
| | | AGACAUCCUGGAAGACAUCGUCCUGACACUGACACUGUUCGAA |
| | | GACAGAGAAAUGAUCGAAGAAAGACUGAAGACAUACGCACACC |
| | | UGUUCGACGACAAGGUCAUGAAGCAGCUGAAGAGAAGAAGAUA |
| | | CACAGGAUGGGGAAGACUGAGCAGAAAGCUGAUCAACGGAAUC |
| | | AGAGACAAGCAGAGCGGAAAGACAAUCCUGGACUUCCUGAAGA |
| | | GCGACGGAUUCGCAAACAGAAACUUCAUGCAGCUGAUCCACGA |
| | | CGACAGCCUGACAUUCAAGGAAGACAUCCAGAAGGCACAGGUC |
| | | AGCGGACAGGGAGACAGCCUGCACGAACACAUCGCAAACCUGGC |
| | | AGGAAGCCCGGCAAUCAAGAAGGGAAUCCUGCAGACAGUCAAG |
| | | GUCGUCGACGAACUGGUCAAGGUCAUGGGAAGACACAAGCCGG |
| | | AAAACAUCGUCAUCGAAAUGGCAAGAGAAAACCAGACAACACA |
| | | GAAGGGACAGAAGAACAGCAGAGAAAGAAUGAAGAGAAUCGAA |
| | | GAAGGAAUCAAGGAACUGGGAAGCCAGAUCCUGAAGGAACACC |
| | | CGGUCGAAAACACACAGCUGCAGAACGAAAAGCUGUACCUGUA |
| | | CUACCUGCAGAACGGAAGAGACAUGUACGUCGACCAGGAACUG |
| | | GACAUCAACAGACUGAGCGACUACGACGUCGACCACAUCGUCCC |
| | | GCAGAGCUUCCUGAAGGACGACAGCAUCGACAACAAGGUCCUG |
| | | ACAAGAAGCGACAAGAACAGAGGAAAGAGCGACAACGUCCCGA |
| | | GCGAAGAAGUCGUCAAGAAGAUGAAGAACUACUGGAGACAGCU |
| | | GCUGAACGCAAAGCUGAUCACACAGAGAAAGUUCGACAACCUG |
| | | ACAAAGGCAGAGAGAGGAGGACUGAGCGAACUGGACAAGGCAG |
| | | GAUUCAUCAAGAGACAGCUGGUCGAAACAAGACAGAUCACAAA |
| | | GCACGUCGCACAGAUCCUGGACAGCAGAAUGAACACAAAGUAC |
| | | GACGAAAACGACAAGCUGAUCAGAGAAGUCAAGGUCAUCACAC |
| | | UGAAGAGCAAGCUGGUCAGCGACUUCAGAAAGGACUUCCAGUU |
| | | CUACAAGGUCAGAGAAAUCAACAACUACCACCACGCACACGACG |
| | | CAUACCUGAACGCAGUCGUCGGAACAGCACUGAUCAAGAAGUA |
| | | CCCGAAGCUGGAAAGCGAAUUCGUCUACGGAGACUACAAGGUC |
| | | UACGACGUCAGAAAGAUGAUCGCAAAGAGCGAACAGGAAAUCG |
| | | GAAAGGCAACAGCAAAGUACUUCUUCUACAGCAACAUCAUGAA |
| | | CUUCUUCAAGACAGAAAUCACACUGGCAAACGGAGAAAUCAGA |
| | | AAGAGACCGCUGAUCGAAACAAACGGAGAAACAGGAGAAAUCG |
| | | UCUGGGACAAGGGAAGAGACUUCGCAACAGUCAGAAAGGUCCU |
| | | GAGCAUGCCGCAGGUCAACAUCGUCAAGAAGACAGAAGUCCAG |
| | | ACAGGAGGAUUCAGCAAGGAAAGCAUCCUGCCGAAGAGAAACA |
| | | GCGACAAGCUGAUCGCAAGAAAGAAGGACUGGGACCCGAAGAA |
| | | GUACGGAGGAUUCGACAGCCCGACAGUCGCAUACAGCGUCCUG |
| | | GUCGUCGCAAAGGUCGAAAAGGGAAAGAGCAAGAAGCUGAAGA |
| | | GCGUCAAGGAACUGCUGGGAAUCACAAUCAUGGAAAGAAGCAG |
| | | CUUCGAAAAGAACCCGAUCGACUUCCUGGAAGCAAAGGGAUAC |
| | | AAGGAAGUCAAGAAGGACCUGAUCAUCAAGCUGCCGAAGUACA |
| | | GCCUGUUCGAACUGGAAAACGGAAGAAAGAGAAUGCUGGCAAG |
| | | CGCAGGAGAACUGCAGAAGGGAAACGAACUGGCACUGCCGAGC |
| | | AAGUACGUCAACUUCCUGUACCUGGCAAGCCACUACGAAAAGC |
| | | UGAAGGGAAGCCCGGAAGACAACGAACAGAAGCAGCUGUUCGU |
| | | CGAACAGCACAAGCACUACCUGGACGAAAUCAUCGAACAGAUC |
| | | AGCGAAUUCAGCAAGAGAGUCAUCCUGGCAGACGCAAACCUGG |
| | | ACAAGGUCCUGAGCGCAUACAACAAGCACAGAGACAAGCCGAU |
| | | CAGAGAACAGGCAGAAAACAUCAUCCACCUGUUCACACUGACA |
| | | AACCUGGGAGCACCGGCAGCAUUCAAGUACUUCGACACAACAA |
| | | UCGACAGAAAGAGAUACACAAGCACAAAGGAAGUCCUGGACGC |
| | | AACACUGAUCCACCAGAGCAUCACAGGACUGUACGAAACAAGA |
| | | AUCGAUCUGAGCCAGCUGGGAGGAGACAGCGGAGGAAGCACAA |
| | | ACCUGAGCGACAUCAUCGAAAAGGAAACAGGAAAGCAGCUGGU |
| | | CAUCCAGGAAAGCAUCCUGAUGCUGCCGGAAGAAGUCGAAGAA |
| | | GUCAUCGGAAACAAGCCGGAAAGCGACAUCCUGGUCCACACAGC |
| | | AUACGACGAAAGCACAGACGAAAACGUCAUGCUGCUGACAAGC |
| | | GACGCACCGGAAUACAAGCCGUGGGCACUGGUCAUCCAGGACA |
| | | GCAACGGAGAAAACAAGAUCAAGAUGCUGAGCGGAGGAAGCCC |
| | | GAAGAAGAAGAGAAAGGUCUAAUAGUCUAGACAUCACAUUUAA |
| | | AAGCAUCUCAGCCUACCAUGAGAAUAAGAGAAAGAAAAUGAAG |
| | | AUCAAUAGCUUAUUCAUCUCUUUUUCUUUUUCGUUGGUGUAAA |
| | | GCCAACACCCUGUCUAAAAAACAUAAAUUUCUUUAAUCAUUUU |
| | | GCCUCUUUUCUCUGUGCUUCAAUUAAUAAAAAAUGGAAAGAAC |
| | | CUCGAGAAAAAAAAAAAAAAAAAAAAAAAAAAAAAAGCGAAAA |
| | | AAAAAAAAAAAAAAAAAAAAAAAAAACCGAAAAAAAAAAAAAA |
| | | AAAAAAAAAAAAAAAAAAAAAAAAAU |
| mRNA | 975 | GGGAGACCCAAGCUGGCUAGCUCCCGCAGUCGGCGUCCAGCGGC |
| encoding | | UCUGCUUGUUCGUGUGUGUGUCGUUGCAGGCCUUAUUCGGAUC |
| UGI | | CGCCACCAUGGGACCGAAGAAGAAGAGAAAGGUCGGAGGAGGA |
| | | AGCACAAACCUGUCGGACAUCAUCGAAAAGGAAACAGGAAAGC |
| | | AGCUGGUCAUCCAGGAAUCGAUCCUGAUGCUGCCGGAAGAAGU |
| | | CGAAGAAGUCAUCGGAAACAAGCCGGAAUCGGACAUCCUGGUC |
| | | CACACAGCAUACGACGAAUCGACAGACGAAAACGUCAUGCUGC |
| | | UGACAUCGGACGCACCGGAAUACAAGCCGUGGGCACUGGUCAU |
| | | CCAGGACUCGAACGGAGAAAACAAGAUCAAGAUGCUGUGAUAG |
| | | UCUAGACAUCACAUUUAAAAGCAUCUCAGCCUACCAUGAGAAU |
| | | AAGAGAAAGAAAAUGAAGAUCAAUAGCUUAUUCAUCUCUUUUU |
| | | CUUUUUCGUUGGUGUAAAGCCAACACCCUGUCUAAAAAACAUA |
| | | AAUUUCUUUAAUCAUUUUGCCUCUUUUCUCUGUGCUUCAAUUA |
| | | AUAAAAAAUGGAAAGAACCUCGAGUCUAG |
| Guide | 976 | NNNNNNNNNNNNNNNNNNNNGUUUUAGAGCUAGAAAUAG |
| Scaffold | | CAAGUUAAAAUAAGGCUAGUCCGUUAUCACGAAAGGGCA |
| | | CCGAGUCGGUGCU |
| Guide | 977 | mN*mN*mN*NNNNNNNNNNNNNNNNNGUUUUAGAmGmCmU |
| scaffold | | mAmGmAmAmAmUmAmGmCAAGUUAAAAUAAGGCUAGU |
| | | CCGUUAUCACGAAAGGGCACCGAGUCGGmUmGmC*mU |
| Guide | 978 | GUUUUAGAmGmCmUmAmGmAmAmAmUmAmGmCAAGUUA |
| scaffold | | AAAUAAGGCUAGUCCGUUAUCAmAmAmAmUmGmGmCmA |
| 88-mer | | mCmCmGmAmGmUmCmGmG*mU*mG*mC |
| with | | |
| modification | | |
| Guide | 979 | GUUUUAGAmGmCmUmAmGmAmAmAmUmAmGmCAAGUUA |
| scaffold | | AAAUAAGGCUAGUCCGUUAUCAmAmCmUmUmGmAmAmAm |
| with | | AmAmGmUmGmGmCmAmCmCmGmAmGmUmCmGmGmUmGm |
| modification | | CmU*mU*mU*mU |
| Guide | 980 | mN*mN*mN*NNNNNNNNNNNNNNNNNGUUUUAGAmGmC |
| scaffold | | mUmAmGmAmAmAmUmAmGmCAAGUUAAAAUAAGGCUAGU |
| with | | CCGUUAUCAmAmCmUmUmGmAmAmAmAmAmGmUmGmGmC |
| modification | | mAmCmCmGmAmGmUmCmGmGmUmGmCmU*mU*mU*mU |

In the following table and throughout, the terms “mA,” “mC,” “mU,” or “mG” are used to denote a nucleotide that has been modified with 2′-O-Me. In the following table, a “*” is used to depict a PS modification. In this application, the terms A*, C*, U*, or G* may be used to denote a nucleotide that is linked to the next (e.g., 3′) nucleotide with a PS bond. It is understood that if a DNA sequence (comprising Ts) is referenced with respect to an RNA, then Ts should be replaced with Us (which may be modified or unmodified depending on the context), and vice versa. In the following table, single amino acid letter code is used to provide peptide sequences.

1. An engineered cell, which has reduced or eliminated surface expression of MHC class II relative to an unmodified cell, comprising a genetic modification in the CIITA gene, wherein the genetic modification comprises at least one nucleotide of a splice site within the genomic coordinates chr16:10902171-10923242.

2. The engineered cell of claim 1, wherein the genetic modification comprises a modification of at least one nucleotide of a splice acceptor site, optionally wherein the one nucleotide is A or G; or wherein the genetic modification comprises a modification of at least one nucleotide of a splice donor site, optionally wherein the one nucleotide is G or T.

3. (canceled)

4. The engineered cell of claim 1, wherein the genetic modification comprises a modification of a splice site boundary nucleotide; or wherein the genetic modification comprises at least 5, 6, 7, 8, 9, or 10 contiguous nucleotides within the genomic coordinates chr16:10902171-10923242: or wherein the genetic modification comprises at least one C to T substitution or at least one A to G substitution within the genomic coordinates chr16:10902171-10923242.

5-6. (canceled)

7. The engineered cell of claim 1, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, chr16:10916450-10916470, chr16:10923222-10923242, chr16:10916449-10916469, and chr16:10923214-10923234; or
wherein the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150; or
wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-10906507.

8-9. (canceled)

10. An engineered cell, which has reduced or eliminated surface expression of MHC class II relative to an unmodified cell, comprising a genetic modification in the CIITA gene, wherein the genetic modification comprises an indel, a C to T substitution, or an A to G substitution within the genomic coordinates chosen from: chr16:10895410-10895430, chr16:10898649-10898669, chr16:10898658-10898678, chr16:10902171-10902191, chr16:10902173-10902193, chr16:10902174-10902194, chr16:10902179-10902199, chr16:10902183-10902203, chr16:10902184-10902204, chr16:10902644-10902664, chr16:10902779-10902799, chr16:10902788-10902808, chr16:10902789-10902809, chr16:10902790-10902810, chr16:10902795-10902815, chr16:10902799-10902819, chr16:10903708-10903728, chr16:10903713-10903733, chr16:10903718-10903738, chr16:10903721-10903741, chr16:10903723-10903743, chr16:10903724-10903744, chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242.

11. The engineered cell of claim 10, wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, chr16:10916450-10916470, chr16:10923222-10923242, chr16:10916449-10916469, and chr16:10923214-10923234; or
wherein the genetic modification comprises at least one nucleotide of a splice site within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150: or
wherein the genetic modification comprises at least one nucleotide of a splice site within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-10906507.

12-13. (canceled)

14. The engineered cell of claim 1, wherein the genetic modification comprises at least 5, 6, 7, 8, 9, or 10 contiguous nucleotides within the genomic coordinates; or wherein the genetic modification comprises at least one C to T substitution or at least one A to G substitution within the genomic coordinates.

15. (canceled)

16. The engineered cell of claim 1, wherein the MHC class II expression is reduced or eliminated by a gene editing system that binds to a CIITA genomic target sequence comprising at least 5 contiguous nucleotides within the genomic coordinates chosen from: chr16:10895410-10895430, chr16:10898649-10898669, chr16:10898658-10898678, chr16:10902171-10902191, chr16:10902173-10902193, chr16:10902174-10902194, chr16:10902179-10902199, chr16:10902183-10902203, chr16:10902184-10902204, chr16:10902644-10902664, chr16:10902779-10902799, chr16:10902788-10902808, chr16:10902789-10902809, chr16:10902790-10902810, chr16:10902795-10902815, chr16:10902799-10902819, chr16:10903708-10903728, chr16:10903713-10903733, chr16:10903718-10903738, chr16:10903721-10903741, chr16:10903723-10903743, chr16:10903724-10903744, chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242.

17. The engineered cell of claim 1, wherein the MHC class II expression is reduced or eliminated by a gene editing system that binds to a CIITA genomic target sequence comprising at least 5 contiguous nucleotides within the genomic coordinates chosen from: chr16:10903873-10903893, chr16:10903878-10903898, chr16:10903905-10903925, chr16:10903906-10903926, chr16:10904736-10904756, chr16:10904790-10904810, chr16:10904811-10904831, chr16:10906481-10906501, chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242; or
wherein the MHC class II expression is reduced or eliminated by a gene editing system that binds to a CIITA genomic target sequence comprising at least 5 contiguous nucleotides within the genomic coordinates chosen from: chr16:10906485-10906505, chr16:10906486-10906506, chr16:10906487-10906507, chr16:10906492-10906512, chr16:10908127-10908147, chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242; or
wherein the MHC class II expression is reduced or eliminated by a gene editing system that binds to a CIITA genomic target sequence comprising at least 5 contiguous nucleotides within the genomic coordinates chosen from: chr16:10908130-10908150, chr16:10908131-10908151, chr16:10908132-10908152, chr16:10908137-10908157, chr16:10908138-10908158, chr16:10908139-10908159, chr16:10909006-10909026, chr16:10909007-10909027, chr16:10909018-10909038, chr16:10909021-10909041, chr16:10909022-10909042, chr16:10909172-10909192, chr16:10910165-10910185, chr16:10910176-10910196, chr16:10910186-10910206, chr16:10915547-10915567, chr16:10915551-10915571, chr16:10915552-10915572, chr16:10915567-10915587, chr16:10916348-10916368, chr16:10916359-10916379, chr16:10916362-10916382, chr16:10916449-10916469, chr16:10916450-10916470, chr16:10916455-10916475, chr16:10916456-10916476, chr16:10918423-10918443, chr16:10918504-10918524, chr16:10918511-10918531, chr16:10918512-10918532, chr16:10918539-10918559, chr16:10922153-10922173, chr16:10922478-10922498, chr16:10922487-10922507, chr16:10922499-10922519, chr16:10923205-10923225, chr16:10923214-10923234, chr16:10923218-10923238, chr16:10923219-10923239, chr16:10923220-10923240, chr16:10923221-10923241, and chr16:10923222-10923242; or
wherein the MHC class II expression is reduced or eliminated by a gene editing system that binds to a CIITA genomic target sequence comprising at least 5 contiguous nucleotides within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, chr16:10922478-10922498, chr16:10895747-10895767, chr16:10916348-10916368, chr16:10910186-10910206, chr16:10906481-10906501, chr16:10909007-10909027, chr16:10895410-10895430, and chr16:10908130-10908150; or
wherein the MHC class II expression is reduced or eliminated by a gene editing system that binds to a CIITA genomic target sequence comprising at least 5 contiguous nucleotides within the genomic coordinates chosen from: chr16:10908132-10908152, chr16:10908131-10908151, chr16:10916456-10916476, chr16:10918504-10918524, chr16:10909022-10909042, chr16:10918512-10918532, chr16:10918511-10918531, chr16:10895742-10895762, chr16:10916362-10916382, chr16:10916455-10916475, chr16:10909172-10909192, chr16:10906492-10906512, chr16:10909006-10909026, and chr16:10922478-10922498; or
wherein the MHC class II expression is reduced or eliminated by a gene editing system that binds to a CIITA genomic target sequence comprising at least 5 contiguous nucleotides within the genomic coordinates chosen from: chr16:10918504-10918524, chr16:10923218-10923238, chr16:10923219-10923239, chr16:10923221-10923241, chr16:10906486-10906506, chr16:10906485-10906505, chr16:10903873-10903893, chr16:10909172-10909192, chr16:10918423-10918443, chr16:10916362-10916382, chr16:10916450-10916470, chr16:10922153-10922173, chr16:10923222-10923242, chr16:10910176-10910196, chr16:10895742-10895762, chr16:10916449-10916469, chr16:10923214-10923234, chr16:10906492-10906512, and chr16:10906487-10906507; or
wherein the MHC class II expression is reduced or eliminated by a gene editing system that binds to a CIITA genomic target sequence comprising at least 5 contiguous nucleotides within the genomic coordinates chosen from: chr16:10918504-10918524, chr16:10923218-10923238, chr16:10923219-10923239, chr16:10923221-10923241, chr16:10906485-10906505, chr16:10916359-10916379, chr16:10903873-10903893, chr16:10909172-10909192, chr16:10922153-10922173, chr16:10916450-10916470, chr16:10923222-10923242, chr16:10916449-10916469, and chr16:10923214-10923234.

18-23. (canceled)

24. The engineered cell of claim 16, wherein the CIITA genomic target sequence comprises at least 10 or at least 15 contiguous nucleotides within the genomic coordinates: or wherein the gene editing system comprises an RNA-guided DNA-binding agent.

25. (canceled)

26. The engineered cell of claim 1, wherein the engineered cell further has reduced or eliminated surface expression of MHC class I; or wherein the engineered cell comprises a genetic modification in the beta-2-microglobulin (B2M) gene: or wherein the engineered cell comprises a genetic modification in an HLA-A gene.

27-28. (canceled)

29. The engineered cell of claim 1, wherein the engineered cell comprises an exogenous nucleic acid encoding a targeting receptor that is expressed on the surface of the engineered cell, and optionally wherein the targeting receptor is a CAR, a T-cell receptor (TCR), or a WT1 TCR: or wherein the engineered cell further comprises an exogenous nucleic acid encoding a polypeptide that is secreted by the engineered cell.

30-31. (canceled)

32. The engineered cell of claim 1, wherein the engineered cell is a T cell and further has reduced or eliminated expression of an endogenous T-cell receptor (TCR) protein relative to an unmodified cell, and optionally wherein the engineered cell has reduced or eliminated expression of a TRAC protein or a TRBC protein relative to an unmodified cell.

33-34. (canceled)

35. A population of cells comprising the engineered cell of claim 1.

36. A pharmaceutical composition comprising a population of cells, wherein the population of cells comprises the engineered cell of claim 1.

37. The population of cells of claim 35, wherein the population of cells is at least 65%, at least 70%, at least 80%, at least 90%, at least 92%, at least 93%, at least 94%, or at least 95% MHC class II negative as measured by flow cytometry; or wherein the population of cells is at least 95%, at least 97%, at least 98%, or at least 99% endogenous TCR protein negative as measured by flow cytometry.

38. (canceled)

39. A method of administering the engineered cell of claim 1 to a subject in need thereof, to a subject for treating the subject with a cancer, an infectious disease, or an autoimmune disease, or to a subject as an adoptive cell transfer (ACT) therapy.

40. (canceled)

41. A composition comprising:
a CIITA guide RNA comprising a guide sequence that
a. targets a CIITA genomic target sequence that comprises at least one nucleotide of a splice site, or
b. directs an RNA-guided DNA binding agent to make a cut in a CIITA genomic target sequence that is 5 nucleotides or less from a splice site boundary nucleotide;
wherein the CIITA guide RNA targets a CIITA genomic target sequence comprising at least 10 contiguous nucleotides within the genomic coordinates chr16:10902171-10923242.

42. A composition comprising:
a. a CIITA guide RNA (gRNA) comprising
i. a guide sequence selected from SEQ ID NOs: 1-101; or
ii. at least 17, 18, 19, or 20 contiguous nucleotides of a sequence selected from SEQ ID NOs: 1-101; or
iii. a guide sequence at least 95%, 90%, or 85% identical to a sequence selected from SEQ ID NOs: 1-101; or
iv. a sequence that comprises 10 contiguous nucleotides ±10 nucleotides of a genomic coordinate listed in Table 1; or
v. at least 17, 18, 19, or 20 contiguous nucleotides of a sequence from (iv); or
vi. a guide sequence that is at least 95%, 90%, or 85% identical to a sequence selected from (v), and
optionally wherein the CIITA gRNA is a single-guide RNA (sgRNA).

43. (canceled)

44. The composition of claim 41, further comprising an RNA-guided DNA binding agent or nucleic acid encoding an RNA-guided DNA binding agent, and optionally further comprising a uracil glycosylase inhibitor (UGI), and optionally wherein the RNA-guided DNA binding agent comprises a deaminase region, and optionally wherein the RNA-guided DNA binding agent comprises an APOBEC3A deaminase (A3A) and an RNA-guided nickase.

45-47. (canceled)

48. The composition of claim 45, wherein the RNA-guided DNA binding agent generates a cytosine (C) to thymine (T) conversion with the CIITA genomic target sequence; or wherein the RNA-guided DNA binding agent generates an adenine (A) to guanine (G) conversion with the CIITA genomic target sequence.

49. (canceled)

50. The composition of claim 41, wherein the CIITA guide RNA targets a CIITA genomic target sequence that comprises at least one nucleotide of a splice acceptor site or a splice donor site, and optionally wherein the one nucleotide is the splice site boundary nucleotide at the splice acceptor site or the splice site boundary nucleotide at the splice donor site.

51. (canceled)

52. The composition of claim 41, wherein the CIITA guide RNA comprises a guide sequence that directs an RNA-guided DNA binding agent to make a cut in a CIITA genomic target sequence that is 4 nucleotides or less, is 3 nucleotides or less, is 2 nucleotides or less, or is 1 nucleotide from a splice site boundary nucleotide; or wherein the CIITA guide RNA comprises a guide sequence that directs an RNA-guided DNA binding agent to make a cut in a CIITA genomic target sequence at a splice site boundary nucleotide.

53. (canceled)

54. A method of making an engineered cell, which has reduced or eliminated surface expression of MHC class II protein relative to an unmodified cell, or method of reducing surface expression of MHC class II protein in an engineered cell relative to an unmodified cell, the method comprising contacting a cell with the composition of claim 41.

55. (canceled)

56. The method of claim 54, further comprising reducing or eliminating the surface expression of MHC class I protein in the cell relative to an unmodified cell: or further comprising reducing or eliminating the surface expression of B2M protein in the cell relative to an unmodified cell: or further comprising reducing or eliminating the surface expression of HLA-A protein in the cell relative to an unmodified cell: or further comprising reducing or eliminating the surface expression of a TCR protein in the cell relative to an unmodified cell.

57-59. (canceled)

60. The method of claim 54, further comprising contacting the cell with an exogenous nucleic acid, and optionally wherein the exogenous nucleic acid encodes a targeting receptor or a polypeptide that is secreted by the cell.

61. (canceled)

62. The engineered cell of claim 1, comprising an exogenous nucleic acid, wherein the exogenous nucleic acid encodes an NK cell inhibitor molecule, and optionally wherein the NK cell inhibitor molecule binds to an inhibitory receptor on an NK cell, or optionally wherein the NK cell inhibitor molecule binds to NKG2A on an NK cell, or optionally wherein the NK cell inhibitor molecule is a non-classical MHC class I molecule, or optionally wherein the NK cell inhibitor molecule is HLA-E, or optionally wherein the NK cell inhibitor molecule is a fusion protein, or optionally wherein the NK cell inhibitor molecule is a fusion protein comprising HLA-E and B2M.

63. (canceled)

64. The engineered cell of claim 1, comprising an exogenous nucleic acid encoding a polypeptide that is secreted by the cell, wherein the secreted polypeptide is an antibody or antibody fragment; or wherein the secreted polypeptide is a full-length IgG antibody, a single chain antibody, or a neutralizing antibody: or wherein the secreted polypeptide is a therapeutic polypeptide; or wherein the secreted polypeptide is an enzyme, a cytokine, or a chemokine.

65-67. (canceled)

68. The engineered cell of claim 1, comprising an exogenous nucleic acid encoding a targeting receptor or contacting the cell with an exogenous nucleic acid encoding a targeting receptor, wherein the targeting receptor is a T cell receptor (TCR), the WT1 TCR, or a CAR.

69. The method of claim 60, wherein the CIITA guide RNA, the RNA-guided DNA binding agent, and/or the exogenous nucleic acid is provided to the cell in a vector, optionally wherein the CIITA guide RNA and the RNA-guided DNA binding agent are provided in the same vector, and optionally wherein the vector is a lentiviral vector, or optionally wherein the vector is an AAV.

70-71. (canceled)

72. The method of claim 60, wherein the guide RNA or the exogenous nucleic acid is provided to the cell in a lipid nucleic acid assembly composition, optionally in the same lipid nucleic acid assembly composition as an RNA-guided DNA binding agent, and optionally wherein the lipid nucleic acid assembly composition is a lipid nanoparticle (LNP).

73. (canceled)

74. The composition of claim 41, wherein the CIITA guide RNA comprises a nucleotide sequence chosen from: SEQ ID NO: 87, SEQ ID NO: 97, SEQ ID NO: 98, SEQ ID NO: 100, SEQ ID NO: 55, SEQ ID NO: 80, SEQ ID NO: 47, SEQ ID NO: 71, SEQ ID NO: 91, SEQ ID NO: 83, SEQ ID NO: 101, SEQ ID NO: 82, and SEQ ID NO: 96, and optionally wherein the CIITA guide RNA comprises at least one modification, wherein the at least one modification includes (i) a 2′-O-methyl (2′-O-Me) modified nucleotide, (ii) a phosphorothioate (PS) bond between nucleotides, (iii) a 2′-fluoro (2′-F) modified nucleotide, (iv) a modification at one or more of the first five nucleotides at the 5′ end of the guide RNA, (v) a modification at one or more of the last five nucleotides at the 3′ end of the guide RNA, (vi) a PS bond between the first four nucleotides of the guide RNA, (vii) a PS bond between the last four nucleotides of the guide RNA, (viii) a 2′-O-Me modified nucleotide at the first three nucleotides at the 5′ end of the guide RNA, (ix) a 2′-O-Me modified nucleotide at the last three nucleotides at the 3′ end of the guide RNA, or combinations of one or more of (i)-(ix).

75. (canceled)

76. An engineered cell or population of cells comprising a genetic modification that includes an indel within the genomic region targeted by the CIITA guide RNA of claim 41; or comprising a genetic modification that includes a C to T substitution or an A to G substitution within the genomic region targeted by the CIITA guide RNA of claim 41.

77-80. (canceled)

81. The engineered cell of claim 1, wherein the genetic modification comprises an indel: or wherein the genetic modification comprises a C to T substitution: or wherein the genetic modification comprises an A to G substitution.

82-83. (canceled)

84. The engineered cell of claim 1, wherein the cell is homozygous for HLA-B and homozygous for HLA-C; or wherein the cell further comprises a genetic modification in an HLA-A gene, and wherein the genetic modification in the HLA-A gene comprises at least one nucleotide within the genomic coordinates chosen from:
a. chr6:29942854 to chr6:29942913, and
b. chr6:29943518 to chr6: 29943619: or
wherein the cell further comprises a genetic modification in an HLA-A gene, and wherein the genetic modification in the HLA-A gene comprises at least one nucleotide within the genomic coordinates chosen from: chr6:29942864 to chr6: 29942903 or chr6:29943528 to chr6:29943609.

85-86. (canceled)

87. A method of making an engineered cell, which has reduced or eliminated surface expression of MHC class II protein and HLA-A protein relative to an unmodified cell, comprising:
a. contacting the cell with a CIITA guide RNA, wherein the guide RNA comprises a guide sequence selected from SEQ ID NOs: 1-101;
b. contacting the cell with an HLA-A guide RNA, wherein the HLA-A guide RNA comprises a guide sequence selected from any one of SEQ ID NOs: 2001-2095; and
c. optionally contacting the cell with an RNA-guided DNA binding agent or nucleic acid encoding an RNA-guided DNA binding agent;
thereby reducing or eliminating the surface expression of MHC class II protein and HLA-A protein in the cell relative to an unmodified cell.
